(19) United States
(12) Patent Application Publication
(10) Pub. No.: US 2026/0239875 A1
(43) Pub. Date: Aug. 13, 2026

(54) COMPOUND, LIGHT-EMITTING MATERIAL, DELAYED FLUORESCENT SUBSTANCE, AND ORGANIC LIGHT-EMITTING DEVICE

(71) Applicant: KYULUX, INC., Fukuoka-shi (JP)

(72) Inventors: Yong Joo Cho, Fukuoka-shi (JP); Yu Yamane, Fukuoka-shi (JP); Ryohei Yagi, Fukuoka-shi (JP); Naomi Shimamura, Fukuoka-shi (JP); Yoshitake Suzuki, Fukuoka-shi (JP); Kei Morimoto, Fukuoka-shi (JP); Yuka Kodama, Fukuoka-shi (JP); Kousei Kanahara, Fukuoka-shi (JP)

(73) Assignee: KYULUX, INC., Fukuoka-shi (JP)

(21) Appl. No.: 19/470,979

(22) PCT Filed: Apr. 2, 2024
(86) PCT No.: PCT/JP2024/013607

§ 371 Date: Sep. 30, 2025

(30) Foreign Application Priority Data
Apr. 4, 2023 (JP) 2023-061050

(51) Int. Cl. H10K 85/60; C07D 403/04; C07D 403/10; C07D 413/10; C09K 11/02; C09K 11/06; H10K 50/12

(52) U.S. Cl. CPC H10K 85/654; C07D 403/04; C07D 403/10; C07D 413/10; C09K 11/02; C09K 11/06; H10K 85/657; H10K 85/6572; C09K 2211/1007; C09K 2211/1018; H10K 50/12

(57) ABSTRACT

The compound of the following general formula has excellent light emission characteristics. R¹ is H or D; one of R² and R³ is a triazinyl group or the like substituted with Ar³ and Ar⁴, two of the other of R² and R³ and R⁴ and R⁵ are donor groups, and the remaining one is an aryl group; and each of Ar¹ to Ar⁴ is an aryl group or a heteroaryl group

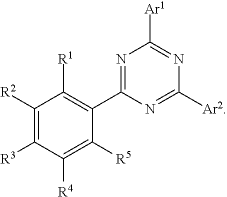

### TECHNICAL FIELD

The present invention relates to a compound having a skeleton in which a triazine ring and a benzene ring are bonded, and a light-emitting material and a delayed fluorescent substance using the compound. The present invention also relates to an organic light-emitting device such as an organic electroluminescent device using the compound.

### BACKGROUND ART

An organic light-emitting device is a light-emitting device using an organic material, which can be produced by coating and which does not use a rare element, and therefore, attention has recently been paid to the organic light-emitting device. Above all, an organic electroluminescent device (organic EL device) emits self-luminous light and does not require a backlight, and is therefore advantageous in that it can be a lightweight and flexible device. In addition, it has features of high responsiveness and high visibility, and is expected as a next generation light source. Consequently, studies relating to development of materials useful for organic light-emitting devices such as organic electroluminescent devices have been promoted actively. In particular, studies relating to light-emitting materials have been carried out actively. Fluorescent materials, phosphorescent materials, and fluorescent materials have been known as light-emitting materials for a long time. However, fluorescent materials have a problem of low light emission efficiency, and phosphorescent materials have a problem of being expensive because they contain rare metals and being difficult to emit deep blue light. In recent years, a delayed fluorescent material has been developed as a light-emitting material that addresses these problems.

A delayed fluorescent material is a material which, in an excited state, after having undergone reverse intersystem crossing from an excited triplet state to an excited singlet state, emits fluorescence when returning back from the excited singlet state to a ground state thereof. Fluorescence through the route is observed later than fluorescence from the excited singlet state directly occurring from the ground state (ordinary fluorescence), and is therefore referred to as delayed fluorescence. Here, for example, in the case where a light-emitting compound is excited through carrier injection thereinto, the occurring probability of the excited singlet state to the excited triplet state is statistically 25%/75%, and therefore improvement of light emission efficiency by the fluorescence alone from the directly occurring excited singlet state is limited. On the other hand, in a delayed fluorescent material, not only the excited singlet state thereof but also the excited triplet state can be utilized for fluorescent emission through the route via the above-mentioned reverse intersystem crossing, and therefore as compared with an ordinary fluorescent material, a delayed fluorescent material can realize a higher light emission efficiency.

Since such a principle has been clarified, various studies have led to the discovery of various delayed fluorescent materials. Among these, many compounds in which a benzene ring is substituted with a donor group and an acceptor group are included. For example, it has been proposed to use a compound having a skeleton, in which the benzene ring is substituted with a carbazol-9-yl group of a donor group and with a cyano group and a substituted triazinyl group of acceptor groups, in an organic light-emitting device (see PTL 1).

### CITATION LIST

### Patent Literature

PTL 1: WO2019/191665A1

### SUMMARY OF INVENTION

### Technical Problem

Even when a material emits delayed fluorescence, one having extremely good characteristics and having no problem in practical use has not yet been provided. Therefore, it is more useful if a delayed fluorescent material having more excellent characteristics can be provided. However, the improvement of delayed fluorescent materials is in the stage of trial and error, and it is not easy to generalize the chemical structure of useful light-emitting materials.

Under such circumstances, the present inventors have conducted research for the purpose of providing a compound more useful as a delayed fluorescent material for a light-emitting device. Then, the present inventors have conducted intensive studies for the purpose of deriving and generalizing a general formula of a compound more useful as a delayed fluorescent material and using such a compound in an organic light-emitting device.

### Solution to Problem

As a result of intensive studies to achieve the above-mentioned object, the present inventors have found that a compound having a structure satisfying specific conditions is useful as a light-emitting material and can provide an excellent organic light-emitting device. The present invention has been proposed based on these findings, and specifically has the following configuration.

[1]A compound represented by the following general formula (1):

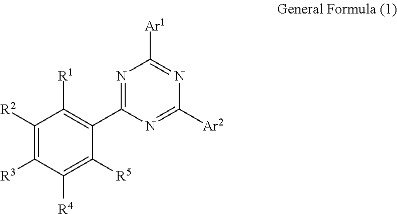

in which R¹ represents a hydrogen atom or a deuterium atom; one of R² and R³ is a group represented by the following general formula (2), two selected from R⁴, R⁵ and the other of R² and R³ are donor groups, and the remaining one is a substituted or unsubstituted aryl group; Ar¹ and Ar² each independently represent a substituted or unsubstituted aryl group, or a substituted or unsubstituted heteroaryl group;

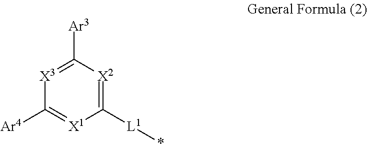

in which X¹ to X³ each independently represent N or C(R), provided that at least one of X¹ to X³ is N; R represents a hydrogen atom, a deuterium atom, or a substituent; Ar³ and Ar⁴ each independently represent a substituted or unsubstituted aryl group, or a substituted or unsubstituted heteroaryl group; L¹ represents a single bond or a divalent linking group; and * represents a bonding position.

[2] The compound according to [1], in which R² is a group represented by the general formula (2).

[3] The compound according to [1], in which R³ is a group represented by the general formula (2).

[4] The compound according to any one of [1] to [3], in which at least one of Ar¹ to Ar⁴ is a substituted or unsubstituted heteroaryl group (excluding a nitrogen-containing 6-membered ring group).

[5] The compound according to [4], in which at least one of Ar¹ to Ar⁴ is a heteroaryl group having a 5-membered ring bonded via a nitrogen atom.

[6] The compound according to any one of [1] to [5], in which the donor group is a substituted or unsubstituted carbazol-9-yl group.

[7] The compound according to any one of [1] to [6], in which the donor group is a donor group having a fused ring structure of four or more rings.

[8] The compound according to any one of [1] to [7], in which R⁵ is a donor group.

[9] The compound according to any one of [1] to [8], in which R⁴ is a substituted or unsubstituted aryl group.

[10] The compound according to any one of [1] to [9], in which the substituted or unsubstituted aryl group is an unsubstituted aryl group or an aryl group substituted with an aryl group.

[11] The compound according to any one of [1] to [10], in which X¹ to X³ are N.

[12] The compound according to any one of [1] to [11], in which L¹ is a single bond.

[13] The compound according to anyone of [1] to [12], in which R¹ is a hydrogen atom.

[14] The compound according to any one of [1] to [13], in which the compound has at least one deuterium atom.

[15]A light-emitting material including the compound according to any one of [1] to [14].

[16]A delayed fluorescent substance including the compound according to any one of [1] to [14].

[17] An organic light-emitting device including the compound according to any one of [1] to [14] and a host material or a dopant material in the same layer.

[18] The organic light-emitting device according to [17], in which the layer contains the dopant material.

[19] The organic light-emitting device according to [17] or [18], in which an amount of light emitted from the dopant material is larger than an amount of light emitted from the compound.

[20] The organic light-emitting device according to any one of [17] to [19], in which the layer contains the host material.

[21] The organic light-emitting device according to [20], in which the host material is a compound represented by the following general formula (4):

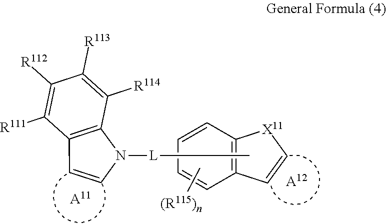

in which X¹¹ represents O, S, N(R^{A}), or C(R^{B})(R^{C}); A¹¹ and A¹² each independently represent a benzene ring, a furan ring, a thiol ring, a pyrrole ring, or a cyclopentadiene ring, which may be further fused with another ring or substituted; R . . . to R¹¹⁴, R^{B}, and R^{C} each independently represent a hydrogen atom, a deuterium atom, a substituted or unsubstituted aryl group, a substituted or unsubstituted heteroaryl group, a substituted or unsubstituted alkyl group, or a cyano group; each of R¹¹⁵'s independently represents a hydrogen atom, a deuterium atom, a substituted or unsubstituted aryl group, a substituted or unsubstituted heteroaryl group, a substituted or unsubstituted alkyl group, a cyano group, or a bond with L; R^{A} represents a substituted or unsubstituted aryl group, a substituted or unsubstituted heteroaryl group, a substituted or unsubstituted alkyl group, or a bond with L; R¹¹¹ and R¹¹², R¹¹² and R¹¹³, R¹¹³ and R¹¹⁴, two adjacent R¹¹⁵'s, and R^{B} and R^{C} may be bonded to each other to form a cyclic structure; n represents an integer of 3 or 4; and L represents a single bond, a substituted or unsubstituted arylene group, a substituted or unsubstituted heteroarylene group, or a linking group in which two or more of these groups are bonded.

[22] The organic light-emitting device according to any one of [17] to [21], in which an amount of light emitted from the compound is the largest among the materials contained in the layer.

[23] The organic light-emitting device according to any one of [17] to [22], which emits delayed fluorescence.

### Advantageous Effects of Invention

The compound and the organic light-emitting device of the present invention exhibit excellent light emission characteristics.

### DESCRIPTION OF EMBODIMENTS

The contents of the present invention will be described in detail below. The descriptions on constituent elements to be described below may be made on the basis of representative embodiments or specific examples of the present invention, but the present invention is not limited to such embodiments or specific examples. In the description herein, a numerical range expressed using “to” means a range that includes the numerical values described before and after “to” as the lower limit and the upper limit. A part or all of hydrogen atoms existing in the molecule of the compound for use in the present invention can be substituted with deuterium atoms (²H, deuterium D). In the chemical structural formulae in the description herein, the hydrogen atom is expressed as H, or the expression thereof is omitted. For example, when expression of the atoms bonding to the ring skeleton-constituting carbon atoms of a benzene ring is omitted, H is considered to bond to the ring skeleton-constituting carbon atom at the site having the omitted expression. In the description herein, the term “substituent” means an atom or an atomic group except a hydrogen atom and a deuterium atom. The term “substituted or unsubstituted” means that a hydrogen atom may be substituted with a deuterium atom or a substituent.

### [Compound Represented by General Formula (1)]

The compound represented by the following general formula (1) is described.

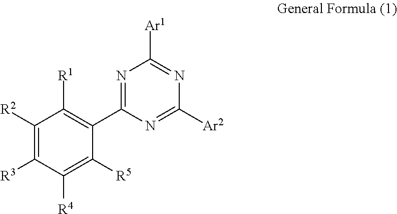

In the general formula (1), R¹ represents a hydrogen atom or a deuterium atom. one of R² and R³ is a group represented by the following general formula (2), two selected from R⁴, R⁵ and the other of R² and R³ are donor groups, and the remaining one is a substituted or unsubstituted aryl group. Ar¹ and Ar² each independently represent a substituted or unsubstituted aryl group, or a substituted or unsubstituted heteroaryl group.

In one aspect of the present invention, R¹ is a hydrogen atom. In one aspect of the present invention, R¹ is a deuterium atom.

In one aspect of the present invention, R² is a group represented by the following general formula (2), two of R³, R⁴, and R⁵ are donor groups, and the remaining one is a substituted or unsubstituted aryl group. In one aspect of the present invention, R² is a group represented by the following general formula (2), R³ and R⁵ are donor groups, and R⁴ is a substituted or unsubstituted aryl group. In one aspect of the present invention, R² is a group represented by the following general formula (2), R⁴ and R⁵ are donor groups, and R³ is a substituted or unsubstituted aryl group.

In one aspect of the present invention, R³ is a group represented by the following general formula (2), two of R², R⁴, and R⁵ are donor groups, and the remaining one is a substituted or unsubstituted aryl group. In one aspect of the present invention, R³ is a group represented by the following general formula (2), R² and R⁵ are donor groups, and R⁴ is a substituted or unsubstituted aryl group. In one aspect of the present invention, R³ is a group represented by the following general formula (2), R⁴ and R⁵ are donor groups, and R² is a substituted or unsubstituted aryl group. In one aspect of the present invention, R³ is a group represented by the following general formula (2), R² and R⁴ are donor groups, and R⁵ is a substituted or unsubstituted aryl group.

The two donor groups present in the general formula (1) may be the same as or different from each other.

The aryl group that R² to R⁵ and Ar¹ and Ar² can adopt each may be a monocyclic ring or may be a fused ring in which two or more rings are fused. In the case of a fused ring, the number of fused rings is preferably 2 to 6, and can be selected from, for example, 2 to 4. Specific examples of the ring include a benzene ring, a naphthalene ring, an anthracene ring, a phenanthrene ring, and a triphenylene ring. In one aspect of the present invention, the aryl group is a substituted or unsubstituted phenyl group, a substituted or unsubstituted naphthalen-1-yl group, or a substituted or unsubstituted naphthalen-2-yl group, and is preferably a substituted or unsubstituted phenyl group. For example, the substituent for the aryl group may be selected from Substituent Group A, may be selected from Substituent Group B, may be selected from Substituent Group C, may be selected from Substituent Group D, or may be selected from Substituent Group E. In one aspect of the present invention, the substituent for the aryl group is one or more selected from the group consisting of an alkyl group, an aryl group, and a deuterium atom. In one preferred aspect of the present invention, the aryl group is substituted with at least one deuterium atom. In one aspect of the present invention, the aryl group is unsubstituted.

Specific examples of the substituted or unsubstituted aryl group that R² to R⁵ and Ar¹ and Ar² can adopt are shown below. Here, the aryl group which can be employed in the present invention should not be limitatively interpreted by the following specific examples. In the following specific examples, * indicates a bonding position. The expression of a methyl group is omitted. Consequently, Ar2 to Ar7 represent structures substituted with a methyl group.

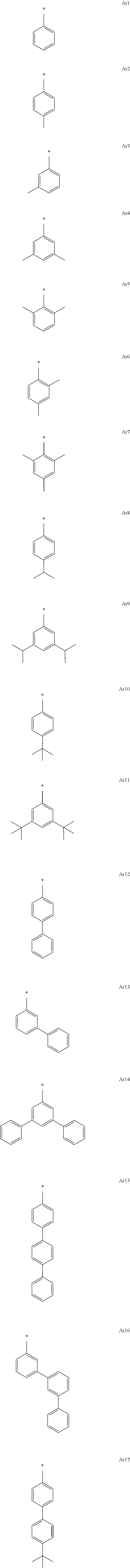

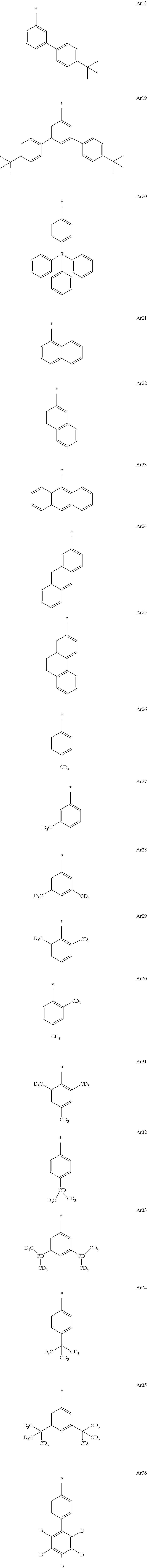

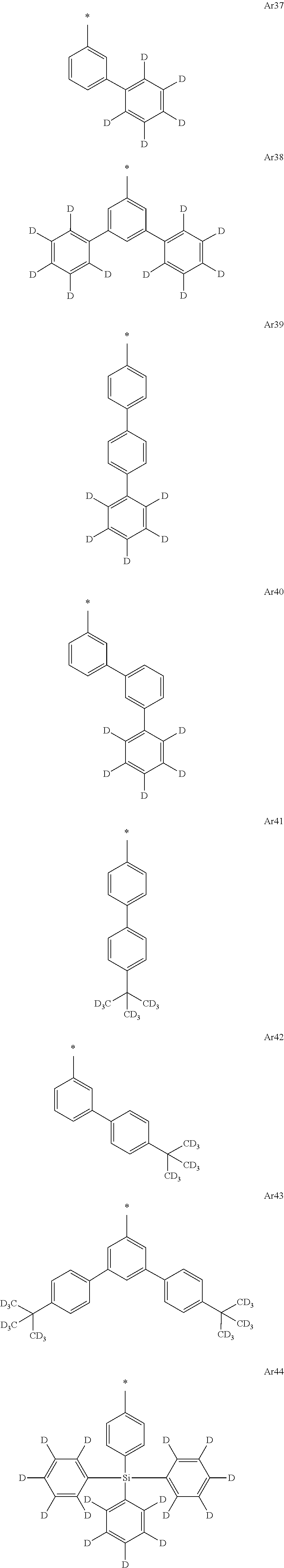

In addition to the above-mentioned specific examples, groups obtained by substituting all hydrogen atoms present in Ar1 to Ar25 with deuterium atoms are exemplified as Ar45 to Ar69, respectively.

In one aspect of the present invention, the aryl group that R² to R⁵ can adopt is selected from the group consisting of Ar1 to Ar69. In one aspect of the present invention, the aryl group that R² to R⁵ can adopt is Ar1 or Ar45. In one aspect of the present invention, the aryl group that R² to R⁵ can adopt is selected from the group consisting of Ar2 to Ar11, Ar26 to Ar35, and Ar46 to Ar55. In one aspect of the present invention, the aryl group that R² to R⁵ can adopt is selected from the group consisting of Ar12 to Ar19, Ar36 to Ar43, and Ar56 to Ar63. In one aspect of the present invention, the aryl group that R² to R⁵ can adopt is selected from the group consisting of Ar21 to Ar25 and Ar65 to Ar69. In one preferred aspect of the present invention, the aryl group that R² to R⁵ can adopt is selected from the group consisting of Ar1, Ar12 to Ar14, Ar23, Ar36 to Ar38, Ar45, Ar56 to Ar58, and Ar67.

In one aspect of the present invention, the aryl group that Ar¹ or Ar² can adopt is selected from the group consisting of Ar1 to Ar69. In one aspect of the present invention, the aryl group that Ar¹ or Ar² can adopt is Ar1 or Ar45. In one aspect of the present invention, the aryl group that Ar¹ or Ar² can adopt is selected from the group consisting of Ar2 to Ar11, Ar26 to Ar35, and Ar46 to Ar55. In one aspect of the present invention, the aryl group that Ar¹ or Ar² can adopt is selected from the group consisting of Ar12 to Ar19, Ar36 to Ar43, and Ar56 to Ar63. In one preferred aspect of the present invention, the aryl group that R² to R⁵ can adopt is selected from the group consisting of Ar1, Ar12 to Ar14, Ar36 to Ar38, Ar45, and Ar56 to Ar58.

Two of R² to R⁵ in the general formula (1) are donor groups. The donor group that R² to R⁵ can adopt does not include a substituted or unsubstituted aryl group.

The “donor group” can be selected from groups having a negative Hammett's op value. The “acceptor group” can be selected from groups having a positive Hammett's op value. The Hammett's ap value is proposed by L. P. Hammett and quantifies the influence of a substituent on the reaction rate or equilibrium of a para-substituted benzene derivative. Specifically, the value is a constant (ap) specific to the substituent in the following equations that is established between substituents and reaction rate constants or equilibrium constants in para-substituted benzene derivatives:

$$log ( k / {k}_{0} ) = ρ σ p or$$

$$log ( K / {K}_{0} ) = ρ σ p .$$

In the above equations, ko represents a rate constant of a benzene derivative not having a substituent; k represents a rate constant of a benzene derivative substituted with a substituent; Ko represents an equilibrium constant of a benzene derivative not having a substituent; K represents an equilibrium constant of a benzene derivative substituted with a substituent; and p represents a reaction constant to be determined by the type and the condition of reaction. Regarding the description relating to the “Hammett's op value” and the numerical value of each substituent in the present invention, reference can be made to the description relating to ap value in Hansch, C. et. al., Chem. Rev., 91, 165-195 (1991).

The donor group that R² to R⁵ can adopt preferably has op of −0.3 or less, more preferably −0.5 or less, and still more preferably −0.7 or less. For example, the value may be selected from a range of −0.9 or less, or from a range of −1.1 or less.

The donor group in the present invention is preferably a group containing a substituted amino group. The donor group may be a substituted amino group, or may be a substituted amino group-bonded aryl group, especially a substituted amino group-bonded phenyl group. In one preferred aspect of the present invention, the donor group is a substituted amino group.

The substituent bonding to the nitrogen atom of a substituted amino group is preferably a substituted or unsubstituted alkyl group, a substituted or unsubstituted alkenyl group, a substituted or unsubstituted aryl group, or a substituted or unsubstituted heteroaryl group, and more preferably a substituted or unsubstituted aryl group or a substituted or unsubstituted heteroaryl group. Especially, the substituted amino group is preferably a substituted or unsubstituted diarylamino group, or a substituted or unsubstituted diheteroarylamino group. The two aryl groups constituting the diarylamino group referred to herein may be bonded to each other, and the two heteroaryl groups constituting the diheteroarylamino group may be bonded to each other.

The donor group that R² to R⁵ can adopt is preferably a group represented by the following general formula (a).

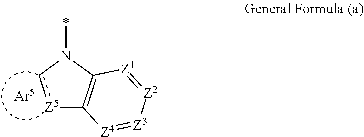

In the general formula (a), Z¹ represents C—R¹⁴ or N, Z² represents C—R¹⁵ or N, Z³ represents C—R¹⁶ or N, and Z⁴ represents C—R¹⁷ or N. Z⁵ represents C or N, Ar⁵ represents a substituted or unsubstituted aromatic ring or a substituted or unsubstituted heteroaromatic ring. R¹⁴ and R¹⁵, R¹⁵ and R¹⁶, and R¹⁶ and R¹⁷ each may be bonded to each other to form a cyclic structure.

Among Z¹ to Z⁴, the number of groups represented by N is preferably 0 to 3, and preferably 0 to 2. In one aspect of the present invention, among Z¹ to Z⁴, the number of groups represented by N is 1. In one aspect of the present invention, among Z¹ to Z⁴, the number of groups represented by N is 0.

R¹⁴ to R¹⁷ each independently represent a hydrogen atom, a deuterium atom, or a substituent.

For example, the substituent may be selected from Substituent Group A, may be selected from Substituent Group B, may be selected from Substituent Group C, may be selected from Substituent Group D, or may be selected from Substituent Group E. When two or more of R¹⁴ to R¹⁷ represent substituents, the two or more substituents may be the same or different. Zero to two of R¹⁴ to R¹⁷ are preferably substituents, and for example, one may be a substituent, or zero may be a substituent (R¹⁴ to R¹⁷ are hydrogen atoms or deuterium atoms).

R¹⁴ and R¹⁵, R¹⁵ and R¹⁶, and R¹⁶ and R¹⁷ each may be bonded to each other to form a cyclic structure. The cyclic structure may be any of an aromatic ring, a heteroaromatic ring, an aliphatic hydrocarbon ring, and an aliphatic heterocyclic ring, and may be a ring obtained by fusing these rings. The structure is preferably an aromatic ring or a heteroaromatic ring. Examples of the aromatic ring can include a substituted or unsubstituted benzene ring. Another benzene ring may be further fused to the benzene ring, and a heterocyclic ring such as a pyridine ring may be fused to the benzene ring. The heteroaromatic ring means a ring containing a hetero atom as a ring skeleton-constituting atom and exhibiting aromaticity, and is preferably a 5- to 7-membered ring, and for example, a 5-membered ring or a 6-membered ring can be employed. In one aspect of the present invention, a furan ring, a thiophene ring, or a pyrrole ring can be employed as the heteroaromatic ring. In one preferred aspect of the present invention, the cyclic structure is a furan ring of a substituted or unsubstituted benzofuran, a thiophene ring of a substituted or unsubstituted benzothiophene, or a pyrrole ring of a substituted or unsubstituted indole. The benzofuran, benzothiophene, and indole referred to herein may be unsubstituted, or may be substituted with a substituent selected from Substituent Group A, may be substituted with a substituent selected from Substituent Group B, may be substituted with a substituent selected from Substituent Group C, may be substituted with a substituent selected from Substituent Group D, and may be substituted with a substituent selected from Substituent Group E. It is preferable that a substituted or unsubstituted aryl group is bonded to the nitrogen atom constituting the pyrrole ring of indole, and examples of the substituent include a substituent selected from any of Substituent Group A to Substituent Group E. The cyclic structure may be a substituted or unsubstituted cyclopentadiene ring. In one aspect of the present invention, a pair of R¹⁴ and R¹⁵, R¹⁵ and R¹⁶, and R¹⁶ and R¹⁷ are bonded to each other to form a cyclic structure. In one aspect of the present invention, none of R¹⁴ and R¹⁵, R¹⁵ and R¹⁶, and R¹⁶ and R¹⁷ are bonded to each other to form a cyclic structure.

In the general formula (a), Z⁵ represents C or N, and Ar⁵ represents a substituted or unsubstituted aromatic ring or a substituted or unsubstituted heteroaromatic ring. In one aspect of the present invention, Z⁵ represents C, and Ar⁵ represents a substituted or unsubstituted aromatic ring or a substituted or unsubstituted heteroaromatic ring. In one aspect of the present invention, Z⁵ represents N, and Ar⁵ represents a substituted or unsubstituted heteroaromatic ring.

Examples of the aromatic ring that Ar⁵ can adopt include a benzene ring. Another benzene ring may be further fused to the benzene ring, and a heterocyclic ring such as a pyridine ring may be fused to the benzene ring. The heteroaromatic ring that Ar⁵ can adopt is preferably a 5- to 7-membered ring, and for example, a 5-membered ring or a 6-membered ring can be employed. In one aspect of the present invention, as the heteroaromatic ring, a furan ring, a thiophene ring, a pyrrole ring, an imidazole ring, a pyridine ring, a pyridazine ring, a pyrimidine ring, or a pyrazine ring can be employed. In one aspect of the present invention, Z⁵ represents C, and the heteroaromatic ring is a furan ring of a substituted or unsubstituted benzofuran, a thiophene ring of a substituted or unsubstituted benzothiophene, a pyridine ring of a substituted or unsubstituted quinoline, or a pyridine ring of a substituted or unsubstituted isoquinoline. In one aspect of the present invention, Z⁵ represents N, and the heteroaromatic ring is a pyrrole ring of a substituted or unsubstituted indole, or an imidazole ring of a substituted or unsubstituted benzimidazole. The benzofuran, benzothiophene, quinoline, isoquinoline, indole, and benzimidazole referred to herein may be unsubstituted, or may be substituted with a substituent selected from Substituent Group A, may be substituted with a substituent selected from Substituent Group B, may be substituted with a substituent selected from Substituent Group C, may be substituted with a substituent selected from Substituent Group D, and may be substituted with a substituent selected from Substituent Group E.

When Z⁵ in the general formula (a) represents C, a group represented by the following general formula (b) is preferred.

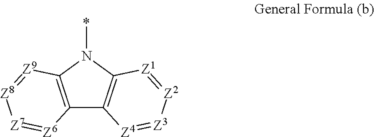

In the general formula (b), Z¹ represents C—R¹⁴ or N, Z² represents C—R¹⁵ or N, Z³ represents C—R¹⁶ or N, Z⁴ represents C—R¹⁷ or N, Z⁶ represents C—R¹⁸ or N, Z⁷ represents C—R¹⁹ or N, Z⁸ represents C—R²⁰ or N, and Z⁹ represents C—R²¹ or N. R¹⁴ and R¹⁵, R¹⁵ and R¹⁶, R¹⁶ and R¹⁷, R¹⁸ and R¹⁹, R¹⁹ and R²⁰, and R²⁰ and R²¹ each may be bonded to each other to form a cyclic structure.

Regarding Z¹ to Z⁴ and R¹⁴ to R¹⁷ in the general formula (b), reference can be made to the corresponding description of the general formula (a). Z⁶ to Z⁹ and R¹⁸ to R²¹ in the general formula (b) correspond to Z¹ to Z⁴ and R¹⁴ to R¹⁷ in the general formula (a), respectively, and regarding the contents thereof, reference can be made to the descriptions of Z¹ to Z⁴ and R¹⁴ to R¹⁷ in the general formula (a).

In one aspect of the present invention, among Z¹ to Z⁴ and Z⁶ to Z⁹, the number of groups represented by N is preferably 0 to 2, and more preferably 0 or 1. In one aspect of the present invention, among Z¹ to Z⁴ and Z⁶ to Z⁹, the number of groups represented by N is 1. In one preferred aspect of the present invention, among Z¹ to Z⁴ and Z⁶ to Z⁹, the number of groups represented by N is 0. When the number is 0, the formula represents a substituted or unsubstituted carbazol-9-yl group.

The donor group that R² to R⁵ can adopt is preferably a substituted or unsubstituted carbazol-9-yl group. The carbazol-9-yl group referred to herein may be unsubstituted, or may be substituted with a substituent selected from Substituent Group A, may be substituted with a substituent selected from Substituent Group B, may be substituted with a substituent selected from Substituent Group C, may be substituted with a substituent selected from Substituent Group D, and may be substituted with a substituent selected from Substituent Group E. One or more rings may be fused to the two benzene rings constituting the carbazol-9-yl group. In one preferred aspect of the present invention, the donor group that R² to R⁵ can adopt is a carbazol-9-yl group optionally substituted with a group selected from Substituent Group E, and optionally fused with one or more rings. In the case where the carbazol-9-yl group not fused with a ring is substituted, the substitution site is not specifically limited, but is preferably at least one of 2 to 7-positions, more preferably at least one of 3 to 6-positions, and further preferably a 3-position and a 6-position.

In one aspect of the present invention, the donor group that R² to R⁵ can adopt is a carbazol-9-yl group fused with one or more rings, and hereinafter, this will be referred to as a “ring-fused carbazol-9-yl group”. The ring-fused carbazol-9-yl group that R² to R⁵ can adopt may be unsubstituted, or may be substituted with a substituent selected from Substituent Group A, may be substituted with a substituent selected from Substituent Group B, may be substituted with a substituent selected from Substituent Group C, may be substituted with a substituent selected from Substituent Group D, and may be substituted with a substituent selected from Substituent Group E. Preferably, the group is unsubstituted, or substituted with a substituent selected from Substituent Group E. In one aspect of the present invention, the ring-fused carbazol-9-yl group is unsubstituted. In one preferred aspect of the present invention, the ring-fused carbazol-9-yl group is substituted with an aryl group optionally substituted with one atom or group selected from the group consisting of a deuterium atom, an alkyl group and an aryl group or with a group formed by combining two or more thereof.

The total number of rings constituting the fused ring in the ring-fused carbazol-9-yl group is 4 or more, preferably 5 or more, more preferably 5 to 9, and still more preferably 5 to 7. In one preferred aspect of the present invention, the number of rings constituting the fused ring is 5. Here, the number of rings includes the number of rings of carbazole to be fused (i.e. 3).

The ring-fused carbazol-9-yl group is a group that bonds via the nitrogen atom constituting the ring skeleton of carbazole, and has a structure in which a ring is fused to at least one of the two benzene rings constituting carbazole. The fused ring may be any of an aromatic hydrocarbon ring, an aromatic heterocyclic ring, an aliphatic hydrocarbon ring, and an aliphatic heterocyclic ring, and may be a ring obtained by further fusing these rings. An aromatic hydrocarbon ring and an aromatic heterocyclic ring are preferable. Examples of the aromatic hydrocarbon ring include a substituted or unsubstituted benzene ring. Another benzene ring may be further fused to the benzene ring, and a heterocyclic ring such as a pyridine ring may be fused to the benzene ring. The aromatic heterocyclic ring means a ring containing a hetero atom as a ring skeleton-constituting atom and exhibiting aromaticity, and is preferably a 5- to 7-membered ring, and for example, a 5-membered ring or a 6-membered ring can be employed. In one aspect of the present invention, a furan ring, a thiophene ring, or a pyrrole ring can be employed as the aromatic heterocyclic ring. In one aspect of the present invention, the fused ring is a furan ring of a substituted or unsubstituted benzofuran, a thiophene ring of a substituted or unsubstituted benzothiophene, or a pyrrole ring of a substituted or unsubstituted indole. It is preferable that a substituent selected from Substituent Group E (but except for a deuterium atom alone) is bonded to the nitrogen atom of the pyrrole ring, and it is more preferable that an aryl group which may be substituted with an alkyl group or an aryl group is bonded. In the present invention, it is preferable to employ a carbazol-9-yl group in which a ring having one or more atoms selected from the group consisting of an oxygen atom, a sulfur atom, and a nitrogen atom as a ring skeleton-constituting atom is fused. Above all, preferably employed are a benzofuro structure-fused carbazol-9-yl group, a benzothieno structure-fused carbazol-9-yl group, and an indolo structure-fused carbazol-9-yl group. In one aspect of the present invention, the compound has at least one benzofuro structure-fused carbazol-9-yl group, and for example, has two or more such groups. In one aspect of the present invention, the compound has at least one benzothieno structure-fused carbazol-9-yl group, and for example, has two or more such groups.

The ring-fused carbazol-9-yl group employable herein includes a substituted or unsubstituted benzofuro[2,3-a]carbazol-12-yl group, a substituted or unsubstituted benzofuro[3,2-a]carbazol-12-yl group, a substituted or unsubstituted benzofuro[2,3-b]carbazol-7-yl group, a substituted or unsubstituted benzofuro[3,2-b]carbazol-11-yl group, a substituted or unsubstituted benzofuro[2,3-c]carbazol-8-yl group, and a substituted or unsubstituted benzofuro[3,2-c]carbazol-5-yl group. Further, the ring-fused carbazol-9-yl group employable herein includes a substituted or unsubstituted benzothieno[2,3-a]carbazol-12-yl group, a substituted or unsubstituted benzothieno[3,2-a]carbazol-12-yl group, a substituted or unsubstituted benzothieno[2,3-b]carbazol-7-yl group, a substituted or unsubstituted benzothieno[3,2-b]carbazol-11-yl group, a substituted or unsubstituted benzothieno[2,3-c]carbazol-8-yl group, and a substituted or unsubstituted benzothieno[3,2-c]carbazol-5-yl group. Furthermore, the ring-fused carbazol-9-yl group employable herein includes a substituted or unsubstituted 11-phenylindolo[2,3-a]carbazol-12-yl group, a substituted or unsubstituted 5-phenylindolo[3,2-a]carbazol-12-yl group, a substituted or unsubstituted 5-phenylindolo[2,3-b]carbazol-7-yl group, a substituted or unsubstituted 5-phenylindolo[3,2-b]carbazol-11-yl group, a substituted or unsubstituted 5-phenylindolo[2,3-c]carbazol-8-yl group, and a substituted or unsubstituted 12-phenylindolo[3,2-a]carbazol-5-yl group.

The number of the substituents substituted on the ring-fused carbazol-9-yl group is preferably 1 to 10, more preferably 1 to 6, further preferably 1 to 4, and may be, for example 1, or may be, for example 2. In one preferred aspect of the present invention, any of the 3-position or the 6-position of the ring-fused carbazol-9-yl group is substituted. In one preferred aspect of the present invention, the compound has at least one substituent on the para-position of the benzene ring viewed from the hetero atom present in the ring-fused carbazol-9-yl group. In one preferred aspect of the present invention, the compound has at least one substituent only on the para-position of the benzene ring viewed from the hetero atom present in the ring-fused carbazol-9-yl group. In one preferred aspect of the present invention, the compound has substituents on all the substitutable para-positions of the benzene ring viewed from the hetero atom present in the ring-fused carbazol-9-yl group.

Specific examples of the donor group that R² to R⁵ in the general formula (1) can adopt are shown below. Here, the donor group which can be employed in the present invention shall not be construed as being limited by the following specific examples.

First, specific examples of the substituted or unsubstituted carbazol-9-yl group in which the donor group has a fused ring structure of three rings are shown below. In the following specific examples, Ph represents a phenyl group (C₆H₅), and * indicates a bonding position. Since the expression of a methyl group is omitted, for example, D2 has one methyl group. Here, a deuterated methyl group is expressed as CD₃. C₆D₅ represents a phenyl group in which all hydrogen atoms are deuterated. D represents a deuterium atom.

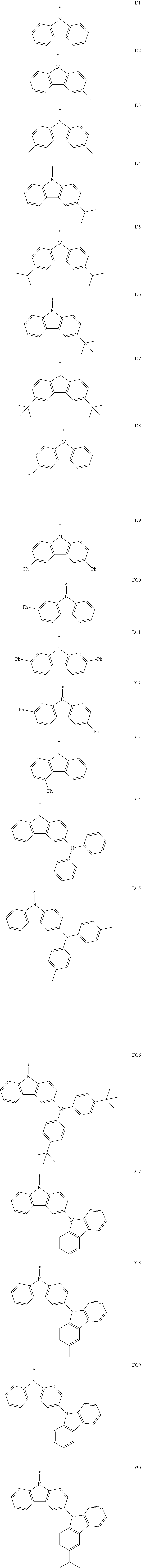

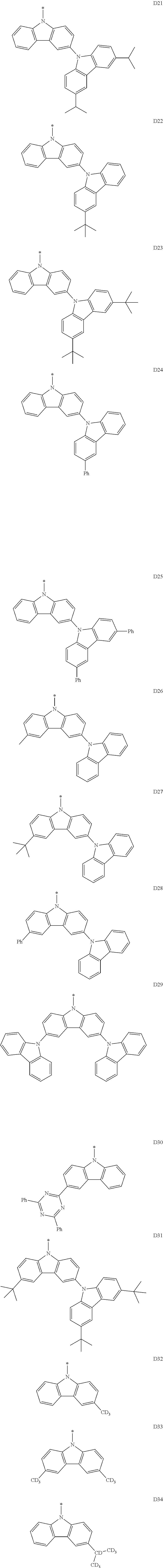

[chemical structure]

Groups obtained by substituting all hydrogen atoms present in the above D1 to D31 with deuterium atoms are disclosed as D55 to D85.

Next, specific examples of the donor group having a fused ring structure of four or more rings are shown below.

[chemical structure]

[chemical structure]

[chemical structure]

[chemical structure]

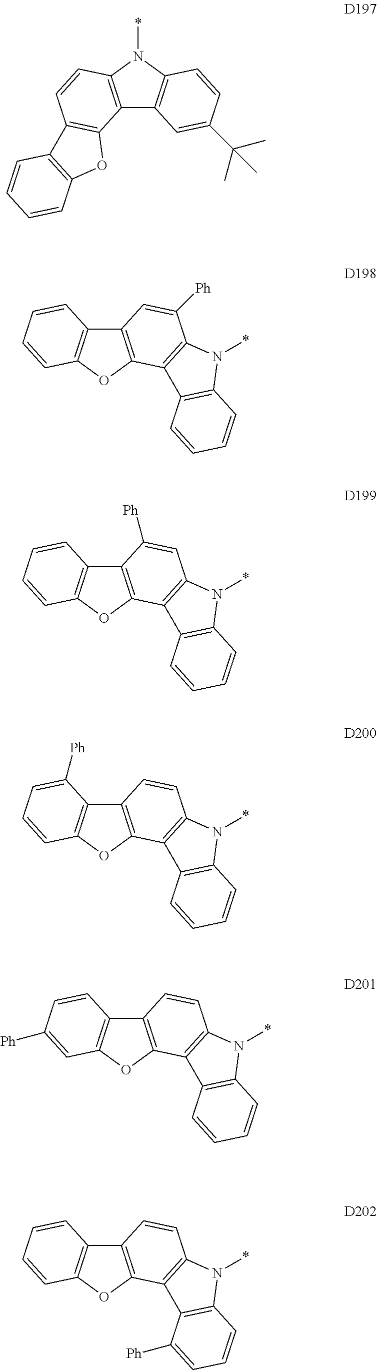 [SMILES: *n1c2ccc(C(C)(C)C)cc2c2c3oc4ccccc4c3ccc21 ; *n1c2ccccc2c2c3oc4ccccc4c3cc(-c3ccccc3)c21 ; *n1c2ccccc2c2c3oc4ccccc4c3c(-c3ccccc3)cc21 ; *n1c2ccccc2c2c3oc4cccc(-c5ccccc5)c4c3ccc21 ; *n1c2ccccc2c2c3oc4cc(-c5ccccc5)ccc4c3ccc21 ; *n1c2cccc(-c3ccccc3)c2c2c3oc4ccccc4c3ccc21]

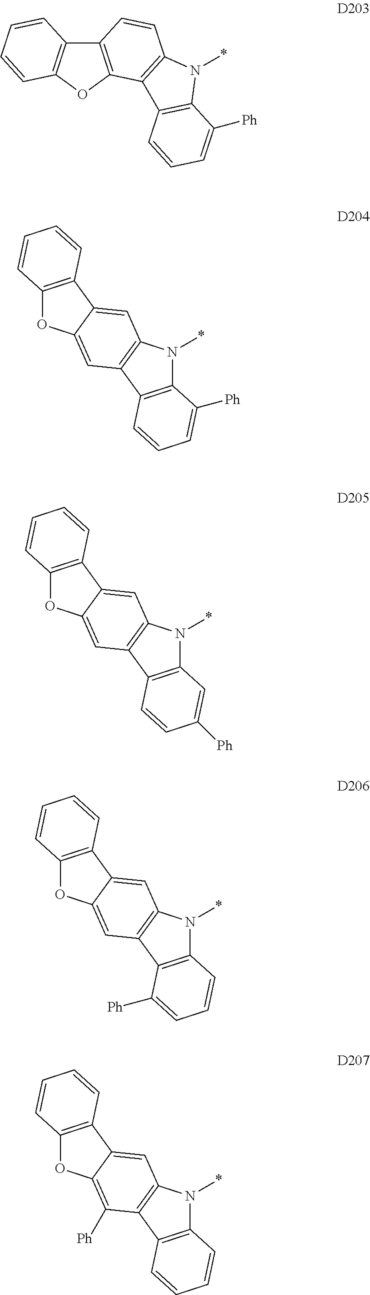 [SMILES: *n1c2ccc3c4ccccc4oc3c2c2cccc(-c3ccccc3)c21 ; *n1c2cc3c(cc2c2cccc(-c4ccccc4)c21)oc1ccccc13 ; *n1c2cc(-c3ccccc3)ccc2c2cc3oc4ccccc4c3cc21 ; *n1c2cc3c(cc2c2c(-c4ccccc4)cccc21)oc1ccccc13 ; *n1c2ccccc2c2c(-c3ccccc3)c3oc4ccccc4c3cc21]

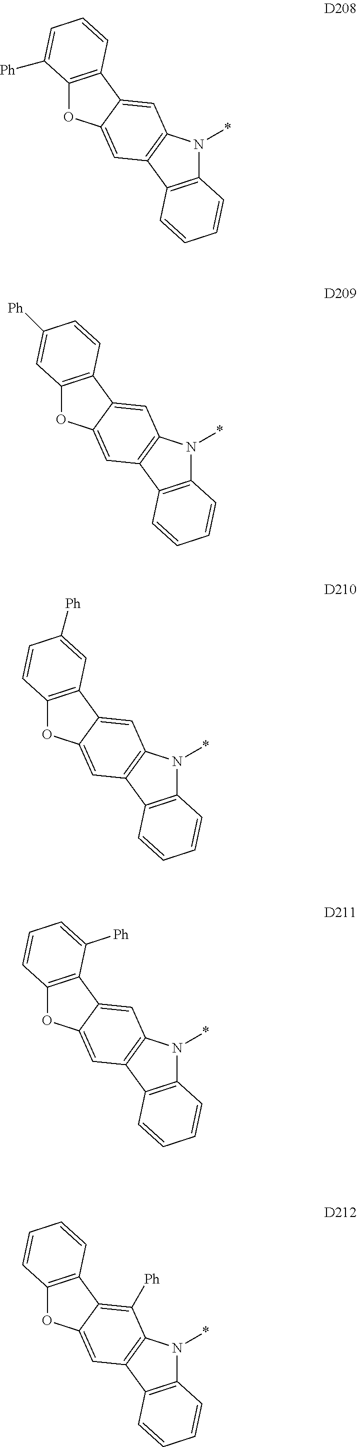 [SMILES: *n1c2ccccc2c2cc3oc4c(-c5ccccc5)cccc4c3cc21 ; *n1c2ccccc2c2cc3oc4cc(-c5ccccc5)ccc4c3cc21 ; *n1c2ccccc2c2cc3oc4ccc(-c5ccccc5)cc4c3cc21 ; *n1c2ccccc2c2cc3oc4cccc(-c5ccccc5)c4c3cc21 ; *n1c2ccccc2c2cc3oc4ccccc4c3c(-c3ccccc3)c21]

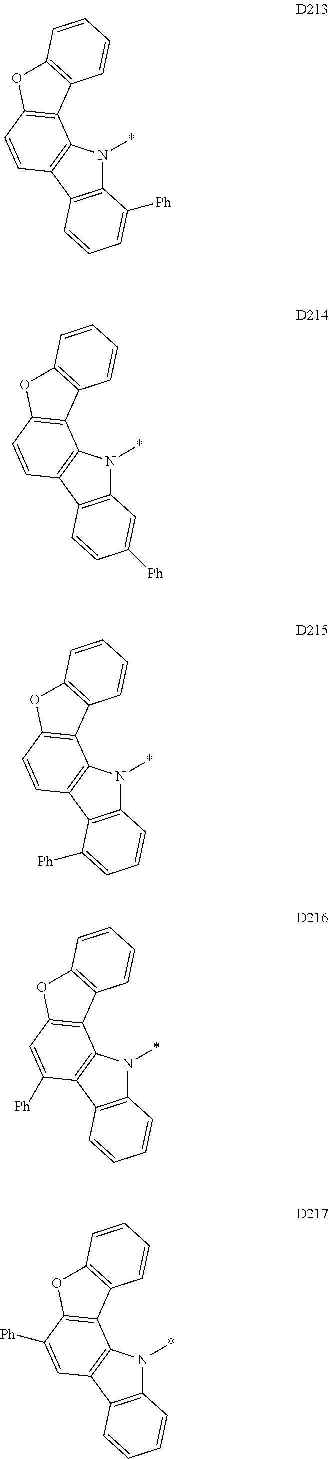 [SMILES: *n1c2c(-c3ccccc3)cccc2c2ccc3oc4ccccc4c3c21 ; *n1c2cc(-c3ccccc3)ccc2c2ccc3oc4ccccc4c3c21 ; *n1c2cccc(-c3ccccc3)c2c2ccc3oc4ccccc4c3c21 ; *n1c2ccccc2c2c(-c3ccccc3)cc3oc4ccccc4c3c21 ; *n1c2ccccc2c2cc(-c3ccccc3)c3oc4ccccc4c3c21]

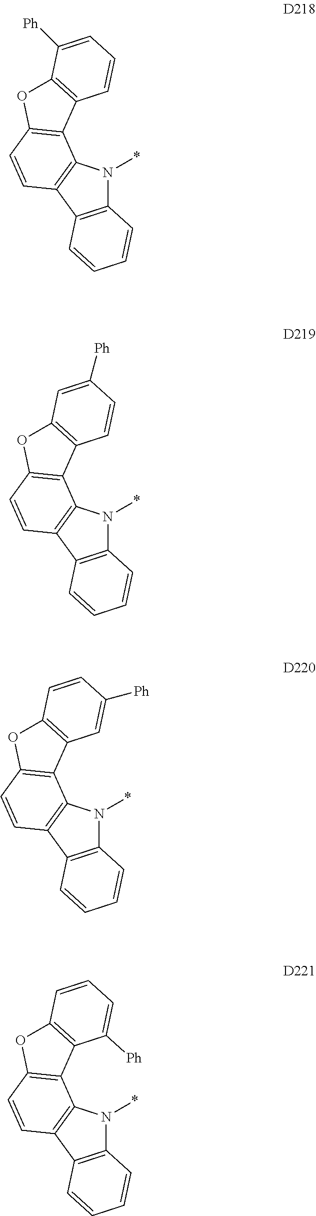 [SMILES: *n1c2ccccc2c2ccc3oc4c(-c5ccccc5)cccc4c3c21 ; *n1c2ccccc2c2ccc3oc4cc(-c5ccccc5)ccc4c3c21 ; *n1c2ccccc2c2ccc3oc4ccc(-c5ccccc5)cc4c3c21 ; *n1c2ccccc2c2ccc3oc4cccc(-c5ccccc5)c4c3c21]

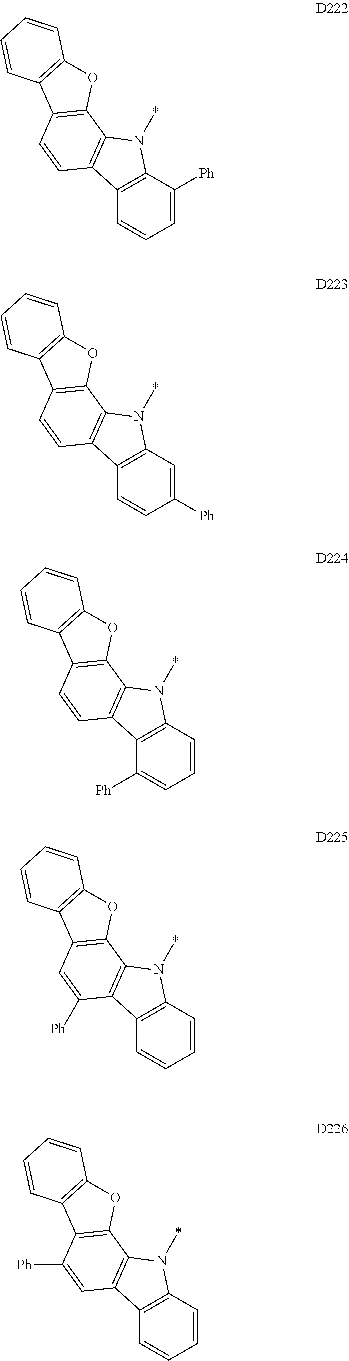 [SMILES: *n1c2c(-c3ccccc3)cccc2c2ccc3c4ccccc4oc3c21 ; *n1c2cc(-c3ccccc3)ccc2c2ccc3c4ccccc4oc3c21 ; *n1c2cccc(-c3ccccc3)c2c2ccc3c4ccccc4oc3c21 ; *n1c2ccccc2c2c(-c3ccccc3)cc3c4ccccc4oc3c21 ; *n1c2ccccc2c2cc(-c3ccccc3)c3c4ccccc4oc3c21]

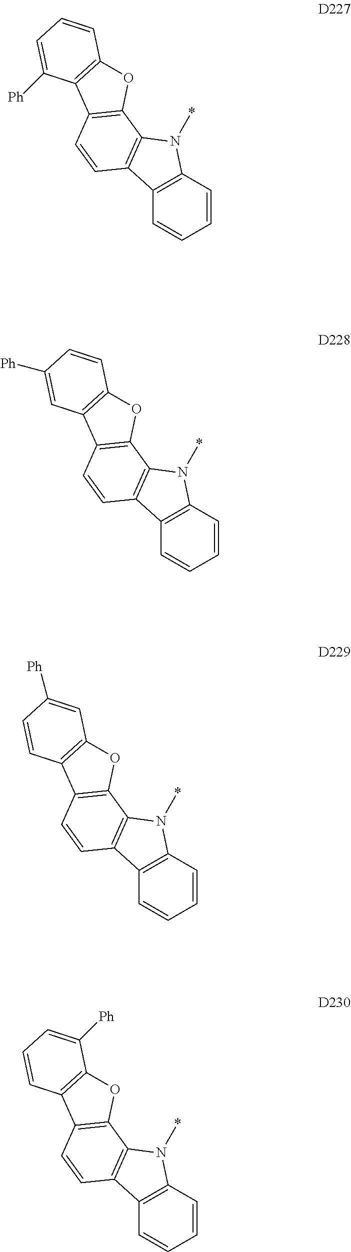 [SMILES: *n1c2ccccc2c2ccc3c(oc4cccc(-c5ccccc5)c43)c21 ; *n1c2ccccc2c2ccc3c4cc(-c5ccccc5)ccc4oc3c21 ; *n1c2ccccc2c2ccc3c4ccc(-c5ccccc5)cc4oc3c21 ; *n1c2ccccc2c2ccc3c4cccc(-c5ccccc5)c4oc3c21]

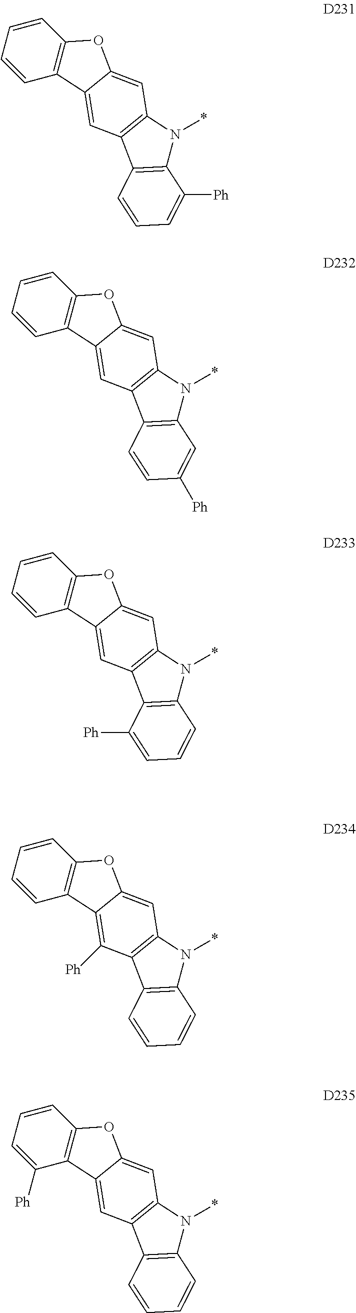 [SMILES: *n1c2cc3oc4ccccc4c3cc2c2cccc(-c3ccccc3)c21 ; *n1c2cc(-c3ccccc3)ccc2c2cc3c(cc21)oc1ccccc13 ; *n1c2cc3oc4ccccc4c3cc2c2c(-c3ccccc3)cccc21 ; *n1c2ccccc2c2c(-c3ccccc3)c3c(cc21)oc1ccccc13 ; *n1c2ccccc2c2cc3c(cc21)oc1cccc(-c2ccccc2)c13]

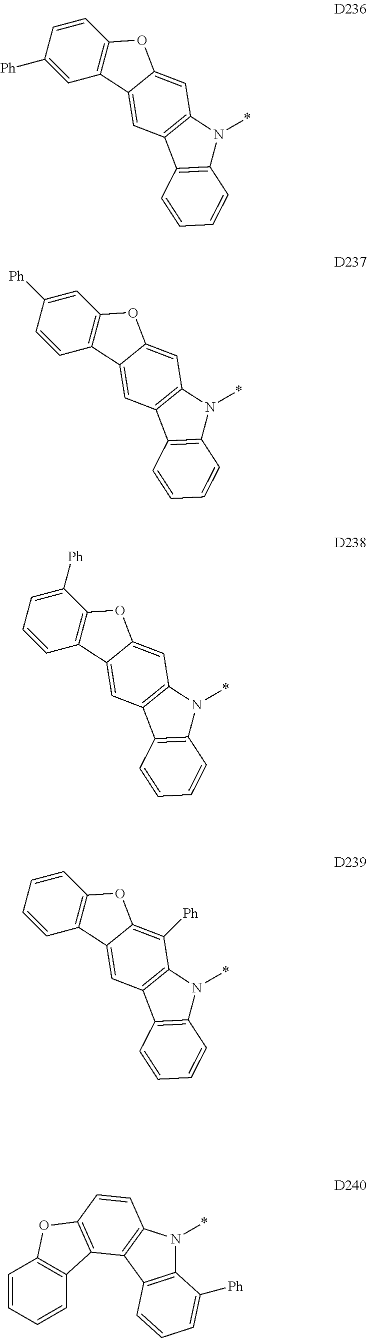 [SMILES: *n1c2ccccc2c2cc3c(cc21)oc1ccc(-c2ccccc2)cc13 ; *n1c2ccccc2c2cc3c(cc21)oc1cc(-c2ccccc2)ccc13 ; *n1c2ccccc2c2cc3c(cc21)oc1c(-c2ccccc2)cccc13 ; *n1c2ccccc2c2cc3c(oc4ccccc43)c(-c3ccccc3)c21 ; *n1c2ccc3oc4ccccc4c3c2c2cccc(-c3ccccc3)c21]

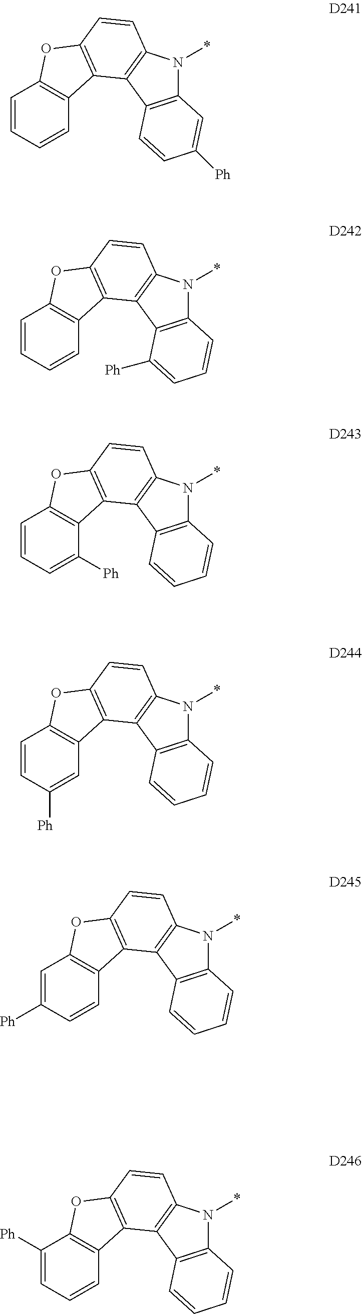 [SMILES: *n1c2cc(-c3ccccc3)ccc2c2c3c(ccc21)oc1ccccc13 ; *n1c2cccc(-c3ccccc3)c2c2c3c(ccc21)oc1ccccc13 ; *n1c2ccccc2c2c3c(ccc21)oc1cccc(-c2ccccc2)c13 ; *n1c2ccccc2c2c3c(ccc21)oc1ccc(-c2ccccc2)cc13 ; *n1c2ccccc2c2c3c(ccc21)oc1cc(-c2ccccc2)ccc13 ; *n1c2ccccc2c2c3c(ccc21)oc1c(-c2ccccc2)cccc13]

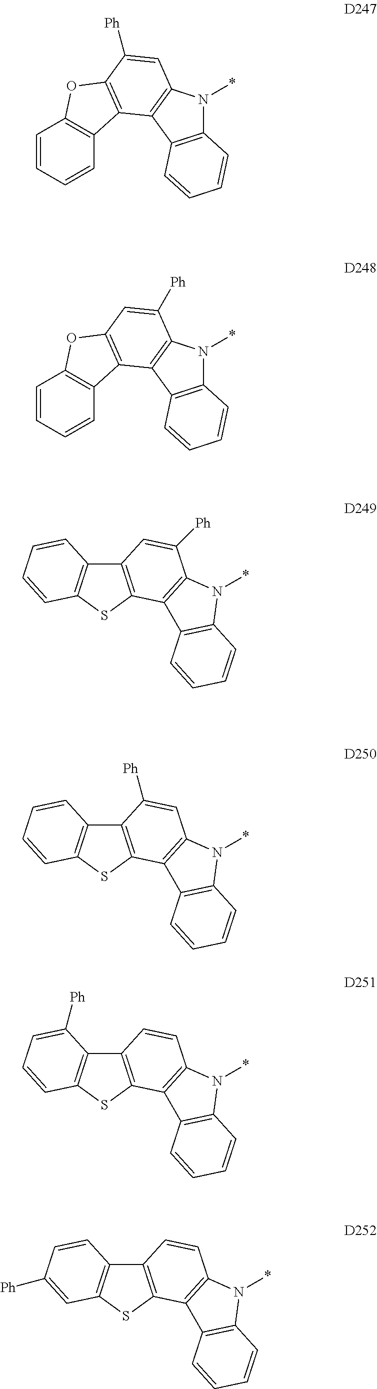 [SMILES: *n1c2ccccc2c2c3c(oc4ccccc43)c(-c3ccccc3)cc21 ; *n1c2ccccc2c2c3c(cc(-c4ccccc4)c21)oc1ccccc13 ; *n1c2ccccc2c2c3sc4ccccc4c3cc(-c3ccccc3)c21 ; *n1c2ccccc2c2c3sc4ccccc4c3c(-c3ccccc3)cc21 ; *n1c2ccccc2c2c3sc4cccc(-c5ccccc5)c4c3ccc21 ; *n1c2ccccc2c2c3sc4cc(-c5ccccc5)ccc4c3ccc21]

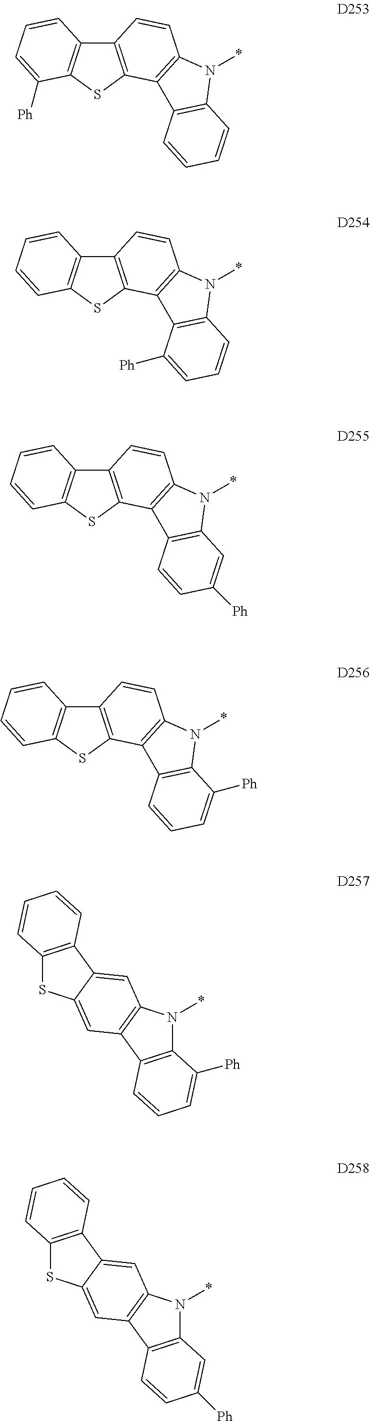 [SMILES: *n1c2ccccc2c2c3sc4c(-c5ccccc5)cccc4c3ccc21 ; *n1c2cccc(-c3ccccc3)c2c2c3sc4ccccc4c3ccc21 ; *n1c2cc(-c3ccccc3)ccc2c2c3sc4ccccc4c3ccc21 ; *n1c2ccc3c4ccccc4sc3c2c2cccc(-c3ccccc3)c21 ; *n1c2cc3c(cc2c2cccc(-c4ccccc4)c21)sc1ccccc13 ; *n1c2cc(-c3ccccc3)ccc2c2cc3sc4ccccc4c3cc21]

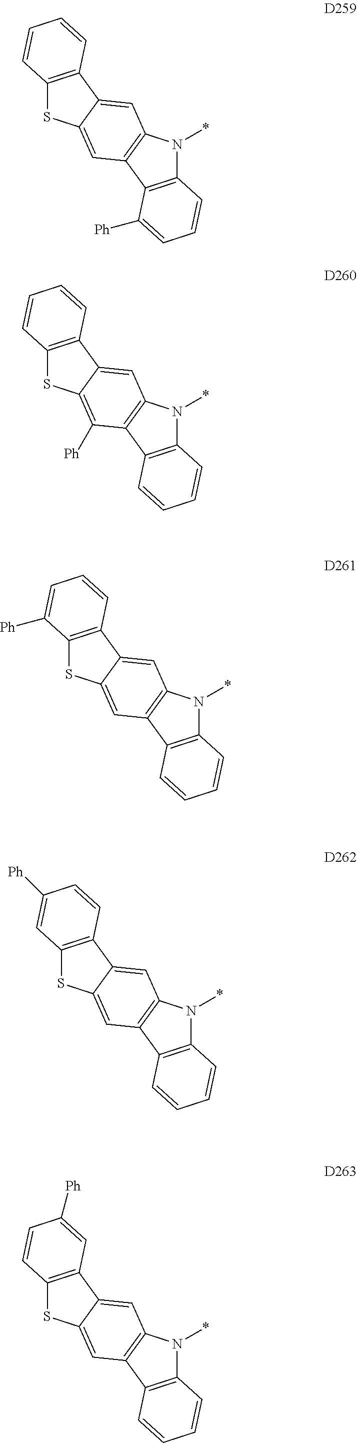 [SMILES: *n1c2cc3c(cc2c2c(-c4ccccc4)cccc21)sc1ccccc13 ; *n1c2ccccc2c2c(-c3ccccc3)c3sc4ccccc4c3cc21 ; *n1c2ccccc2c2cc3sc4c(-c5ccccc5)cccc4c3cc21 ; *n1c2ccccc2c2cc3sc4cc(-c5ccccc5)ccc4c3cc21 ; *n1c2ccccc2c2cc3sc4ccc(-c5ccccc5)cc4c3cc21]

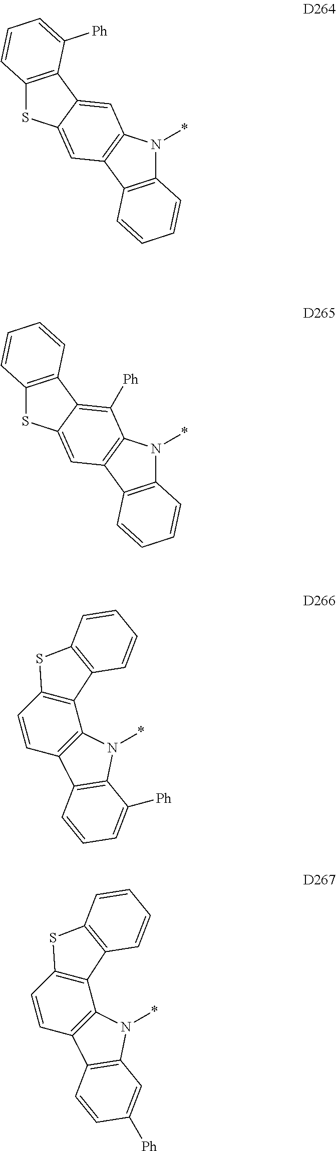 [SMILES: *n1c2ccccc2c2cc3sc4cccc(-c5ccccc5)c4c3cc21 ; *n1c2ccccc2c2cc3sc4ccccc4c3c(-c3ccccc3)c21 ; *n1c2c(-c3ccccc3)cccc2c2ccc3sc4ccccc4c3c21 ; *n1c2cc(-c3ccccc3)ccc2c2ccc3sc4ccccc4c3c21]

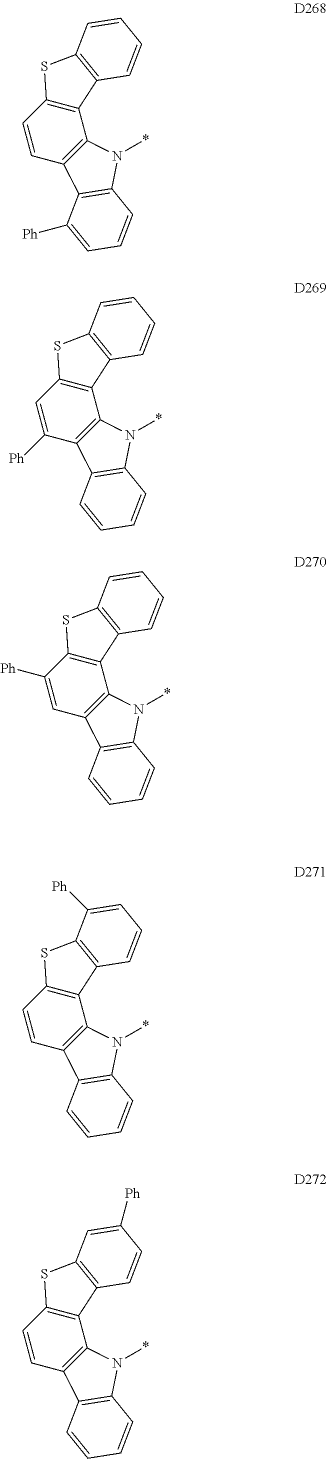 [SMILES: *n1c2cccc(-c3ccccc3)c2c2ccc3sc4ccccc4c3c21 ; *n1c2ccccc2c2c(-c3ccccc3)cc3sc4ccccc4c3c21 ; *n1c2ccccc2c2cc(-c3ccccc3)c3sc4ccccc4c3c21 ; *n1c2ccccc2c2ccc3sc4c(-c5ccccc5)cccc4c3c21 ; *n1c2ccccc2c2ccc3sc4cc(-c5ccccc5)ccc4c3c21]

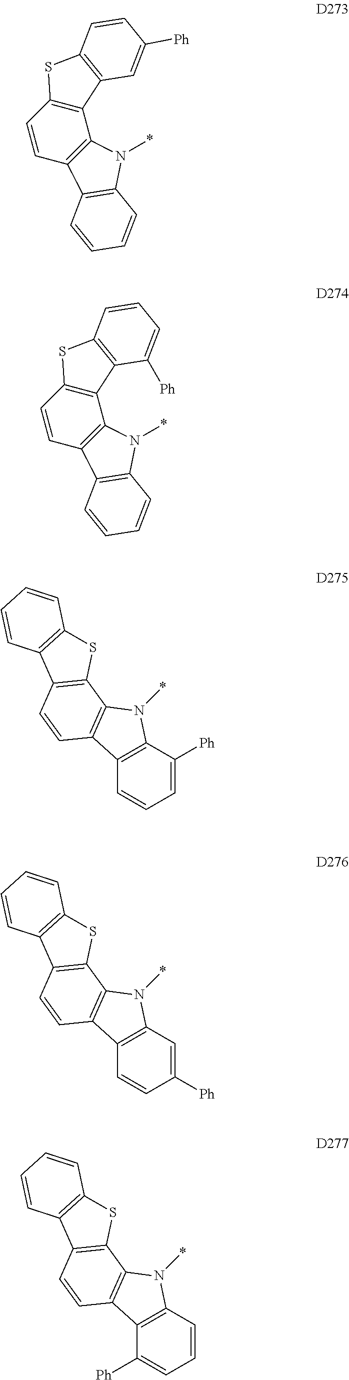 [SMILES: *n1c2ccccc2c2ccc3sc4ccc(-c5ccccc5)cc4c3c21 ; *n1c2ccccc2c2ccc3sc4cccc(-c5ccccc5)c4c3c21 ; *n1c2c(-c3ccccc3)cccc2c2ccc3c4ccccc4sc3c21 ; *n1c2cc(-c3ccccc3)ccc2c2ccc3c4ccccc4sc3c21 ; *n1c2cccc(-c3ccccc3)c2c2ccc3c4ccccc4sc3c21]

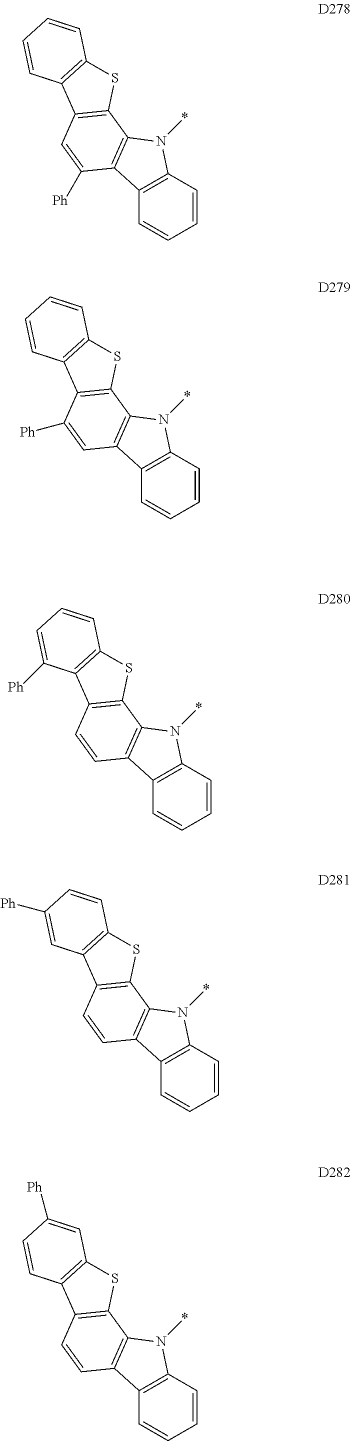 [SMILES: *n1c2ccccc2c2c(-c3ccccc3)cc3c4ccccc4sc3c21 ; *n1c2ccccc2c2cc(-c3ccccc3)c3c4ccccc4sc3c21 ; *n1c2ccccc2c2ccc3c(sc4cccc(-c5ccccc5)c43)c21 ; *n1c2ccccc2c2ccc3c4cc(-c5ccccc5)ccc4sc3c21 ; *n1c2ccccc2c2ccc3c4ccc(-c5ccccc5)cc4sc3c21]

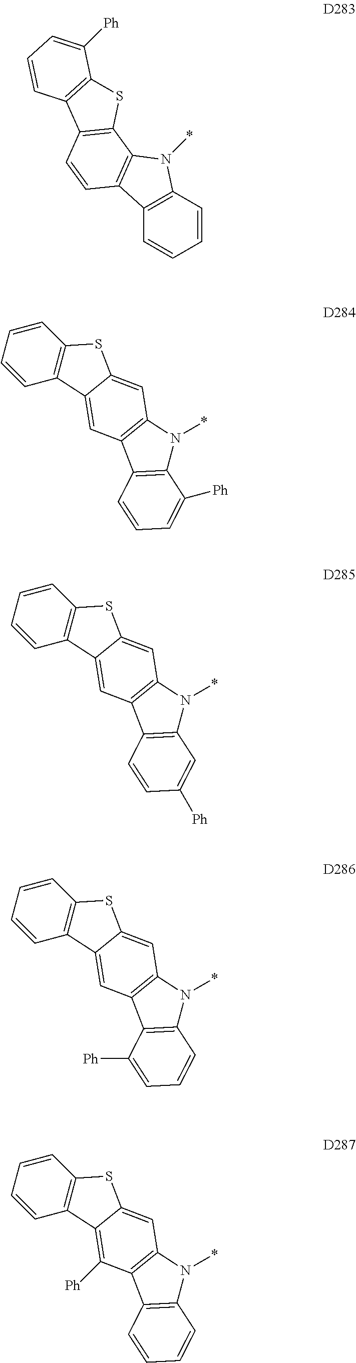 [SMILES: *n1c2ccccc2c2ccc3c4cccc(-c5ccccc5)c4sc3c21 ; *n1c2cc3sc4ccccc4c3cc2c2cccc(-c3ccccc3)c21 ; *n1c2cc(-c3ccccc3)ccc2c2cc3c(cc21)sc1ccccc13 ; *n1c2cc3sc4ccccc4c3cc2c2c(-c3ccccc3)cccc21 ; *n1c2ccccc2c2c(-c3ccccc3)c3c(cc21)sc1ccccc13]

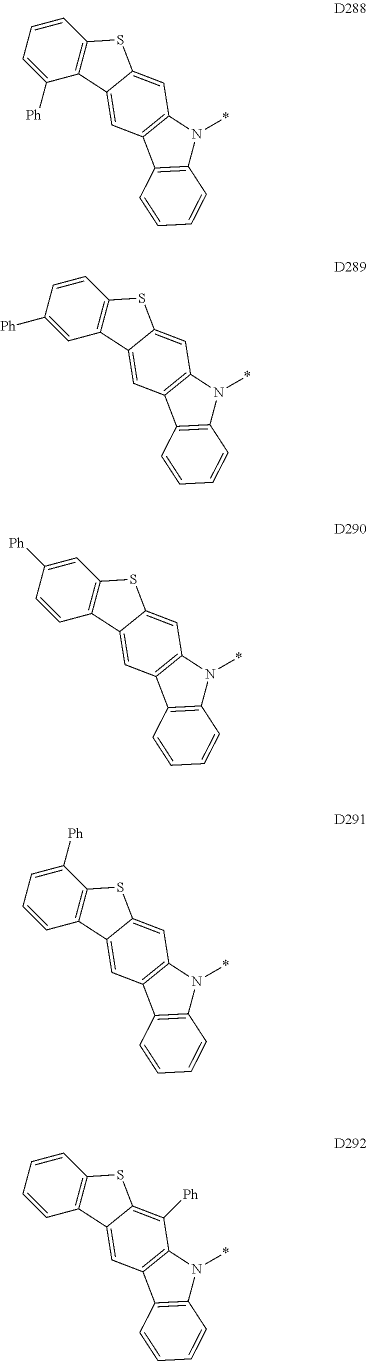 [SMILES: *n1c2ccccc2c2cc3c(cc21)sc1cccc(-c2ccccc2)c13 ; *n1c2ccccc2c2cc3c(cc21)sc1ccc(-c2ccccc2)cc13 ; *n1c2ccccc2c2cc3c(cc21)sc1cc(-c2ccccc2)ccc13 ; *n1c2ccccc2c2cc3c(cc21)sc1c(-c2ccccc2)cccc13 ; *n1c2ccccc2c2cc3c(sc4ccccc43)c(-c3ccccc3)c21]

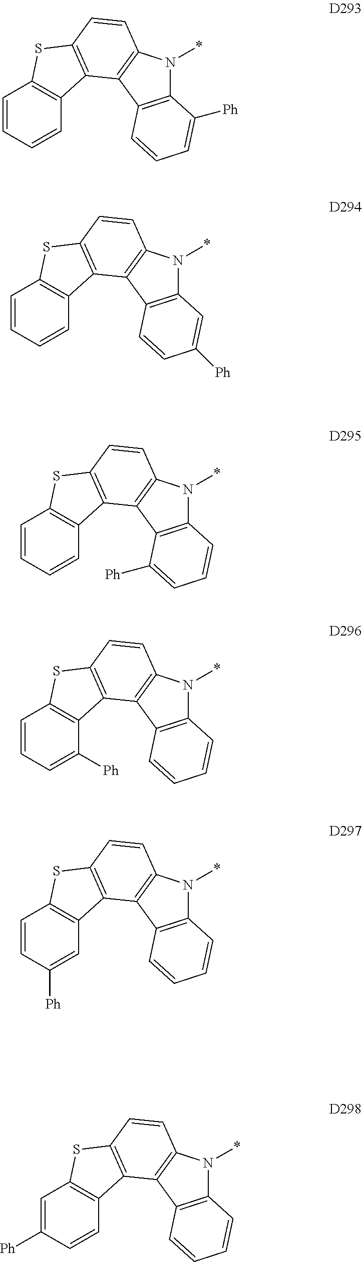 [SMILES: *n1c2ccc3sc4ccccc4c3c2c2cccc(-c3ccccc3)c21 ; *n1c2cc(-c3ccccc3)ccc2c2c3c(ccc21)sc1ccccc13 ; *n1c2cccc(-c3ccccc3)c2c2c3c(ccc21)sc1ccccc13 ; *n1c2ccccc2c2c3c(ccc21)sc1cccc(-c2ccccc2)c13 ; *n1c2ccccc2c2c3c(ccc21)sc1ccc(-c2ccccc2)cc13 ; *n1c2ccccc2c2c3c(ccc21)sc1cc(-c2ccccc2)ccc13]

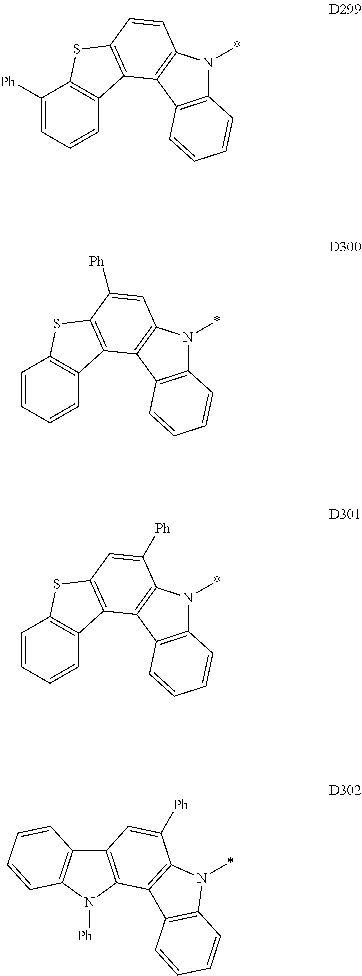 [SMILES: *n1c2ccccc2c2c3c(ccc21)sc1c(-c2ccccc2)cccc13 ; *n1c2ccccc2c2c3c(sc4ccccc43)c(-c3ccccc3)cc21 ; *n1c2ccccc2c2c3c(cc(-c4ccccc4)c21)sc1ccccc13 ; *n1c2ccccc2c2c1c(-c1ccccc1)cc1c3ccccc3n(-c3ccccc3)c12]

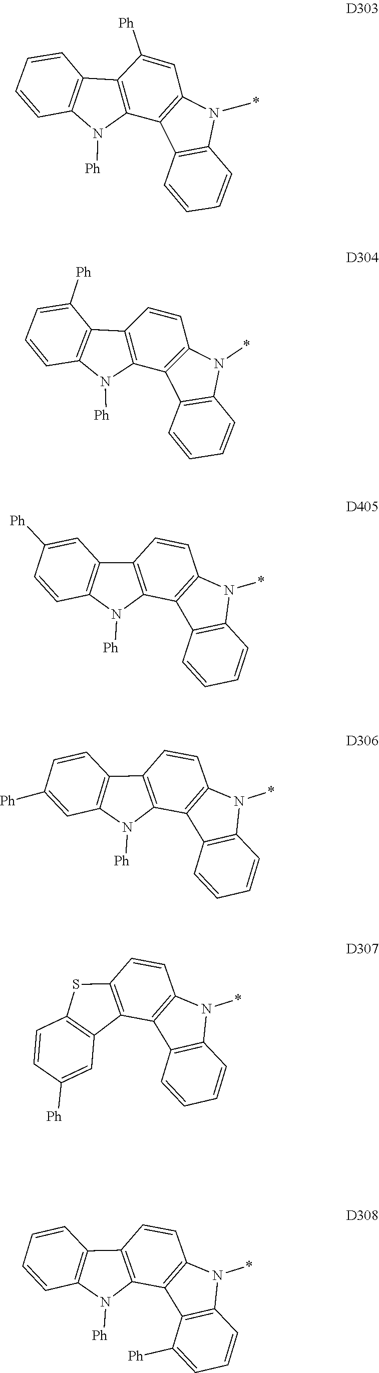 [SMILES: *n1c2c(c3c1cc(-c1ccccc1)c1c4ccccc4n(-c4ccccc4)c13)C=CCC=C2 ; *n1c2ccccc2c2c1ccc1c3c(-c4ccccc4)cccc3n(-c3ccccc3)c12 ; *n1c2ccccc2c2c1ccc1c3cc(-c4ccccc4)ccc3n(-c3ccccc3)c12 ; *n1c2ccccc2c2c1ccc1c3ccc(-c4ccccc4)cc3n(-c3ccccc3)c12 ; *n1c2ccccc2c2c3c(ccc21)sc1ccc(-c2ccccc2)cc13 ; *n1c2cccc(-c3ccccc3)c2c2c1ccc1c3ccccc3n(-c3ccccc3)c12]

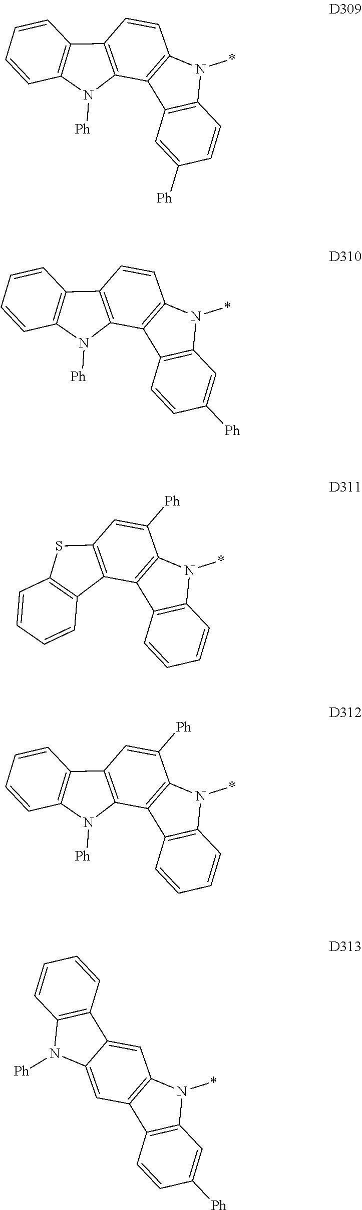 [SMILES: *n1c2ccc(-c3ccccc3)cc2c2c1ccc1c3ccccc3n(-c3ccccc3)c12 ; *n1c2cc(-c3ccccc3)ccc2c2c1ccc1c3ccccc3n(-c3ccccc3)c12 ; *n1c2ccccc2c2c3c(cc(-c4ccccc4)c21)sc1ccccc13 ; *n1c2ccccc2c2c1c(-c1ccccc1)cc1c3ccccc3n(-c3ccccc3)c12 ; *n1c2cc(-c3ccccc3)ccc2c2cc3c(cc21)c1ccccc1n3-c1ccccc1]

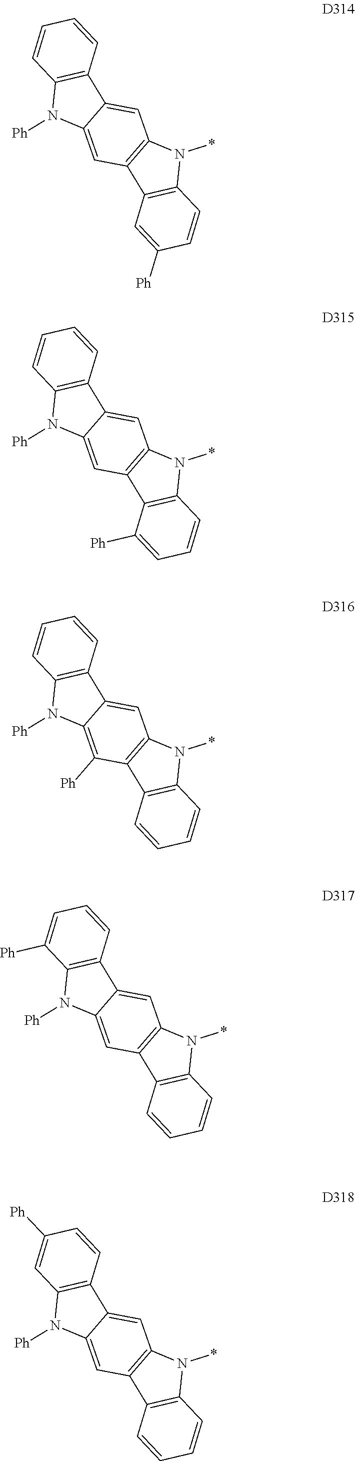 [SMILES: *n1c2ccc(-c3ccccc3)cc2c2cc3c(cc21)c1ccccc1n3-c1ccccc1 ; *n1c2cc3c4ccccc4n(-c4ccccc4)c3cc2c2c(-c3ccccc3)cccc21 ; *n1c2ccccc2c2c(-c3ccccc3)c3c(cc21)c1ccccc1n3-c1ccccc1 ; *n1c2ccccc2c2cc3c(cc21)c1cccc(-c2ccccc2)c1n3-c1ccccc1 ; *n1c2ccccc2c2cc3c(cc21)c1ccc(-c2ccccc2)cc1n3-c1ccccc1]

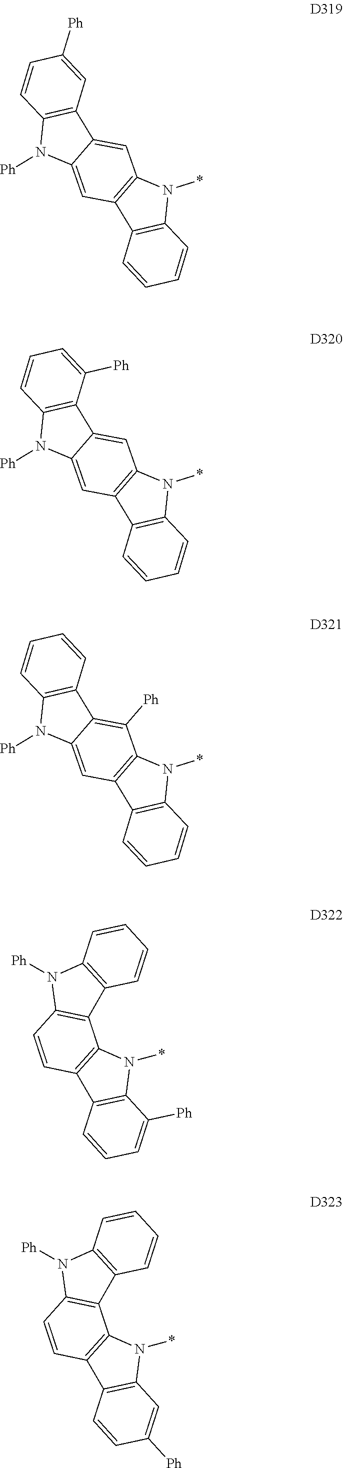 [SMILES: *n1c2ccccc2c2cc3c(cc21)c1cc(-c2ccccc2)ccc1n3-c1ccccc1 ; *n1c2ccccc2c2cc3c(cc21)c1c(-c2ccccc2)cccc1n3-c1ccccc1 ; *n1c2ccccc2c2cc3c(c(-c4ccccc4)c21)c1ccccc1n3-c1ccccc1 ; *n1c2c(-c3ccccc3)cccc2c2ccc3c(c4ccccc4n3-c3ccccc3)c21 ; *n1c2cc(-c3ccccc3)ccc2c2ccc3c(c4ccccc4n3-c3ccccc3)c21]

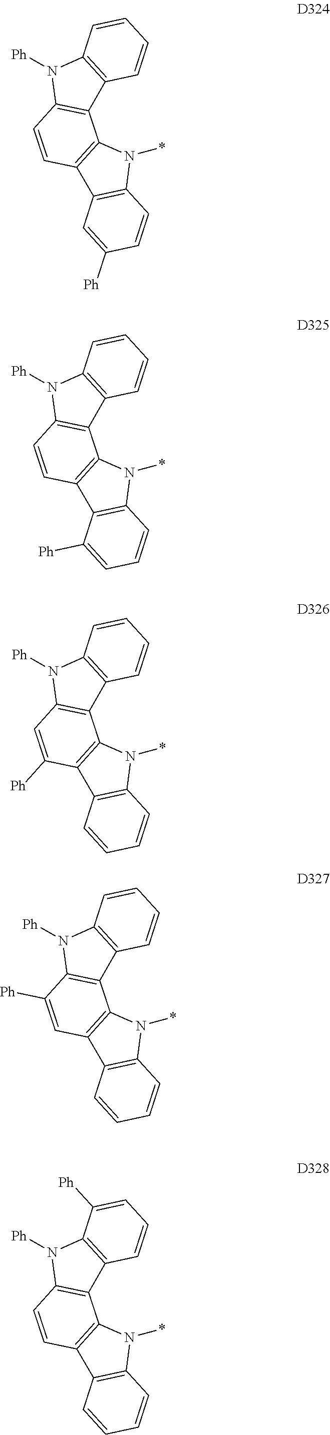

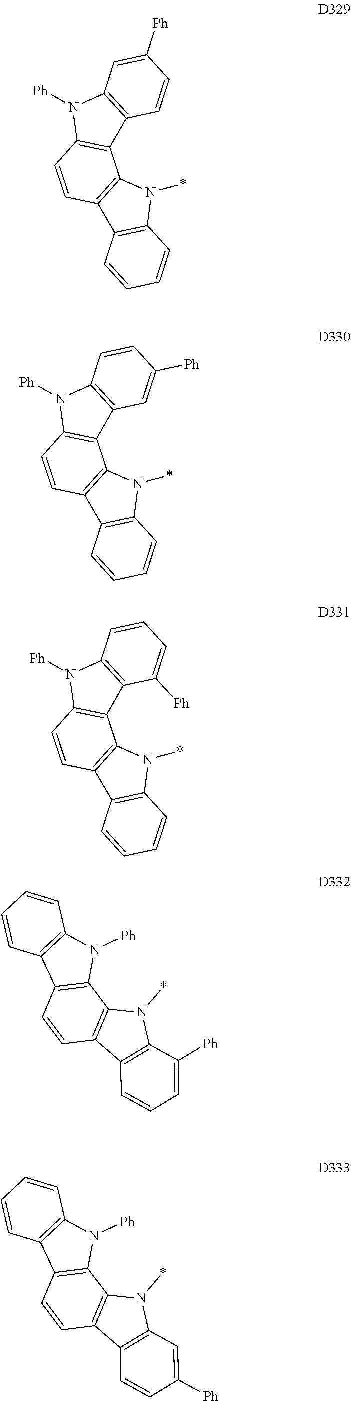 [SMILES: *n1c2ccccc2c2ccc3c(c4ccc(-c5ccccc5)cc4n3-c3ccccc3)c21 ; *n1c2ccccc2c2ccc3c(c4cc(-c5ccccc5)ccc4n3-c3ccccc3)c21 ; *n1c2ccccc2c2ccc3c(c4c(-c5ccccc5)cccc4n3-c3ccccc3)c21 ; *n1c2c(-c3ccccc3)cccc2c2ccc3c4ccccc4n(-c4ccccc4)c3c21 ; *n1c2cc(-c3ccccc3)ccc2c2ccc3c4ccccc4n(-c4ccccc4)c3c21]

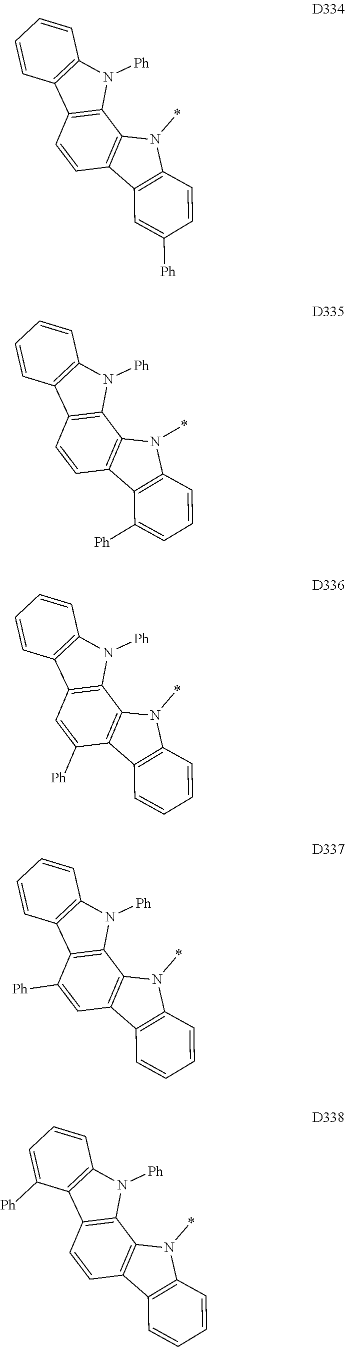 [SMILES: *n1c2ccc(-c3ccccc3)cc2c2ccc3c4ccccc4n(-c4ccccc4)c3c21 ; *n1c2cccc(-c3ccccc3)c2c2ccc3c4ccccc4n(-c4ccccc4)c3c21 ; *n1c2ccccc2c2c(-c3ccccc3)cc3c4ccccc4n(-c4ccccc4)c3c21 ; *n1c2ccccc2c2cc(-c3ccccc3)c3c4ccccc4n(-c4ccccc4)c3c21 ; *n1c2ccccc2c2ccc3c4c(-c5ccccc5)cccc4n(-c4ccccc4)c3c21]

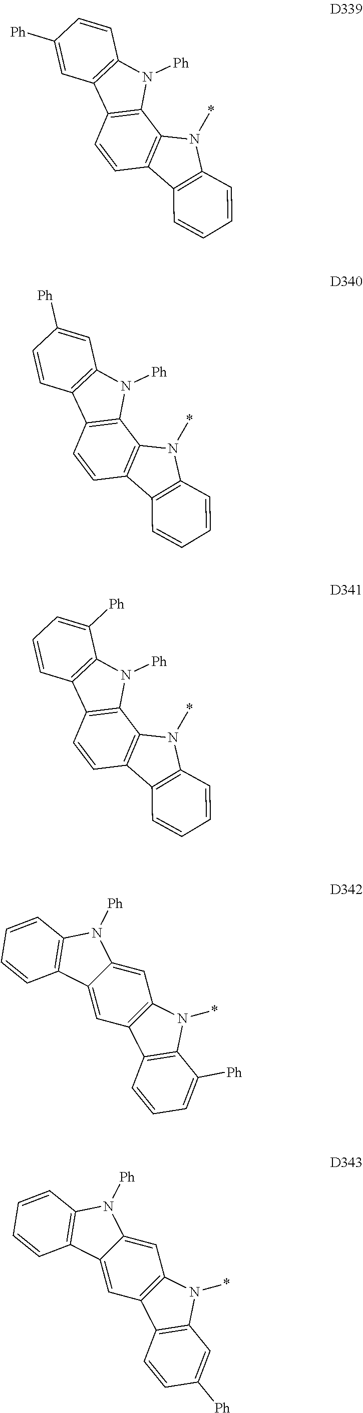 [SMILES: *n1c2ccccc2c2ccc3c4cc(-c5ccccc5)ccc4n(-c4ccccc4)c3c21 ; *n1c2ccccc2c2ccc3c4ccc(-c5ccccc5)cc4n(-c4ccccc4)c3c21 ; *n1c2ccccc2c2ccc3c4cccc(-c5ccccc5)c4n(-c4ccccc4)c3c21 ; *n1c2cc3c(cc2c2cccc(-c4ccccc4)c21)c1ccccc1n3-c1ccccc1 ; *n1c2cc(-c3ccccc3)ccc2c2cc3c4ccccc4n(-c4ccccc4)c3cc21]

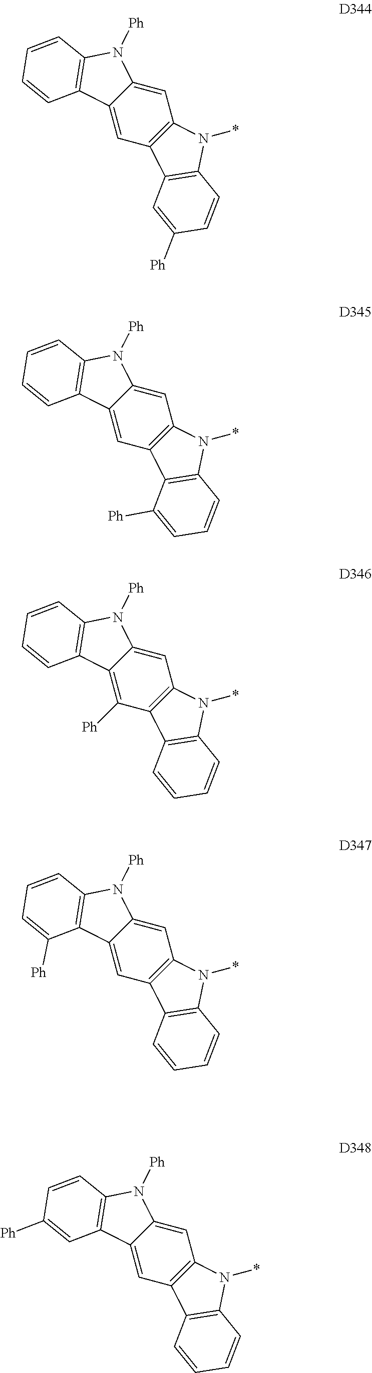 [SMILES: *n1c2ccc(-c3ccccc3)cc2c2cc3c4ccccc4n(-c4ccccc4)c3cc21 ; *n1c2cc3c(cc2c2c(-c4ccccc4)cccc21)c1ccccc1n3-c1ccccc1 ; *n1c2ccccc2c2c(-c3ccccc3)c3c4ccccc4n(-c4ccccc4)c3cc21 ; *n1c2ccccc2c2cc3c4c(-c5ccccc5)cccc4n(-c4ccccc4)c3cc21 ; *n1c2ccccc2c2cc3c4cc(-c5ccccc5)ccc4n(-c4ccccc4)c3cc21]

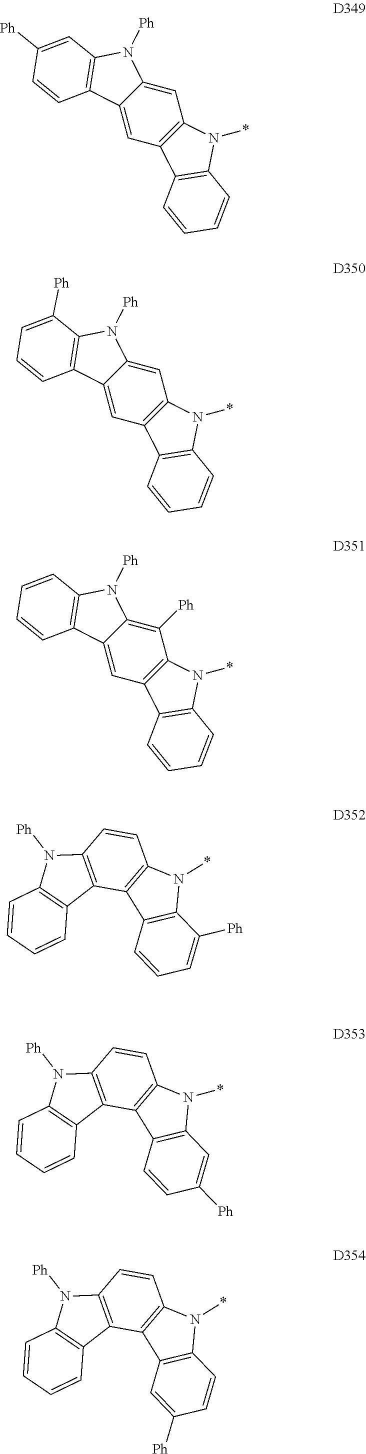 [SMILES: *n1c2ccccc2c2cc3c4ccc(-c5ccccc5)cc4n(-c4ccccc4)c3cc21 ; *n1c2ccccc2c2cc3c4cccc(-c5ccccc5)c4n(-c4ccccc4)c3cc21 ; *n1c2ccccc2c2cc3c4ccccc4n(-c4ccccc4)c3c(-c3ccccc3)c21 ; *n1c2ccc3c(c4ccccc4n3-c3ccccc3)c2c2cccc(-c3ccccc3)c21 ; *n1c2cc(-c3ccccc3)ccc2c2c3c4ccccc4n(-c4ccccc4)c3ccc21 ; *n1c2ccc(-c3ccccc3)cc2c2c3c4ccccc4n(-c4ccccc4)c3ccc21]

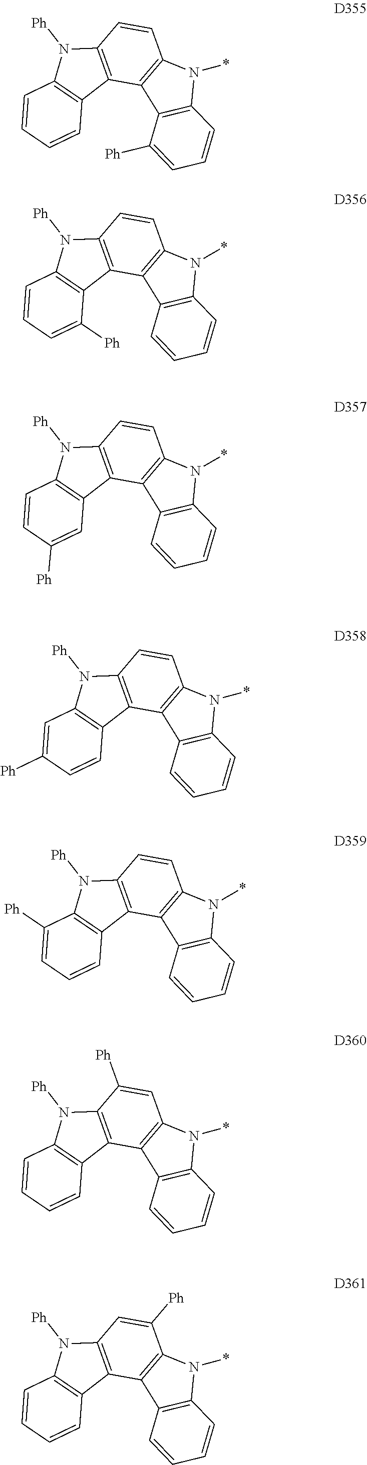

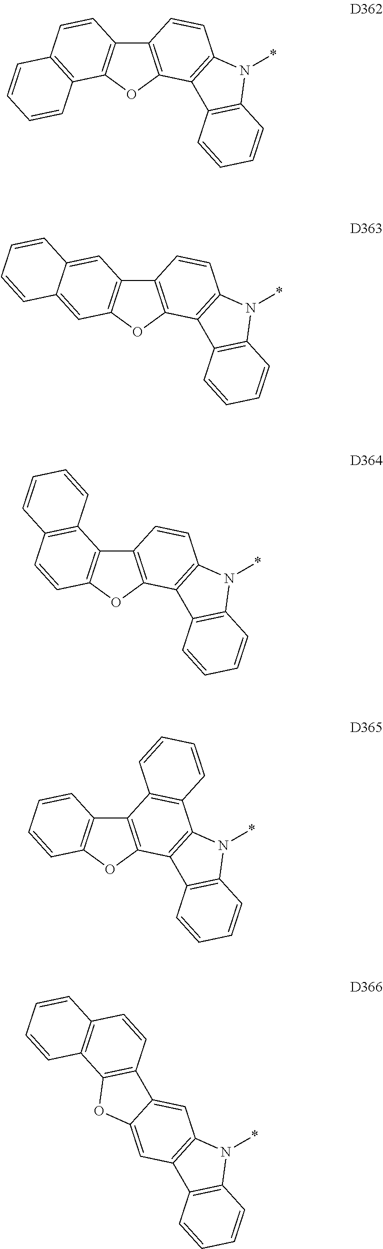 [SMILES: *n1c2ccccc2c2c3oc4c5ccccc5ccc4c3ccc21 ; *n1c2ccccc2c2c3oc4cc5ccccc5cc4c3ccc21 ; *n1c2ccccc2c2c3oc4ccc5ccccc5c4c3ccc21 ; *n1c2ccccc2c2c3oc4ccccc4c3c3ccccc3c21 ; *n1c2ccccc2c2cc3oc4c5ccccc5ccc4c3cc21]

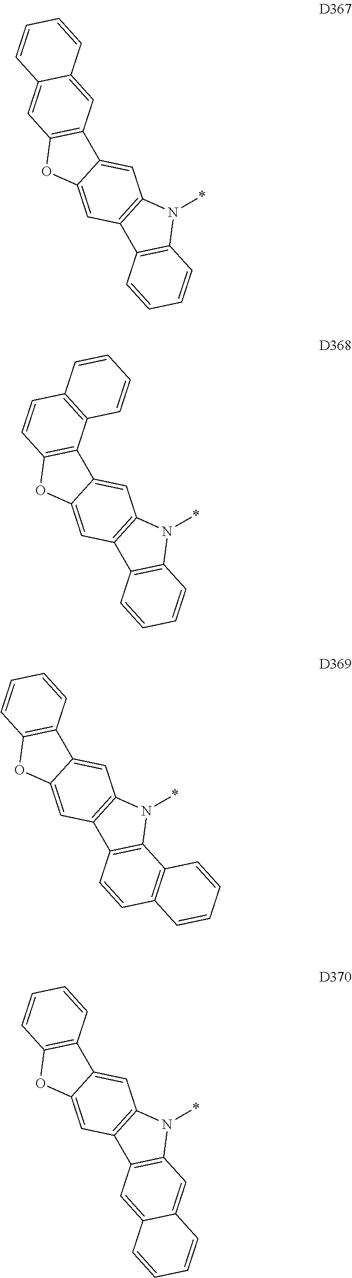 [SMILES: *n1c2ccccc2c2cc3oc4cc5ccccc5cc4c3cc21 ; *n1c2ccccc2c2cc3oc4ccc5ccccc5c4c3cc21 ; *n1c2cc3c(cc2c2ccc4ccccc4c21)oc1ccccc13 ; *n1c2cc3ccccc3cc2c2cc3oc4ccccc4c3cc21]

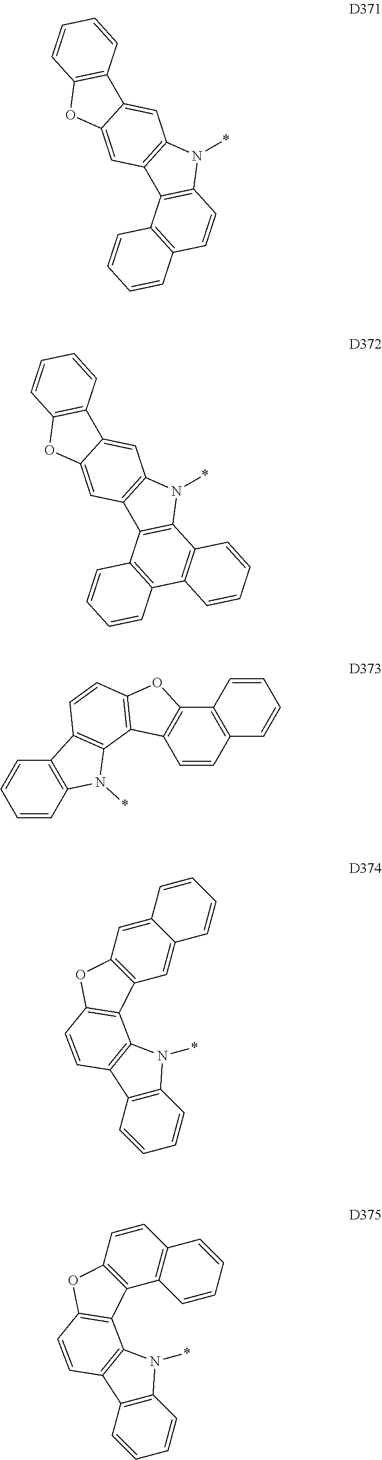 [SMILES: *n1c2cc3c(cc2c2c4ccccc4ccc21)oc1ccccc13 ; *n1c2cc3c(cc2c2c4ccccc4c4ccccc4c21)oc1ccccc13 ; *n1c2ccccc2c2ccc3oc4c5ccccc5ccc4c3c21 ; *n1c2ccccc2c2ccc3oc4cc5ccccc5cc4c3c21 ; *n1c2ccccc2c2ccc3oc4ccc5ccccc5c4c3c21]

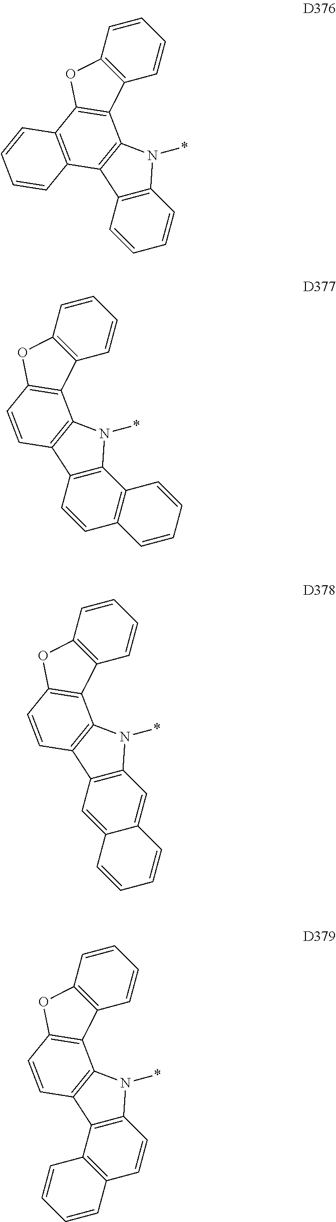 [SMILES: *n1c2ccccc2c2c3ccccc3c3oc4ccccc4c3c21 ; *n1c2c3ccccc3ccc2c2ccc3oc4ccccc4c3c21 ; *n1c2cc3ccccc3cc2c2ccc3oc4ccccc4c3c21 ; *n1c2ccc3ccccc3c2c2ccc3oc4ccccc4c3c21]

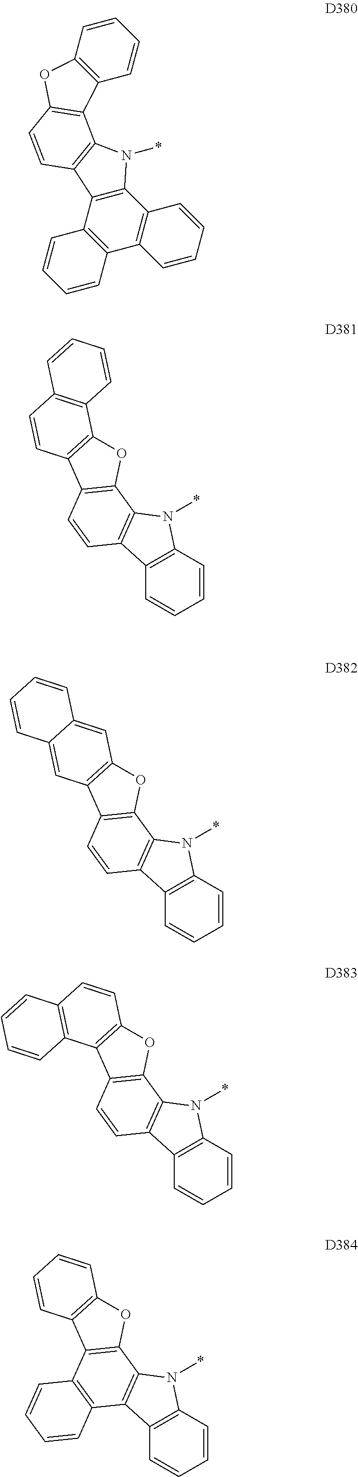

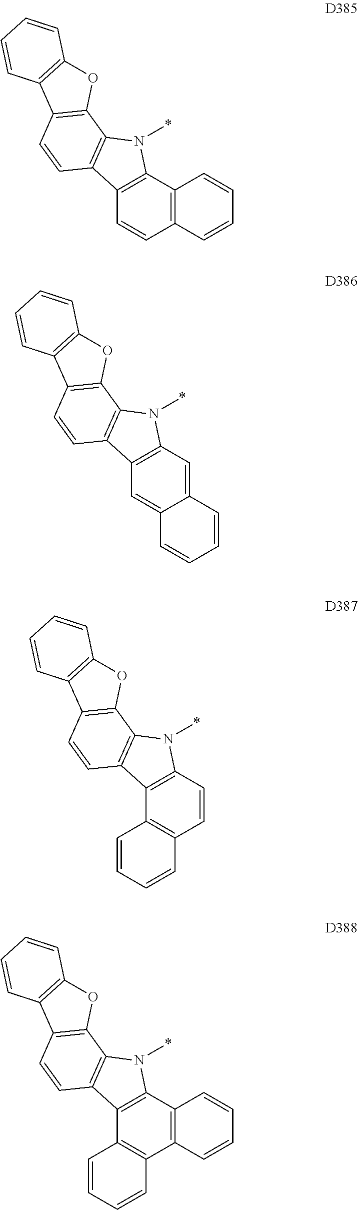 [SMILES: *n1c2c3ccccc3ccc2c2ccc3c4ccccc4oc3c21 ; *n1c2cc3ccccc3cc2c2ccc3c4ccccc4oc3c21 ; *n1c2ccc3ccccc3c2c2ccc3c4ccccc4oc3c21 ; *n1c2c(ccc3c4ccccc4oc32)c2c3ccccc3c3ccccc3c21]

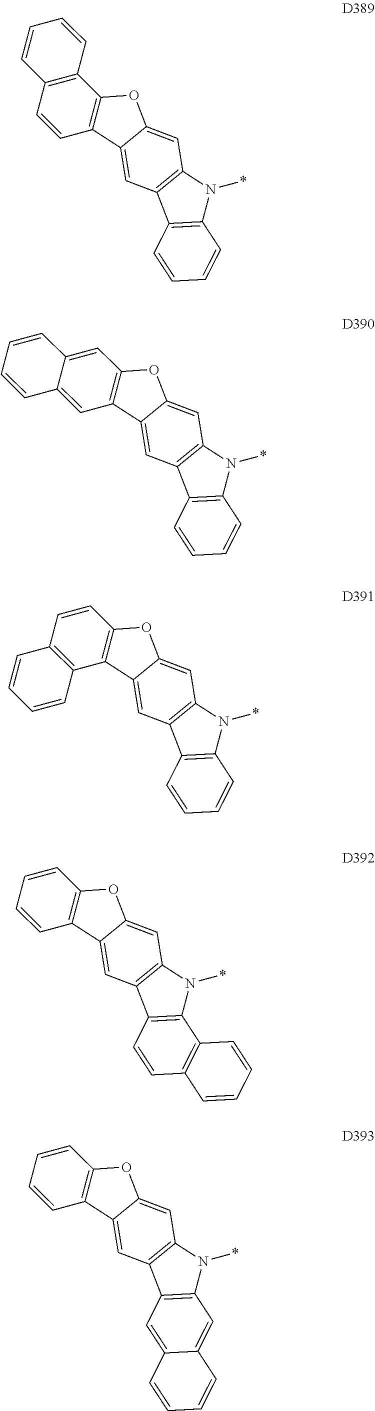 [SMILES: *n1c2ccccc2c2cc3c(cc21)oc1c2ccccc2ccc31 ; *n1c2ccccc2c2cc3c(cc21)oc1cc2ccccc2cc13 ; *n1c2ccccc2c2cc3c(cc21)oc1ccc2ccccc2c13 ; *n1c2cc3oc4ccccc4c3cc2c2ccc3ccccc3c21 ; *n1c2cc3ccccc3cc2c2cc3c(cc21)oc1ccccc13]

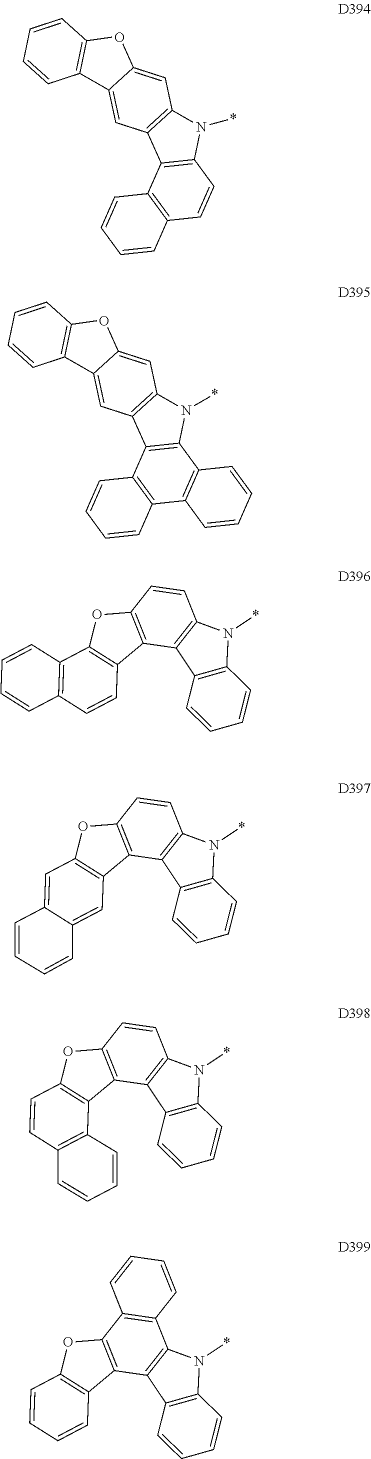 [SMILES: *n1c2cc3oc4ccccc4c3cc2c2c3ccccc3ccc21 ; *n1c2cc3oc4ccccc4c3cc2c2c3ccccc3c3ccccc3c21 ; *n1c2ccccc2c2c3c(ccc21)oc1c2ccccc2ccc13 ; *n1c2ccccc2c2c3c(ccc21)oc1cc2ccccc2cc13 ; *n1c2ccccc2c2c3c(ccc21)oc1ccc2ccccc2c13 ; *n1c2ccccc2c2c3c4ccccc4oc3c3ccccc3c21]

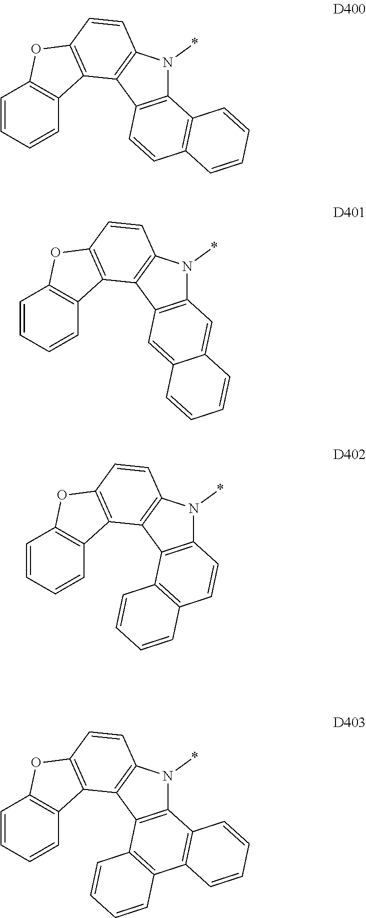 [SMILES: *n1c2ccc3oc4ccccc4c3c2c2ccc3ccccc3c21 ; *n1c2cc3ccccc3cc2c2c3c(ccc21)oc1ccccc13 ; *n1c2ccc3ccccc3c2c2c3c(ccc21)oc1ccccc13 ; *n1c2ccc3oc4ccccc4c3c2c2c3ccccc3c3ccccc3c21]

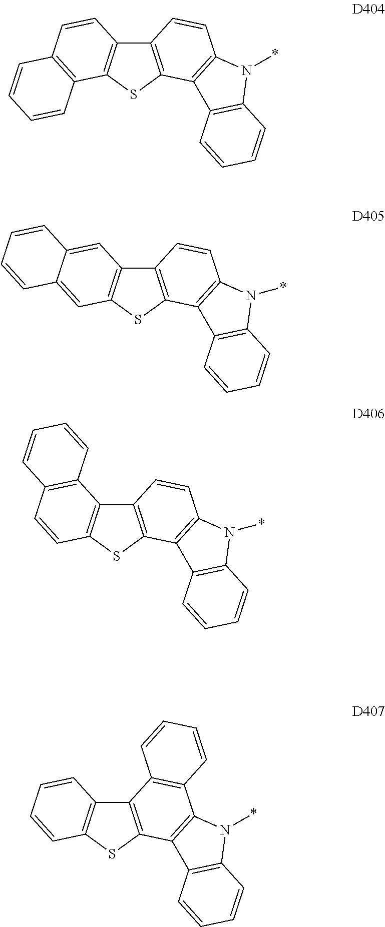 [SMILES: *n1c2ccccc2c2c3sc4c5ccccc5ccc4c3ccc21 ; *n1c2ccccc2c2c3sc4cc5ccccc5cc4c3ccc21 ; *n1c2ccccc2c2c3sc4ccc5ccccc5c4c3ccc21 ; *n1c2ccccc2c2c3sc4ccccc4c3c3ccccc3c21]

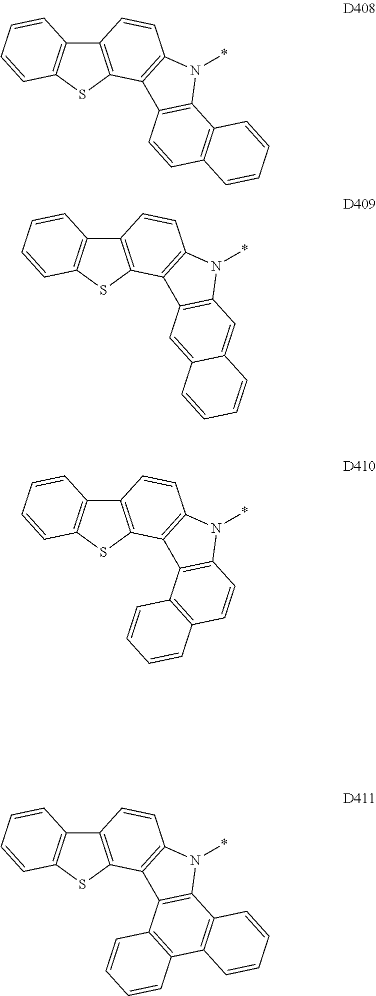 [SMILES: *n1c2ccc3c4ccccc4sc3c2c2ccc3ccccc3c21 ; *n1c2cc3ccccc3cc2c2c3sc4ccccc4c3ccc21 ; *n1c2ccc3ccccc3c2c2c3sc4ccccc4c3ccc21 ; *n1c2ccc3c4ccccc4sc3c2c2c3ccccc3c3ccccc3c21]

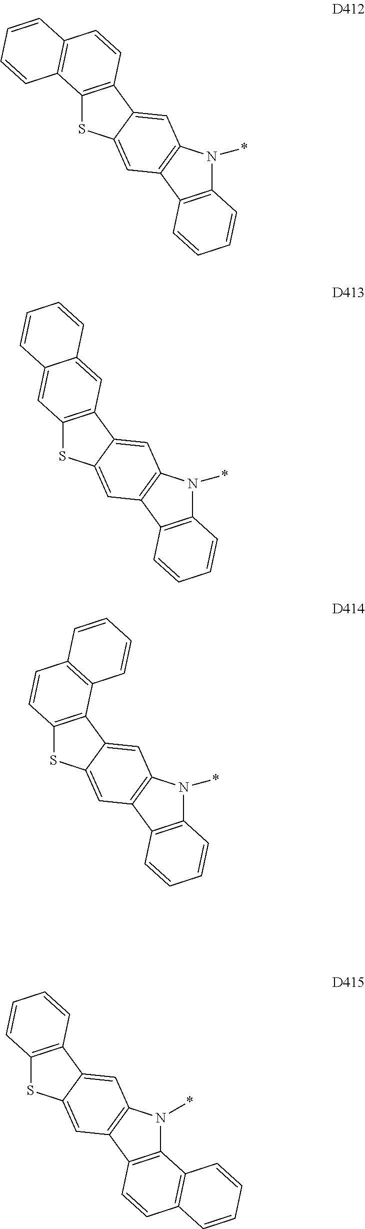 [SMILES: *n1c2ccccc2c2cc3sc4c5ccccc5ccc4c3cc21 ; *n1c2ccccc2c2cc3sc4cc5ccccc5cc4c3cc21 ; *n1c2ccccc2c2cc3sc4ccc5ccccc5c4c3cc21 ; *n1c2cc3c(cc2c2ccc4ccccc4c21)sc1ccccc13]

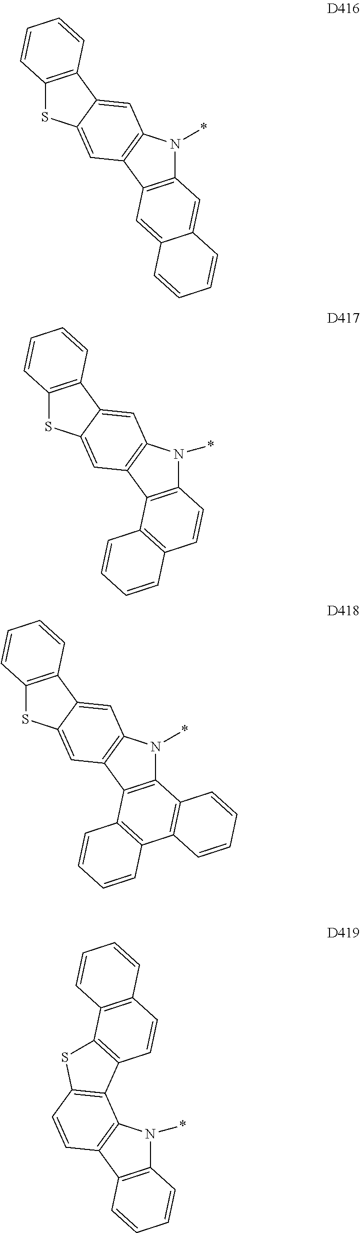 [SMILES: *n1c2cc3ccccc3cc2c2cc3sc4ccccc4c3cc21 ; *n1c2cc3c(cc2c2c4ccccc4ccc21)sc1ccccc13 ; *n1c2cc3c(cc2c2c4ccccc4c4ccccc4c21)sc1ccccc13 ; *n1c2ccccc2c2ccc3sc4c5ccccc5ccc4c3c21]

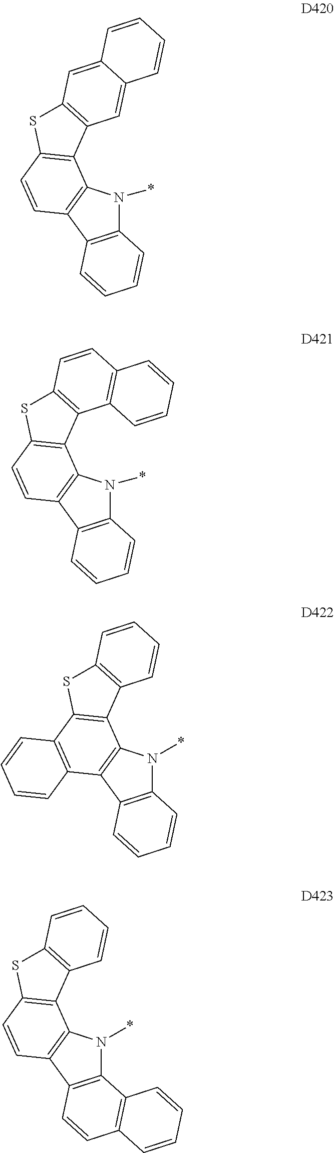 [SMILES: *n1c2ccccc2c2ccc3sc4cc5ccccc5cc4c3c21 ; *n1c2ccccc2c2ccc3sc4ccc5ccccc5c4c3c21 ; *n1c2ccccc2c2c3ccccc3c3sc4ccccc4c3c21 ; *n1c2c3ccccc3ccc2c2ccc3sc4ccccc4c3c21]

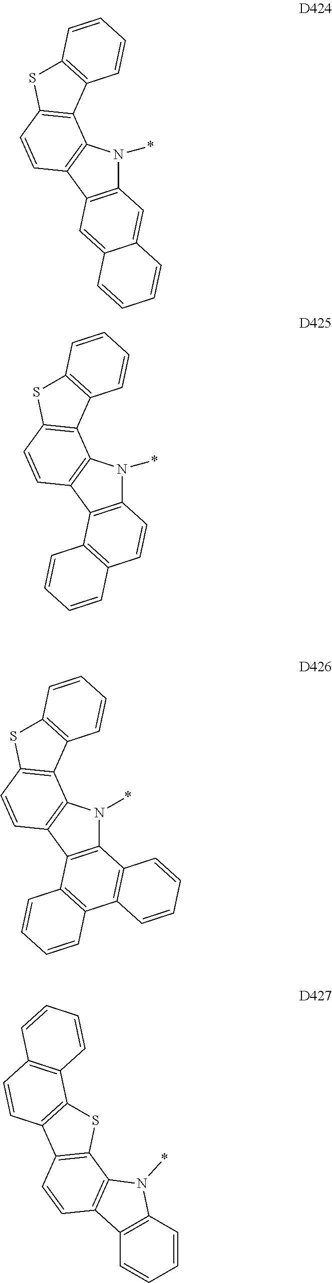

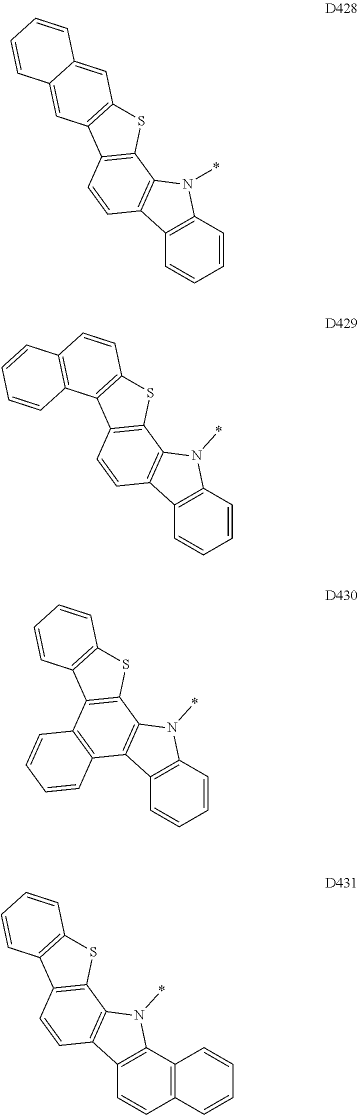 [SMILES: *n1c2ccccc2c2ccc3c4cc5ccccc5cc4sc3c21 ; *n1c2ccccc2c2ccc3c(sc4ccc5ccccc5c43)c21 ; *n1c2ccccc2c2c3ccccc3c3c4ccccc4sc3c21 ; *n1c2c3ccccc3ccc2c2ccc3c4ccccc4sc3c21]

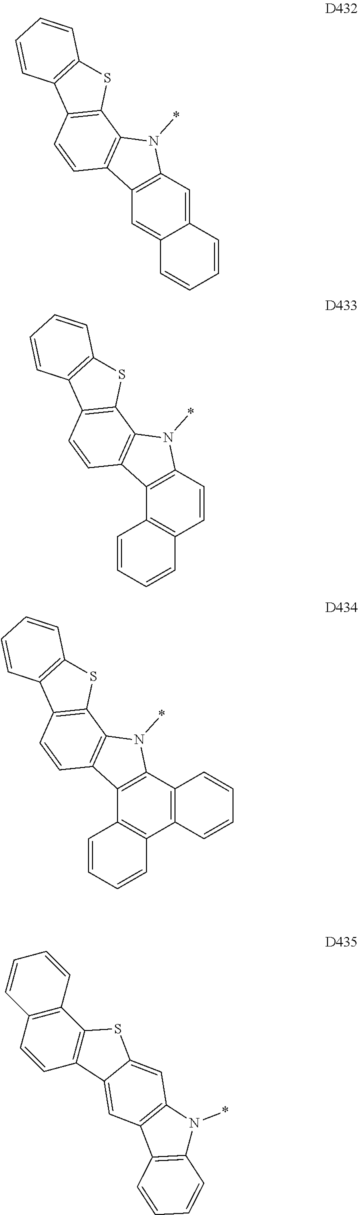 [SMILES: *n1c2cc3ccccc3cc2c2ccc3c4ccccc4sc3c21 ; *n1c2ccc3ccccc3c2c2ccc3c4ccccc4sc3c21 ; *n1c2c(ccc3c4ccccc4sc32)c2c3ccccc3c3ccccc3c21 ; *n1c2ccccc2c2cc3c(cc21)sc1c2ccccc2ccc31]

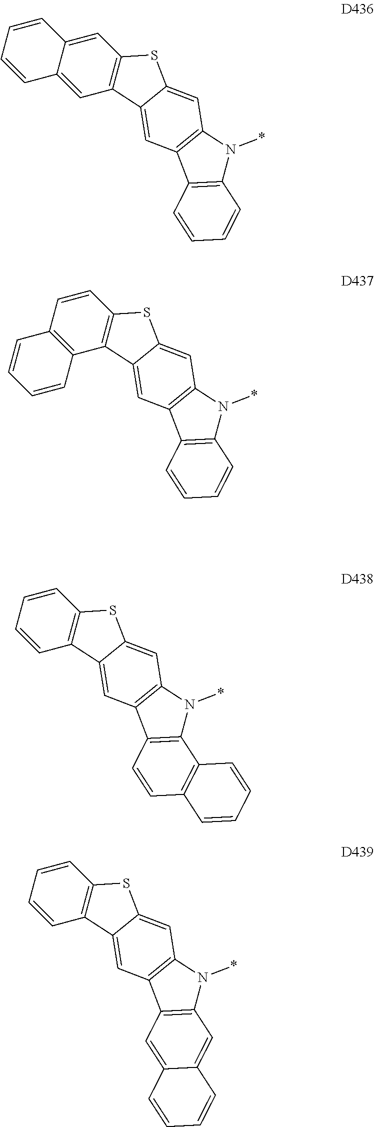 [SMILES: *n1c2ccccc2c2cc3c(cc21)sc1cc2ccccc2cc13 ; *n1c2ccccc2c2cc3c(cc21)sc1ccc2ccccc2c13 ; *n1c2cc3sc4ccccc4c3cc2c2ccc3ccccc3c21 ; *n1c2cc3ccccc3cc2c2cc3c(cc21)sc1ccccc13]

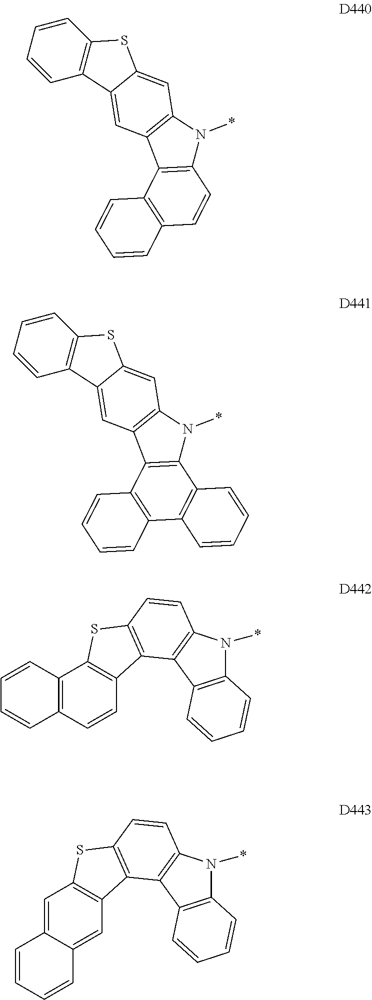 [SMILES: *n1c2cc3sc4ccccc4c3cc2c2c3ccccc3ccc21 ; *n1c2cc3sc4ccccc4c3cc2c2c3ccccc3c3ccccc3c21 ; *n1c2ccccc2c2c3c(ccc21)sc1c2ccccc2ccc13 ; *n1c2ccccc2c2c3c(ccc21)sc1cc2ccccc2cc13]

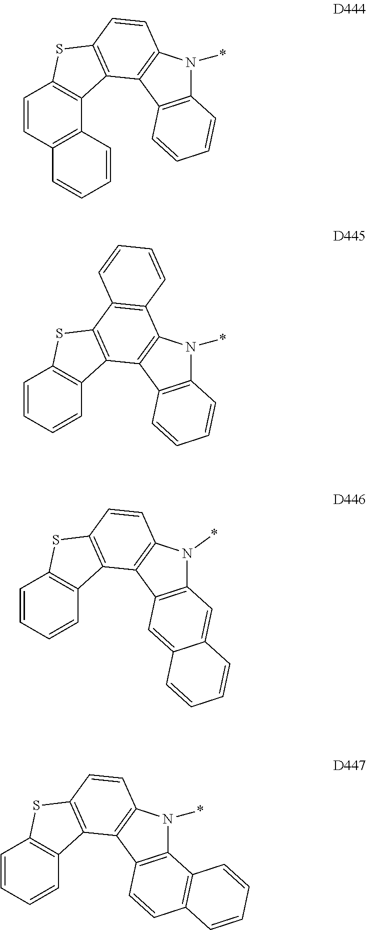 [SMILES: *n1c2ccccc2c2c3c(ccc21)sc1ccc2ccccc2c13 ; *n1c2ccccc2c2c3c4ccccc4sc3c3ccccc3c21 ; *n1c2cc3ccccc3cc2c2c3c(ccc21)sc1ccccc13 ; *n1c2ccc3sc4ccccc4c3c2c2ccc3ccccc3c21]

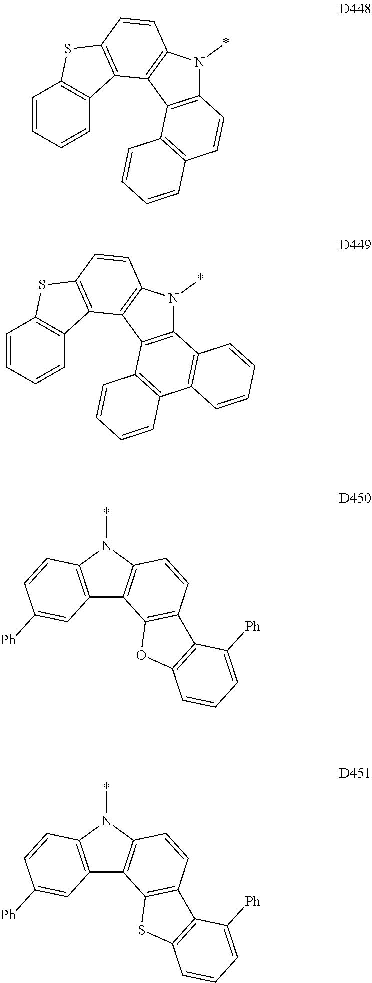 [SMILES: *n1c2ccc3ccccc3c2c2c3c(ccc21)sc1ccccc13 ; *n1c2ccc3sc4ccccc4c3c2c2c3ccccc3c3ccccc3c21 ; *n1c2ccc(-c3ccccc3)cc2c2c3oc4cccc(-c5ccccc5)c4c3ccc21 ; *n1c2ccc(-c3ccccc3)cc2c2c3sc4cccc(-c5ccccc5)c4c3ccc21]

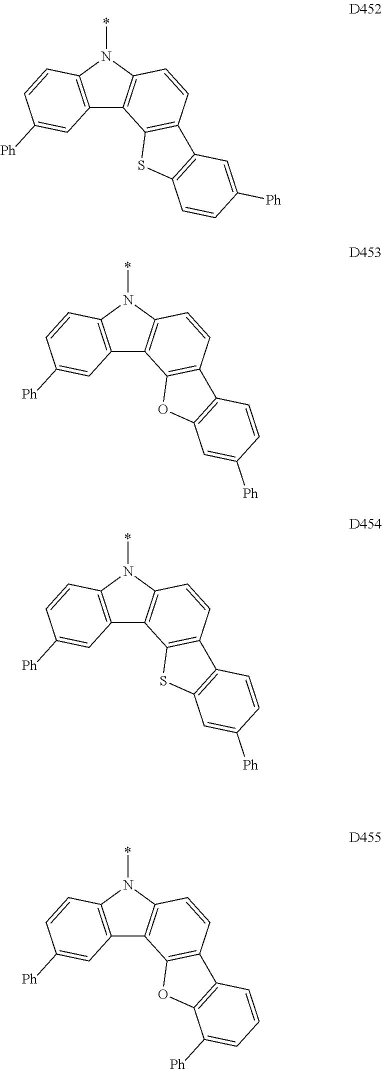 [SMILES: *n1c2ccc(-c3ccccc3)cc2c2c3sc4ccc(-c5ccccc5)cc4c3ccc21 ; *n1c2ccc(-c3ccccc3)cc2c2c3oc4cc(-c5ccccc5)ccc4c3ccc21 ; *n1c2ccc(-c3ccccc3)cc2c2c3sc4cc(-c5ccccc5)ccc4c3ccc21 ; *n1c2ccc(-c3ccccc3)cc2c2c3oc4c(-c5ccccc5)cccc4c3ccc21]

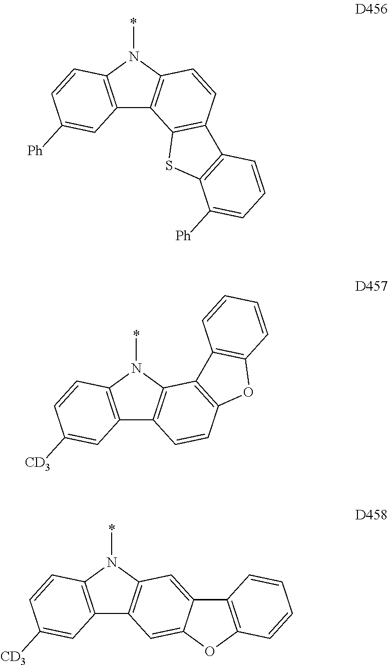 [SMILES: *n1c2ccc(-c3ccccc3)cc2c2c3sc4c(-c5ccccc5)cccc4c3ccc21 ; *n1c2ccc(C([2H])([2H])[2H])cc2c2ccc3oc4ccccc4c3c21 ; *n1c2ccc(C([2H])([2H])[2H])cc2c2cc3oc4ccccc4c3cc21]

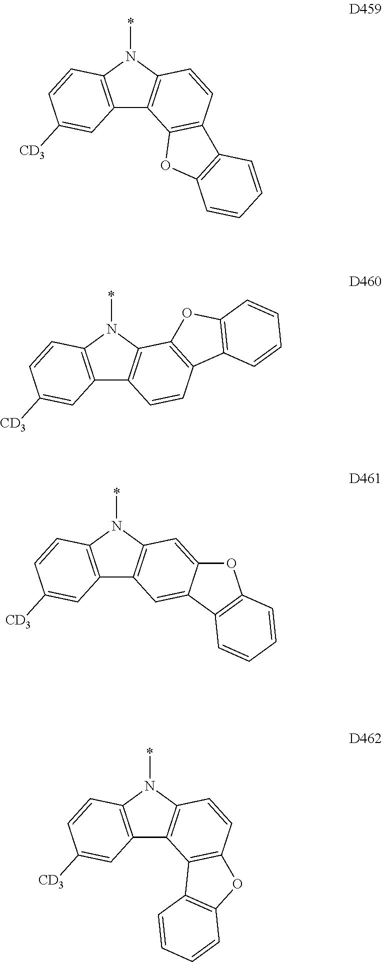 [SMILES: *n1c2ccc(C([2H])([2H])[2H])cc2c2c3oc4ccccc4c3ccc21 ; *n1c2ccc(C([2H])([2H])[2H])cc2c2ccc3c4ccccc4oc3c21 ; *n1c2ccc(C([2H])([2H])[2H])cc2c2cc3c(cc21)oc1ccccc13 ; *n1c2ccc(C([2H])([2H])[2H])cc2c2c3c(ccc21)oc1ccccc13]

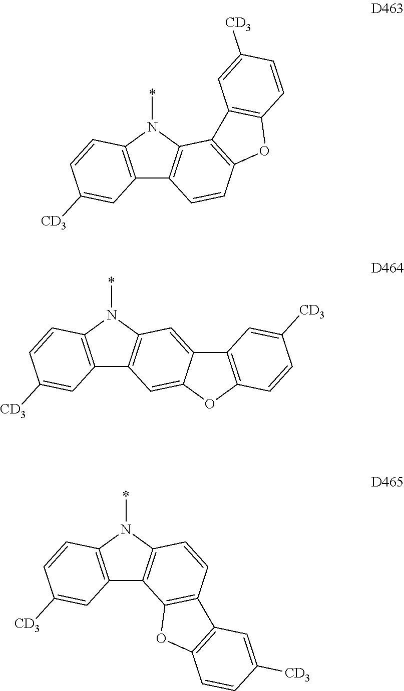 [SMILES: *n1c2ccc(C([2H])([2H])[2H])cc2c2ccc3oc4ccc(C([2H])([2H])[2H])cc4c3c21 ; *n1c2ccc(C([2H])([2H])[2H])cc2c2cc3oc4ccc(C([2H])([2H])[2H])cc4c3cc21 ; *n1c2ccc(C([2H])([2H])[2H])cc2c2c3oc4ccc(C([2H])([2H])[2H])cc4c3ccc21]

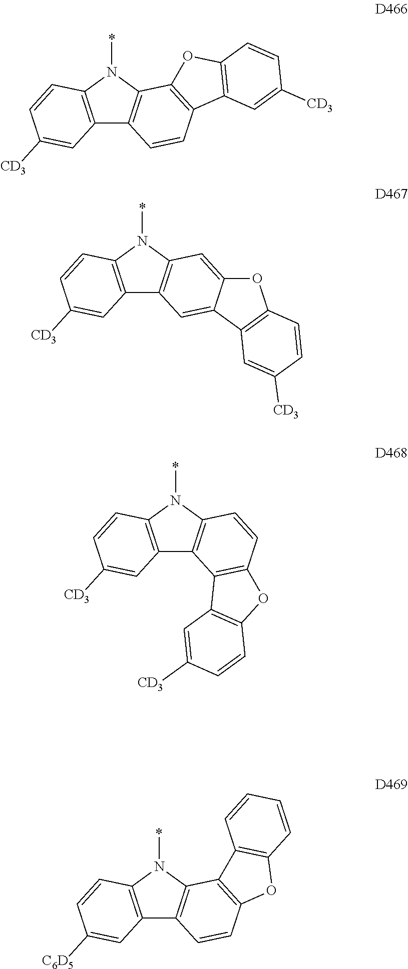

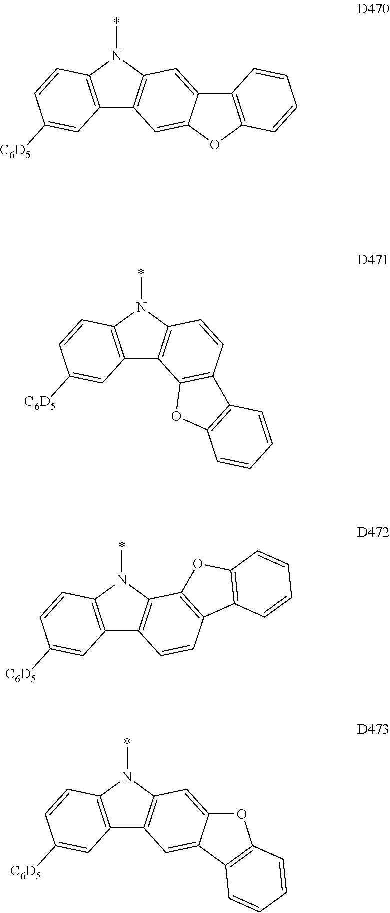

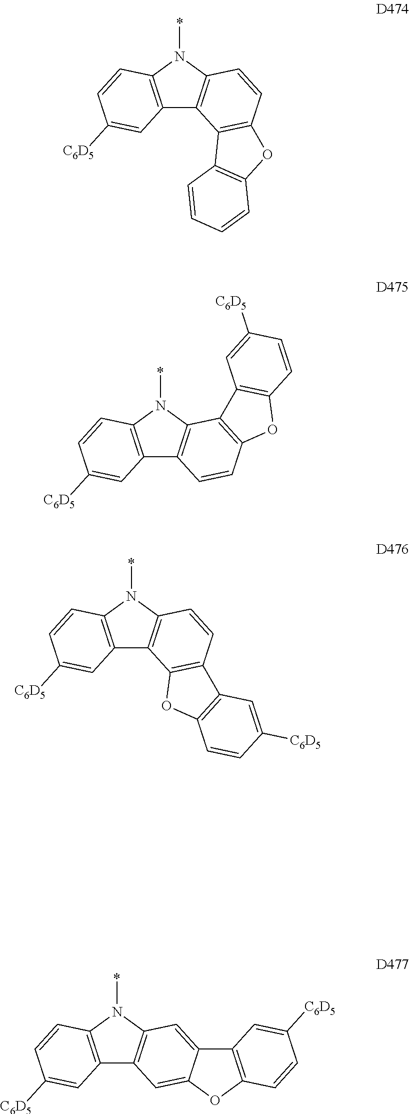

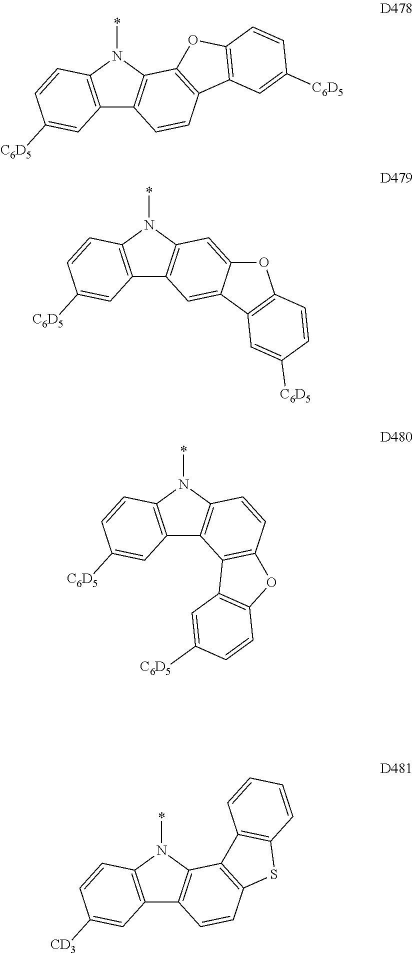

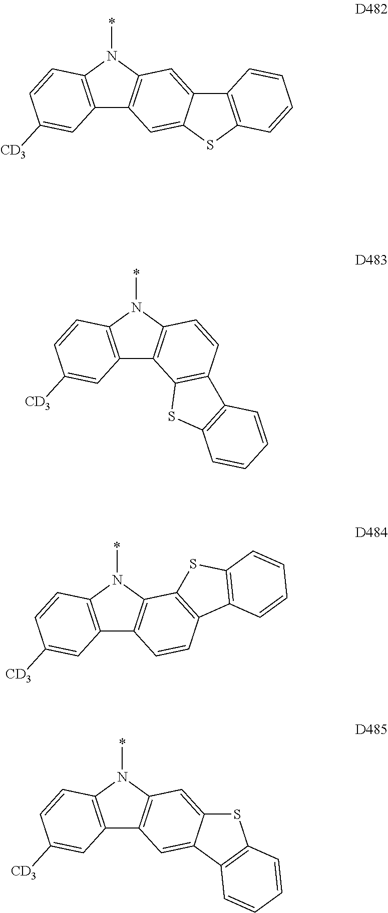 [SMILES: *n1c2ccc(C([2H])([2H])[2H])cc2c2cc3sc4ccccc4c3cc21 ; *n1c2ccc(C([2H])([2H])[2H])cc2c2c3sc4ccccc4c3ccc21 ; *n1c2ccc(C([2H])([2H])[2H])cc2c2ccc3c4ccccc4sc3c21 ; *n1c2ccc(C([2H])([2H])[2H])cc2c2cc3c(cc21)sc1ccccc13]

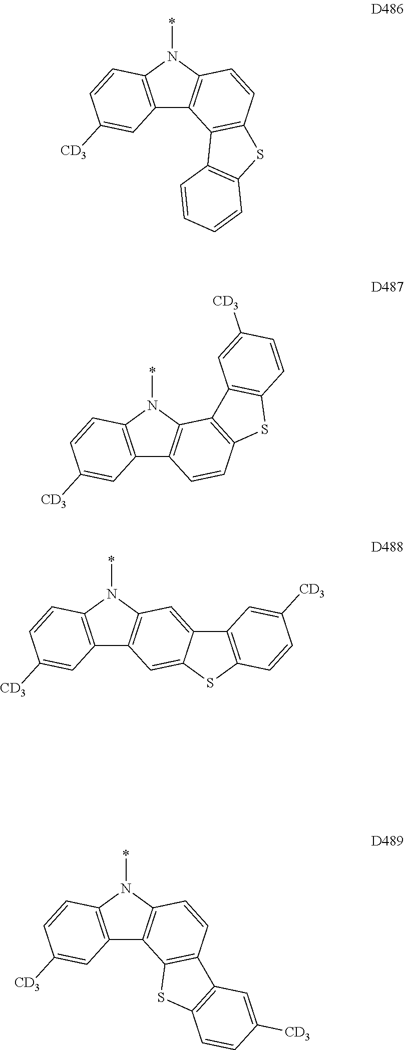 [SMILES: *n1c2ccc(C([2H])([2H])[2H])cc2c2c3c(ccc21)sc1ccccc13 ; *n1c2ccc(C([2H])([2H])[2H])cc2c2ccc3sc4ccc(C([2H])([2H])[2H])cc4c3c21 ; *n1c2ccc(C([2H])([2H])[2H])cc2c2cc3sc4ccc(C([2H])([2H])[2H])cc4c3cc21 ; *n1c2ccc(C([2H])([2H])[2H])cc2c2c3sc4ccc(C([2H])([2H])[2H])cc4c3ccc21]

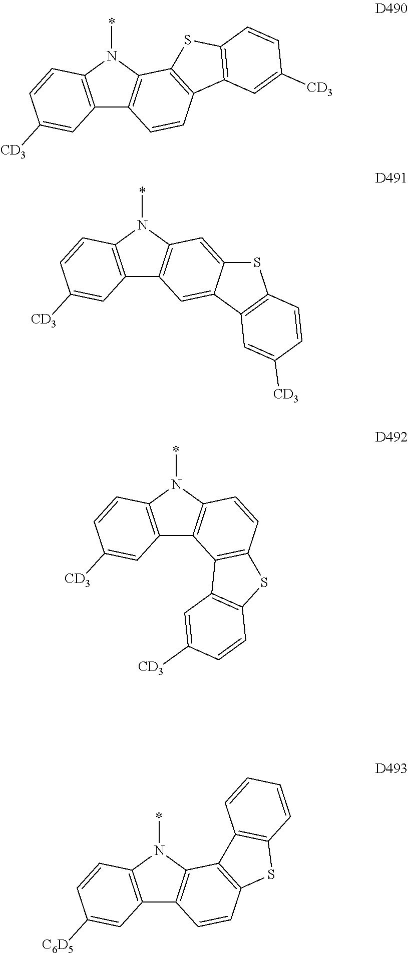

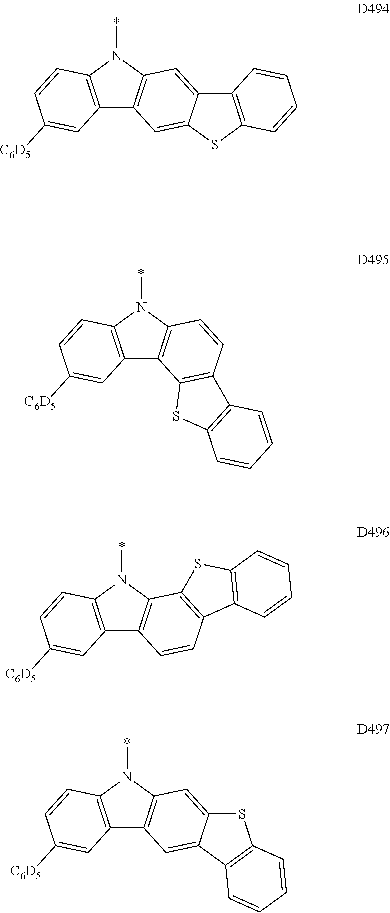

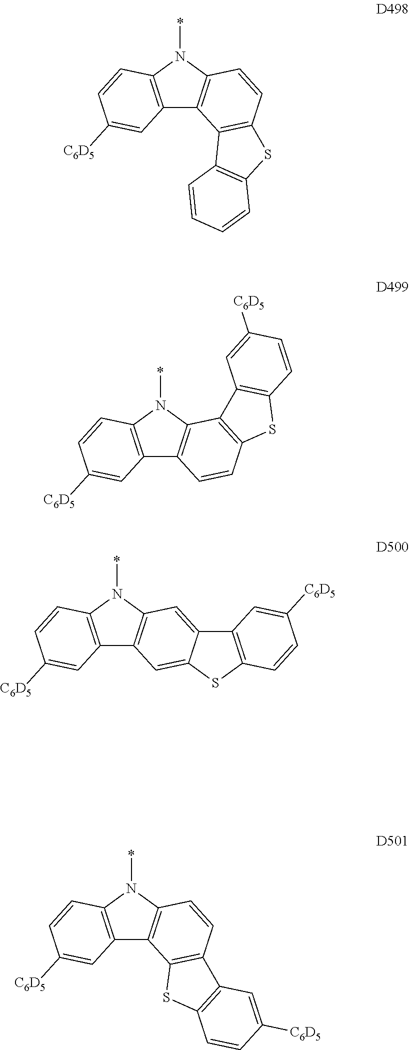

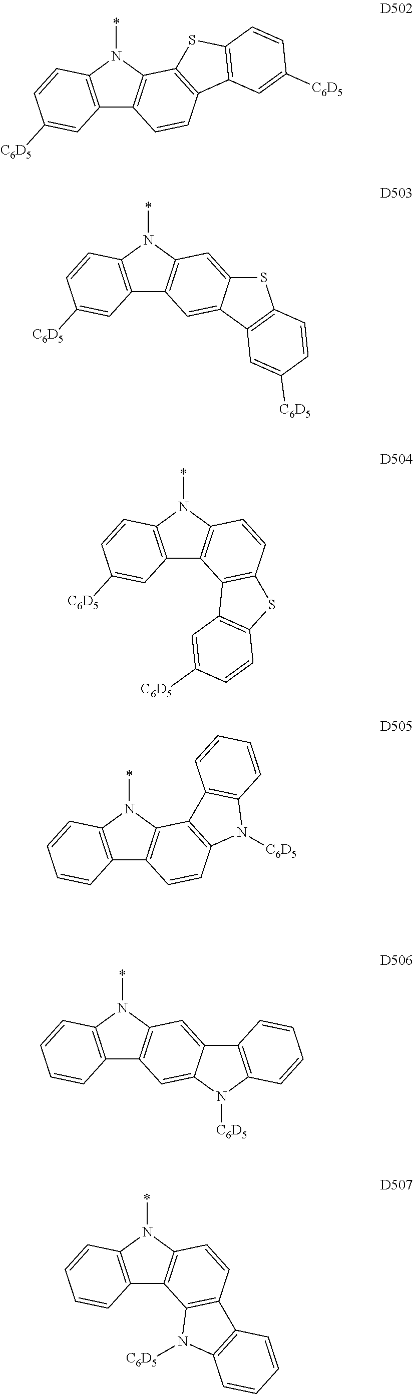

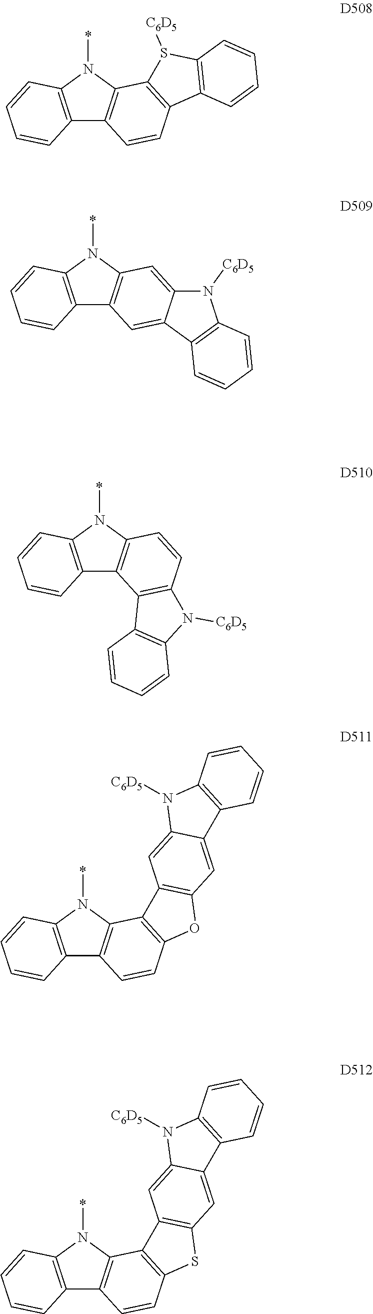

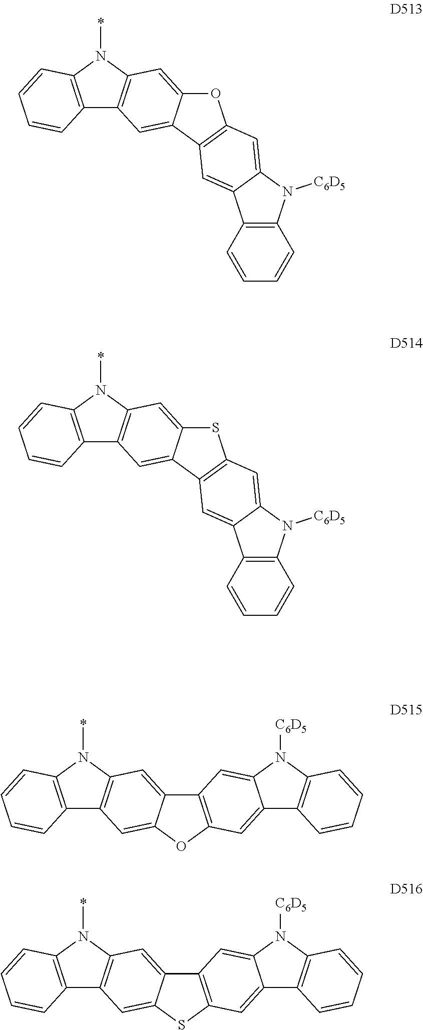

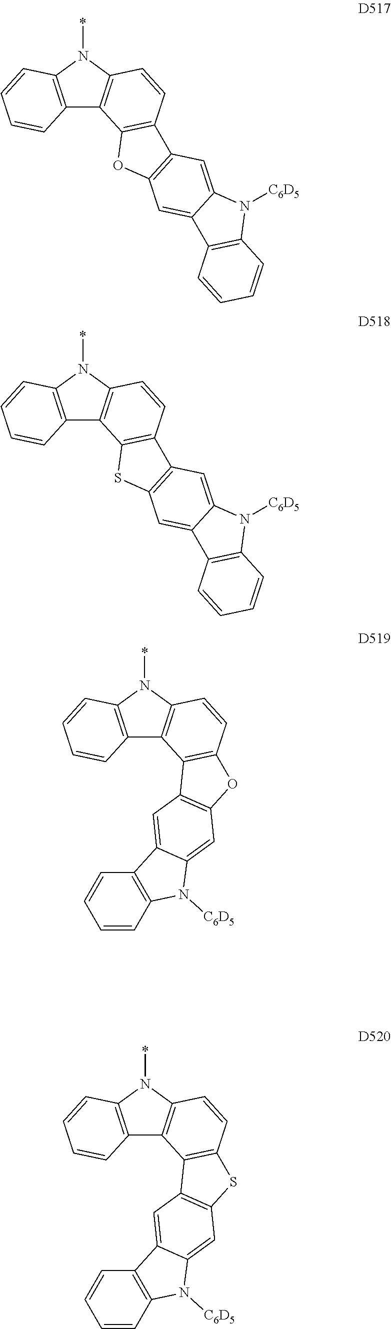

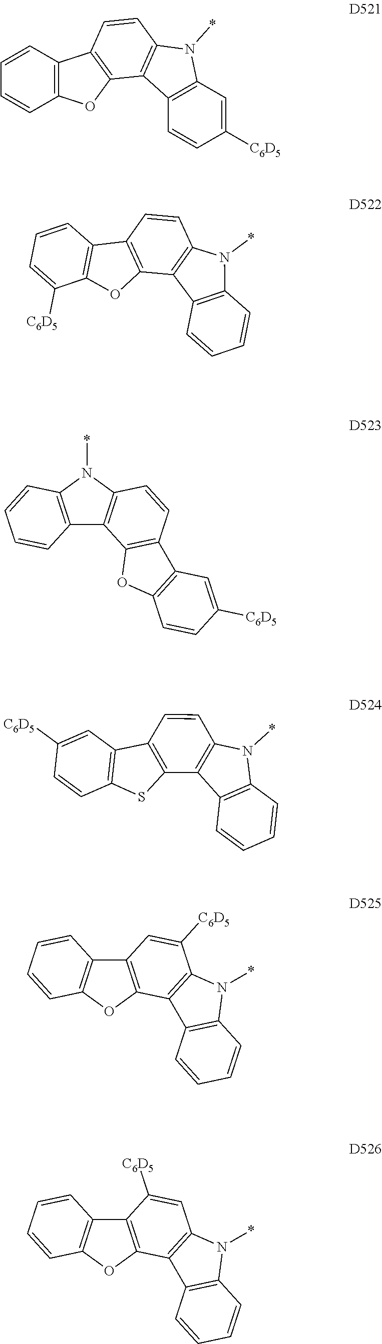

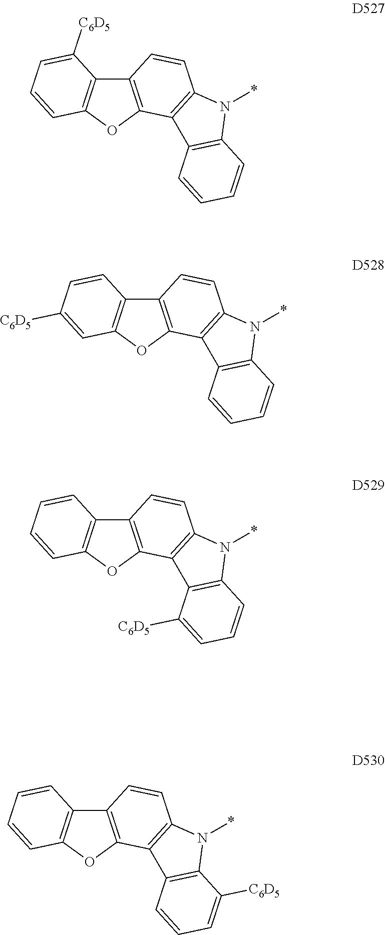

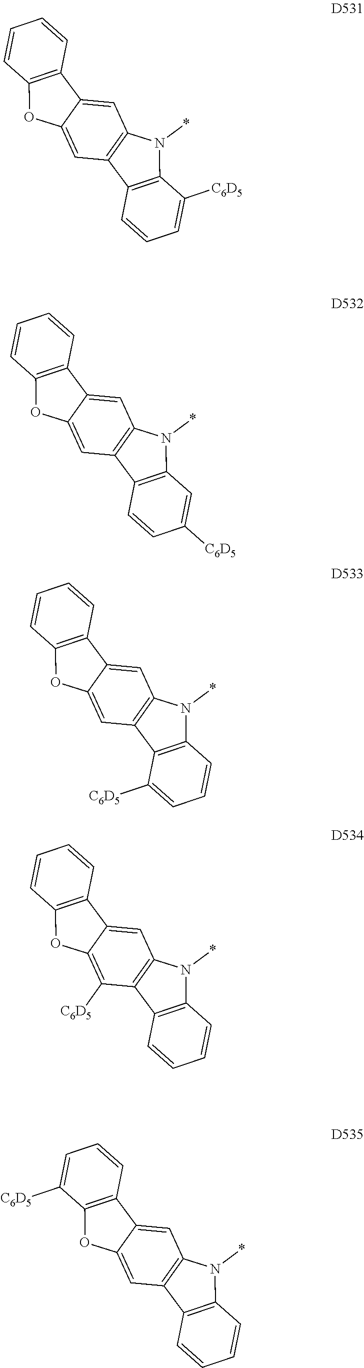

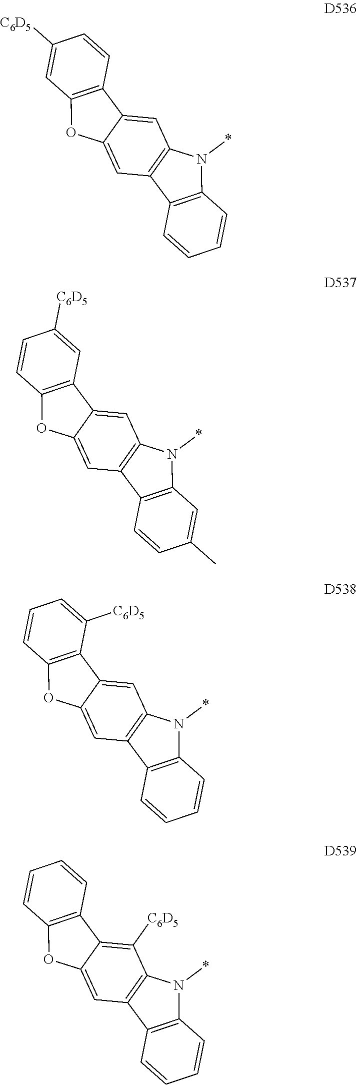

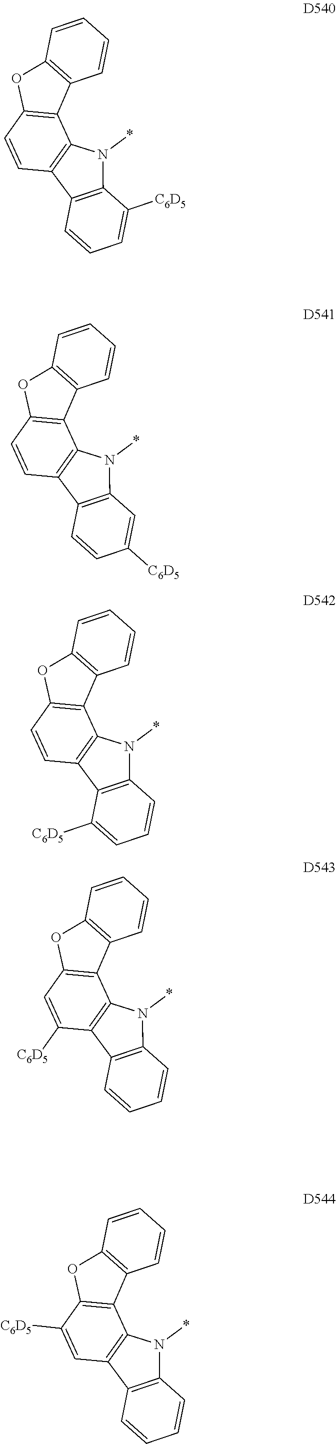

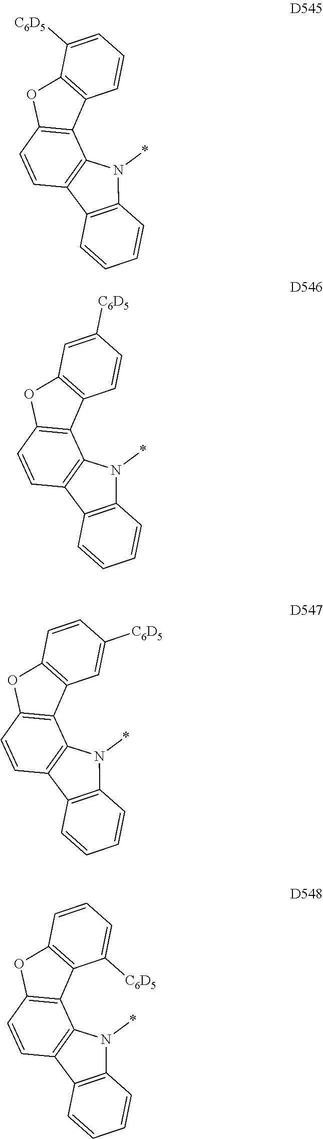

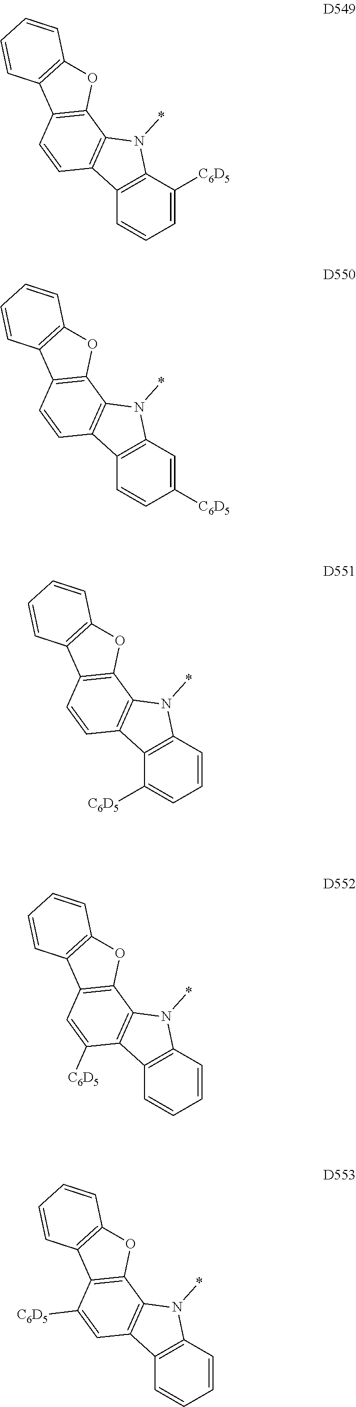

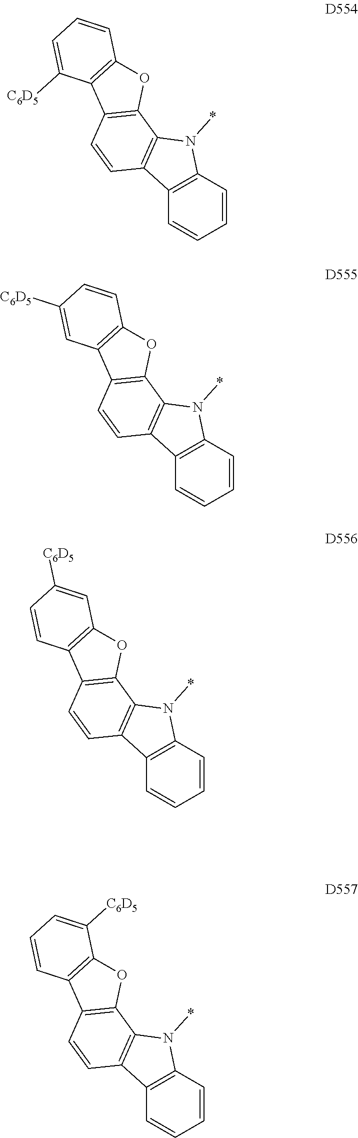

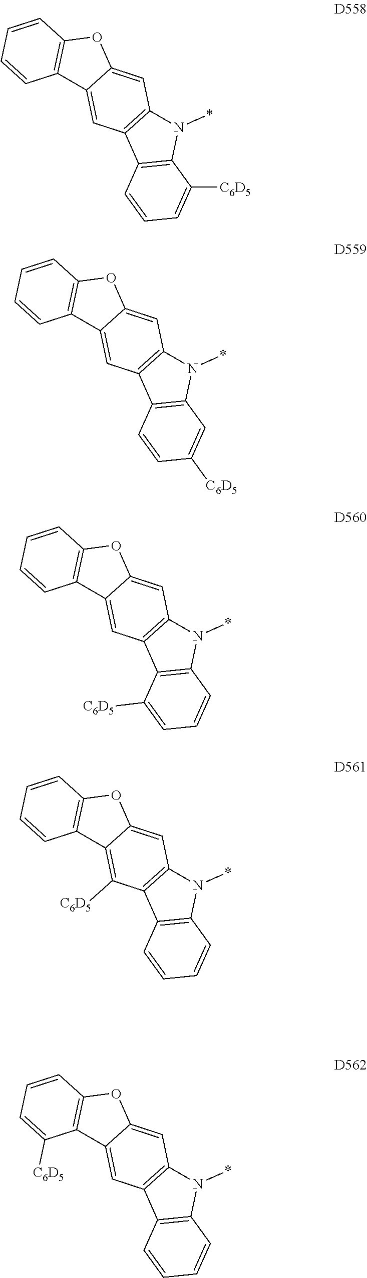

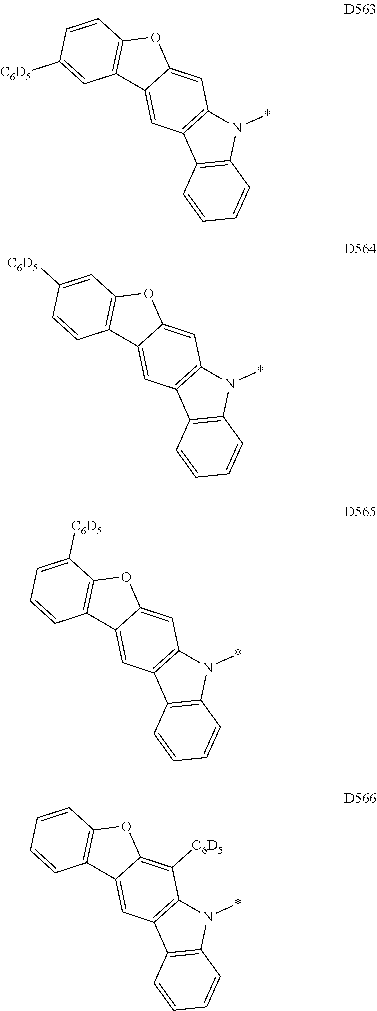

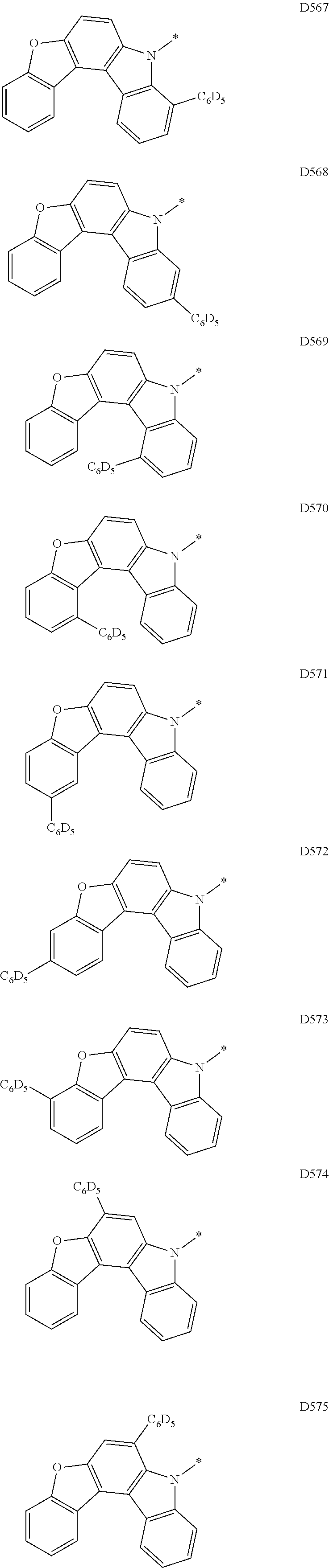

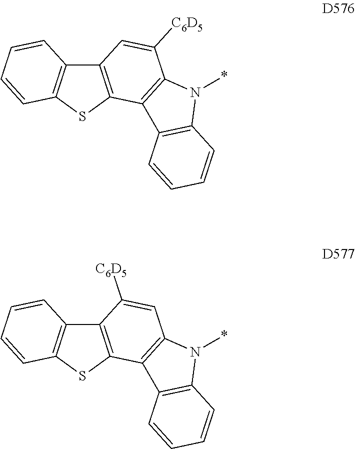

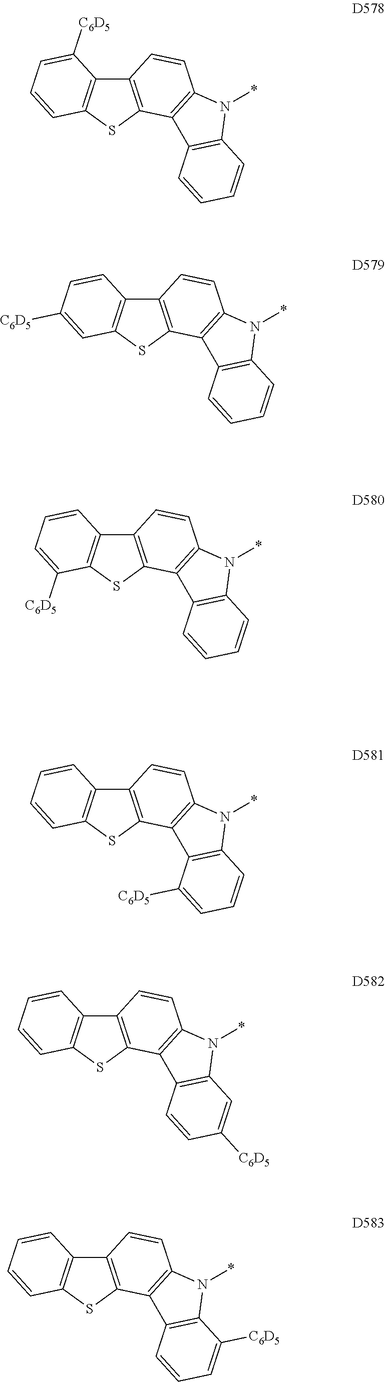

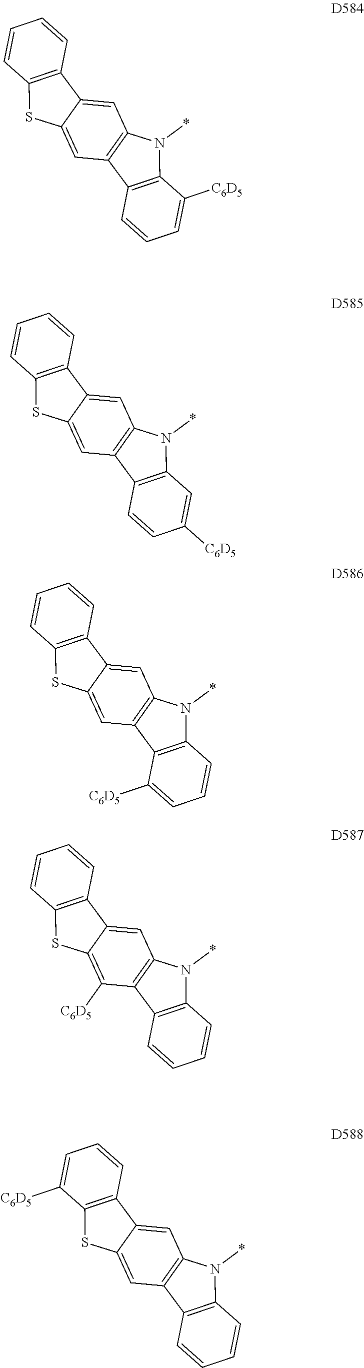

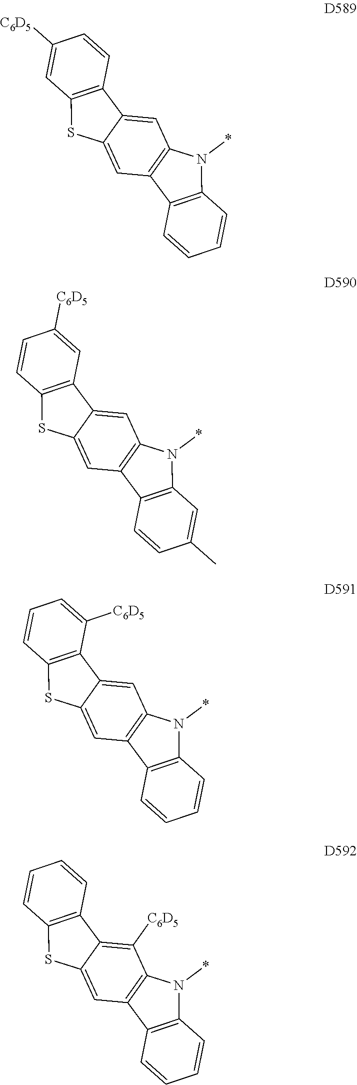

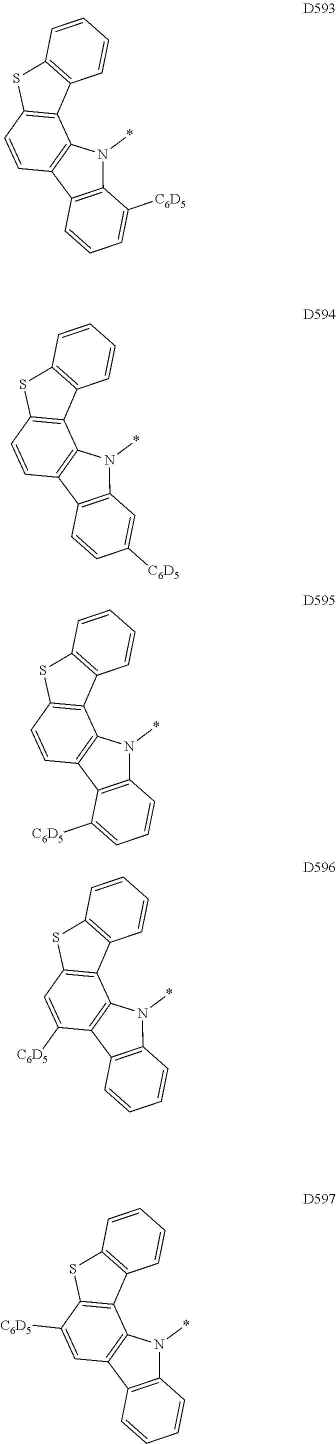

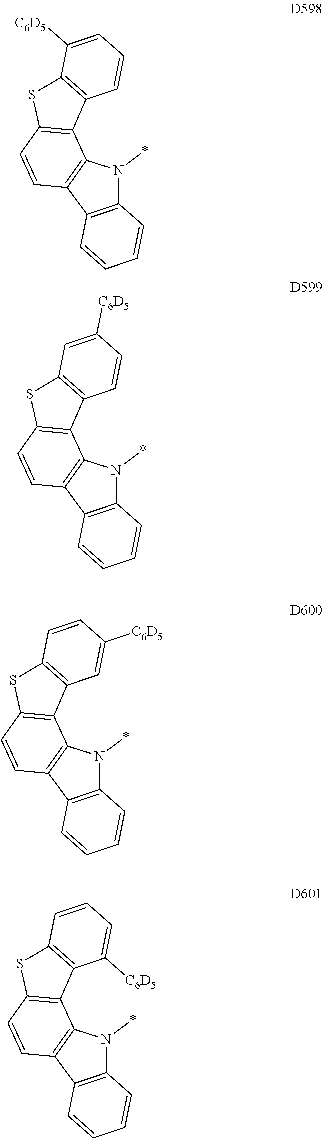

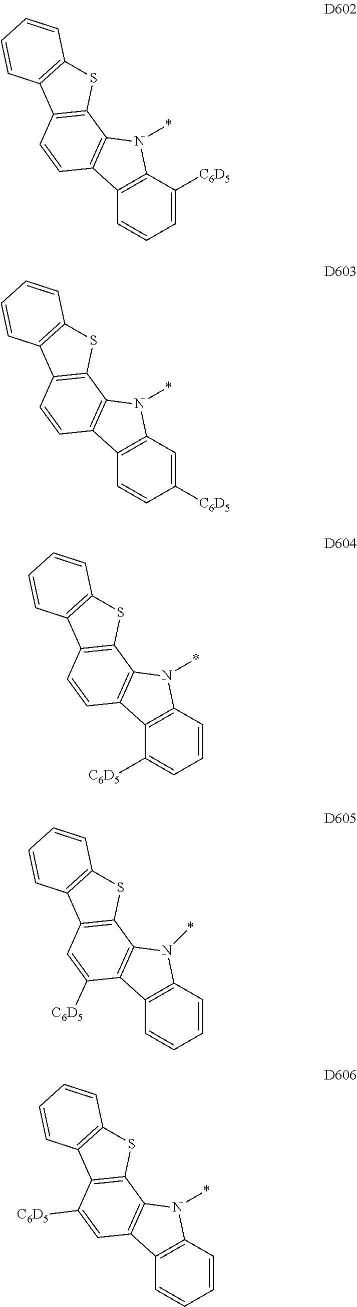

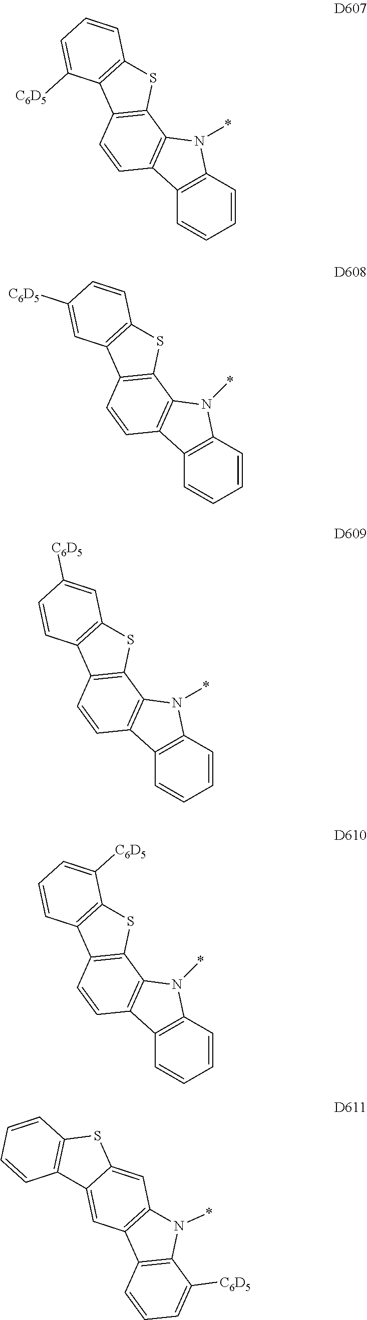

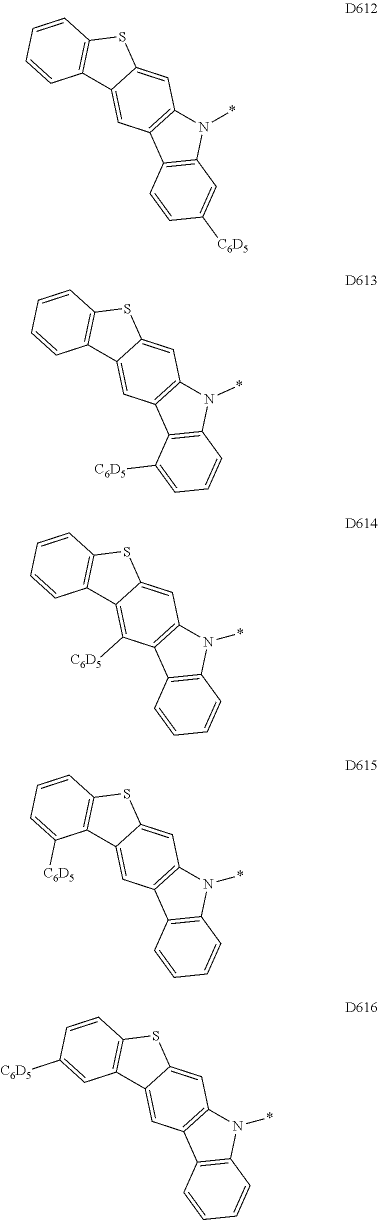

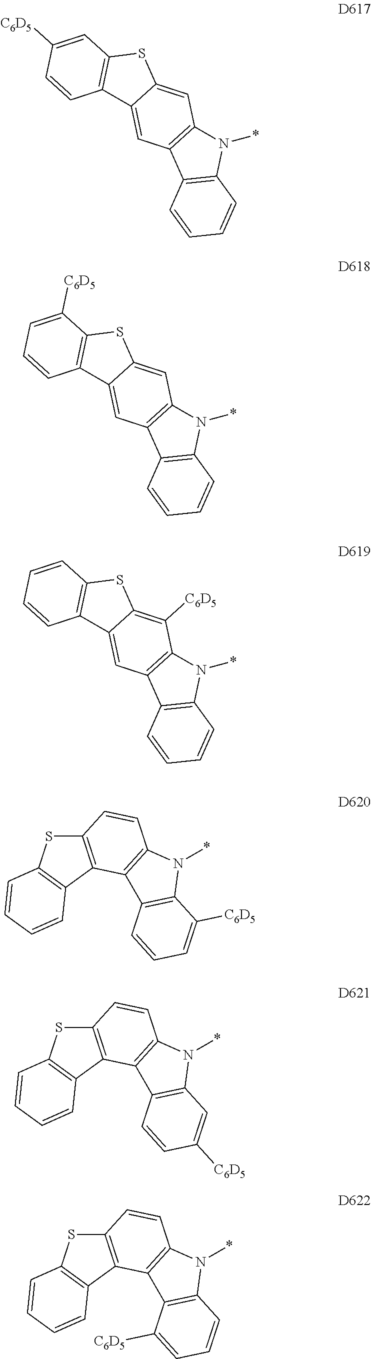

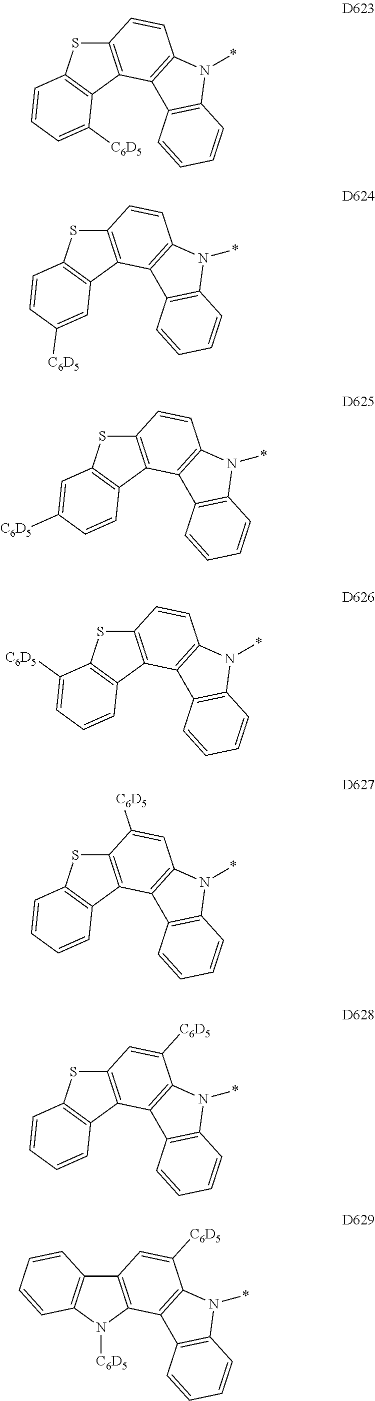

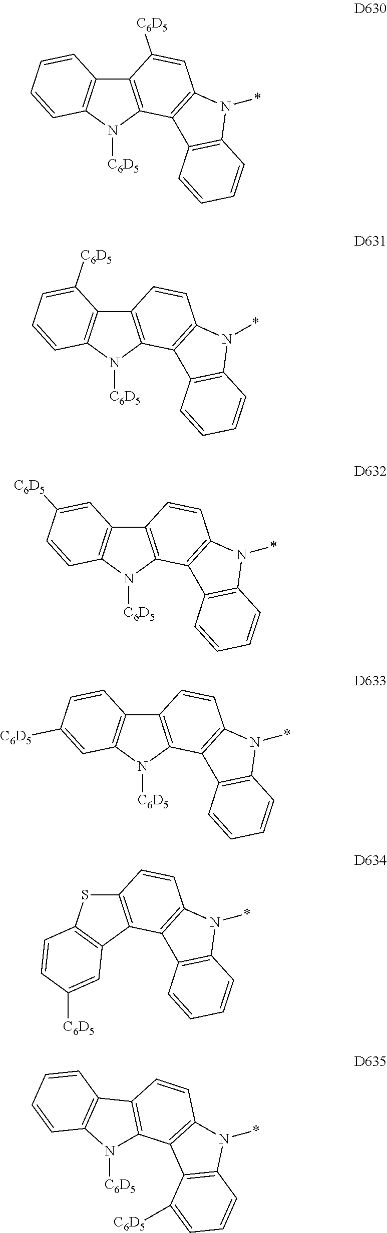

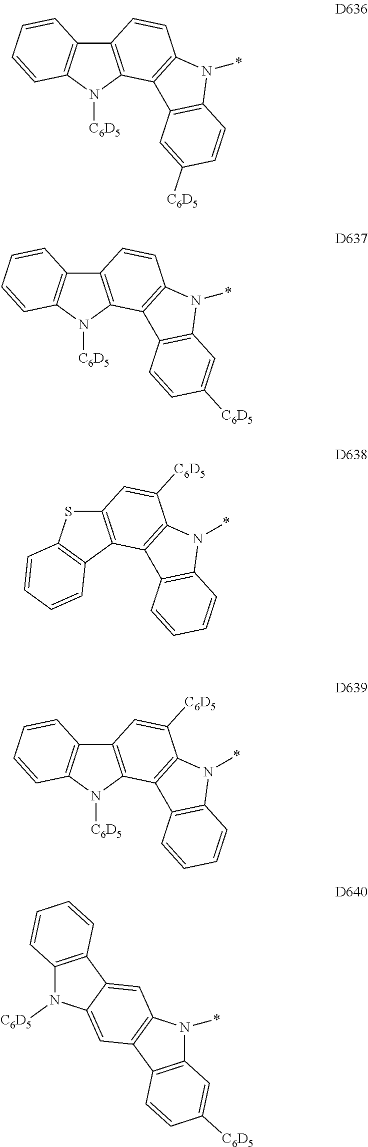

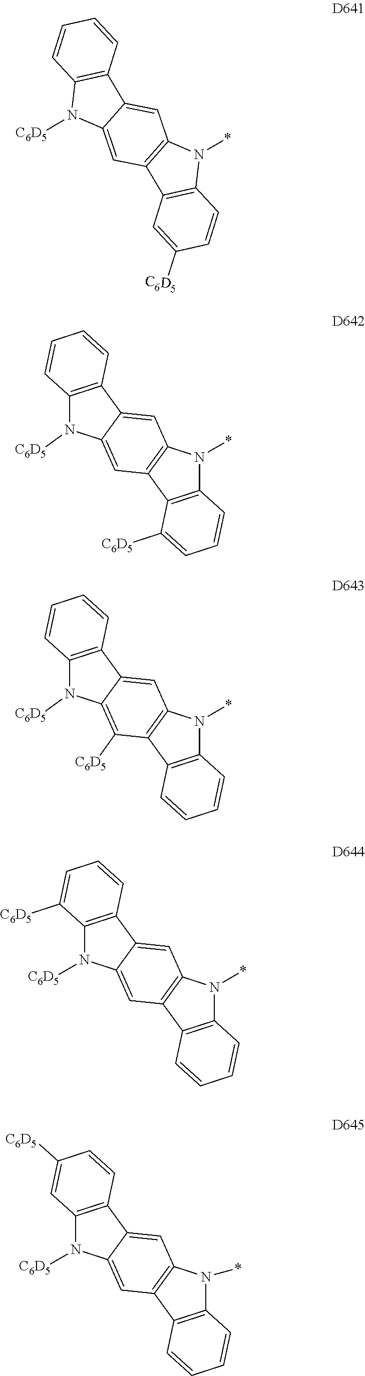

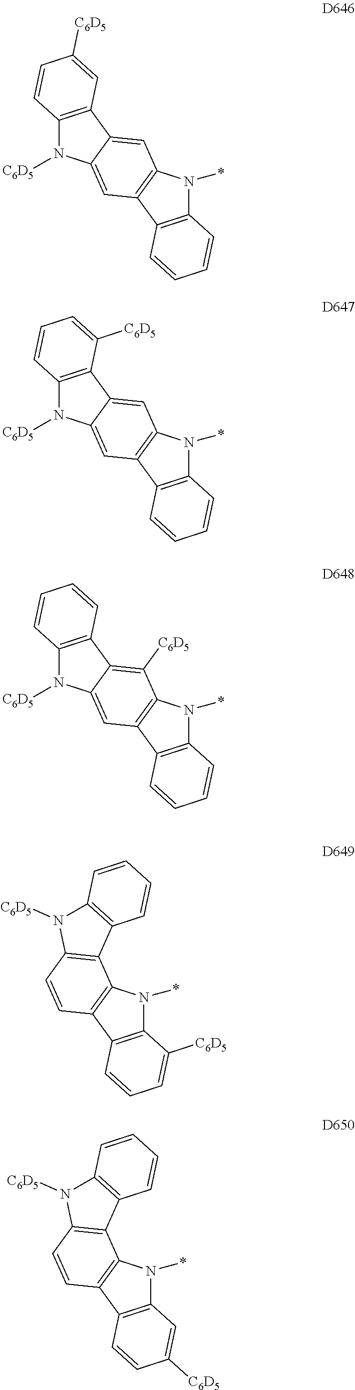

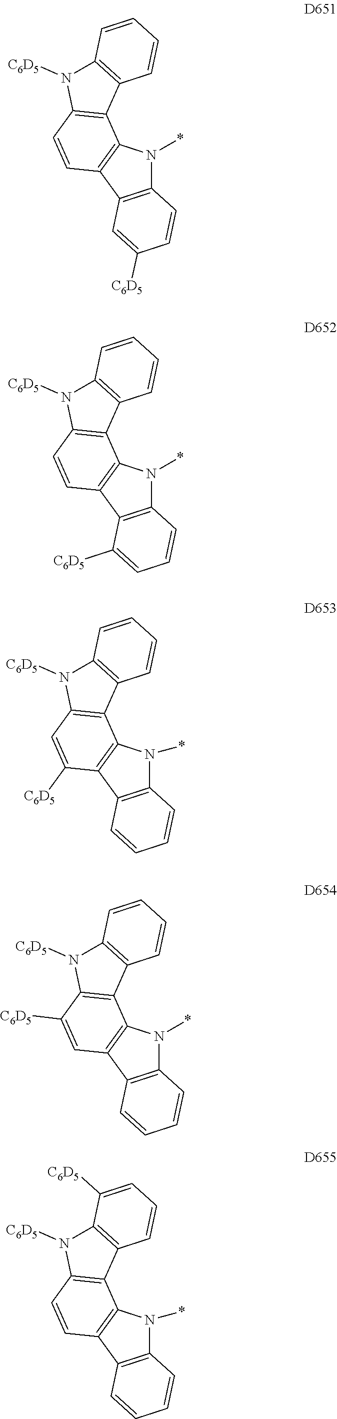

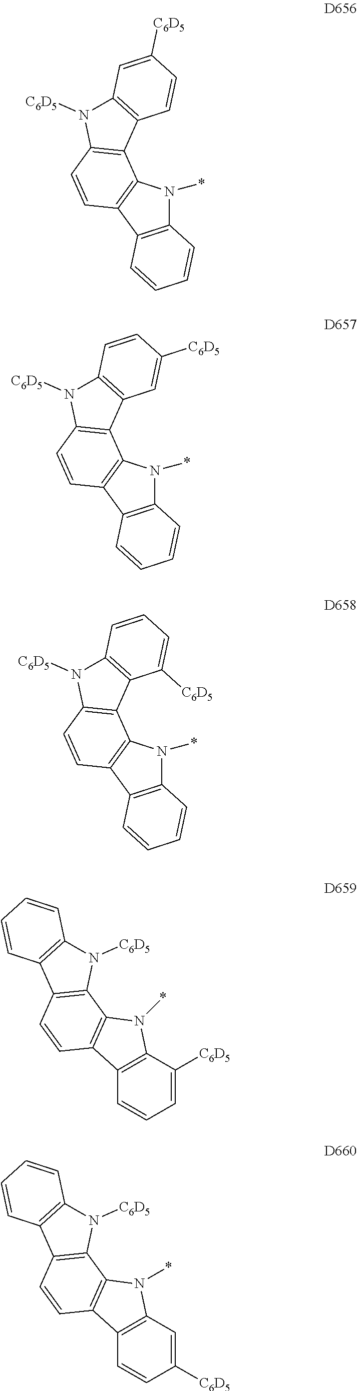

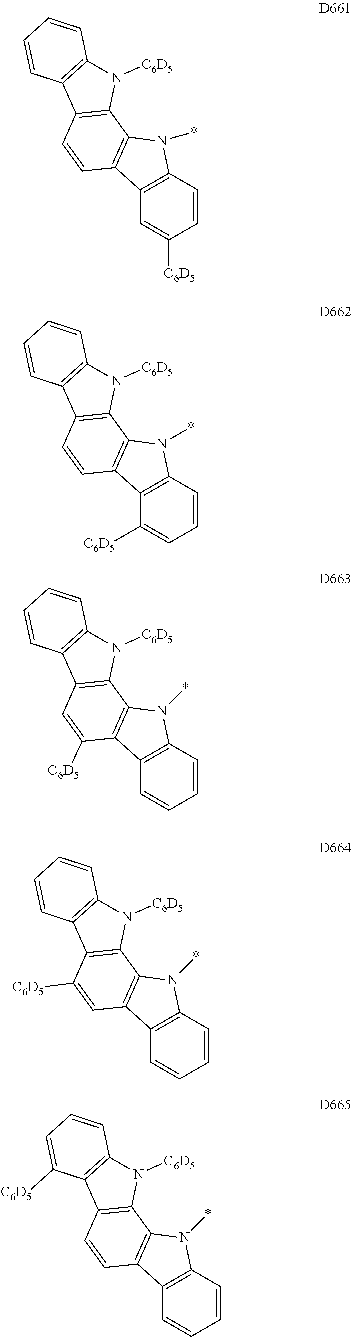

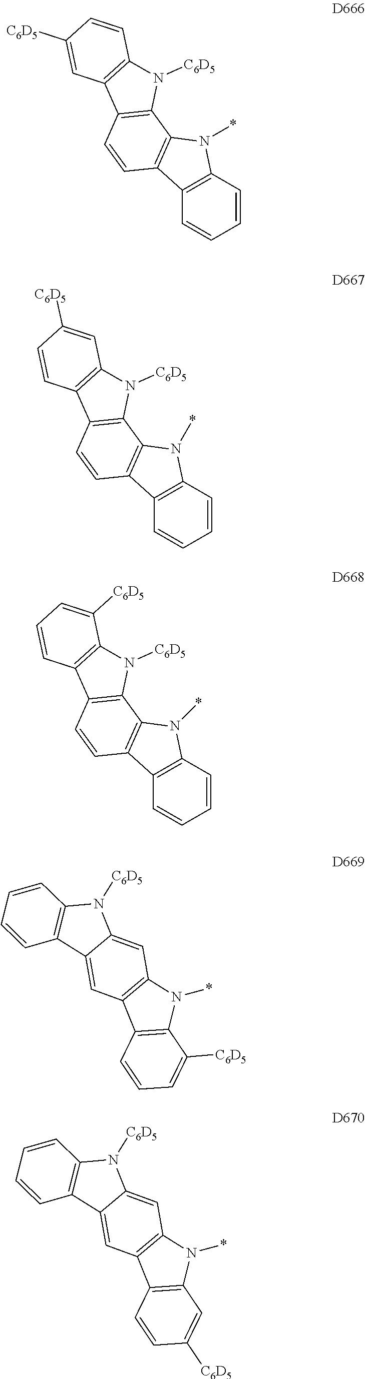

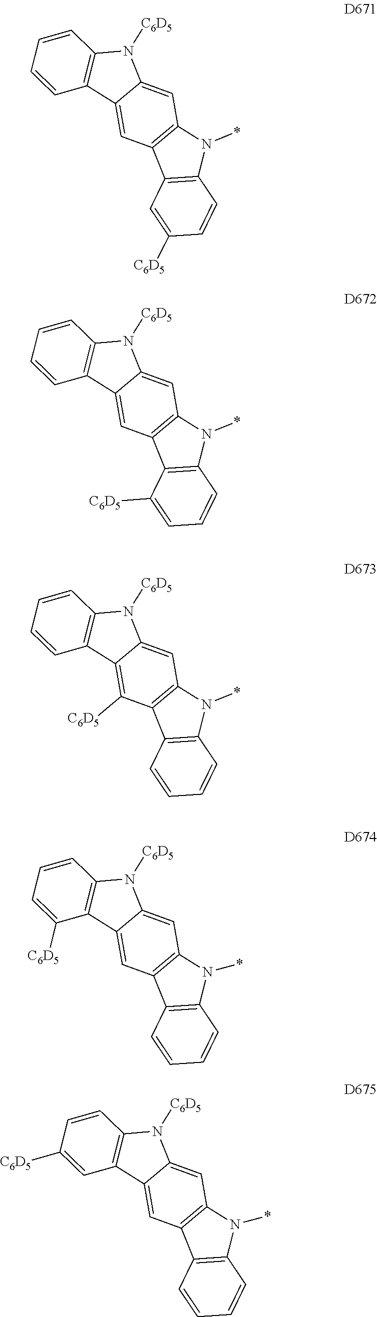

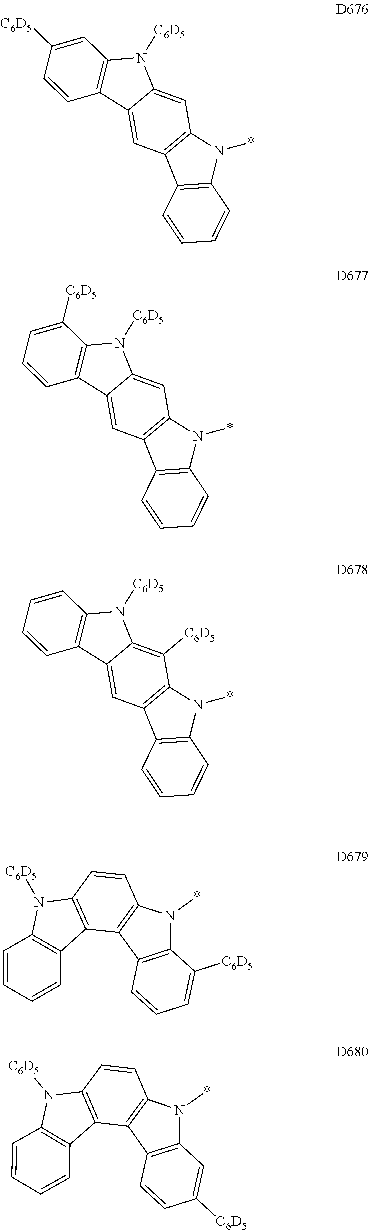

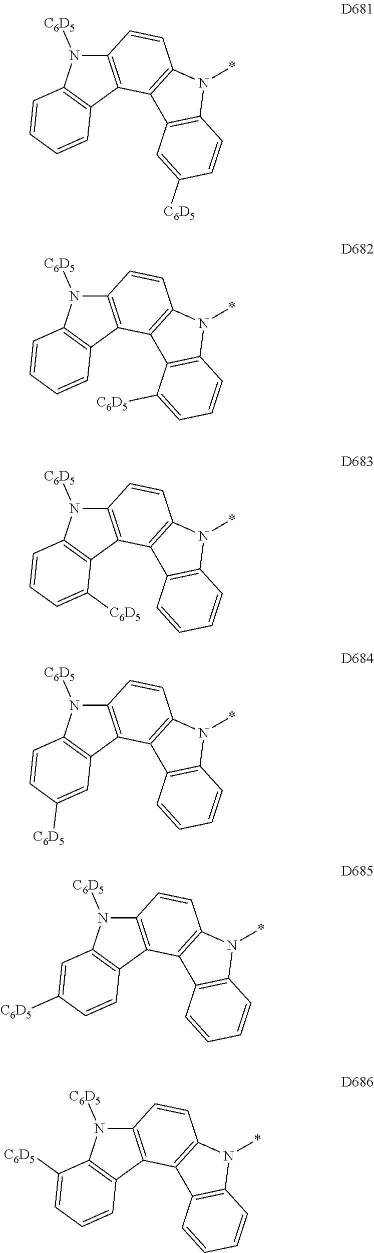

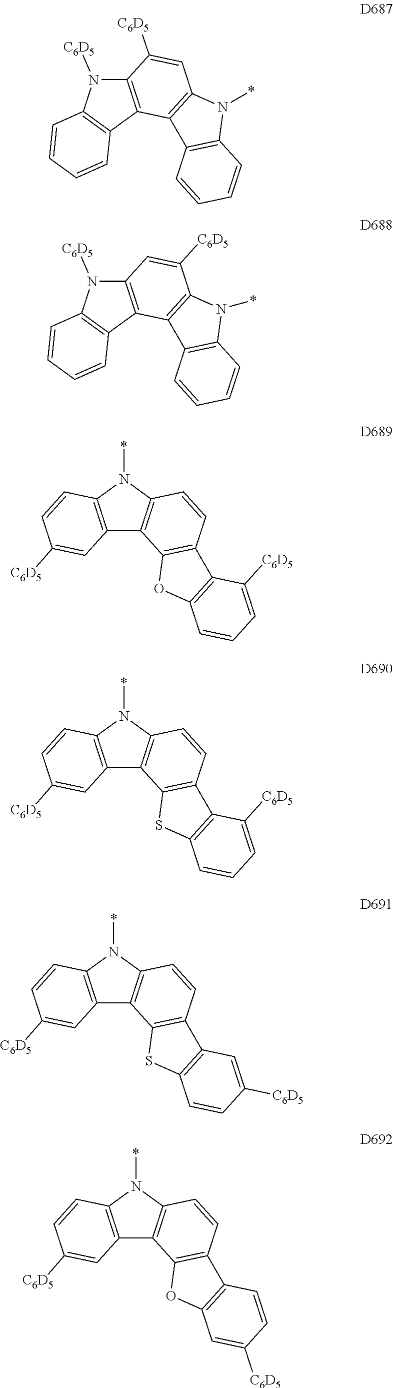

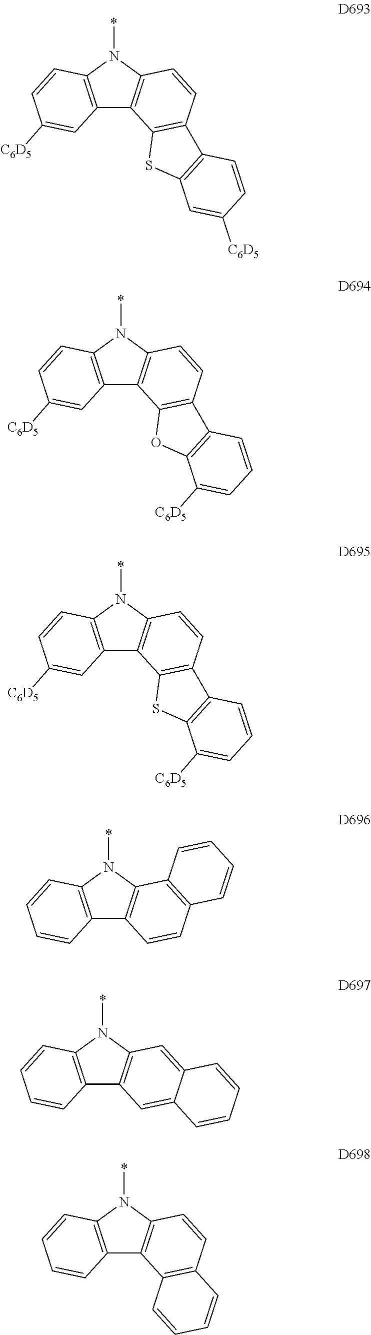

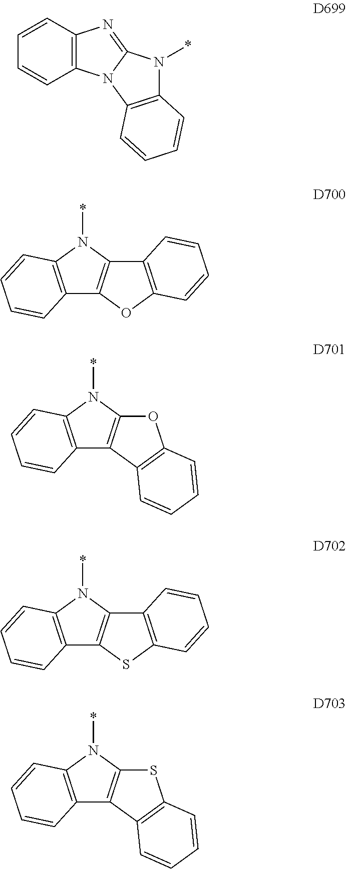 [SMILES: *n1c2ccccc2n2c3ccccc3nc12 ; *n1c2ccccc2c2oc3ccccc3c21 ; *n1c2ccccc2c2c3ccccc3oc21 ; *n1c2ccccc2c2sc3ccccc3c21 ; *n1c2ccccc2c2c3ccccc3sc21]

Groups obtained by substituting all hydrogen atoms present in the above D86 to D456 and D696 to D703 with deuterium atoms are disclosed as D704 to D1082.

In one aspect of the present invention, the donor group that R² to R⁵ can adopt is selected from the group consisting of D1 to D1082. In one aspect of the present invention, the donor group that R² to R⁵ can adopt is selected from the group consisting of D1 to D85. In one aspect of the present invention, the donor group that R² to R⁵ can adopt is selected from the group consisting of D86 to D1082. In one aspect of the present invention, the donor group that R² to R⁵ can adopt is selected from the group consisting of D86 to D695, and D704 to D1074. In one aspect of the present invention, the donor group that R² to R⁵ can adopt is selected from the group consisting of D696 to D703, and D1075 to D1082.

In one aspect of the present invention, at least R² is a donor group. In one aspect of the present invention, at least R³ is a donor group. In one aspect of the present invention, at least R⁴ is a donor group. In one aspect of the present invention, at least R⁵ is a donor group. In one aspect of the present invention, R³ and R⁵ are donor groups. In one aspect of the present invention, R⁴ and R⁵ are donor groups. In one aspect of the present invention, R² and R⁵ are donor groups. In one aspect of the present invention, R² and R⁴ are donor groups. In one aspect of the present invention, R² and R⁵ are donor groups.

The heteroaryl group that Ar¹ and Ar² in the general formula (1) can adopt may be a monocyclic ring or may be a fused ring in which two or more rings are fused. In the case of a fused ring, the number of fused rings is preferably 2 to 6, and can be selected from, for example, 2 to 4. Specific examples of the ring include a pyridine ring, a pyrimidine ring, and a pyrrole ring, and these rings may be fused with any other ring. Specific examples of the heteroaryl group include a 2-pyridyl group, a 3-pyridyl group, a 4-pyridyl group, a carbazol-9-yl group, a carbazol-1-yl group, a carbazol-2-yl group, a carbazol-3-yl group, and a carbazol-4-yl group. The number of the ring skeleton-constituting atoms of the heteroaryl group is preferably 4 to 40, more preferably 5 to 20, and may be selected from a range of 5 to 16, or may be selected from a range of 5 to 12.

In one aspect of the present invention, Ar¹ and Ar² are substituted or unsubstituted aryl groups.

In the general formula (1), R¹ or R³ is a group represented by the following general formula (2). In addition, R⁴ and R⁵ can also adopt a group represented by the following general formula (2).

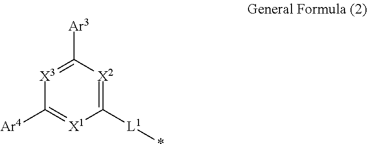

In the general formula (2), X¹ to X³ each independently represent N or C(R), and at least one of X¹ to X³ is N. R represents a hydrogen atom, a deuterium atom, or a substituent. Ar³ and Ar⁴ each independently represent a substituted or unsubstituted aryl group, or a substituted or unsubstituted heteroaryl group. L¹ represents a single bond, a substituted or unsubstituted arylene group, or a substituted or unsubstituted heteroarylene group. * represents a bonding position.

In the general formula (2), L¹ represents a single bond or a divalent linking group. Examples of the divalent linking group include a substituted or unsubstituted arylene group, and a substituted or unsubstituted heteroarylene group. In one preferred aspect of the present invention, L¹ represents a single bond. In one aspect of the present invention, L¹ represents a substituted or unsubstituted arylene group. In one aspect of the present invention, L¹ represents a substituted or unsubstituted heteroarylene group. Regarding the aryl moiety constituting the arylene group, reference can be made to the description and the preferred range of the aryl group in the description section of R² to R⁵ mentioned above. Examples of the heteroarylene group include a linking group formed by substituting at least one ring skeleton carbon atom constituting the arylene group with a nitrogen atom.

Specific examples of L¹ are shown below. Here, L¹ which can be employed in the present invention shall not be construed as being limited by these specific examples. In the following specific examples, the expression of a methyl group is omitted. Therefore, for example, L3 to L5 are substituted with a methyl group. * indicates a bonding position. L1 is a single bond.

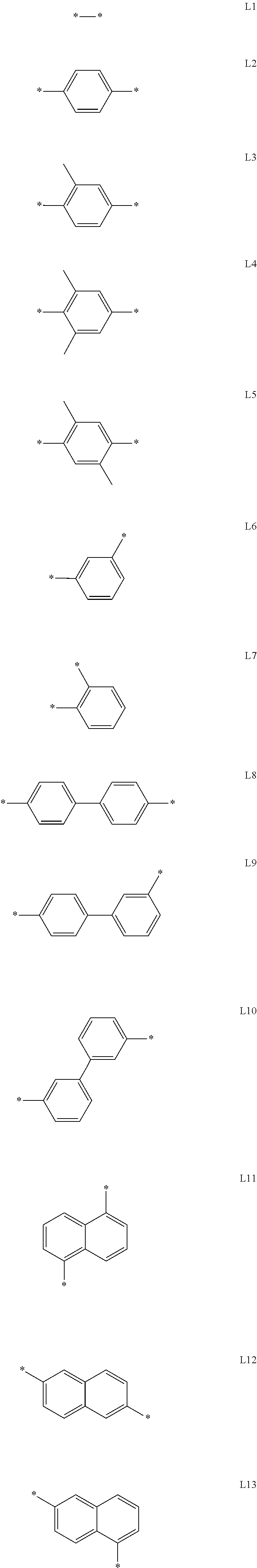

Groups obtained by substituting all hydrogen atoms present in the above L2 to L13 with deuterium atoms are disclosed as L14 to L25. In one aspect of the present invention, L¹ is selected from the group consisting of L1 to L25. In one aspect of the present invention, L¹ is selected from the group consisting of L1 to L7, and L14 to L19. In one aspect of the present invention, L¹ is selected from the group consisting of L1, L8 to L13, and L20 to L25. In one aspect of the present invention, L¹ is selected from the group consisting of L2 to L25.

In the general formula (2), X¹ to X³ each independently represent N or C(R). Here, at least one of X¹ to X³ is N. R represents a hydrogen atom, a deuterium atom, or a substituent. The substituent referred to herein may be selected from Substituent Group A, may be selected from Substituent Group B, may be selected from Substituent Group C, may be selected from Substituent Group D, or may be selected from Substituent Group E. In one preferred aspect of the present invention, X¹ to X³ are N. In one aspect of the present invention, X¹ and X³ are N, and X² is C(R). In one aspect of the present invention, X¹ and X² are N, and X³ is C(R). In one aspect of the present invention, X¹ is N, and X² and X³ are C(R). In one aspect of the present invention, X² is N, and X¹ and X³ are C(R). In one aspect of the present invention, R is a hydrogen atom or a deuterium atom. In one aspect of the present invention, R is an alkyl group optionally substituted with a deuterium atom. In one aspect of the present invention, R is an aryl group optionally substituted with a deuterium atom, an alkyl group, or an aryl group.

Regarding specific examples and preferred ranges of Ar³ and Ar⁴ in the general formula (2), reference can be made to the corresponding description of Ar¹ and Ar² in the general formula (1). In one aspect of the present invention, Ar¹ and Ar³ are the same. In one preferred aspect of the present invention, Ar¹ and Ar³ are the same, and Ar² and Ar⁴ are the same. In one aspect of the present invention, Ar¹ to Ar⁴ are the same.

In one preferred aspect of the present invention, X¹ to X³ are N, and L¹ is a single bond. In one aspect of the present invention, X¹ to X³ are N, and L¹ is a substituted or unsubstituted arylene group, preferably a substituted or unsubstituted phenylene group, and further preferably an unsubstituted phenylene group (for example, L2, for example, L6).

In one aspect of the present invention, X¹ to X³ are N, L¹ is a single bond, and Ar³ and Ar⁴ are the same. In one aspect of the present invention, X¹ to X³ are N, L¹ is a single bond, and Ar³ and Ar⁴ are each independently a substituted or unsubstituted aryl group. In one aspect of the present invention, X¹ to X³ are N, L¹ is a single bond, and Ar³ and Ar⁴ are each independently a substituted or unsubstituted heteroaryl group (excluding a nitrogen-containing 6-membered ring group), preferably a donor heteroaryl group bonded via a nitrogen atom, and more preferably a substituted or unsubstituted carbazol-9-yl group. In one aspect of the present invention, X¹ to X³ are N, L¹ is a single bond, Ar³ is a substituted or unsubstituted heteroaryl group (excluding a nitrogen-containing 6-membered ring group), preferably a donor heteroaryl group bonded via a nitrogen atom, and more preferably a substituted or unsubstituted carbazol-9-yl group, and Ar⁴ is a substituted or unsubstituted aryl group.

In one preferred aspect of the present invention, only R² is a group represented by the general formula (2). In one preferred aspect of the present invention, only R³ is a group represented by the general formula (2). In one aspect of the present invention, only one of R³, R⁴, and R⁵ and R² are each independently a group represented by the general formula (2). In one aspect of the present invention, only one of R², R⁴, and R⁵ and R³ are each independently a group represented by the general formula (2).

In one preferred aspect of the present invention, at least one of R² to R⁵ is a donor group having a fused ring structure of four or more rings. The “donor group having a fused ring structure of four or more rings” as referred to herein is a donor group having a fused ring structure of four or more rings and bonding via one atom of the ring skeleton-constituting atoms of the fused ring structure. The one atom is preferably a carbon atom or a nitrogen atom, and more preferably a nitrogen atom. The donor group as referred to herein means a group having a negative Hammett's op value.

The donor group having a fused ring structure of four or more rings preferably has a fused ring structure of five or more rings, and more preferably has a fused ring structure of five to seven rings. Examples thereof include groups having a fused ring structure of five rings and groups having a fused ring structure of seven rings.

As the donor group having a fused ring structure of four or more rings, a ring-fused carbazol-9-yl group is preferable. Specific examples thereof include D86 to D1141.

In particular, a group having a structure represented by the following general formula (3) is preferable.

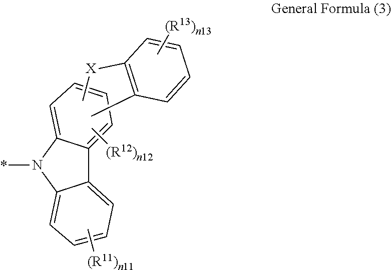

In the general formula (3), X represents O, S, or N—R¹⁴. R¹¹ to R¹³ each independently represent a deuterium atom, or a substituent. R¹⁴ represents an aryl group optionally substituted with one or more selected from the group consisting of a deuterium atom, an alkyl group and an aryl group, or an alkyl group optionally substituted with one or more selected from the group consisting of a deuterium atom and an aryl group. R¹¹'s, R¹²'s, and R¹³'s each may be bonded to each other to form a cyclic structure. n11 and n13 each independently represent an integer of 0 to 4, and n12 represents an integer of 0 to 2.

In the general formula (3), X represents O, S, or N—R¹⁴. R¹⁴ represents an aryl group optionally substituted with one or more selected from the group consisting of a deuterium atom, an alkyl group and an aryl group, or an alkyl group optionally substituted with one or more selected from the group consisting of a deuterium atom and an aryl group. Here, the aryl group as a substituent for the aryl group and the alkyl group can be selected from, for example, an aryl group having 6 to 22 carbon atoms, and the alkyl group as a substituent for the alkyl group and the aryl group can be selected from, for example, an alkyl group having 1 to 20 carbon atoms. In one preferred aspect of the present invention, X is O. In one preferred aspect of the present invention, X is N—R¹⁴. For example R¹⁴ of N—R¹⁴ is an aryl group (for example, having 6 to 22 carbon atoms). The aryl group may be not substituted with or may be substituted with at least one atom or group selected from the group consisting of a deuterium atom, an alkyl group (for example, having 1 to 20 carbon atoms), and an aryl group (for example, having 6 to 22 carbon atoms). X may be an oxygen atom or a sulfur atom.

In the general formula (3), the two bonds bonding to one benzene ring of the carbazole ring are bonded to the neighboring positions of the benzene ring to form a fused ring structure of the carbazole ring and the hetero-fused ring containing X. For example, in the case where X is O, a benzofurocarbazole ring is formed as a fused ring structure, in the case where X is S, a benzothienocarbazole ring is formed as a fused ring structure, and in the case where X is N—R¹⁴, an indolocarbazole ring is formed as a fused ring structure. The positions to which the two bonds are bonded may be the 1-position and the 2-position of the carbazole ring, or may be the 2-position and the 3-position of the carbazole ring, or may be the 3-position and the 4-position of the carbazole ring. In the case where the bonding positions of the two bonds are the 1-position and the 2-position, the position at which the bond of X bonds may be the 1-position or the 2-position, in the case where the bonding positions of the two bonds are the 2-position and the 3-position, the position at which the bond of X bonds may be the 2-position or the 3-position, and in the case where the bonding positions of the two bonds are the 3-position and the 4-position, the position at which the bond of X bonds may be the 3-position or the 4-position.

In the general formula (3), * represents a bonding position.

In the general formula (3), R¹¹ to R¹³ each independently represent a deuterium atom or a substituent. R¹¹'s, R¹²'s, and R¹³'s each may be bonded to each other to form a cyclic structure, but R¹¹ is not bonded to any one of R¹² to R¹⁴ to form a cyclic structure, R¹² is not bonded to any one of R¹³ and R¹⁴ to form a cyclic structure, and R¹³ is not bonded to R¹⁴ to form a cyclic structure. For example, the substituent may be selected from Substituent Group A, may be selected from Substituent Group B, may be selected from Substituent Group C, may be selected from Substituent Group D, or may be selected from Substituent Group E. In one preferred aspect of the present invention, the substituent means one group or a combination of two or more groups selected from the group consisting of an alkyl group (for example, having 1 to 20 carbon atoms), and an aryl group (for example, having 6 to 22 carbon atoms).

n11 and n13 each independently represent an integer of 0 to 4, and n12 represents an integer of 0 to 2. When n11 is 2 or more, two or more R¹¹'s may be the same or different. When n13 is 2 or more, two or more R¹³'s may be the same or different. When n12 is 2, two R¹²'s may be the same or different. n11 and n13 may be any number of 0, 1, 2, 3, or 4, and n12 may be any number of 0, 1, or 2. When n11 is 1, R¹¹ may be a deuterium atom, or may be a substituent. When n11 is 2 or more, all two or more R¹¹'s may be deuterium atoms, or all may be substituents, or a part thereof may be deuterium atoms and the remaining ones may be substituents. When n13 is 1, R¹³ may be a deuterium atom, or may be a substituent. When n13 is 2 or more, all two or more R¹¹'s may be deuterium atoms, or all may be substituents, or a part thereof may be deuterium atoms and the remaining ones may be substituents. When n12 is 1, R¹² may be a deuterium atom, or may be a substituent. When n12 is 2, both two R¹²'s may be deuterium atoms, or both may be substituents, or one of the two may be a deuterium atom and the other may be a substituent.

In one aspect of the present invention, two donor groups of R² to R⁵ are each a donor group having a fused ring structure of four or more rings, and more preferably a donor group represented by the general formula (3). The two donor groups may be the same or different, but are preferably the same. In one aspect of the present invention, at least R² is a donor group having a fused ring structure of four or more rings. In one aspect of the present invention, at least R³ is a donor group having a fused ring structure of four or more rings. In one aspect of the present invention, at least R⁴ is a donor group having a fused ring structure of four or more rings. In one aspect of the present invention, at least R⁵ is a donor group having a fused ring structure of four or more rings. In one aspect of the present invention, R³ and R⁵ are donor groups having a fused ring structure of four or more rings. In one aspect of the present invention, R⁴ and R⁵ are donor groups having a fused ring structure of four or more rings. In one aspect of the present invention, R² and R⁵ are donor groups having a fused ring structure of four or more rings. In one aspect of the present invention, R² and R⁴ are donor groups having a fused ring structure of four or more rings. In one aspect of the present invention, R³ and R⁴ are donor groups having a fused ring structure of four or more rings.

In one preferred aspect of the present invention, at least one of Ar¹ to Ar⁴ is a substituted or unsubstituted heteroaryl group (excluding a nitrogen-containing 6-membered ring group). The number of the substituted or unsubstituted heteroaryl groups (excluding a nitrogen-containing 6-membered ring group) may be only one, may be two, may be three, or may be all of Ar¹ to Ar⁴. When there are two or more, they may be the same or different, but are preferably the same. The substituted or unsubstituted heteroaryl group (excluding a nitrogen-containing 6-membered ring group) is preferably a heteroaryl group bonded via a nitrogen atom which is one of ring skeleton-constituting atoms, more preferably a heteroaryl group having a 5-membered ring bonded via a nitrogen atom (a group bonded via a nitrogen atom of a pyrrole ring, and the pyrrole ring may be substituted and is preferably fused), and still more preferably a substituted or unsubstituted carbazol-9-yl group. In one preferred aspect of the present invention, the carbazol-9-yl group as referred to herein may be substituted with an alkyl group or an aryl group optionally substituted with a group selected from the group consisting of a deuterium atom, an alkyl group and an aryl group, and can also be substituted with a deuterium atom. For the description and details of the carbazol-9-yl group, reference can be made to the description of the carbazol-9-yl group that R² to R⁵ can adopt, the description of the ring-fused carbazol-9-yl group, and specific examples D1 to D1141.

In a case where Ar¹ to Ar⁴ in the general formula (1) are phenyl groups optionally substituted with an alkyl group, it is preferable that at least one of R² to R⁵ is a donor group having a fused ring structure of four or more rings, and it is more preferable that two of R² to R⁵ are both donor groups having a fused ring structure of four or more rings.

In one preferred aspect of the present invention, at least one of Ar¹ to Ar⁴ is a substituted or unsubstituted heteroaryl group (excluding a nitrogen-containing 6-membered ring group), or at least one of R² to R⁵ is a donor group having a fused ring structure of four or more rings.

The compound represented by the general formula (1) preferably does not contain a metal atom, and may be a compound composed only of atoms selected from the group consisting of a carbon atom, a hydrogen atom, a deuterium atom, a nitrogen atom, an oxygen atom, and a sulfur atom. In one preferred aspect of the present invention, the compound represented by the general formula (1) is composed only of atoms selected from the group consisting of a carbon atom, a hydrogen atom, a deuterium atom, a nitrogen atom, and an oxygen atom. In addition, the compound represented by the general formula (1) may be a compound composed only of atoms selected from the group consisting of a carbon atom, a hydrogen atom, a deuterium atom, a nitrogen atom, and a sulfur atom. The compound represented by the general formula (1) may be a compound composed only of atoms selected from the group consisting of a carbon atom, a hydrogen atom, a deuterium atom, and a nitrogen atom. The compound represented by the general formula (1) may be a compound composed only of atoms selected from the group consisting of a carbon atom, a hydrogen atom, and a nitrogen atom. Further, the compound represented by the general formula (1) may be a compound which does not contain a hydrogen atom but contains a deuterium atom.

In the description herein, the term “Substituent Group A” means one atom or group or a combination of two or more thereof selected from the group consisting of a deuterium atom, a hydroxyl group, a halogen atom (for example, a fluorine atom, a chlorine atom, a bromine atom, and an iodine atom), an alkyl group (for example, having 1 to 40 carbon atoms), an alkoxy group (for example, having 1 to 40 carbon atoms), an alkylthio group (for example, having 1 to 40 carbon atoms), an aryl group (for example, having 6 to 30 carbon atoms), an aryloxy group (for example, having 6 to 30 carbon atoms), an arylthio group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), a heteroarylthio group (for example, having 5 to 30 ring skeleton-constituting atoms), an acyl group (for example, having 1 to 40 carbon atoms), an alkenyl group (for example, having 1 to 40 carbon atoms), an alkynyl group (for example, having 1 to 40 carbon atoms), an alkoxycarbonyl group (for example, having 1 to 40 carbon atoms), an aryloxycarbonyl group (for example, having 1 to 40 carbon atoms), a heteroaryloxycarbonyl group (for example, having 1 to 40 carbon atoms), a silyl group (for example, a trialkylsilyl group having 1 to 40 carbon atoms), and a nitro group.

In the description herein, the term “Substituent Group B” means one atom or group or a combination of two or more thereof selected from the group consisting of a deuterium atom, an alkyl group (for example, having 1 to 40 carbon atoms), an alkoxy group (for example, having 1 to 40 carbon atoms), an aryl group (for example, having 6 to 30 carbon atoms), an aryloxy group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), and a diarylaminoamino group (for example, having 0 to 20 carbon atoms).

In the description herein, the term “Substituent Group C” means one atom or group or a combination of two or more thereof selected from the group consisting of a deuterium atom, an alkyl group (for example, having 1 to 20 carbon atoms), an aryl group (for example, having 6 to 22 carbon atoms), a heteroaryl group (for example, having 5 to 20 ring skeleton-constituting atoms), and a diarylamino group (for example, having 12 to 20 carbon atoms).

In the description herein, the term “Substituent Group D” means one atom or group or a combination of two or more thereof selected from the group consisting of a deuterium atom, an alkyl group (for example, having 1 to 20 carbon atoms), an aryl group (for example, having 6 to 22 carbon atoms), and a heteroaryl group (for example, having 5 to 20 ring skeleton-constituting atoms).

In the description herein, the term “Substituent Group E” means one atom or group or a combination of two or more groups selected from the group consisting of a deuterium atom, an alkyl group (for example, having 1 to 20 carbon atoms), and an aryl group (for example, having 6 to 22 carbon atoms).

In the description herein, the substituent meant by an expression of “substituted or unsubstituted” or “optionally substituted” may be selected, for example, from Substituent Group A, may be selected from Substituent Group B, may be selected from Substituent Group C, may be selected from Substituent Group D, or may be selected from Substituent Group E.

Specific examples of the compound represented by the general formula (1) are shown in the following Tables 1 to 4. Here, the compound represented by the general formula (1) that can be used in the present invention should not be construed as being limited by these specific examples.

In Table 1, structures of the compounds are individually shown by specifying R³ to R⁵ of the following general formula (1a) for each compound. That is, structures in which R is a group represented by the general formula (2), Ar¹ and Ar³ are perdeuterated carbazol-9-yl groups (D55), Ar² and Ar⁴ are perdeuterated phenyl groups (Ar45), X¹ to X³ are nitrogen atoms (N), L¹ is a single bond (L1), R¹ is a hydrogen atom, and R³ to R⁵ are groups specified in Table 1 are individually shown as structures of Compounds 1 to 170.

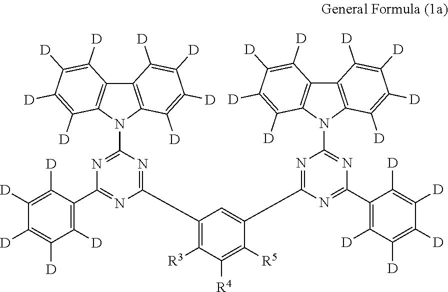

TABLE 1

| No. | R³ | R⁴ | R⁵ |
|---|---|---|---|
| 1 | D1 | Ar1 | D1 |
| 2 | D2 | Ar1 | D2 |
| 3 | D3 | Ar1 | D3 |
| 4 | D4 | Ar1 | D4 |
| 5 | D5 | Ar1 | D5 |
| 6 | D6 | Ar1 | D6 |
| 7 | D7 | Ar1 | D7 |
| 8 | D8 | Ar1 | D8 |
| 9 | D9 | Ar1 | D9 |
| 10 | D10 | Ar1 | D10 |
| 11 | D11 | Ar1 | D11 |
| 12 | D12 | Ar1 | D12 |
| 13 | D13 | Ar1 | D13 |
| 14 | D14 | Ar1 | D14 |
| 15 | D15 | Ar1 | D15 |
| 16 | D16 | Ar1 | D16 |
| 17 | D17 | Ar1 | D17 |
| 18 | D18 | Ar1 | D18 |
| 19 | D19 | Ar1 | D19 |
| 20 | D20 | Ar1 | D20 |
| 21 | D21 | Ar1 | D21 |
| 22 | D22 | Ar1 | D22 |
| 23 | D23 | Ar1 | D23 |
| 24 | D24 | Ar1 | D24 |
| 25 | D25 | Ar1 | D25 |
| 26 | D26 | Ar1 | D26 |
| 27 | D27 | Ar1 | D27 |
| 28 | D28 | Ar1 | D28 |
| 29 | D29 | Ar1 | D29 |
| 30 | D30 | Ar1 | D30 |
| 31 | D31 | Ar1 | D31 |
| 32 | D32 | Ar1 | D32 |
| 33 | D33 | Ar1 | D33 |
| 34 | D34 | Ar1 | D34 |
| 35 | D35 | Ar1 | D35 |
| 36 | D36 | Ar1 | D36 |
| 37 | D37 | Ar1 | D37 |
| 38 | D38 | Ar1 | D38 |
| 39 | D39 | Ar1 | D39 |
| 40 | D40 | Ar1 | D40 |
| 41 | D41 | Ar1 | D41 |
| 42 | D42 | Ar1 | D42 |
| 43 | D43 | Ar1 | D43 |
| 44 | D44 | Ar1 | D44 |
| 45 | D45 | Ar1 | D45 |
| 46 | D46 | Ar1 | D46 |
| 47 | D47 | Ar1 | D47 |
| 48 | D48 | Ar1 | D48 |
| 49 | D49 | Ar1 | D49 |
| 50 | D50 | Ar1 | D50 |
| 51 | D51 | Ar1 | D51 |
| 52 | D52 | Ar1 | D52 |
| 53 | D53 | Ar1 | D53 |
| 54 | D54 | Ar1 | D54 |
| 55 | D55 | Ar1 | D55 |
| 56 | D56 | Ar1 | D56 |
| 57 | D57 | Ar1 | D57 |
| 58 | D58 | Ar1 | D58 |
| 59 | D59 | Ar1 | D59 |
| 60 | D60 | Ar1 | D60 |
| 61 | D61 | Ar1 | D61 |
| 62 | D62 | Ar1 | D62 |
| 63 | D63 | Ar1 | D63 |
| 64 | D64 | Ar1 | D64 |
| 65 | D65 | Ar1 | D65 |
| 66 | D66 | Ar1 | D66 |
| 67 | D67 | Ar1 | D67 |
| 68 | D68 | Ar1 | D68 |
| 69 | D69 | Ar1 | D69 |
| 70 | D70 | Ar1 | D70 |
| 71 | D71 | Ar1 | D71 |
| 72 | D72 | Ar1 | D72 |
| 73 | D73 | Ar1 | D73 |
| 74 | D74 | Ar1 | D74 |
| 75 | D75 | Ar1 | D75 |
| 76 | D76 | Ar1 | D76 |
| 77 | D77 | Ar1 | D77 |
| 78 | D78 | Ar1 | D78 |
| 79 | D79 | Ar1 | D79 |
| 80 | D80 | Ar1 | D80 |
| 81 | D81 | Ar1 | D81 |
| 82 | D82 | Ar1 | D82 |
| 83 | D83 | Ar1 | D83 |
| 84 | D84 | Ar1 | D84 |
| 85 | D85 | Ar1 | D85 |
| 86 | D1 | Ar1 | D1 |
| 87 | D2 | Ar1 | D2 |
| 88 | D3 | Ar1 | D3 |
| 89 | D4 | Ar1 | D4 |
| 90 | D5 | Ar1 | D5 |
| 91 | D6 | Ar1 | D6 |
| 92 | D7 | Ar1 | D7 |
| 93 | D8 | Ar1 | D8 |
| 94 | D9 | Ar1 | D9 |
| 95 | D10 | Ar1 | D10 |
| 96 | D11 | Ar1 | D11 |
| 97 | D12 | Ar1 | D12 |
| 98 | D13 | Ar1 | D13 |
| 99 | D14 | Ar1 | D14 |
| 100 | D15 | Ar1 | D15 |
| 101 | D16 | Ar1 | D16 |
| 102 | D17 | Ar1 | D17 |
| 103 | D18 | Ar1 | D18 |
| 104 | D19 | Ar1 | D19 |
| 105 | D20 | Ar1 | D20 |
| 106 | D21 | Ar1 | D21 |
| 107 | D22 | Ar1 | D22 |
| 108 | D23 | Ar1 | D23 |
| 109 | D24 | Ar1 | D24 |
| 110 | D25 | Ar1 | D25 |
| 111 | D26 | Ar1 | D26 |
| 112 | D27 | Ar1 | D27 |
| 113 | D28 | Ar1 | D28 |
| 114 | D29 | Ar1 | D29 |
| 115 | D30 | Ar1 | D30 |
| 116 | D31 | Ar1 | D31 |
| 117 | D32 | Ar1 | D32 |
| 118 | D33 | Ar1 | D33 |
| 119 | D34 | Ar1 | D34 |
| 120 | D35 | Ar1 | D35 |
| 121 | D36 | Ar1 | D36 |
| 122 | D37 | Ar1 | D37 |
| 123 | D38 | Ar1 | D38 |
| 124 | D39 | Ar1 | D39 |
| 125 | D40 | Ar1 | D40 |
| 126 | D41 | Ar1 | D41 |
| 127 | D42 | Ar1 | D42 |
| 128 | D43 | Ar1 | D43 |
| 129 | D44 | Ar1 | D44 |
| 130 | D45 | Ar1 | D45 |
| 131 | D46 | Ar1 | D46 |
| 132 | D47 | Ar1 | D47 |
| 133 | D48 | Ar1 | D48 |
| 134 | D49 | Ar1 | D49 |
| 135 | D50 | Ar1 | D50 |
| 136 | D51 | Ar1 | D51 |
| 137 | D52 | Ar1 | D52 |
| 138 | D53 | Ar1 | D53 |
| 139 | D54 | Ar1 | D54 |
| 140 | D55 | Ar1 | D55 |
| 141 | D56 | Ar1 | D56 |
| 142 | D57 | Ar1 | D57 |
| 143 | D58 | Ar1 | D58 |
| 144 | D59 | Ar1 | D59 |
| 145 | D60 | Ar1 | D60 |
| 146 | D61 | Ar1 | D61 |
| 147 | D62 | Ar1 | D62 |
| 148 | D63 | Ar1 | D63 |
| 149 | D64 | Ar1 | D64 |
| 150 | D65 | Ar1 | D65 |
| 151 | D66 | Ar1 | D66 |
| 152 | D67 | Ar1 | D67 |
| 153 | D68 | Ar1 | D68 |
| 154 | D69 | Ar1 | D69 |
| 155 | D70 | Ar1 | D70 |
| 156 | D71 | Ar1 | D71 |
| 157 | D72 | Ar1 | D72 |
| 158 | D73 | Ar1 | D73 |
| 159 | D74 | Ar1 | D74 |
| 160 | D75 | Ar1 | D75 |
| 161 | D76 | Ar1 | D76 |
| 162 | D77 | Ar1 | D77 |
| 163 | D78 | Ar1 | D78 |
| 164 | D79 | Ar1 | D79 |
| 165 | D80 | Ar1 | D80 |
| 166 | D81 | Ar1 | D81 |
| 167 | D82 | Ar1 | D82 |
| 168 | D83 | Ar1 | D83 |
| 169 | D84 | Ar1 | D84 |
| 170 | D85 | Ar1 | D85 |

In Table 2, structures of Compounds 1 to 149316 are shown by collectively displaying R³ to R⁵ of a plurality of compounds in each row. For example, in the row of Compounds 1 to 170 in Table 2, compounds in which R⁴ is fixed to Ar1 (phenyl group), R³ and R⁵ are the same, and D1 to D170 are referred to as Compounds 1 to 170, respectively. That is, the row of Compounds 1 to 170 in Table 2 collectively represents Compounds 1 to 170 specified in Table 1. Further, in the row of Compounds 171 to 1082 in Table 2, compounds in which R⁴ is fixed to Ar1, R³ and R⁵ are the same, and D171 to D1082 are referred to as Compounds 171 to 1082, respectively. Similarly, in the row of Compounds 1083 to 2164 in Table 2, compounds in which R⁴ is fixed to Ar2, R³ and R⁵ are the same, and D1 to D1082 are referred to as Compounds 1083 to 2164, respectively. In the same manner, Compounds 2165 to 149316 in Table 2 are also specified.

TABLE 2

| No. | R³ | R⁴ | R⁵ | = |
|---|---|---|---|---|
| 1~170 | D1~D170 | Ar1 | D1~D170 | R³ = R⁵ |
| 171~1082 | D171~D1082 | Ar1 | D171~D1082 | R³ = R⁵ |
| 1083~2164 | D1~D1082 | Ar2 | D1~D1082 | |
| 2165~3246 | D1~D1082 | Ar3 | D1~D1082 | |
| 3247~4328 | D1~D1082 | Ar4 | D1~D1082 | |
| 4329~5410 | D1~D1082 | Ar5 | D1~D1082 | |
| 5411~6492 | D1~D1082 | Ar6 | D1~D1082 | |
| 6493~7574 | D1~D1082 | Ar7 | D1~D1082 | |
| 7575~8656 | D1~D1082 | Ar8 | D1~D1082 | |
| 8657~9738 | D1~D1082 | Ar9 | D1~D1082 | |
| 9739~10820 | D1~D1082 | Ar10 | D1~D1082 | |
| 10821~11902 | D1~D1082 | Ar11 | D1~D1082 | |
| 11903~12984 | D1~D1082 | Ar12 | D1~D1082 | |
| 12985~14066 | D1~D1082 | Ar13 | D1~D1082 | |
| 14067~15148 | D1~D1082 | Ar14 | D1~D1082 | |
| 15149~16230 | D1~D1082 | Ar15 | D1~D1082 | |
| 16231~17312 | D1~D1082 | Ar16 | D1~D1082 | |
| 17313~18394 | D1~D1082 | Ar17 | D1~D1082 | |
| 18395~19476 | D1~D1082 | Ar18 | D1~D1082 | |
| 19477~20558 | D1~D1082 | Ar19 | D1~D1082 | |
| 20559~21640 | D1~D1082 | Ar20 | D1~D1082 | |
| 21641~22722 | D1~D1082 | Ar21 | D1~D1082 | |
| 22723~23804 | D1~D1082 | Ar22 | D1~D1082 | |
| 23805~24886 | D1~D1082 | Ar23 | D1~D1082 | |
| 24887~25968 | D1~D1082 | Ar24 | D1~D1082 | |
| 25969~27050 | D1~D1082 | Ar25 | D1~D1082 | |
| 27051~28132 | D1~D1082 | Ar26 | D1~D1082 | |
| 28133~29214 | D1~D1082 | Ar27 | D1~D1082 | |
| 29215~30296 | D1~D1082 | Ar28 | D1~D1082 | |
| 30297~31378 | D1~D1082 | Ar29 | D1~D1082 | |
| 31379~32460 | D1~D1082 | Ar30 | D1~D1082 | |
| 32461~33542 | D1~D1082 | Ar31 | D1~D1082 | |
| 33543~34624 | D1~D1082 | Ar32 | D1~D1082 | |
| 34625~35706 | D1~D1082 | Ar33 | D1~D1082 | |
| 35707~36788 | D1~D1082 | Ar34 | D1~D1082 | |
| 36789~37870 | D1~D1082 | Ar35 | D1~D1082 | |
| 37871~38952 | D1~D1082 | Ar36 | D1~D1082 | |
| 38953~40034 | D1~D1082 | Ar37 | D1~D1082 | |
| 40035~41116 | D1~D1082 | Ar38 | D1~D1082 | |
| 41117~42198 | D1~D1082 | Ar39 | D1~D1082 | |
| 42199~43280 | D1~D1082 | Ar40 | D1~D1082 | |
| 43281~44362 | D1~D1082 | Ar41 | D1~D1082 | |
| 44363~45444 | D1~D1082 | Ar42 | D1~D1082 | |
| 45445~46526 | D1~D1082 | Ar43 | D1~D1082 | |
| 46527~47608 | D1~D1082 | Ar44 | D1~D1082 | |
| 47609~48690 | D1~D1082 | Ar45 | D1~D1082 | |
| 48691~49772 | D1~D1082 | Ar46 | D1~D1082 | |
| 49773~50854 | D1~D1082 | Ar47 | D1~D1082 | |
| 50855~51936 | D1~D1082 | Ar48 | D1~D1082 | |
| 51937~53018 | D1~D1082 | Ar49 | D1~D1082 | |
| 53019~54100 | D1~D1082 | Ar50 | D1~D1082 | |
| 54101~55182 | D1~D1082 | Ar51 | D1~D1082 | |
| 55183~56264 | D1~D1082 | Ar52 | D1~D1082 | |
| 56265~57346 | D1~D1082 | Ar53 | D1~D1082 | |
| 57347~58428 | D1~D1082 | Ar54 | D1~D1082 | |
| 58429~59510 | D1~D1082 | Ar55 | D1~D1082 | |
| 59511~60592 | D1~D1082 | Ar56 | D1~D1082 | |
| 60593~61674 | D1~D1082 | Ar57 | D1~D1082 | |
| 61675~62756 | D1~D1082 | Ar58 | D1~D1082 | |
| 62757~63838 | D1~D1082 | Ar59 | D1~D1082 | |
| 63839~64920 | D1~D1082 | Ar60 | D1~D1082 | |
| 64921~66002 | D1~D1082 | Ar61 | D1~D1082 | |
| 66003~67084 | D1~D1082 | Ar62 | D1~D1082 | |
| 67085~68166 | D1~D1082 | Ar63 | D1~D1082 | |
| 68167~69248 | D1~D1082 | Ar64 | D1~D1082 | |
| 69249~70330 | D1~D1082 | Ar65 | D1~D1082 | |
| 70331~71412 | D1~D1082 | Ar66 | D1~D1082 | |
| 71413~72494. | D1~D1082 | Ar67 | D1~D1082 | |
| 72495~73576 | D1~D1082 | Ar68 | D1~D1082 | |
| 73577~74658 | D1~D1082 | Ar69 | D1~D1082 | |
| 74659~75740 | Ar1 | D1~D1082 | D1~D1082 | R⁴ = R⁵ |
| 75741~76822 | Ar2 | D1~D1082 | D1~D1082 | R⁴ = R⁵ |
| 76823~77904 | Ar3 | D1~D1082 | D1~D1082 | |
| 77905~78986 | Ar4 | D1~D1082 | D1~D1082 | |
| 78987~80068 | Ar5 | D1~D1082 | D1~D1082 | |
| 80069~81150 | Ar6 | D1~D1082 | D1~D1082 | |
| 81151~82232 | Ar7 | D1~D1082 | D1~D1082 | |
| 82233~83314 | Ar8 | D1~D1082 | D1~D1082 | |
| 83315~84396 | Ar9 | D1~D1082 | D1~D1082 | |
| 84397~85478 | Ar10 | D1~D1082 | D1~D1082 | |
| 85479~86560 | Ar11 | D1~D1082 | D1~D1082 | |
| 86561~87642 | Ar12 | D1~D1082 | D1~D1082 | |
| 87643~88724 | Ar13 | D1~D1082 | D1~D1082 | |
| 88725~89806 | Ar14 | D1~D1082 | D1~D1082 | |
| 89807~90888 | Ar15 | D1~D1082 | D1~D1082 | |
| 90889~91970 | Ar16 | D1~D1082 | D1~D1082 | |
| 91971~93052 | Ar17 | D1+D1082 | D1~D1082 | |
| 93053~94134: | Ar18 | D1+D1082 | D1~D1082 | |
| 94135~95216 | Ar19 | D1~D1082 | D1~D1082 | |
| 95217~96298 | Ar20 | D1~D1082 | D1~D1082 | |
| 96299~97380 | Ar21 | D1~D1082 | D1~D1082 | |
| 97381~98462 | Ar22 | D1~D1082 | D1~D1082 | |
| 98463~99544 | Ar23 | D1~D1082 | D1~D1082 | |
| 99545~100626 | Ar24 | D1~D1082 | D1~D1082 | |
| 100627~101708 | Ar25 | D1~D1082 | D1~D1082 | |
| 101709~102790 | Ar26 | D1~D1082 | D1~D1082 | |
| 102791~103872 | Ar27 | D1~D1082 | D1~D1082 | |
| 103873~104954 | Ar28 | D1~D1082 | D1~D1082 | |
| 104955~106036 | Ar29 | D1~D1082 | D1~D1082 | |
| 106037~107118 | Ar30 | D1~D1082 | D1~D1082 | |
| 107119~108200 | Ar31 | D1~D1082 | D1~D1082 | |
| 108201~109282 | Ar32 | D1~D1082 | D1~D1082 | |
| 109283~110364 | Ar33 | D1~D1082 | D1~D1082 | |
| 110365~111446 | Ar34 | D1+D1082 | D1~D1082 | |
| 111447~112528 | Ar35 | D1~D1082 | D1~D1082 | |
| 112529~113610 | Ar36 | D1~D1082 | D1~D1082 | |
| 113611~114692 | Ar37 | D1~D1082 | D1~D1082 | |
| 114693~115774 | Ar38 | D1~D1082 | D1~D1082 | |
| 115775~116856 | Ar39 | D1~D1082 | D1~D1082 | |
| 116857~117938 | Ar40 | D1~D1082 | D1~D1082 | |
| 117939~119020 | Ar41 | D1~D1082 | D1~D1082 | |
| 119021~120102 | Ar42 | D1~D1082 | D1~D1082 | |
| 120103~121184 | Ar43 | D1~D1082 | D1~D1082 | |
| 121185~122266 | Ar44 | D1~D1082 | D1~D1082 | |
| 122267~123348 | Ar45 | D1~D1082 | D1~D1082 | |
| 123349~124430 | Ar46 | D1~D1082 | D1~D1082 | |
| 124431~125512 | Ar47 | D1~D1082 | D1~D1082 | |
| 125513~126594 | Ar48 | D1~D1082 | D1~D1082 | |
| 126595~127676 | Ar49 | D1~D1082 | D1~D1082 | |
| 127677~128758 | Ar50 | D1~D1082 | D1~D1082 | |
| 128759~129840 | Ar51 | D1~D1082 | D1~D1082 | |
| 129841~130922 | Ar52 | D1~D1082 | D1~D1082 | |
| 130923~132004 | Ar53 | D1~D1082 | D1~D1082 | |
| 132005~133086 | Ar54 | D1~D1082 | D1~D1082 | |
| 133087~134168 | Ar55 | D1~D1082 | D1~D1082 | |
| 134169~135250 | Ar56 | D1~D1082 | D1~D1082 | |
| 135251~136332 | Ar57 | D1~D1082 | D1~D1082 | |
| 136333~137414 | Ar58 | D1~D1082 | D1~D1082 | |
| 137415~138496 | Ar59 | D1~D1082 | D1~D1082 | |
| 138497~139578 | Ar60 | D1~D1082 | D1~D1082 | |
| 139579~140660 | Ar61 | D1~D1082 | D1~D1082 | |
| 140661~141742 | Ar62 | D1~D1082 | D1~D1082 | |
| 141743~142824 | Ar63 | D1~D1082 | D1~D1082 | |
| 142825~143906 | Ar64 | D1~D1082 | D1~D1082 | |
| 143907~144988 | Ar65 | D1~D1082 | D1~D1082 | |
| 144989~146070 | Ar66 | D1~D1082 | D1~D1082 | |
| 146071~147152 | Ar67 | D1~D1082 | D1~D1082 | |
| 147153~148234 | Ar68 | D1~D1082 | D1~D1082 | |
| 148235~149316 | Ar69 | D1~D1082 | D1~D1082 | |

Next, specific examples of the compound having a structure represented by the following general formula (1b) are shown in Table 3. In Table 3, structures of the compounds are shown in the same manner as in Table 2.

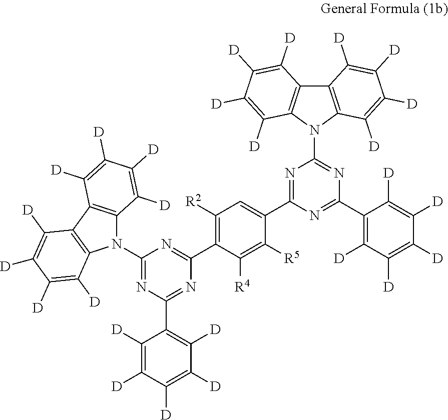

TABLE 3

| No. | R² | R⁴ | R⁵ | = |
|---|---|---|---|---|
| 149317~150398 | D1~D1082 | Ar1 | D1~D1082 | R² = R⁵ |
| 150399~151480 | D1~D1082 | Ar2 | D1~D1082 | R² = R⁵ |
| 151481~152562 | D1~D1082 | Ar3 | D1~D1082 | |
| 152563~153644 | D1~D1082 | Ar4 | D1~D1082 | |
| 153645~154726 | D1~D1082 | Ar5 | D1~D1082 | |
| 154727~155808 | D1~D1082 | Ar6 | D1~D1082 | |
| 155809~156890 | D1~D1082 | Ar7 | D1~D1082 | |
| 156891~157972 | D1~D1082 | Ar8 | D1~D1082 | |
| 157973~159054 | D1~D1082 | Ar9 | D1~D1082 | |
| 159055~160136 | D1~D1082 | Ar10 | D1~D1082 | |
| 160137~161218 | D1~D1082 | Ar11 | D1~D1082 | |
| 161219~162300 | D1~D1082 | Ar12 | D1~D1082 | |
| 162301~163382 | D1~D1082 | Ar13 | D1~D1082 | |
| 163383~164464 | D1~D1082 | Ar14 | D1~D1082 | |
| 164465~165546 | D1~D1082 | Ar15 | D1~D1082 | |
| 165547~166628 | D1~D1082 | Ar16 | D1~D1082 | |
| 166629~167710 | D1~D1082 | Ar17 | D1~D1082 | |
| 167711~168792 | D1~D1082 | Ar18 | D1~D1082 | |
| 168793~169874 | D1~D1082 | Ar19 | D1~D1082 | |
| 169875~170956 | D1~D1082 | Ar20 | D1~D1082 | |
| 170957~172038 | D1~D1082 | Ar21 | D1~D1082 | |
| 172039~173120 | D1~D1082 | Ar22 | D1~D1082 | |
| 173121~174202 | D1~D1082 | Ar23 | D1~D1082 | |
| 174203~175284 | D1~D1082 | Ar24 | D1~D1082 | |
| 175285~176366 | D1~D1082 | Ar25 | D1~D1082 | |
| 176367~177448 | D1~D1082 | Ar26 | D1~D1082 | |
| 177449~178530 | D1~D1082 | Ar27 | D1~D1082 | |
| 178531~179612 | D1~D1082 | Ar28 | D1~D1082 | |
| 179613~180694 | D1~D1082 | Ar29 | D1~D1082 | |
| 180695~181776 | D1~D1082 | Ar30 | D1~D1082 | |
| 181777~182858 | D1~D1082 | Ar31 | D1~D1082 | |
| 182859~183940 | D1~D1082 | Ar32 | D1~D1082 | |
| 183941~185022 | D1~D1082 | Ar33 | D1~D1082 | |
| 185023~186104 | D1~D1082 | Ar34 | D1~D1082 | |
| 186105~187186 | D1~D1082 | Ar35 | D1~D1082 | |
| 187187~188268 | D1~D1082 | Ar36 | D1~D1082 | |
| 188269~189350 | D1~D1082 | Ar37 | D1~D1082 | |
| 189351~190432 | D1~D1082 | Ar38 | D1~D1082 | |
| 190433~191514 | D1~D1082 | Ar39 | D1~D1082 | |
| 191515~192596 | D1~D1082 | Ar40 | D1~D1082 | |
| 192597~193678 | D1~D1082 | Ar41 | D1~D1082 | |
| 193679~194760 | D1~D1082 | Ar42 | D1~D1082 | |
| 194761~195842 | D1~D1082 | Ar43 | D1~D1082 | |
| 195843~196924 | D1~D1082 | Ar44 | D1~D1082 | |
| 196925~198006 | D1~D1082 | Ar45 | D1~D1082 | |
| 198007~199088 | D1~D1082 | Ar46 | D1~D1082 | |
| 199089~200170 | D1~D1082 | Ar47 | D1~D1082 | |
| 200171~201252 | D1~D1082 | Ar48 | D1~D1082 | |
| 201253~202334 | D1~D1082 | Ar49 | D1~D1082 | |
| 202335~203416 | D1~D1082 | Ar50 | D1~D1082 | |
| 203417~204498 | D1~D1082 | Ar51 | D1~D1082 | |
| 204499~205580 | D1~D1082 | Ar52 | D1~D1082 | |
| 205581~206662 | D1~D1082 | Ar53 | D1~D1082 | |
| 206663~207744 | D1~D1082 | Ar54 | D1~D1082 | |
| 207745~208826 | D1~D1082 | Ar55 | D1~D1082 | |
| 208827~209908 | D1~D1082 | Ar56 | D1~D1082 | |
| 209909~210990 | D1~D1082 | Ar57 | D1~D1082 | |
| 210991~212072 | D1~D1082 | Ar58 | D1~D1082 | |
| 212073~213154 | D1~D1082 | Ar59 | D1~D1082 | |
| 213155~214236 | D1~D1082 | Ar60 | D1~D1082 | |
| 214237~215318 | D1~D1082 | Ar61 | D1~D1082 | |
| 215319~216400 | D1~D1082 | Ar62 | D1~D1082 | |
| 216401~217482 | D1~D1082 | Ar63 | D1~D1082 | |
| 217483~218564 | D1~D1082 | Ar64 | D1~D1082 | |
| 218565~219646 | D1~D1082 | Ar65 | D1~D1082 | |
| 219647~220728 | D1~D1082 | Ar66 | D1~D1082 | |
| 220729~221810 | D1~D1082 | Ar67 | D1~D1082 | |
| 221811~222892 | D1~D1082 | Ar68 | D1~D1082 | |
| 222893~223974 | D1~D1082 | Ar69 | D1~D1082 | |
| 223975~225056 | Ar1 | D1~D1082 | D1~D1082 | R⁴ = R⁵ |
| 225057~226138 | Ar2 | D1~D1082 | D1~D1082 | R⁴ = R⁵ |
| 226139~227220 | Ar3 | D1~D1082 | D1~D1082 | |
| 227221~228302 | Ar4 | D1~D1082 | D1~D1082 | |
| 228303~229384 | Ar5 | D1~D1082 | D1~D1082 | |
| 229385~230466 | Ar6 | D1~D1082 | D1~D1082 | |
| 230467~231548 | Ar7 | D1~D1082 | D1~D1082 | |
| 231549~232630 | Ar8 | D1~D1082 | D1~D1082 | |
| 232631~233712 | Ar9 | D1~D1082 | D1~D1082 | |
| 233713~234794 | Ar10 | D1~D1082 | D1~D1082 | |
| 234795~235876 | Ar11 | D1~D1082 | D1~D1082 | |
| 235877~236958 | Ar12 | D1~D1082 | D1~D1082 | |
| 236959~238040 | Ar13 | D1~D1082 | D1~D1082 | |
| 238041~239122 | Ar14 | D1~D1082 | D1~D1082 | |
| 239123~240204 | Ar15 | D1~D1082 | D1~D1082 | |
| 240205~241286 | Ar16 | D1~D1082 | D1~D1082 | |
| 241287~242368 | Ar17 | D1~D1082 | D1~D1082 | |
| 242369~243450 | Ar18 | D1~D1082 | D1~D1082 | |
| 243451~244532 | Ar19 | D1~D1082 | D1~D1082 | |
| 244533~245614 | Ar20 | D1~D1082 | D1~D1082 | |
| 245615~246696 | Ar21 | D1~D1082 | D1~D1082 | |
| 246697~247778 | Ar22 | D1~D1082 | D1~D1082 | |
| 247779~248860 | Ar23 | D1~D1082 | D1~D1082 | |
| 248861~249942 | Ar24 | D1~D1082 | D1~D1082 | |
| 249943~251024 | Ar25 | D1~D1082 | D1~D1082 | |
| 251025~252106 | Ar26 | D1~D1082 | D1~D1082 | |
| 252107~253188 | Ar27 | D1~D1082 | D1~D1082 | |
| 253189~254270 | Ar28 | D1~D1082 | D1~D1082 | |
| 254271~255352 | Ar29 | D1~D1082 | D1~D1082 | |
| 255353~256434 | Ar30 | D1~D1082 | D1~D1082 | |
| 256435~257516 | Ar31 | D1~D1082 | D1~D1082 | |
| 257517~258598 | Ar32 | D1~D1082 | D1~D1082 | |
| 258599~259680 | Ar33 | D1~D1082 | D1~D1082 | |
| 259681~260762 | Ar34 | D1~D1082 | D1~D1082 | |
| 260763~261844 | Ar35 | D1~D1082 | D1~D1082 | |
| 261845~262926 | Ar36 | D1~D1082 | D1~D1082 | |
| 262927~264008 | Ar37 | D1~D1082 | D1~D1082 | |
| 264009~265090 | Ar38 | D1~D1082 | D1~D1082 | |
| 265091~266172 | Ar39 | D1~D1082 | D1~D1082 | |
| 266173~267254 | Ar40 | D1~D1082 | D1~D1082 | |
| 267255~268336 | Ar41 | D1~D1082 | D1~D1082 | |
| 268337~269418 | Ar42 | D1~D1082 | D1~D1082 | |
| 269419~270500 | Ar43 | D1~D1082 | D1~D1082 | |
| 270501~271582 | Ar44 | D1~D1082 | D1~D1082 | |
| 271583~272664 | Ar45 | D1~D1082 | D1~D1082 | |
| 272665~273746 | Ar46 | D1~D1082 | D1~D1082 | |
| 273747~274828 | Ar47 | D1~D1082 | D1~D1082 | |
| 274829~275910 | Ar48 | D1~D1082 | D1~D1082 | |
| 275911~276992 | Ar49 | D1~D1082 | D1~D1082 | |
| 276993~278074 | Ar50 | D1~D1082 | D1~D1082 | |
| 278075~279156 | Ar51 | D1~D1082 | D1~D1082 | |
| 279157~280238 | Ar52 | D1~D1082 | D1~D1082 | |
| 280239~281320 | Ar53 | D1~D1082 | D1~D1082 | |
| 281321~282402 | Ar54 | D1~D1082 | D1~D1082 | |
| 282403~283484 | Ar55 | D1~D1082 | D1~D1082 | |
| 283485~284566 | Ar56 | D1~D1082 | D1~D1082 | |
| 284567~285648 | Ar57 | D1~D1082 | D1~D1082 | |
| 285649~286730 | Ar58 | D1~D1082 | D1~D1082 | |
| 286731~287812 | Ar59 | D1~D1082 | D1~D1082 | |
| 287813~288894 | Ar60 | D1~D1082 | D1~D1082 | |
| 288895~289976 | Ar61 | D1~D1082 | D1~D1082 | |
| 289977~291058 | Ar62 | D1~D1082 | D1~D1082 | |
| 291059~292140 | Ar63 | D1~D1082 | D1~D1082 | |
| 292141~293222 | Ar64 | D1~D1082 | D1~D1082 | |
| 293223~294304 | Ar65 | D1~D1082 | D1~D1082 | |
| 294305~295386 | Ar66 | D1~D1082 | D1~D1082 | |
| 295387~296468 | Ar67 | D1~D1082 | D1~D1082 | |
| 296469~297550 | Ar68 | D1~D1082 | D1~D1082 | |
| 297551~298632 | Ar69 | D1~D1082 | D1~D1082 | |
| 298633~299714 | D1~D1082 | D1~D1082 | Ar1 | R² = R⁴ |
| 299715~300796 | D1~D1082 | D1~D1082 | Ar2 | R² = R⁴ |
| 300797~301878 | D1~D1082 | D1~D1082 | Ar3 | |
| 301879~302960 | D1~D1082 | D1~D1082 | Ar4 | |
| 302961~304042 | D1~D1082 | D1~D1082 | Ar5 | |
| 304043~305124 | D1~D1082 | D1~D1082 | Ar6 | |
| 305125~306206 | D1~D1082 | D1~D1082 | Ar7 | |
| 306207~307288 | D1~D1082 | D1~D1082 | Ar8 | |
| 307289~308370 | D1~D1082 | D1~D1082 | Ar9 | |
| 308371~309452 | D1~D1082 | D1~D1082 | Ar10 | |
| 309453~310534 | D1~D1082 | D1~D1082 | Ar11 | |
| 310535~311616 | D1~D1082 | D1~D1082 | Ar12 | |
| 311617~312698 | D1~D1082 | D1~D1082 | Ar13 | |
| 312699~313780 | D1~D1082 | D1~D1082 | Ar14 | |
| 313781~314862 | D1~D1082 | D1~D1082 | Ar15 | |
| 314863~315944 | D1~D1082 | D1~D1082 | Ar16 | |
| 315945~317026 | D1~D1082 | D1~D1082 | Ar17 | |
| 317027~318108 | D1~D1082 | D1~D1082 | Ar18 | |
| 318109~319190 | D1~D1082 | D1~D1082 | Ar19 | |
| 319191~320272 | D1~D1082 | D1~D1082 | Ar20 | |
| 320273~321354 | D1~D1082 | D1~D1082 | Ar21 | |
| 321355~322436 | D1~D1082 | D1~D1082 | Ar22 | |
| 322437~323518 | D1~D1082 | D1~D1082 | Ar23 | |
| 323519~324600 | D1~D1082 | D1~D1082 | Ar24 | |
| 324601~325682 | D1~D1082 | D1~D1082 | Ar25 | |
| 325683~326764 | D1~D1082 | D1~D1082 | Ar26 | |
| 326765~327846 | D1~D1082 | D1~D1082 | Ar27 | |
| 327847~328928 | D1~D1082 | D1~D1082 | Ar28 | |
| 328929~330010 | D1~D1082 | D1~D1082 | Ar29 | |
| 330011~331092 | D1~D1082 | D1~D1082 | Ar30 | |
| 331093~332174 | D1~D1082 | D1~D1082 | Ar31 | |
| 332175~333256 | D1~D1082 | D1~D1082 | Ar32 | |
| 333257~334338 | D1~D1082 | D1~D1082 | Ar33 | |
| 334339~335420 | D1~D1082 | D1~D1082 | Ar34 | |
| 335421~336502 | D1~D1082 | D1~D1082 | Ar35 | |
| 336503~337584 | D1~D1082 | D1~D1082 | Ar36 | |
| 337585~338666 | D1~D1082 | D1~D1082 | Ar37 | |
| 338667~339748 | D1~D1082 | D1~D1082 | Ar38 | |
| 339749~340830 | D1~D1082 | D1~D1082 | Ar39 | |
| 340831~341912 | D1~D1082 | D1~D1082 | Ar40 | |
| 341913~342994 | D1~D1082 | D1~D1082 | Ar41 | |
| 342995~344076 | D1~D1082 | D1~D1082 | Ar42 | |
| 344077~345158 | D1~D1082 | D1~D1082 | Ar43 | |
| 345159~346240 | D1~D1082 | D1~D1082 | Ar44 | |
| 346241~347322 | D1~D1082 | D1~D1082 | Ar45 | |
| 347323~348404 | D1~D1082 | D1~D1082 | Ar46 | |
| 348405~349486 | D1~D1082 | D1~D1082 | Ar47 | |
| 349487~350568 | D1~D1082 | D1~D1082 | Ar48 | |
| 350569~351650 | D1~D1082 | D1~D1082 | Ar49 | |
| 351651~352732 | D1~D1082 | D1~D1082 | Ar50 | |
| 352733~353814 | D1~D1082 | D1~D1082 | Ar51 | |
| 353815~354896 | D1~D1082 | D1~D1082 | Ar52 | |
| 354897~355978 | D1~D1082 | D1~D1082 | Ar53 | |
| 355979~357060 | D1~D1082 | D1~D1082 | Ar54 | |
| 357061~358142 | D1~D1082 | D1~D1082 | Ar55 | |
| 358143~359224 | D1~D1082 | D1~D1082 | Ar56 | |
| 359225~360306 | D1~D1082 | D1~D1082 | Ar57 | |
| 360307~361388 | D1~D1082 | D1~D1082 | Ar58 | |
| 361389~362470 | D1~D1082 | D1~D1082 | Ar59 | |
| 362471~363552 | D1~D1082 | D1~D1082 | Ar60 | |
| 363553~364634 | D1~D1082 | D1~D1082 | Ar61 | |
| 364635~365716 | D1~D1082 | D1~D1082 | Ar62 | |
| 365717~366798 | D1~D1082 | D1~D1082 | Ar63 | |
| 366799~367880 | D1~D1082 | D1~D1082 | Ar64 | |
| 367881~368962 | D1~D1082 | D1~D1082 | Ar65 | |
| 368963~370044 | D1~D1082 | D1~D1082 | Ar66 | |
| 370045~371126 | D1~D1082 | D1~D1082 | Ar67 | |
| 371127~372208 | D1~D1082 | D1~D1082 | Ar68 | |
| 372209~373290 | D1~D1082 | D1~D1082 | Ar69 | |

In Tables 1 to 3, structures in which Ar¹ and Ar³ in the general formula (1) are perdeuterated carbazol-9-yl groups (D55) and Ar² and Ar⁴ are perdeuterated phenyl groups (Ar45) were specified as structures of Compounds 1 to 373290. In Table 4, for each of Compounds 1 to 373290, compounds obtained by changing Ar¹ to Ar⁴ as shown in Table 4 are shown in order in the form of a table. In Table 4, Compounds 1 to 373290 are also shown in the first row for clarifying the correspondence relationship. In the second row of Table 4, the compounds in which both Ar¹ and Ar³ of Compounds 1 to 373290 are D1 are referred to as Compounds 1(1) to 373290(1), respectively. For example, Compound 1(1) indicates a compound having a structure in which Ar¹ and Ar³ of Compound 1 are substituted with D1. Compound 2(1) indicates a compound having a structure in which Ar¹ and Ar³ of Compound 2 are substituted with D1. Compound 373290(1) indicates a compound having a structure in which Ar¹ and Ar³ of Compound 373290 are substituted with D1. In the third row of Table 4, the compounds in which both Ar¹ and Ar³ of Compounds 1 to 373290 are D7 are referred to as Compounds 1(2) to 373290(2), respectively. In this manner, compounds in which Ar¹ and Ar³, and Ar² and Ar⁴ of Compounds 1 to 373290 are listed in Table 4 are specified in order. X¹ to X³ of the compounds specified in Table 4 all represent a nitrogen atom (N), L¹ represents a single bond (L1), and R¹ represents a hydrogen atom. Ar¹ and Ar³ are the same, and Ar² and Ar⁴ are the same.

TABLE 4

| No. | Ar¹, Ar³ | Ar², Ar⁴ |
|---|---|---|
| 1~373290 | D55 | Ar45 |
| 1(1)~373290(1) | D1 | Ar45 |
| 1(2)~373290(2) | D7 | Ar45 |
| 1(3)~373290(3) | D8 | Ar45 |
| 1(4)~373290(4) | D9 | Ar45 |
| 1(5)~373290(5) | D17 | Ar45 |
| 1(6)~373290(6) | D27 | Ar45 |
| 1(7)~373290(7) | D37 | Ar45 |
| 1(8)~373290(8) | D39 | Ar45 |
| 1(9)~373290(9) | D47 | Ar45 |
| 1(10)~373290(10) | D51 | Ar45 |
| 1(11)~373290(11) | D61 | Ar45 |
| 1(12)~373290(12) | D62 | Ar45 |
| 1(13)~373290(13) | D63 | Ar45 |
| 1(14)~373290(14) | D71 | Ar45 |
| 1(15)~373290(15) | D81 | Ar45 |
| 1(16)~373290(16) | D1 | Ar1 |
| 1(17)~373290(17) | D7 | Ar1 |
| 1(18)~373290(18) | D8 | Ar1 |
| 1(19)~373290(19) | D9 | Ar1 |
| 1(20)~373290(20) | D17 | Ar1 |
| 1(21)~373290(21) | D27 | Ar1 |
| 1(22)~373290(22) | D37 | Ar1 |
| 1(23)~373290(23) | D39 | Ar1 |
| 1(24)~373290(24) | D47 | Ar1 |
| 1(25)~373290(25) | D51 | Ar1 |
| 1(26)~373290(26) | D55 | Ar1 |
| 1(27)~373290(27) | D61 | Ar1 |
| 1(28)~373290(28) | D62 | Ar1 |
| 1(29)~373290(29) | D63 | Ar1 |
| 1(30)~373290(30) | D71 | Ar1 |
| 1(31)~373290(31) | D81 | Ar1 |
| 1(32)~373290(32) | D1 | Ar10 |
| 1(33)~373290(33) | D7 | Ar10 |
| 1(34)~373290(34) | D8 | Ar10 |
| 1(35)~373290(35) | D9 | Ar10 |
| 1(36)~373290(36) | D17 | Ar10 |
| 1(37)~373290(37) | D27 | Ar10 |
| 1(38)~373290(38) | D37 | Ar10 |
| 1(39)~373290(39) | D39 | Ar10 |
| 1(40)~373290(40) | D47 | Ar10 |
| 1(41)~373290(41) | D51 | Ar10 |
| 1(42)~373290(42) | D55 | Ar10 |
| 1(43)~373290(43) | D61 | Ar10 |
| 1(44)~373290(44) | D62 | Ar10 |
| 1(45)~373290(45) | D63 | Ar10 |
| 1(46)~373290(46) | D71 | Ar10 |
| 1(47)~373290(47) | D81 | Ar10 |
| 1(48)~373290(48) | D1 | Ar12 |
| 1(49)~373290(49) | D7 | Ar12 |
| 1(50)~373290(50) | D8 | Ar12 |
| 1(51)~373290(51) | D9 | Ar12 |
| 1(52)~373290(52) | D17 | Ar12 |
| 1(53)~373290(53) | D27 | Ar12 |
| 1(54)~373290(54) | D37 | Ar12 |
| 1(55)~373290(55) | D39 | Ar12 |
| 1(56)~373290(56) | D47 | Ar12 |
| 1(57)~373290(57) | D51 | Ar12 |
| 1(58)~373290(58) | D55 | Ar12 |
| 1(59)~373290(59) | D61 | Ar12 |
| 1(60)~373290(60) | D62 | Ar12 |
| 1(61)~373290(61) | D63 | Ar12 |
| 1(62)~373290(62) | D71 | Ar12 |
| 1(63)~373290(63) | D81 | Ar12 |
| 1(64)~373290(64) | D1 | Ar14 |
| 1(65)~373290(65) | D7 | Ar14 |
| 1(66)~373290(66) | D8 | Ar14 |
| 1(67)~373290(67) | D9 | Ar14 |
| 1(68)~373290(68) | D17 | Ar14 |
| 1(69)~373290(69) | D27 | Ar14 |
| 1(70)~373290(70) | D37 | Ar14 |
| 1(71)~373290(71) | D39 | Ar14 |
| 1(72)~373290(72) | D47 | Ar14 |
| 1(73)~373290(73) | D51 | Ar14 |
| 1(74)~373290(74) | D55 | Ar14 |
| 1(75)~373290(75) | D61 | Ar14 |
| 1(76)~373290(76) | D62 | Ar14 |
| 1(77)~373290(77) | D63 | Ar14 |
| 1(78)~373290(78) | D71 | Ar14 |
| 1(79)~373290(79) | D81 | Ar14 |
| 1(80)~373290(80) | D1 | Ar34 |
| 1(81)~373290(81) | D7 | Ar34 |
| 1(82)~373290(82) | D8 | Ar34 |
| 1(83)~373290(83) | D9 | Ar34 |
| 1(84)~373290(84) | D17 | Ar34 |
| 1(85)~373290(85) | D27 | Ar34 |
| 1(86)~373290(86) | D37 | Ar34 |
| 1(87)~373290(87) | D39 | Ar34 |
| 1(88)~373290(88) | D47 | Ar34 |
| 1(89)~373290(89) | D51 | Ar34 |
| 1(90)~373290(90) | D55 | Ar34 |
| 1(91)~373290(91) | D61 | Ar34 |
| 1(92)~373290(92) | D62 | Ar34 |
| 1(93)~373290(93) | D63 | Ar34 |
| 1(94)~373290(94) | D71 | Ar34 |
| 1(95)~373290(95) | D81 | Ar34 |
| 1(96)~373290(96) | D1 | Ar36 |
| 1(97)~373290(97) | D7 | Ar36 |
| 1(98)~373290(98) | D8 | Ar36 |
| 1(99)~373290(99) | D9 | Ar36 |
| 1(100)~373290(100) | D17 | Ar36 |
| 1(101)~373290(101) | D27 | Ar36 |
| 1(102)~373290(102) | D37 | Ar36 |
| 1(103)~373290(103) | D39 | Ar36 |
| 1(104)~373290(104) | D47 | Ar36 |
| 1(105)~373290(105) | D51 | Ar36 |
| 1(106)~373290(106) | D55 | Ar36 |
| 1(107)~373290(107) | D61 | Ar36 |
| 1(108)~373290(108) | D62 | Ar36 |
| 1(109)~373290(109) | D63 | Ar36 |
| 1(110)~373290(110) | D71 | Ar36 |
| 1(111)~373290(111) | D81 | Ar36 |
| 1(112)~373290(112) | D1 | Ar38 |
| 1(113)~373290(113) | D7 | Ar38 |
| 1(114)~373290(114) | D8 | Ar38 |
| 1(115)~373290(115) | D9 | Ar38 |
| 1(116)~373290(116) | D17 | Ar38 |
| 1(117)~373290(117) | D27 | Ar38 |
| 1(118)~373290(118) | D37 | Ar38 |
| 1(119)~373290(119) | D39 | Ar38 |
| 1(120)~373290(120) | D47 | Ar38 |
| 1(121)~373290(121) | D51 | Ar38 |
| 1(122)~373290(122) | D55 | Ar38 |
| 1(123)~373290(123) | D61 | Ar38 |
| 1(124)~373290(124) | D62 | Ar38 |
| 1(125)~373290(125) | D63 | Ar38 |
| 1(126)~373290(126) | D71 | Ar38 |
| 1(127)~373290(127) | D81 | Ar38 |
| 1(128)~373290(128) | D1 | Ar54 |
| 1(129)~373290(129) | D7 | Ar54 |
| 1(130)~373290(130) | D8 | Ar54 |
| 1(131)~373290(131) | D9 | Ar54 |
| 1(132)~373290(132) | D17 | Ar54 |
| 1(133)~373290(133) | D27 | Ar54 |
| 1(134)~373290(134) | D37 | Ar54 |
| 1(135)~373290(135) | D39 | Ar54 |
| 1(136)~373290(136) | D47 | Ar54 |
| 1(137)~373290(137) | D51 | Ar54 |
| 1(138)~373290(138) | D55 | Ar54 |
| 1(139)~373290(139) | D61 | Ar54 |
| 1(140)~373290(140) | D62 | Ar54 |
| 1(141)~373290(141) | D63 | Ar54 |
| 1(142)~373290(142) | D71 | Ar54 |
| 1(143)~373290(143) | D81 | Ar54 |
| 1(144)~373290(144) | D1 | Ar56 |
| 1(145)~373290(145) | D7 | Ar56 |
| 1(146)~373290(146) | D8 | Ar56 |
| 1(147)~373290(147) | D9 | Ar56 |
| 1(148)~373290(148) | D17 | Ar56 |
| 1(149)~373290(149) | D27 | Ar56 |
| 1(150)~373290(150) | D37 | Ar56 |
| 1(151)~373290(151) | D39 | Ar56 |
| 1(152)~373290(152) | D47 | Ar56 |
| 1(153)~373290(153) | D51 | Ar56 |
| 1(154)~373290(154) | D55 | Ar56 |
| 1(155)~373290(155) | D61 | Ar56 |
| 1(156)~373290(156) | D62 | Ar56 |
| 1(157)~373290(157) | D63 | Ar56 |
| 1(158)~373290(158) | D71 | Ar56 |
| 1(159)~373290(159) | D81 | Ar56 |
| 1(160)~373290(160) | D1 | Ar58 |
| 1(161)~373290(161) | D7 | Ar58 |
| 1(162)~373290(162) | D8 | Ar58 |
| 1(163)~373290(163) | D9 | Ar58 |
| 1(164)~373290(164) | D17 | Ar58 |
| 1(165)~373290(165) | D27 | Ar58 |
| 1(166)~373290(166) | D37 | Ar58 |
| 1(167)~373290(167) | D39 | Ar58 |
| 1(168)~373290(168) | D47 | Ar58 |
| 1(169)~373290(169) | D51 | Ar58 |
| 1(170)~373290(170) | D55 | Ar58 |
| 1(171)~373290(171) | D61 | Ar58 |
| 1(172)~373290(172) | D62 | Ar58 |
| 1(173)~373290(173) | D63 | Ar58 |
| 1(174)~373290(174) | D71 | Ar58 |
| 1(175)~373290(175) | D81 | Ar58 |
| 1(165)~373290(165) | D55 | D1 |
| 1(166)~373290(166) | D55 | D7 |
| 1(167)~373290(167) | D55 | D8 |
| 1(168)~373290(168) | D55 | D9 |
| 1(169)~373290(169) | D55 | D17 |
| 1(170)~373290(170) | D55 | D27 |
| 1(171)~373290(171) | D55 | D37 |
| 1(172)~373290(172) | D55 | D39 |
| 1(173)~373290(173) | D55 | D47 |
| 1(174)~373290(174) | D55 | D51 |
| 1(175)~373290(175) | D55 | D55 |
| 1(176)~373290(176) | D55 | D61 |
| 1(177)~373290(177) | D55 | D62 |
| 1(178)~373290(178) | D55 | D63 |
| 1(179)~373290(179) | D55 | D71 |
| 1(180)~373290(180) | D55 | D81 |
| 2(181)~373290(181) | Ar1 | Ar1 |
| 1(182)~373290(182) | Ar10 | Ar10 |
| 1(183)~373290(183) | Ar12 | Ar12 |
| 1(184)~373290(184) | Ar14 | Ar14 |
| 1(185)~373290(185) | Ar34 | Ar34 |
| 1(186)~373290(186) | Ar36 | Ar36 |
| 1(187)~373290(187) | Ar38 | Ar38 |
| 1(188)~373290(188) | Ar45 | Ar45 |
| 1(189)~373290(189) | Ar54 | Ar54 |
| 1(190)~373290(190) | Ar56 | Ar56 |
| 1(191)~373290(191) | Ar58 | Ar58 |

The compounds specified by the numbers in Tables 1 to 4 are all individually disclosed. In addition, among the specific examples of the compounds, in the case where a rotamer is present, a mixture of rotamers and each separated rotamer are also disclosed in the description herein.

In one aspect of the present invention, the compound is selected from the group of compounds specified in Table 4.

An example of a preferred group of compounds represented by the general formula (1) is given below.

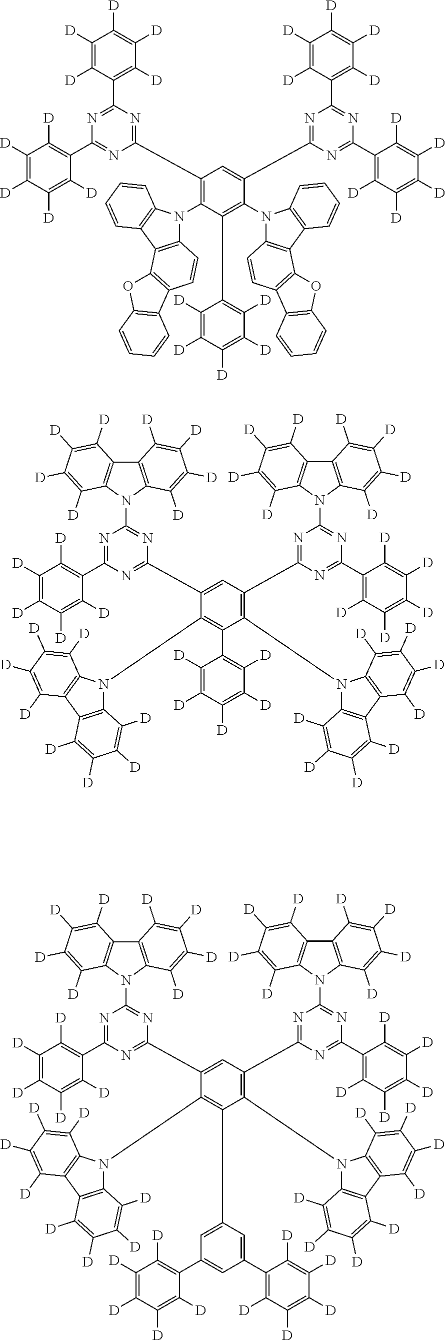

A molecular weight of the compound represented by the general formula (1) is preferably 1500 or less, more preferably 1200 or less, further preferably 1000 or less, and still further preferably 900 or less, for example, in the case where an organic layer containing the compound represented by the general formula (1) is intended to be film-formed and used by a vapor deposition method. The lower limit value of the molecular weight is the molecular weight of the smallest compound represented by the general formula (1).

The compound represented by the general formula (1) may be formed into a film by a coating method regardless of the molecular weight. When the coating method is used, even a compound having a relatively large molecular weight can be formed into a film. The compound represented by the general formula (1) has an advantage of being easily dissolved in an organic solvent. For this reason, the compound represented by the general formula (1) is easily applicable to a coating method and is easily purified to increase its purity.

It is also conceivable to use a compound containing a plurality of structures represented by the general formula (1) in a molecule as a light-emitting material by applying the present invention.

For example, it is conceivable that a polymer obtained by allowing a polymerizable group to be present in the structure represented by the general formula (1) in advance and polymerizing the polymerizable group is used as the light-emitting material. For example, it is conceivable that a polymer having a repeating unit is obtained by preparing a monomer containing a polymerizable functional group at any site of the general formula (1) and polymerizing the monomer alone or copolymerizing the monomer with another monomer, and the polymer is used as the light-emitting material. Alternatively, it is also conceivable to obtain a dimer or a trimer by coupling compounds having a structure represented by the general formula (1) to each other and to use the dimer or the trimer as a light-emitting material.

Examples of the polymer having a repeating unit containing a structure represented by the general formula (1) include polymers containing a structure represented by any one of the following two general formulae.

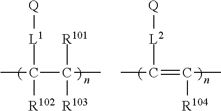

In the above general formulae, Q represents a group containing the structure represented by the general formula (1), and L¹ and L² each represent a linking group. The linking group preferably has 0 to 20 carbon atoms, more preferably 1 to 15 carbon atoms, and further preferably 2 to 10 carbon atoms. The linking group preferably has a structure represented by —X¹¹-L¹¹-. Here, X¹¹ represents an oxygen atom or a sulfur atom, and is preferably an oxygen atom. L¹¹ represents a linking group, and is preferably a substituted or unsubstituted alkylene group, or a substituted or unsubstituted arylene group, more preferably a substituted or unsubstituted alkylene group having 1 to 10 carbon atoms, or a substituted or unsubstituted phenylene group.

In the above general formulae, R¹⁰¹, R¹⁰², R¹⁰³, and R¹⁰⁴ each independently represent a substituent. The substituent is preferably a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and further preferably an unsubstituted alkyl group having 1 to 3 carbon atoms or an unsubstituted alkoxy group having 1 to 3 carbon atoms.

The linking group represented by L¹ and L² can be bonded to any site of the general formula (1) constituting Q. Two or more linking groups may be linked to one Q to form a cross-linked structure or a network structure.

Specific structural examples of the repeating unit include structures represented by the following formulae.

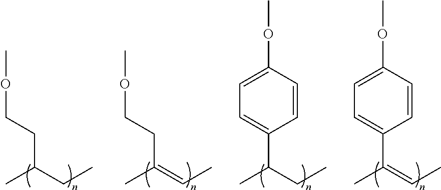

The polymer having a repeating unit including these formulae can be synthesized by introducing a hydroxyl group into any site of the general formula (1), reacting the following compound using the hydroxy group as a linker to introduce a polymerizable group, and polymerizing the polymerizable group.

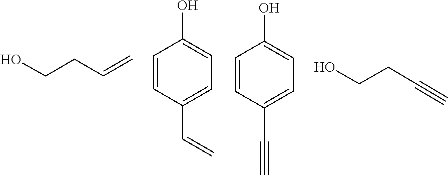

The polymer having a structure represented by the general formula (1) in the molecule may be a polymer having only a repeating unit that has the structure represented by the general formula (1), or may be a polymer containing a repeating unit that has any other structure. The repeating unit having the structure represented by the general formula (1) to be contained in the polymer may be a single kind or two or more kinds. The repeating unit not having the structure represented by the general formula (1) includes those derived from monomers used in general copolymerization. For example, the repeating unit includes repeating units derived from monomers having an ethylenically unsaturated bond, such as ethylene or styrene.

With respect to the use and the like of the compound represented by the general formula (1), reference can be made to [0058] to [0059], [0061], and [0063] of WO2022/168956A1, which are incorporated as a part of this description by reference.

### [Method for Synthesizing Compound Represented by General Formula (1)]

The compound represented by the general formula (1) includes a novel compound.

The compound represented by the general formula (1) can be synthesized by combining known reactions. In the compound represented by the general formula (1), two of R² to R⁵ are donor groups. For example, the compound represented by the general formula (1) in which a substituted or unsubstituted carbazol-9-yl group is a donor group can be synthesized by reacting a substituted or unsubstituted carbazole with a precursor in which the site of the donor group is a fluorine atom. For details of the reaction conditions, Synthesis Examples described later can be referred to.

### [Organic Light-Emitting Device]

The organic light-emitting device of the present invention contains the compound represented by the general formula (1) and a host material or a dopant material in the same layer. Here, the same layer is preferably a light-emitting layer. In one aspect of the present invention, the compound represented by the general formula (1) and the host material are contained in the same layer. The host material is used at a concentration higher than that of the compound represented by the general formula (1), and preferably has a lowest excited singlet energy higher than that of the compound represented by the general formula (1). In one aspect of the present invention, the compound represented by the general formula (1) and the dopant material are contained in the same layer. The dopant material is used at a concentration lower than that of the compound represented by the general formula (1), and preferably has a lowest excited singlet energy lower than that of the compound represented by the general formula (1).

In some embodiments, the compound represented by the general formula (1) functions as a light-emitting material in the organic light-emitting device. In some embodiments, the compound represented by the general formula (1) functions as a light-emitting material that emits delayed fluorescence in the organic light-emitting device.

In some embodiments of the present disclosure, the compound represented by the general formula (1) is, when excited thermally or by an electronic means, able to emit light in a UV region, emit light of blue, green, yellow, orange, or red in a visible spectral region (e.g., about 420 nm to about 500 nm, about 500 nm to about 600 nm, or about 600 nm to about 700 nm) or emit light in a near IR region.

In some embodiments of the present disclosure, the compound represented by the general formula (1) is, when excited thermally or by an electronic means, able to emit light of red or orange in a visible spectral region (e.g., about 620 nm to about 780 nm, about 650 nm).

In some embodiments of the present disclosure, the compound represented by the general formula (1) is, when excited thermally or by an electronic means, able to emit light of orange or yellow in a visible spectral region (e.g., about 570 nm to about 620 nm, about 590 nm, about 570 nm).

In some embodiments of the present disclosure, the compound represented by the general formula (1) is, when excited thermally or by an electronic means, able to emit light of green in a visible spectral region (e.g., about 490 nm to about 575 nm, about 510 nm).

In some embodiments of the present disclosure, the compound represented by the general formula (1) is, when excited thermally or by an electronic means, able to emit light of blue in a visible spectral region (e.g., about 400 nm to about 490 nm, about 475 nm).

In some embodiments of the present disclosure, the compound represented by the general formula (1) is, when excited thermally or by an electronic means, able to emit light in a UV spectral region (e.g., about 280 to 400 nm).

In some embodiments of the present disclosure, the compound represented by the general formula (1) is, when excited thermally or by an electronic means, able to emit light in an IR spectral region (e.g., about 780 nm to 2 μm).

When a layer of an organic light-emitting device is formed, a film forming technique can be used.

In some embodiments, a film containing the compound represented by the general formula (1) can be formed in a wet process. In a wet process, a solution prepared by dissolving a composition containing the compound of the present invention is applied onto a surface, and then the solvent is removed to form a film. The wet process includes a spin coating method, a slit coating method, an ink jet method (a spraying method), a gravure printing method, an offset printing method, and a flexographic printing method, which, however, are not limitative. In the wet process, an appropriate organic solvent capable of dissolving a composition containing the compound of the present invention is selected and used. In some embodiments, a substituent (e.g., an alkyl group) capable of increasing the solubility in an organic solvent can be introduced into the compound contained in the composition.

In some embodiments, a film containing the compound of the present invention can be formed in a dry process. In some embodiments, a vacuum deposition method is employable as a dry process, which, however, is not limitative. In the case where a vacuum deposition method is employed, compounds to constitute a film may be co-deposited from individual vapor deposition sources, or may be co-deposited from a single vapor deposition source formed by mixing the compounds. In the case where a single vapor deposition source is used, a mixed powder prepared by mixing compound powders may be used, or a compression molded body prepared by compression-molding the mixed powder may be used, or a mixture prepared by heating and melting the compounds and cooling the resulting melt may be used. In some embodiments, by co-deposition under the condition where the vapor deposition rate (weight reduction rate) of the plural compounds contained in a single vapor deposition source is the same or is nearly the same, a film having a compositional ratio corresponding to the compositional ratio of the plural compounds contained in the vapor deposition source can be formed. When plural compounds are mixed in the same compositional ratio as the compositional ratio of the film to be formed to prepare a vapor deposition source, a film having a desired compositional ratio can be formed in a simplified manner. In some embodiments, the temperature at which the compounds to be co-deposited have the same weight reduction ratio is specifically defined, and the temperature can be employed as the temperature of co-deposition.

In some embodiments, the organic light-emitting device is an organic photoluminescent device (organic PL device). In some embodiments, the organic light-emitting device is an organic electroluminescent device (organic EL device). In some embodiments, the compound represented by the general formula (1) assists light emission from the other light-emitting materials contained in the light-emitting layer (as a so-called assist dopant). In some embodiments, the compound represented by the general formula (1) contained in the light-emitting layer is in a lowest excited singlet energy level, and is contained between the lowest excited single energy level of the host material contained in the light-emitting layer and the lowest excited singlet energy level of the other light-emitting materials contained in the light-emitting layer.

In some embodiments, the organic photoluminescent device includes at least one light-emitting layer. In some embodiments, the organic electroluminescent device includes at least an anode, a cathode, and an organic layer between the anode and the cathode. In some embodiments, the organic layer includes at least a light-emitting layer. In some embodiments, the organic layer includes only a light-emitting layer. In some embodiments, the organic layer includes one or more organic layers as well as the light-emitting layer. Examples of the organic layer include a hole transport layer, a hole injection layer, an electron barrier layer, a hole barrier layer, an electron injection layer, an electron transport layer, and an exciton barrier layer. In some embodiments, the hole transport layer may be a hole injection transport layer having a hole injection function, and the electron transport layer may be an electron injection transport layer having an electron injection function.

### Light-Emitting Layer:

In some embodiments, the light-emitting layer is a layer where holes and electrons injected from the anode and the cathode, respectively, are recombined to form excitons. In some embodiments, the layer emits light.

In some embodiments, the light-emitting layer contains a light-emitting material that is a dopant material and a host material. In some embodiments, the light-emitting material is a compound represented by the general formula (1). In some embodiments, for improving light emission efficiency of an organic electroluminescent device and an organic photoluminescence device, the singlet exciton and the triplet exciton generated in a light-emitting material are confined inside the light-emitting material. In some embodiments, a host material is used in the light-emitting layer in addition to a light-emitting material. In some embodiments, the host material is an organic compound. In some embodiments, the organic compound has an excited singlet energy and an excited triplet energy, and at least one of them is higher than those in the light-emitting material of the present invention. In some embodiments, the singlet exciton and the triplet exciton generated in the light-emitting material of the present invention are confined in the molecules of the light-emitting material of the present invention. In some embodiments, the singlet and triplet excitons are fully confined for improving light emission efficiency. In some embodiments, although high light emission efficiency is still attained, singlet excitons and triplet excitons are not fully confined, that is, a host material capable of attaining high light emission efficiency can be used in the present invention with no specific limitation. In some embodiments, in the light-emitting material in the light-emitting layer of the device of the present invention, light emission occurs. In some embodiments, emitted light includes both fluorescence and delayed fluorescence. In some embodiments, emitted light includes emitted light from a host material. In some embodiments, emitted light is composed of emitted light from a host material. In some embodiments, emitted light includes emitted light from the compound represented by the general formula (1) and emitted light from a host material. In some embodiments, a TADF molecule and a host material are used. In some embodiments, TADF is an assist dopant and has a lower excited singlet energy than the host material in the light-emitting layer and a higher excited singlet energy than the light-emitting material in the light-emitting layer.

### (Dopant Material)

When the compound represented by the general formula (1) is used as a host material or an assist dopant, various compounds can be employed as a light-emitting material (preferably a fluorescent material) which is a dopant material. As such a light-emitting material, it is possible to use an anthracene derivative, a tetracene derivative, a naphthacene derivative, a pyrene derivative, a perylene derivative, a chrysene derivative, a rubrene derivative, a coumarin derivative, a pyran derivative, a stilbene derivative, a fluorene derivative, an anthryl derivative, a pyrromethene derivative, a terphenyl derivative, a terphenylene derivative, a fluoranthene derivative, an amine derivative, a quinacridone derivative, an oxadiazole derivative, a malononitrile derivative, a pyran derivative, a carbazole derivative, a julolidine derivative, a thiazole derivative, a derivative having a metal (Al, Zn), or the like. These exemplified skeletons may have substituents, or may not have substituents. Further, these exemplified skeletons may be combined with each other.

Hereinafter, dopant materials (light-emitting materials) which can be used in combination with the compound represented by the general formula (1) will be exemplified.

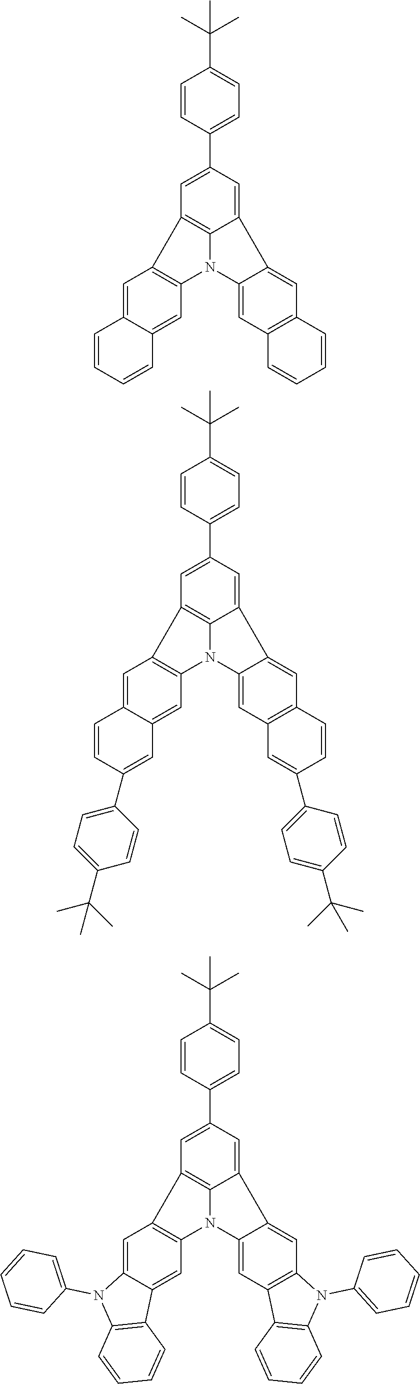

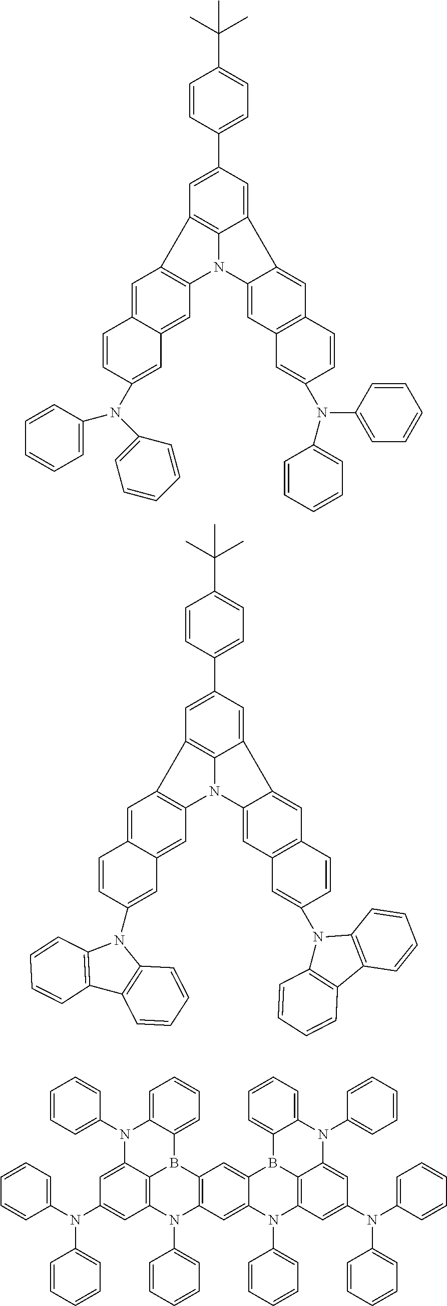

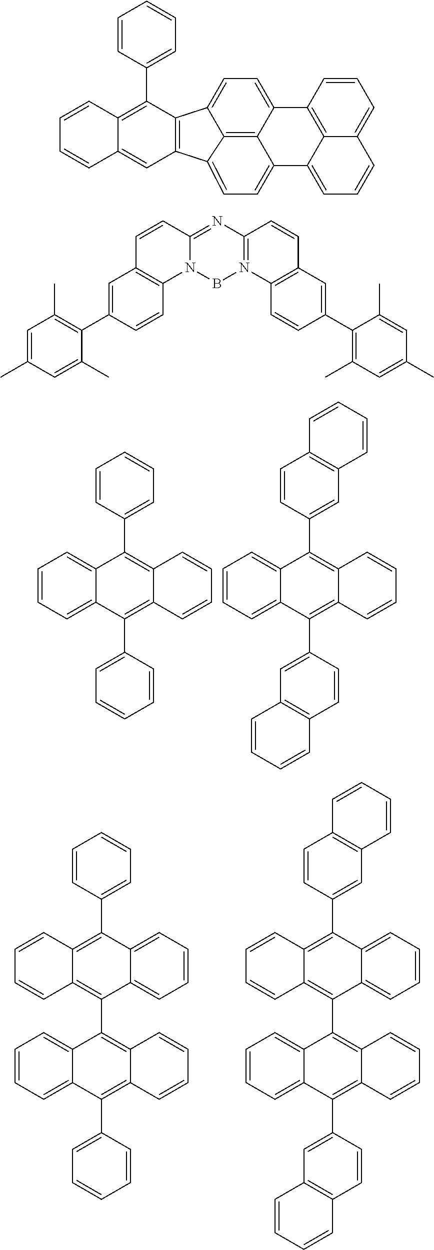

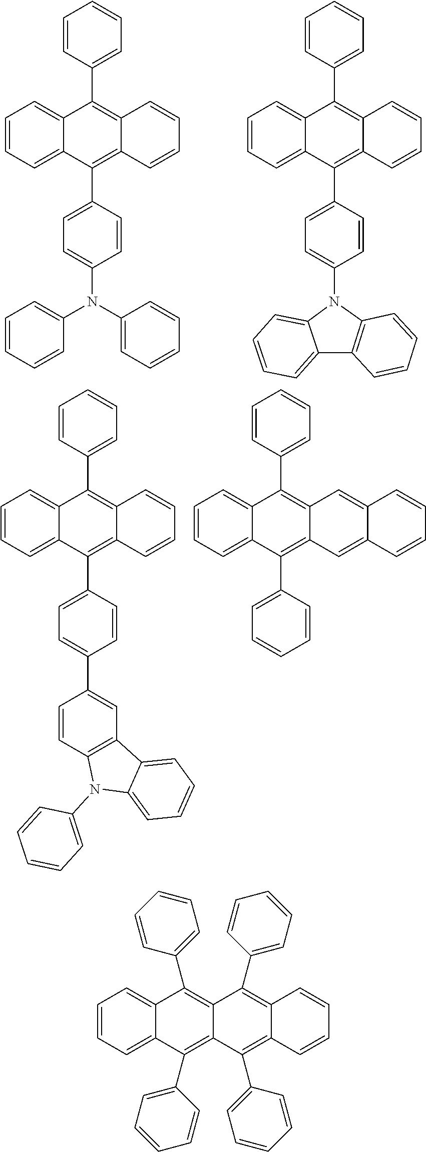

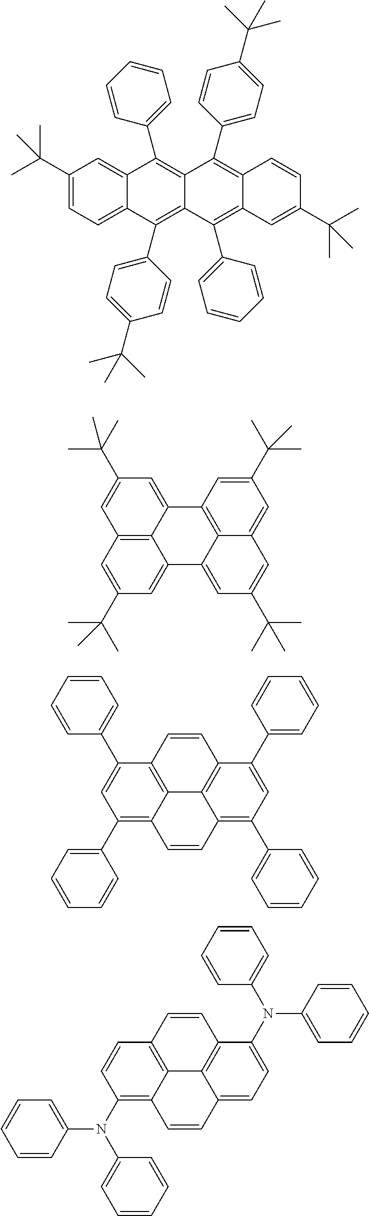

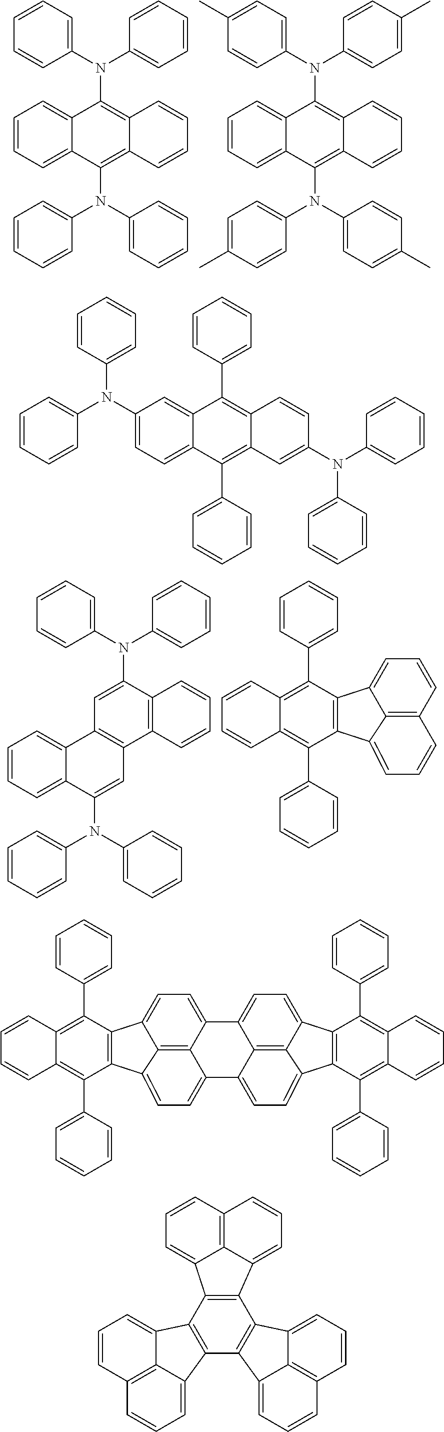

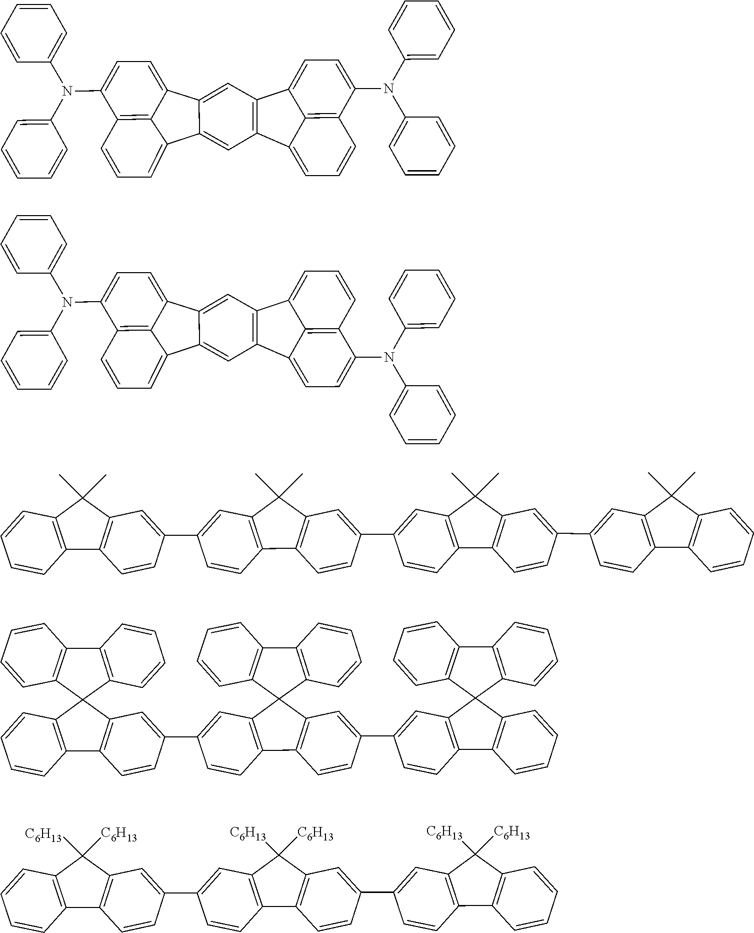

In addition to the above, the compounds described in paragraphs 0220 to 0239 of WO2015/022974 can also be employed as the light-emitting material used together with the assist dopant having the structure represented by the general formula (1).

In some embodiments, the light-emitting layer contains two or more kinds of TADF molecules differing in the structure. For example, the light-emitting layer can contain three kinds of materials of a host material, a first TADF molecule, and a second TADF molecule whose excited singlet energy level is higher in that order. In that case, both the first TADF molecule and the second TADF molecule are preferably such that the difference ΔE_{ST} between the lowest excited singlet energy level and the lowest excited triplet energy level at 77 K is 0.3 eV or less, more preferably 0.25 eV or less, still more preferably 0.2 eV or less, even more preferably 0.15 eV or less, further preferably 0.1 eV or less, still further preferably 0.07 eV or less, further more preferably 0.05 eV or less, still further more preferably 0.03 eV or less, and particularly preferably 0.01 eV or less. The content of the first TADF molecule in the light-emitting layer is preferably larger than the content of the second TADF molecule therein. The content of the host material in the light-emitting layer is preferably larger than the content of the second TADF molecule therein. The content of the first TADF molecule in the light-emitting layer may be larger than or may be smaller than or may be the same as the content of the host material therein. In some embodiments, the composition in the light-emitting layer may be 10% by weight to 70% by weight of a host material, 10% by weight to 80% by weight of a first TADF molecule, and 0.1% by weight to 30% by weight of a second TADF molecule. In some embodiments, the composition in the light-emitting layer may be 20% by weight to 45% by weight of a host material, 50% by weight to 75% by weight of a first TADF molecule, and 5% by weight to 20% by weight of a second TADF molecule. In some embodiments, the photoluminescence quantum yield φPL1(A) by photo-excitation of a co-deposited film of a first TADF molecule and a host material (the content of the first TADF molecule in the co-deposited film=A % by weight) and the photoluminescence quantum yield φPL2(A) by photo-excitation of a co-deposited film of a second TADF molecule and a host material (the content of the second TADF molecule in the co-deposited film=A % by weight) satisfy a relational formula φPL1(A)>φPL2(A). In some embodiments, the photoluminescence quantum yield φPL2(B) by photo-excitation of a co-deposited film of a second TADF molecule and a host material (the content of the second TADF molecule in the co-deposited film=B % by weight) and the photoluminescence quantum yield φPL2(100) by photo-excitation of a single film of a second TADF molecule satisfy a relational formula φPL2(B)>φPL2(100). In some embodiments, the light-emitting layer can contain three kinds of TADF molecules differing in the structure. The compound of the present invention may be any of the plural TADF compounds contained in the light-emitting layer.

In some embodiments, the light-emitting layer can be composed of materials selected from the group consisting of a host material, an assist dopant, and a light-emitting material. In some embodiments, the light-emitting layer does not contain a metal element. In some embodiments, the light-emitting layer can be formed of a material composed of atoms alone selected from the group consisting of a carbon atom, a hydrogen atom, a deuterium atom, a nitrogen atom, an oxygen atom, and a sulfur atom. Alternatively, the light-emitting layer can be formed of a material composed of atoms alone selected from the group consisting of a carbon atom, a hydrogen atom, a deuterium atom, a nitrogen atom, and an oxygen atom. Alternatively, the light-emitting layer can be formed of a material composed of atoms alone selected from the group consisting of a carbon atom, a hydrogen atom, a nitrogen atom, and an oxygen atom.

In the case where the light-emitting layer contains any other TADF material than the compound of the present invention, the TADF material may be a known delayed fluorescent material. As preferred delayed fluorescent materials, there can be mentioned compounds included in the general formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101 to 0121; JP 2014-9352 A, paragraphs 0007 to 0041 and 0060 to 0069; JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; WO2018/047853, paragraphs 0016 to 0044; and especially, exemplary compounds therein capable of emitting delayed fluorescence. In addition, also preferably employable here are light-emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541, and WO2015/159541. These patent publications described in this paragraph are hereby incorporated as a part of this description by reference.

### (Host Material)

When the compound represented by the general formula (1) is used together with a host material, the amount of the compound represented by the general formula (1) is 0.1% by weight or more. In some embodiments where a host material is used, the amount of the compound represented by the general formula (1) contained in a light-emitting layer as a light-emitting material is 1% by weight or more. In some embodiments where a host material is used, the amount of the compound represented by the general formula (1) contained in a light-emitting layer as a light-emitting material is 50% by weight or less. In some embodiments where a host material is used, the amount of the compound represented by the general formula (1) contained in a light-emitting layer as a light-emitting material is 20% by weight or less. In some embodiments where a host material is used, the amount of the compound represented by the general formula (1) contained in a light-emitting layer as a light-emitting material is 10% by weight or less.

In some embodiments, the host material in a light-emitting layer is an organic compound having a hole transporting capability and an electron transporting capability. In some embodiments, the host material in a light-emitting layer is an organic compound that prevents increase in the wavelength of emitted light. In some embodiments, the host material in a light-emitting layer is an organic compound having a high glass transition temperature.

In some embodiments, the host material is selected from the group consisting of the followings:

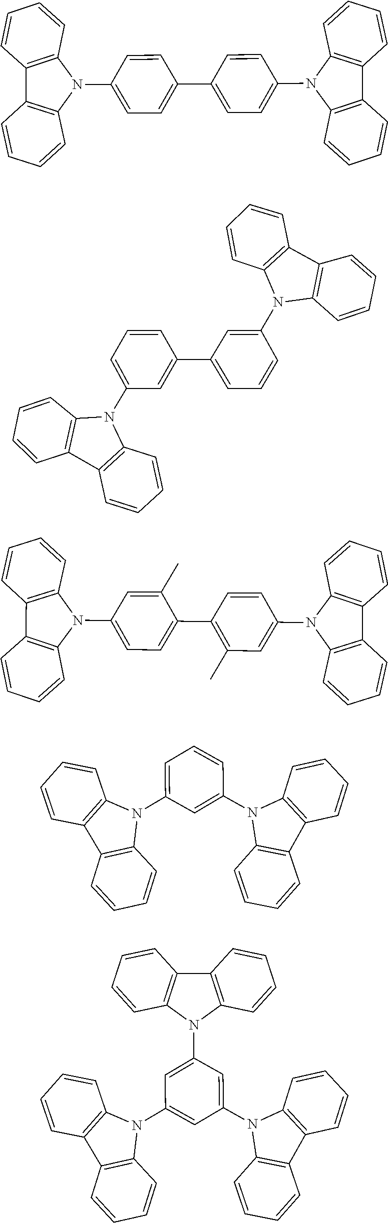

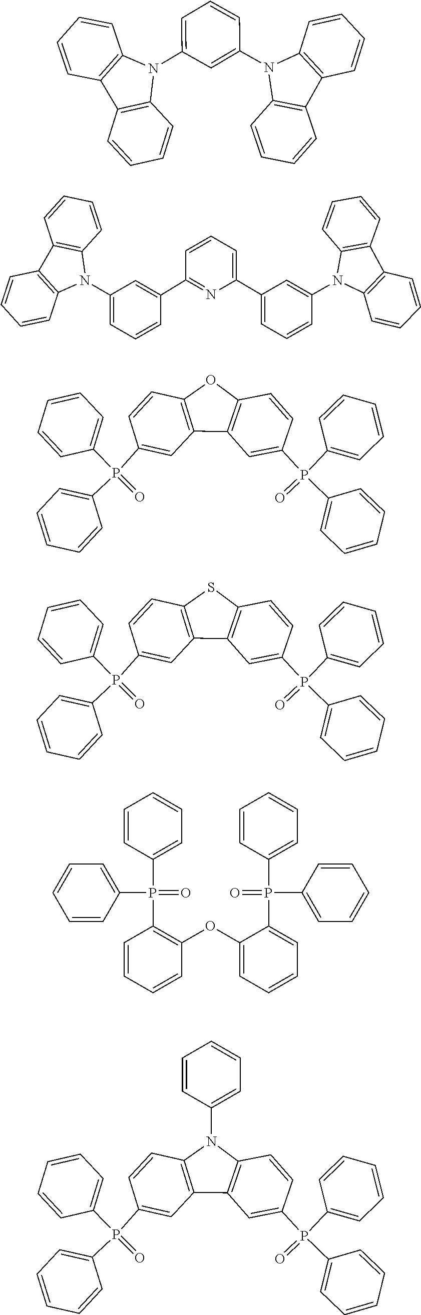 [SMILES: c1cc(-n2c3ccccc3c3ccccc32)cc(-n2c3ccccc3c3ccccc32)c1 ; c1cc(-c2cccc(-c3cccc(-n4c5ccccc5c5ccccc54)c3)n2)cc(-n2c3ccccc3c3ccccc32)c1 ; O=P(c1ccccc1)(c1ccccc1)c1ccc2oc3ccc(P(=O)(c4ccccc4)c4ccccc4)cc3c2c1 ; O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2c(c1)c1cc(P(=O)(c3ccccc3)c3ccccc3)ccc1n2-c1ccccc1]

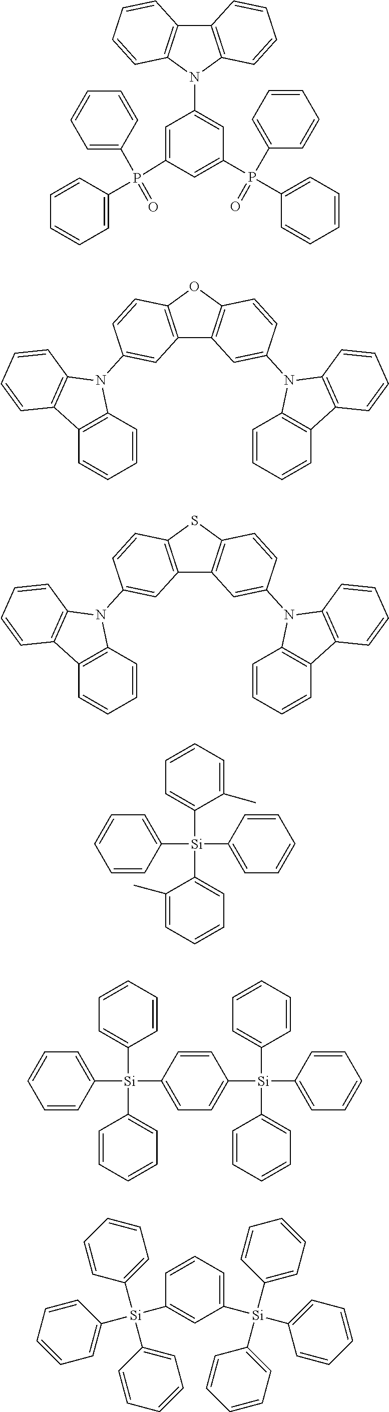

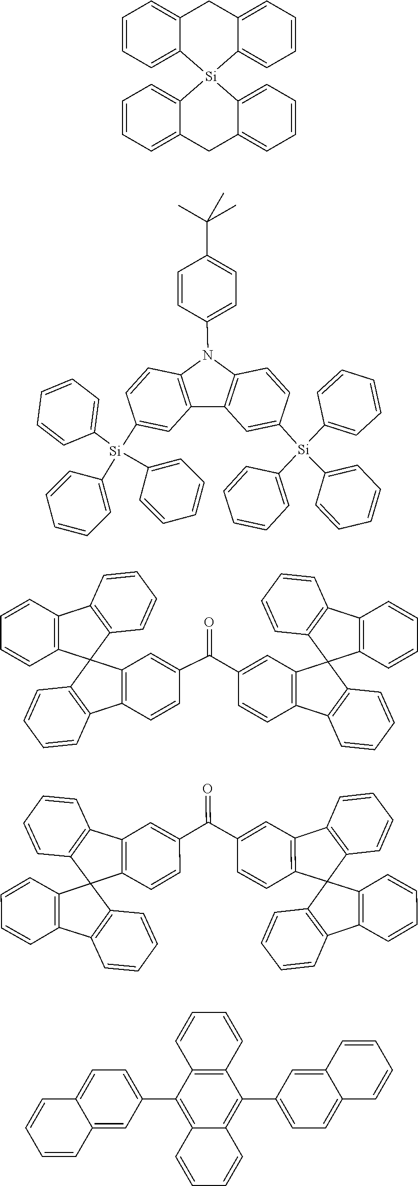

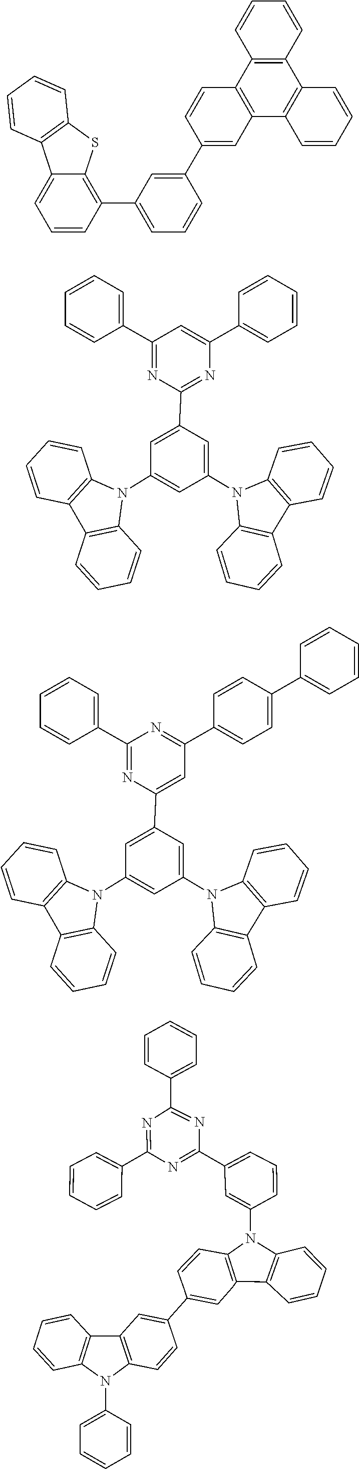

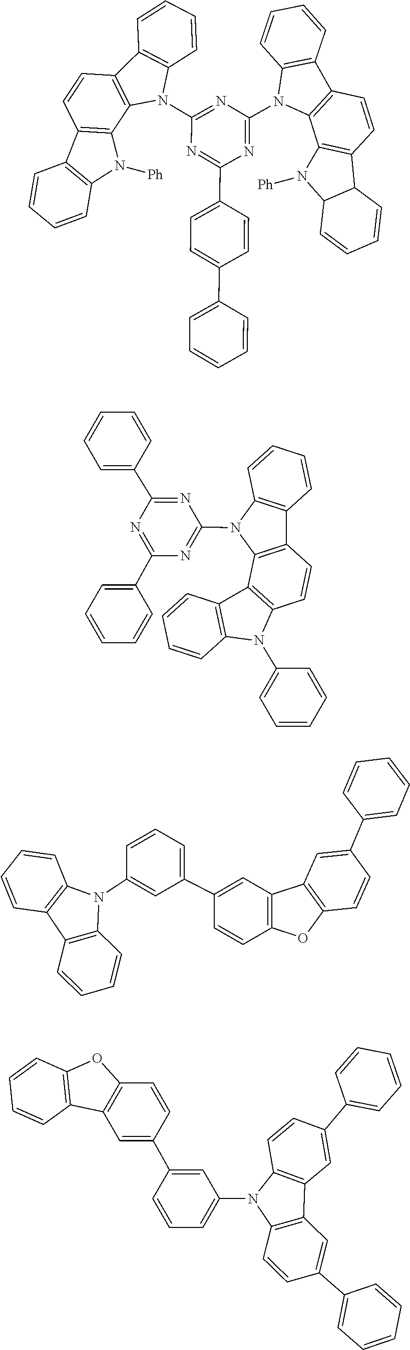 [SMILES: C1=CC2c3ccc4c5ccccc5n(-c5nc(-c6ccc(-c7ccccc7)cc6)nc(-n6c7ccccc7c7ccc8c9ccccc9n(-c9ccccc9)c8c76)n5)c4c3N(c3ccccc3)C2C=C1 ; c1ccc(-c2nc(-c3ccccc3)nc(-n3c4ccccc4c4ccc5c(c6ccccc6n5-c5ccccc5)c43)n2)cc1 ; c1ccc(-c2ccc3oc4ccc(-c5cccc(-n6c7ccccc7c7ccccc76)c5)cc4c3c2)cc1 ; c1ccc(-c2ccc3c(c2)c2cc(-c4ccccc4)ccc2n3-c2cccc(-c3ccc4oc5ccccc5c4c3)c2)cc1]

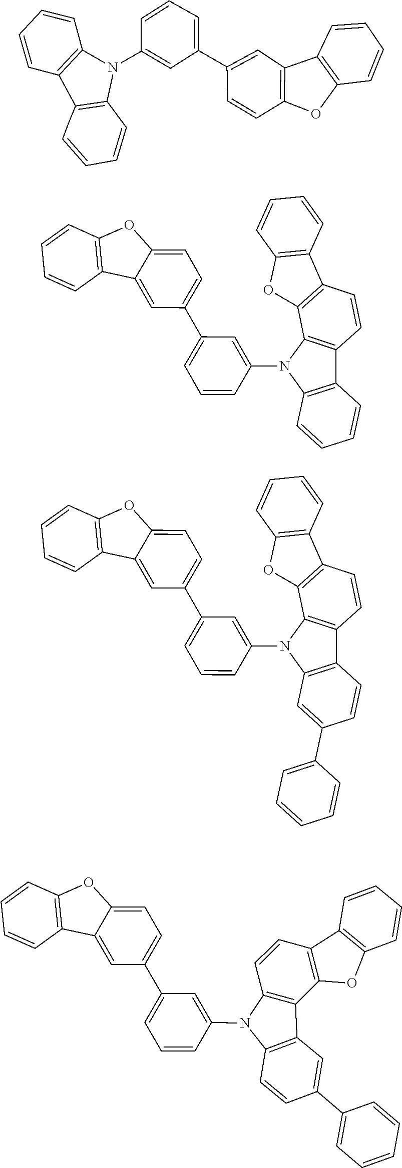

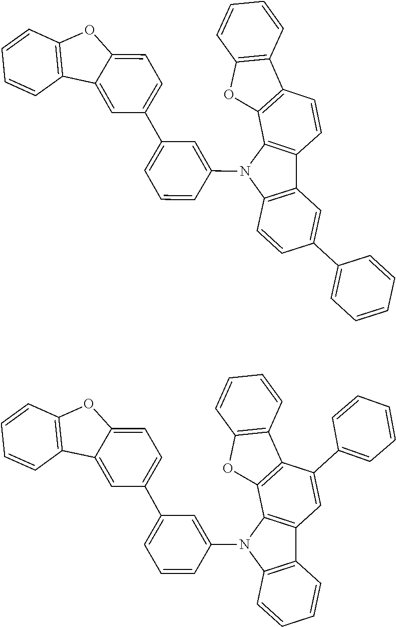 [SMILES: c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

In one preferred aspect of the present invention, the host material used together with the compound represented by the general formula (1) is a compound having a structure represented by the following general formula (4).

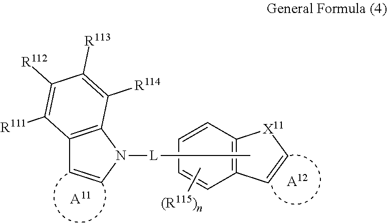

In the general formula (4), X¹¹ represents O, S, N(R^{A}), or C(R^{B})(R^{C}). In one aspect of the present invention, X¹¹ is O, S, or N(R^{A}). In one aspect of the present invention, X¹¹ is O or S. In one aspect of the present invention, X¹¹ is N(R^{A}). In one aspect of the present invention, X¹¹ is O. In one aspect of the present invention, X¹¹ is S. When X¹¹ is O, S, or C(R^{B})(R^{C}), L is bonded to a benzene ring to which (R¹¹⁵)n is bonded. When X¹¹ is N(R^{A}), L is bonded to a benzene ring to which (R¹¹⁵)n is bonded or N represented by X¹¹. As described herein, a bond elongating from L to the right side means being bonded to a benzene ring to which (R¹¹⁵)n is bonded, or being bonded to X¹¹ (that is, N) when X¹¹ is N.

In the general formula (4), A¹¹ and A¹² are each independently a benzene ring, a furan ring, a thiol ring, a pyrrole ring, or a cyclopentadiene ring, which may have other rings further fused in such rings or may be substituted. In one preferred aspect of the present invention, A¹¹ is a benzene ring. In one preferred aspect of the present invention, A¹² is a benzene ring. In a further preferred aspect of the present invention, both A¹¹ and A¹² are benzene rings. In one aspect of the present invention, at least one of A¹¹ and A¹² is a furan ring, a thiol ring, a pyrrole ring, or a cyclopentadiene ring. In one aspect of the present invention, at least one of A¹¹ and A¹² is furan ring. In one aspect of the present invention, at least one of A¹¹ and A¹² is a thiol ring. In one aspect of the present invention, at least one of A¹¹ and A¹² is a pyrrole ring. In one aspect of the present invention, at least one of A¹¹ and A¹² is a cyclopentadiene ring.

The benzene ring, the furan ring, the thiol ring, the pyrrole ring, and the cyclopentadiene ring mentioned herein may have another ring further fused. The fused ring may be any of an aromatic hydrocarbon ring, an aromatic heterocyclic ring, an aliphatic hydrocarbon ring, and an aliphatic heterocyclic ring, or may be a ring in which two or more of these rings are fused. Preferably, the fused ring is an aromatic hydrocarbon ring, an aromatic heterocyclic ring, or a ring in which two or more of these rings are fused. Examples of the aromatic hydrocarbon ring include a benzene ring. The aromatic heterocyclic ring means a ring containing a hetero atom as a ring skeleton-constituting atom and exhibiting aromaticity, and is preferably a 5- to 7-membered ring, and for example, a 5-membered ring or a 6-membered ring can be employed. In one aspect of the present invention, a furan ring, a thiophene ring, or a pyrrole ring can be employed as the aromatic heterocyclic ring. The aliphatic hydrocarbon ring is preferably a hydrocarbon ring that does not exhibit aromaticity, and is preferably a 5- to 7-membered ring, and for example, a 5-membered ring or a 6-membered ring can be employed. For example, a cyclopentadiene ring can be employed. The aliphatic heterocyclic ring means a ring containing a hetero atom as a ring skeleton-constituting atom and not exhibiting aromaticity, and is preferably a 5- to 7-membered ring, and for example, a 5-membered ring or a 6-membered ring can be employed.

In one aspect of the present invention, A¹¹ is a benzene ring, and in the benzene ring, a benzene ring, a furan ring, a thiol ring, a pyrrole ring, or a ring in which two or more of these rings are fused is further fused. In one aspect of the present invention, A is a benzene ring, and in the benzene ring, a benzene ring, a furan ring, a thiol ring, or a ring in which two or more of these rings are fused is further fused. In one aspect of the present invention, A¹¹ is a benzene ring, and in the benzene ring, a furan ring of benzofuran or a thiophene ring of benzothiophene is fused. In one aspect of the present invention, A¹¹ is fused with a furan ring of benzofuran. In one aspect of the present invention, A¹¹ is fused with a thiophene ring of benzothiophene. In one aspect of the present invention, A¹² is a benzene ring, and in the benzene ring, a benzene ring, a furan ring, a thiol ring, a pyrrole ring, or a ring in which two or more of these rings are fused is further fused. In one aspect of the present invention, A¹² is a benzene ring, and in the benzene ring, a benzene ring, a furan ring, a thiol ring, or a ring in which two or more of these rings are fused is further fused. In one aspect of the present invention, A¹² is a benzene ring, and in the benzene ring, a furan ring of benzofuran or a thiophene ring of benzothiophene is fused. In one aspect of the present invention, A¹² is fused with a furan ring of benzofuran. In one aspect of the present invention, A¹² is fused with a thiophene ring of benzothiophene.

The hydrogen atoms of the ring constituting A¹¹ or A¹² may be substituted with a deuterium atom or a substituent. The substituent can be selected from any of Substituent Groups A to E, and for example, is selected from Substituent Group E. In one aspect of the present invention, the ring constituting A¹¹ or A¹² may be substituted with one atom or group selected from the group consisting of a deuterium atom, a substituted or unsubstituted aryl group, a substituted or unsubstituted heteroaryl group, a substituted or unsubstituted alkyl group, and a cyano group or with a group formed by combining two or more thereof. In one aspect of the present invention, the ring constituting A¹¹ or A¹² may be substituted with a deuterium atom, an alkyl group, an aryl group, or a group formed by combining thereof. In one aspect of the present invention, the ring constituting A¹¹ or A¹² may be substituted with at least one of a deuterium atom, an alkyl group, an aryl group, and a group formed by combining thereof. In the case where a pyrrole ring is included as a ring constituting A¹¹ or A¹², it is preferable that an aryl group which may be substituted with a deuterium atom, an alkyl group, or an aryl group is bonded to the ring skeleton-constituting nitrogen atom of the pyrrole ring (the same applies to a nitrogen atom of an indole ring described below). When two or more hydrogen atoms of the ring constituting A¹¹ or A¹² are substituted, they may be substituted with the same atom or group, or may be substituted with different atoms or groups.

In the general formula (4), R¹¹¹ to R¹¹⁴, R^{B}, and R^{C} each independently represent a hydrogen atom, a deuterium atom, a substituted or unsubstituted aryl group, a substituted or unsubstituted heteroaryl group, a substituted or unsubstituted alkyl group, or a cyano group. Each of R¹¹'s independently represents a hydrogen atom, a deuterium atom, a substituted or unsubstituted aryl group, a substituted or unsubstituted heteroaryl group, a substituted or unsubstituted alkyl group, a cyano group, or a bond with L (that is, a single bond to L). R^{A} represents a substituted or unsubstituted aryl group, a substituted or unsubstituted heteroaryl group, a substituted or unsubstituted alkyl group, or a bond with L (that is, a single bond to L). For the aryl group, the heteroaryl group, and the alkyl group, the description on the “aryl group”, the “heteroaryl group”, and the “alkyl group” can be referred to. The aryl group preferably has 6 to 14 carbon atoms, and examples thereof include a phenyl group, a 1-naphthyl group, and a 2-naphthyl group. The heteroaryl group is preferably composed of a 5-membered ring or a 6-membered ring, and examples thereof include a 2-pyridyl group, a 3-pyridyl group, a 4-pyridyl group, a carbazol-9-yl group, a dibenzofuiryl group, and a dibenzothienyl group. The alkyl group preferably has 1 to 6 carbon atoms, and examples thereof include a methyl group, an ethyl group, an isopropyl group, and a tert-butyl group. Such aryl group, heteroaryl group, and alkyl group may be substituted, and in the case of being substituted, such groups are preferably substituted with one atom or group selected from the group consisting of a deuterium atom, an aryl group, a heteroaryl group, an alkyl group, and a cyano group, or a group formed by combining two or more thereof, and more preferably substituted with one atom or group selected from the group consisting of a deuterium atom, an aryl group, a heteroaryl group, and an alkyl group, or a group formed by combining two or more thereof. In one aspect of the present invention, R¹¹² is a substituted or unsubstituted aryl group, a substituted or unsubstituted heteroaryl group, a substituted or unsubstituted alkyl group, or a cyano group. In one aspect of the present invention, R¹¹¹ to R¹¹⁴ are each independently a hydrogen atom or a deuterium atom.

R¹¹¹ and R¹¹², R¹¹² and R¹¹³, R¹¹³ and R¹¹⁴, adjacent two R¹¹⁵'s, and R^{B} and R^{C} each may be bonded to each other to form a cyclic structure. For the cyclic structure mentioned herein, the description on the ring further fused in the benzene ring described for A¹¹ and A¹² can be referred to. In one aspect of the present invention, a pair selected from (i) R¹¹¹ and R¹¹², (ii) R¹¹² and R¹¹³, and (iii) R¹¹³ and R¹¹⁴ are bonded to each other to form a benzofuran ring (fused with a furan ring), a benzothiophene ring (fused with a thiophene ring), or an indole ring (fused with a pyrrole ring).

In one aspect of the present invention, a group bonded to L from the left side in the general formula (4) is a substituted or unsubstituted carbazol-9-yl group. For example, the group is a carbazol-9-yl group in which at least one (preferably both) of the 3-position and the 6-position is substituted with a deuterium atom, an alkyl group, an aryl group, or a group formed by combining thereof. Further, the group may be an unsubstituted carbazol-9-yl group. In one aspect of the present invention, the group bonded to L from the left side in the general formula (4) is a substituted or unsubstituted benzofuro[2,3-a]carbazol-12-yl group, a substituted or unsubstituted benzofuro[3,2-a]carbazol-12-yl group, a substituted or unsubstituted benzofuro[2,3-b]carbazol-7-yl group, a substituted or unsubstituted benzofuro[3,2-b]carbazol-1l-yl group, a substituted or unsubstituted benzofuro[2,3-c]carbazol-8-yl group, or a substituted or unsubstituted benzofuro[3,2-c]carbazol-5-yl group. In one aspect of the present invention, the group bonded to L from the left side in the general formula (4) is a substituted or unsubstituted benzothieno[2,3-a]carbazol-12-yl group, a substituted or unsubstituted benzothieno[3,2-a]carbazol-12-yl group, a substituted or unsubstituted benzothieno[2,3-b]carbazol-7-yl group, a substituted or unsubstituted benzothieno[3,2-b]carbazol-11-yl group, a substituted or unsubstituted benzothieno[2,3-c]carbazol-8-yl group, or a substituted or unsubstituted benzothieno[3,2-c]carbazol-5-yl group. In one aspect of the present invention, the group bonded to L from the left side in the general formula (4) is a substituted or unsubstituted 11-phenylindolo[2,3-a]carbazol-12-yl group, a substituted or unsubstituted 5-phenylindolo[3,2-a]carbazol-12-yl group, a substituted or unsubstituted 5-phenylindolo[2,3-b]carbazol-7-yl group, a substituted or unsubstituted 5-phenylindolo[3,2-b]carbazol-11-yl group, a substituted or unsubstituted 5-phenylindolo[2,3-c]carbazol-8-yl group, or a substituted or unsubstituted 12-phenylindolo[3,2-a]carbazol-5-yl group.

In one aspect of the present invention, as a group bonded to L from the right side in the general formula (4), the group exemplified as the group bonded to L from the left side can be employed. Here, the group bonded to L from the right side is not an unsubstituted carbazol-9-yl group.

Specific examples of the group that can be adopted as the group bonded to L from the left side in the general formula (4) are shown below. Here, the groups that can be employed in the present invention shall not be construed as being limited by these specific examples. In the following specific examples, the expression of a methyl group is omitted. Therefore, for example, Z2 or Z3 is substituted with a methyl group. * indicates a bonding position to L.

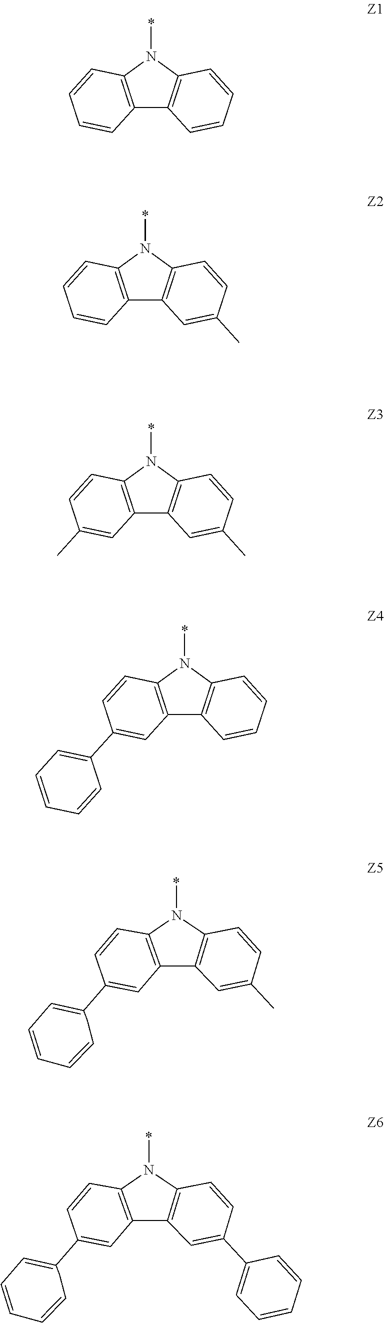

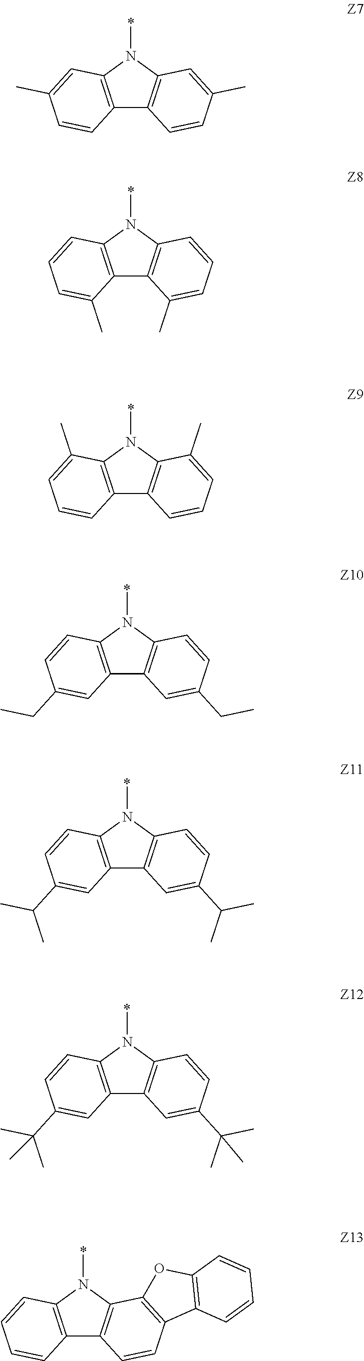

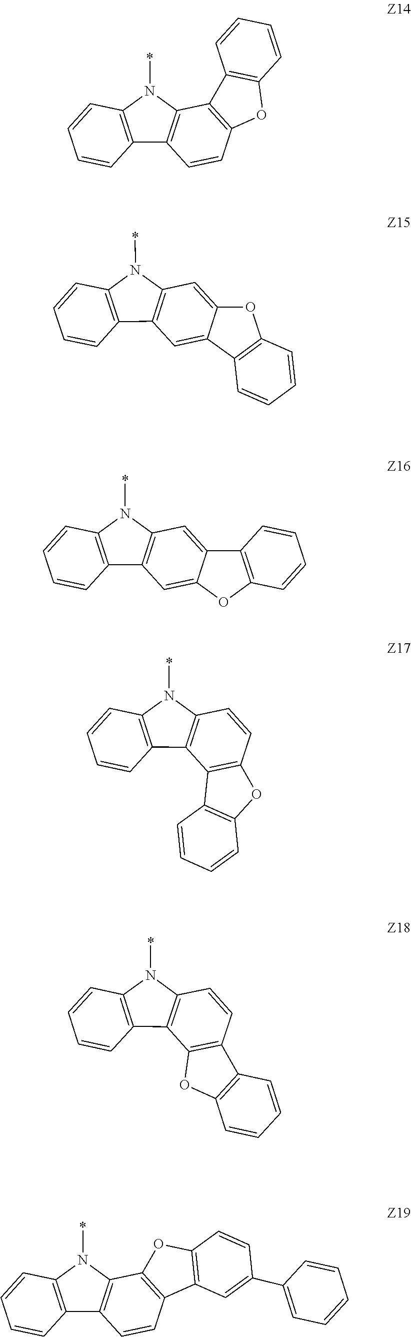 [SMILES: *n1c2ccccc2c2ccc3oc4ccccc4c3c21 ; *n1c2ccccc2c2cc3c(cc21)oc1ccccc13 ; *n1c2ccccc2c2cc3oc4ccccc4c3cc21 ; *n1c2ccccc2c2c3c(ccc21)oc1ccccc13 ; *n1c2ccccc2c2c3oc4ccccc4c3ccc21 ; *n1c2ccccc2c2ccc3c4cc(-c5ccccc5)ccc4oc3c21]

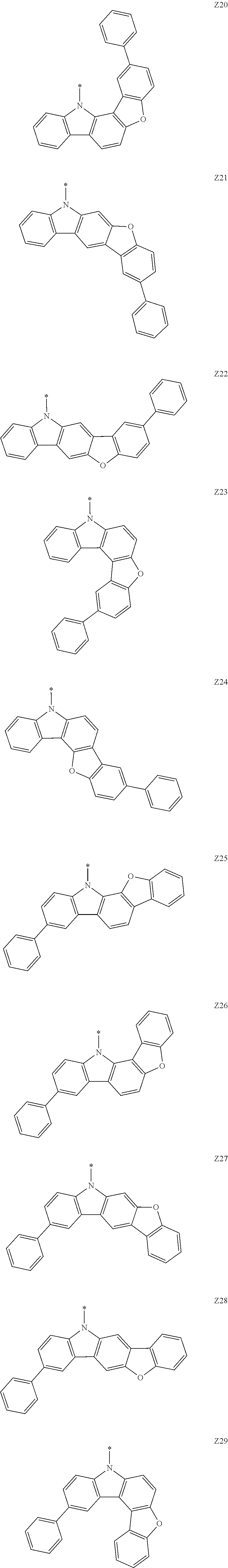

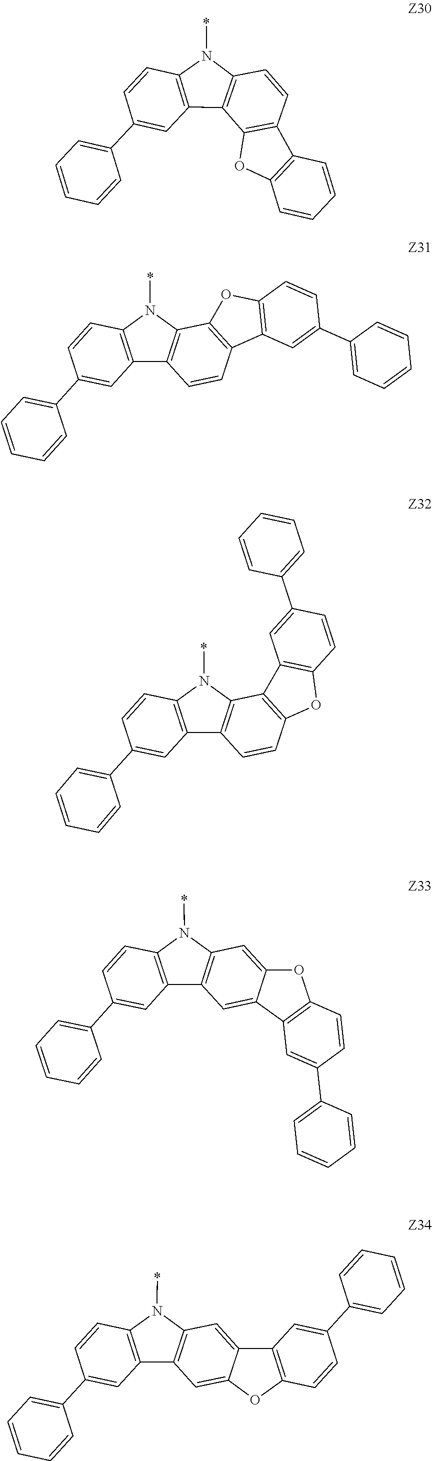 [SMILES: *n1c2ccc(-c3ccccc3)cc2c2c3oc4ccccc4c3ccc21 ; *n1c2ccc(-c3ccccc3)cc2c2ccc3c4cc(-c5ccccc5)ccc4oc3c21 ; *n1c2ccc(-c3ccccc3)cc2c2ccc3oc4ccc(-c5ccccc5)cc4c3c21 ; *n1c2ccc(-c3ccccc3)cc2c2cc3c(cc21)oc1ccc(-c2ccccc2)cc13 ; *n1c2ccc(-c3ccccc3)cc2c2cc3oc4ccc(-c5ccccc5)cc4c3cc21]

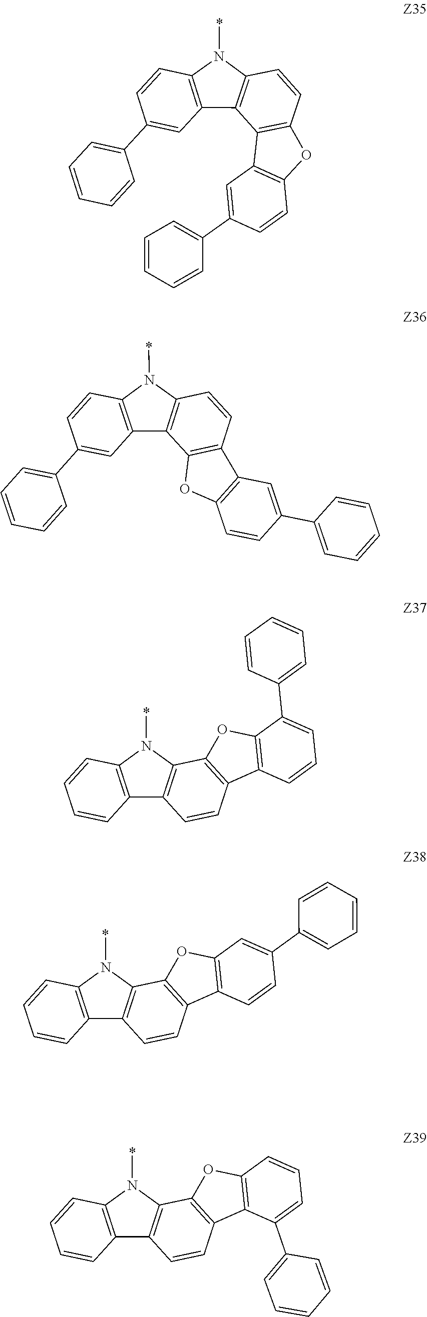

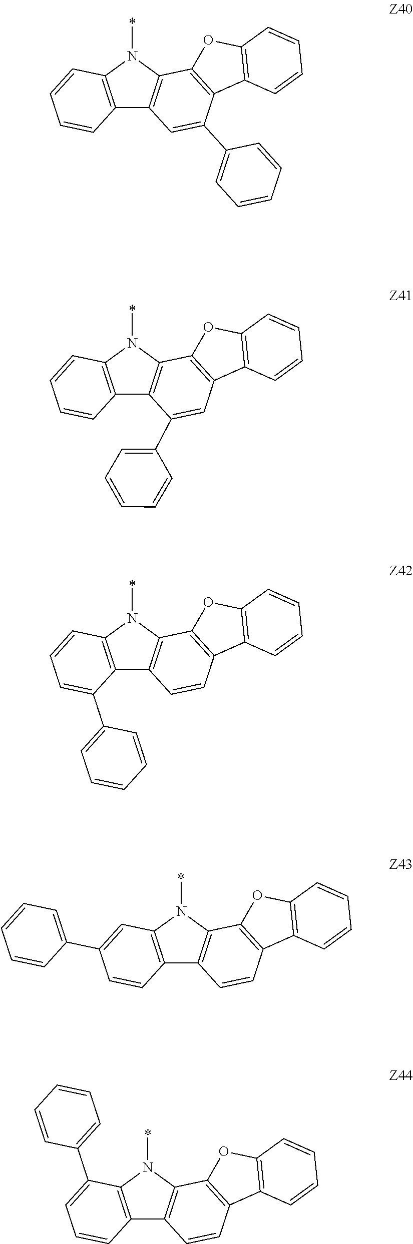 [SMILES: *n1c2ccccc2c2cc(-c3ccccc3)c3c4ccccc4oc3c21 ; *n1c2ccccc2c2c(-c3ccccc3)cc3c4ccccc4oc3c21 ; *n1c2cccc(-c3ccccc3)c2c2ccc3c4ccccc4oc3c21 ; *n1c2cc(-c3ccccc3)ccc2c2ccc3c4ccccc4oc3c21 ; *n1c2c(-c3ccccc3)cccc2c2ccc3c4ccccc4oc3c21]

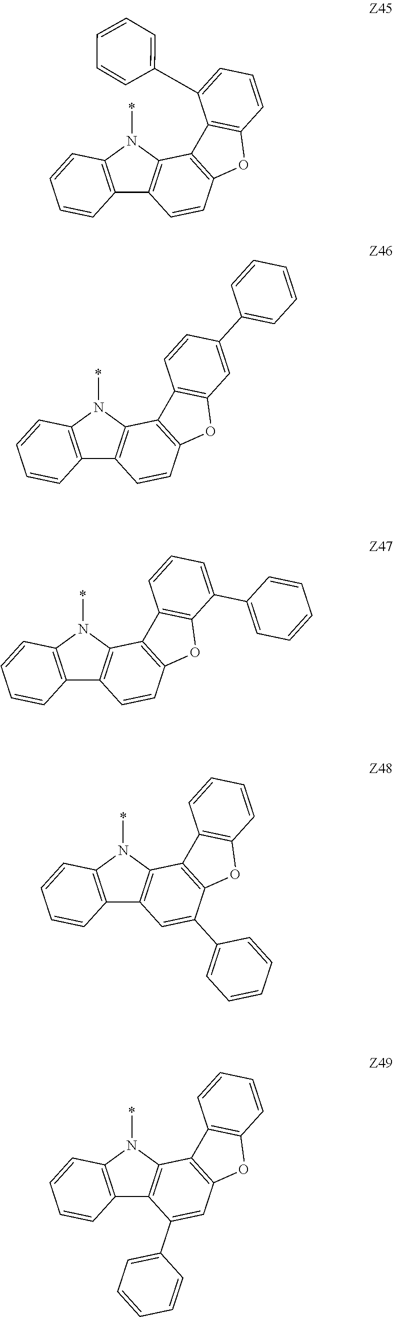 [SMILES: *n1c2ccccc2c2ccc3oc4cccc(-c5ccccc5)c4c3c21 ; *n1c2ccccc2c2ccc3oc4cc(-c5ccccc5)ccc4c3c21 ; *n1c2ccccc2c2ccc3oc4c(-c5ccccc5)cccc4c3c21 ; *n1c2ccccc2c2cc(-c3ccccc3)c3oc4ccccc4c3c21 ; *n1c2ccccc2c2c(-c3ccccc3)cc3oc4ccccc4c3c21]

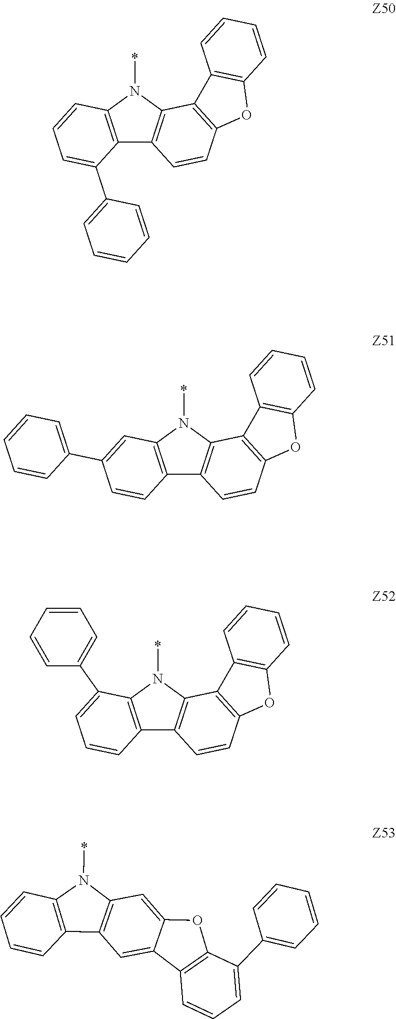 [SMILES: *n1c2cccc(-c3ccccc3)c2c2ccc3oc4ccccc4c3c21 ; *n1c2cc(-c3ccccc3)ccc2c2ccc3oc4ccccc4c3c21 ; *n1c2c(-c3ccccc3)cccc2c2ccc3oc4ccccc4c3c21 ; *n1c2ccccc2c2cc3c(cc21)oc1c(-c2ccccc2)cccc13]

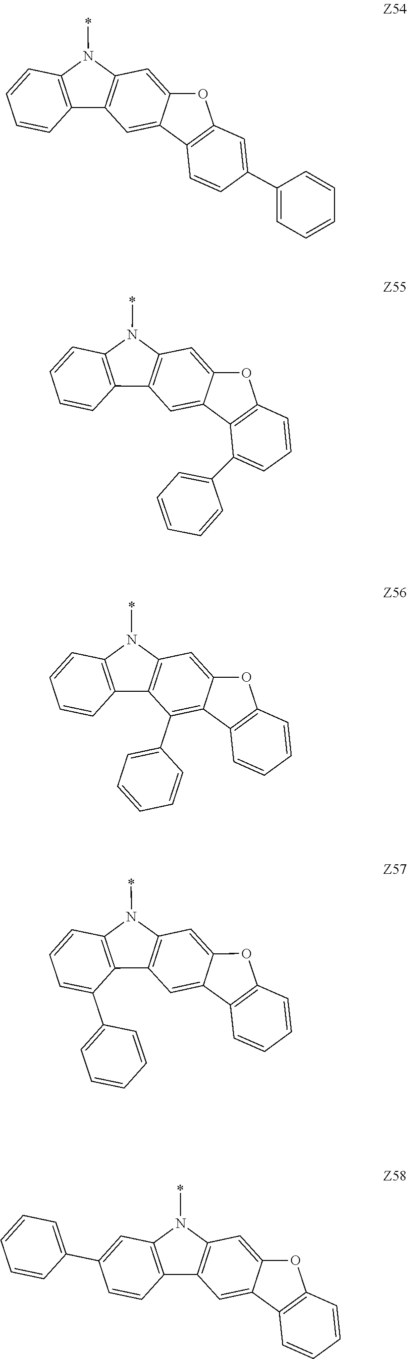 [SMILES: *n1c2ccccc2c2cc3c(cc21)oc1cc(-c2ccccc2)ccc13 ; *n1c2ccccc2c2cc3c(cc21)oc1cccc(-c2ccccc2)c13 ; *n1c2ccccc2c2c(-c3ccccc3)c3c(cc21)oc1ccccc13 ; *n1c2cc3oc4ccccc4c3cc2c2c(-c3ccccc3)cccc21 ; *n1c2cc(-c3ccccc3)ccc2c2cc3c(cc21)oc1ccccc13]

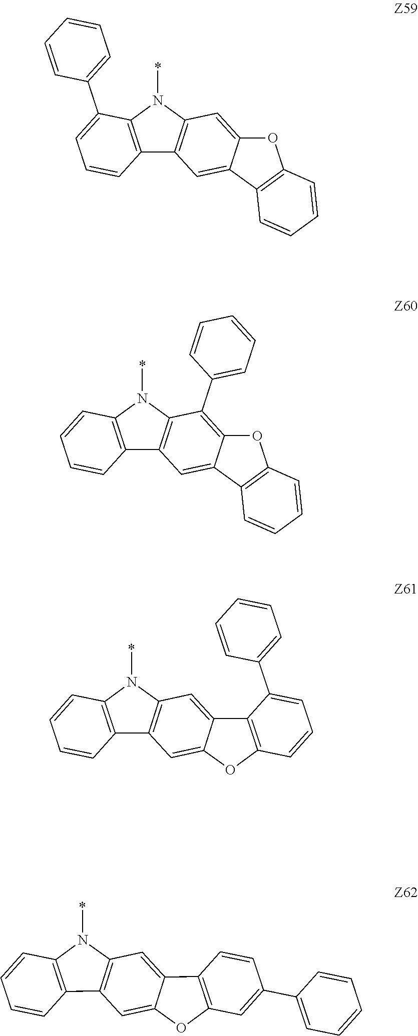 [SMILES: *n1c2cc3oc4ccccc4c3cc2c2cccc(-c3ccccc3)c21 ; *n1c2ccccc2c2cc3c(oc4ccccc43)c(-c3ccccc3)c21 ; *n1c2ccccc2c2cc3oc4cccc(-c5ccccc5)c4c3cc21 ; *n1c2ccccc2c2cc3oc4cc(-c5ccccc5)ccc4c3cc21]

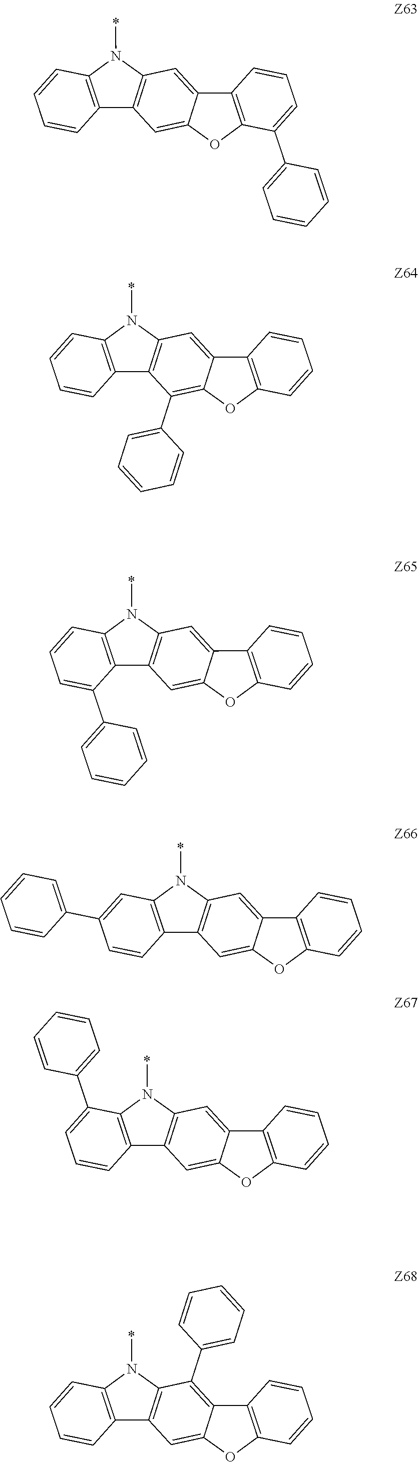 [SMILES: *n1c2ccccc2c2cc3oc4c(-c5ccccc5)cccc4c3cc21 ; *n1c2ccccc2c2c(-c3ccccc3)c3oc4ccccc4c3cc21 ; *n1c2cc3c(cc2c2c(-c4ccccc4)cccc21)oc1ccccc13 ; *n1c2cc(-c3ccccc3)ccc2c2cc3oc4ccccc4c3cc21 ; *n1c2cc3c(cc2c2cccc(-c4ccccc4)c21)oc1ccccc13 ; *n1c2ccccc2c2cc3oc4ccccc4c3c(-c3ccccc3)c21]

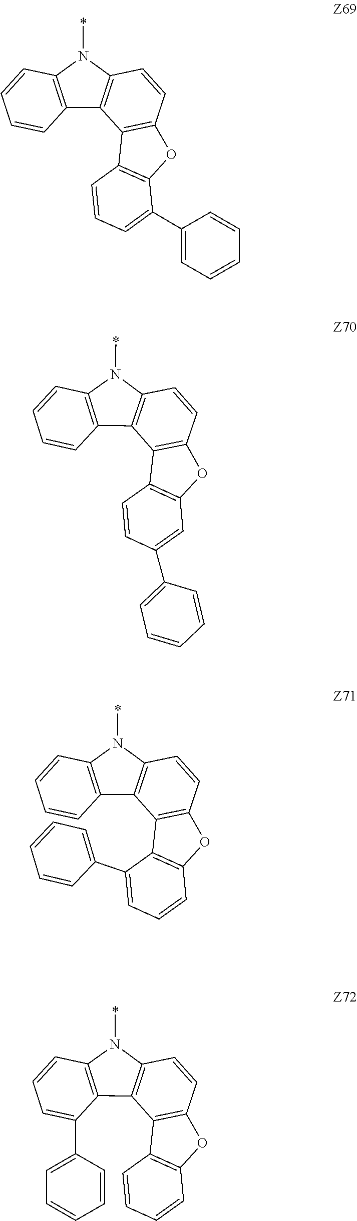 [SMILES: *n1c2ccccc2c2c3c(ccc21)oc1c(-c2ccccc2)cccc13 ; *n1c2ccccc2c2c3c(ccc21)oc1cc(-c2ccccc2)ccc13 ; *n1c2ccccc2c2c3c(ccc21)oc1cccc(-c2ccccc2)c13 ; *n1c2cccc(-c3ccccc3)c2c2c3c(ccc21)oc1ccccc13]

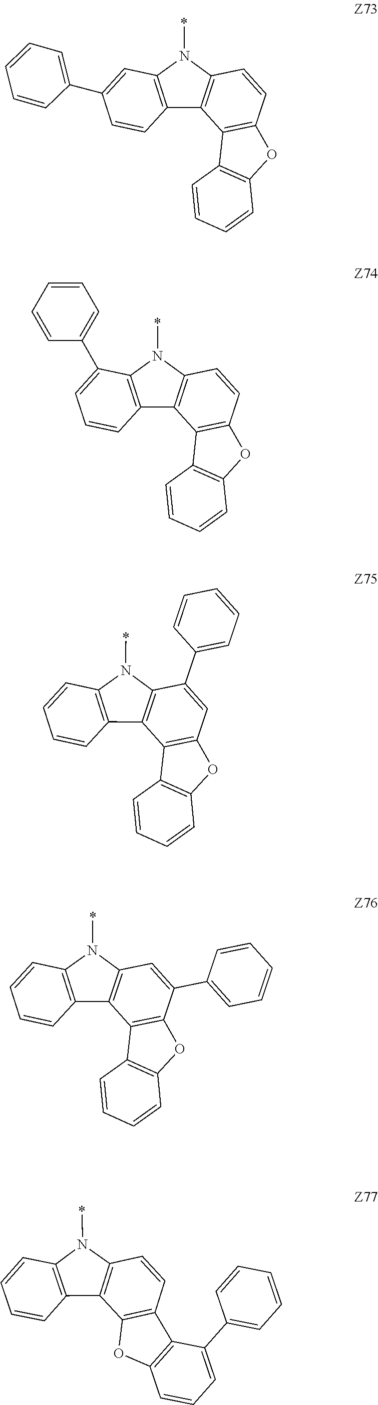 [SMILES: *n1c2cc(-c3ccccc3)ccc2c2c3c(ccc21)oc1ccccc13 ; *n1c2ccc3oc4ccccc4c3c2c2cccc(-c3ccccc3)c21 ; *n1c2ccccc2c2c3c(cc(-c4ccccc4)c21)oc1ccccc13 ; *n1c2ccccc2c2c3c(oc4ccccc43)c(-c3ccccc3)cc21 ; *n1c2ccccc2c2c3oc4cccc(-c5ccccc5)c4c3ccc21]

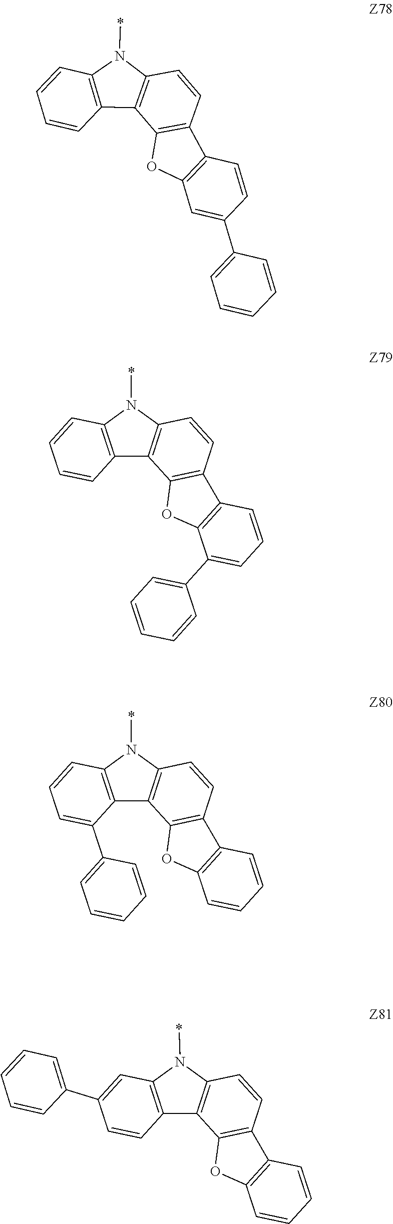 [SMILES: *n1c2ccccc2c2c3oc4cc(-c5ccccc5)ccc4c3ccc21 ; *n1c2ccccc2c2c3oc4c(-c5ccccc5)cccc4c3ccc21 ; *n1c2cccc(-c3ccccc3)c2c2c3oc4ccccc4c3ccc21 ; *n1c2cc(-c3ccccc3)ccc2c2c3oc4ccccc4c3ccc21]

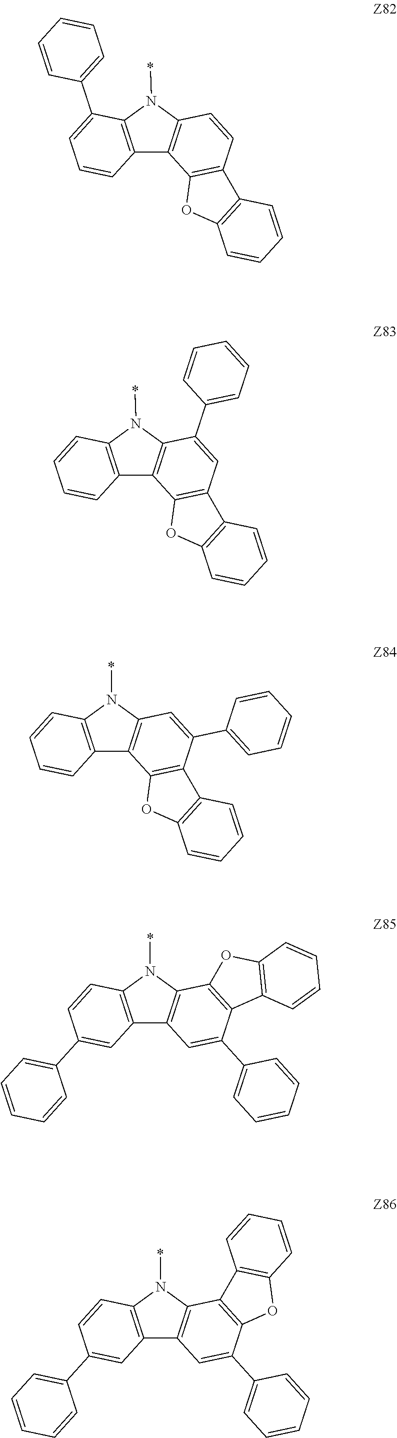 [SMILES: *n1c2ccc3c4ccccc4oc3c2c2cccc(-c3ccccc3)c21 ; *n1c2ccccc2c2c3oc4ccccc4c3cc(-c3ccccc3)c21 ; *n1c2ccccc2c2c3oc4ccccc4c3c(-c3ccccc3)cc21 ; *n1c2ccc(-c3ccccc3)cc2c2cc(-c3ccccc3)c3c4ccccc4oc3c21 ; *n1c2ccc(-c3ccccc3)cc2c2cc(-c3ccccc3)c3oc4ccccc4c3c21]

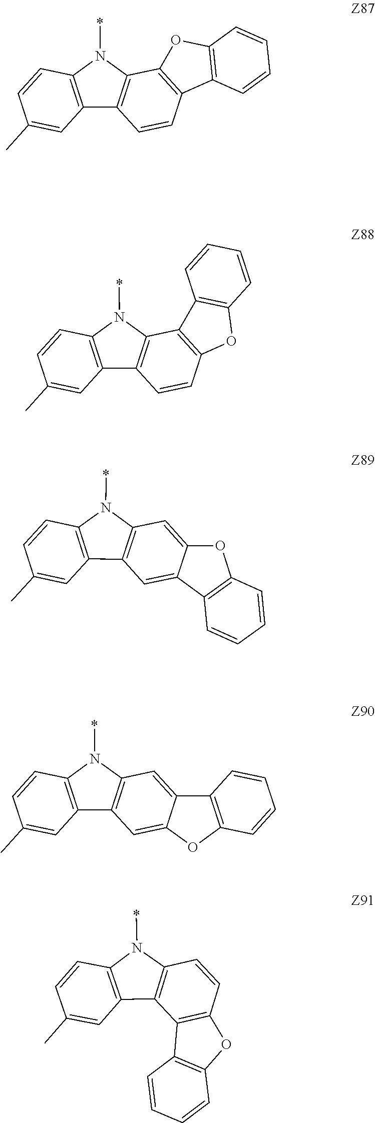 [SMILES: *n1c2ccc(C)cc2c2ccc3c4ccccc4oc3c21 ; *n1c2ccc(C)cc2c2ccc3oc4ccccc4c3c21 ; *n1c2ccc(C)cc2c2cc3c(cc21)oc1ccccc13 ; *n1c2ccc(C)cc2c2cc3oc4ccccc4c3cc21 ; *n1c2ccc(C)cc2c2c3c(ccc21)oc1ccccc13]

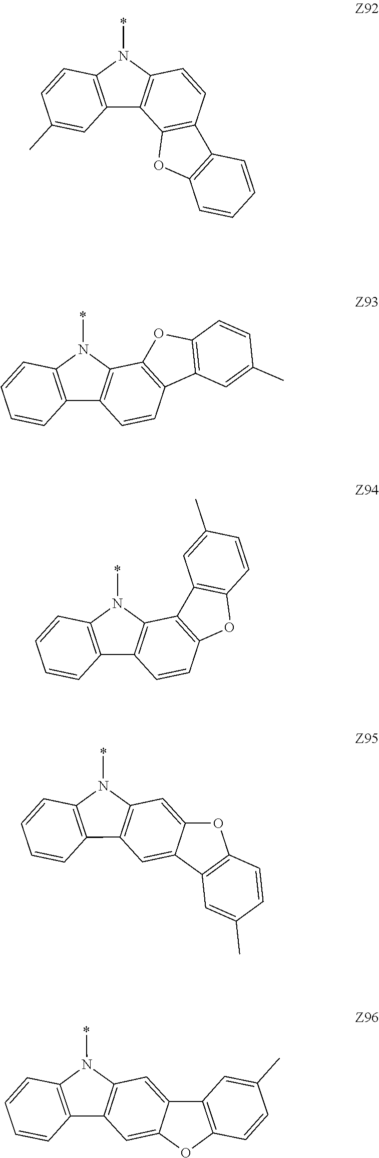 [SMILES: *n1c2ccc(C)cc2c2c3oc4ccccc4c3ccc21 ; *n1c2ccccc2c2ccc3c4cc(C)ccc4oc3c21 ; *n1c2ccccc2c2ccc3oc4ccc(C)cc4c3c21 ; *n1c2ccccc2c2cc3c(cc21)oc1ccc(C)cc13 ; *n1c2ccccc2c2cc3oc4ccc(C)cc4c3cc21]

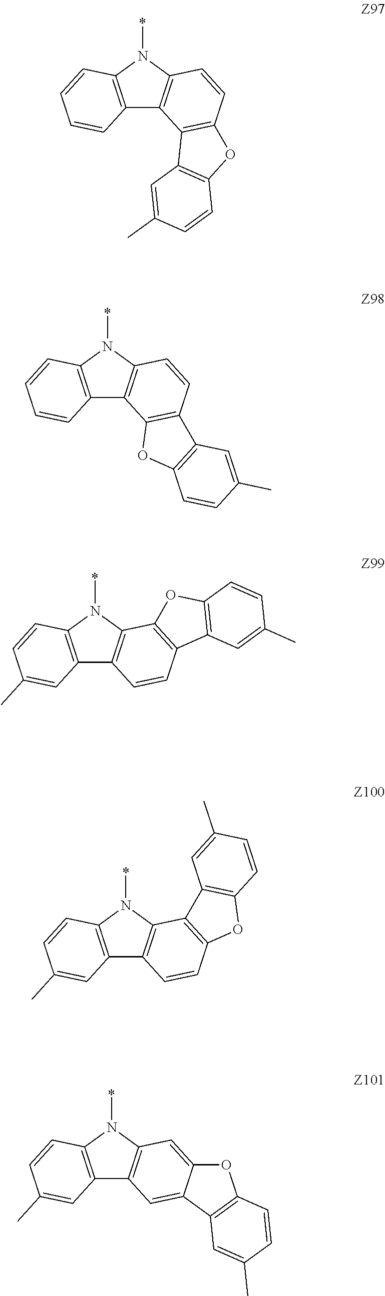

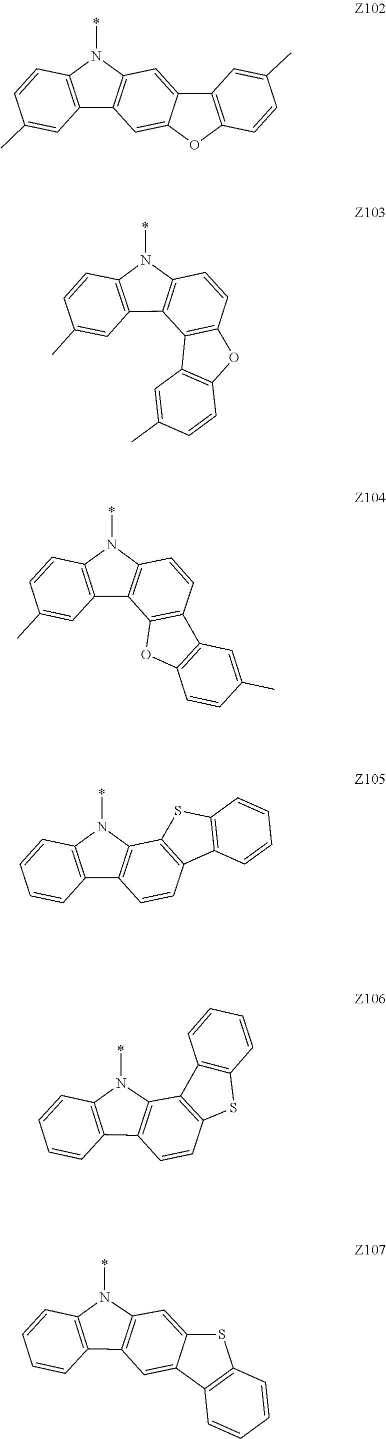

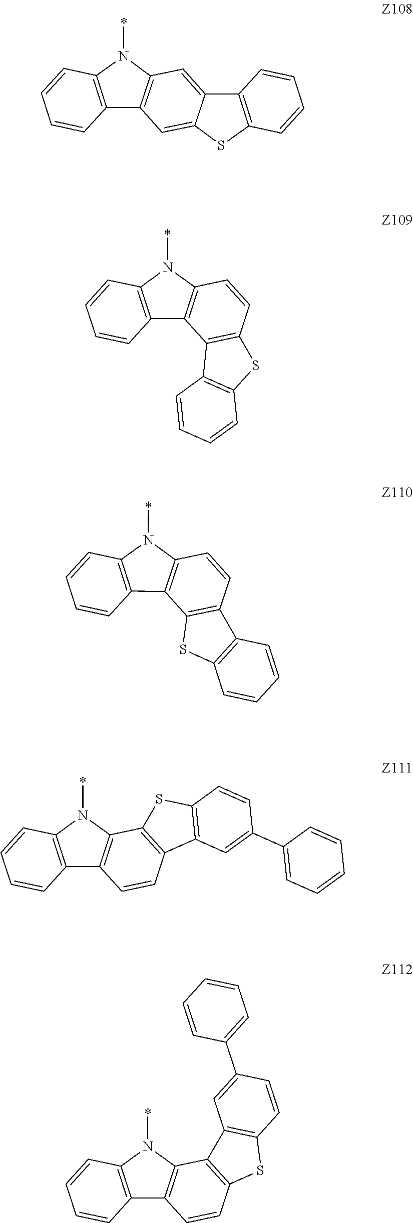 [SMILES: *n1c2ccccc2c2cc3sc4ccccc4c3cc21 ; *n1c2ccccc2c2c3c(ccc21)sc1ccccc13 ; *n1c2ccccc2c2c3sc4ccccc4c3ccc21 ; *n1c2ccccc2c2ccc3c4cc(-c5ccccc5)ccc4sc3c21 ; *n1c2ccccc2c2ccc3sc4ccc(-c5ccccc5)cc4c3c21]

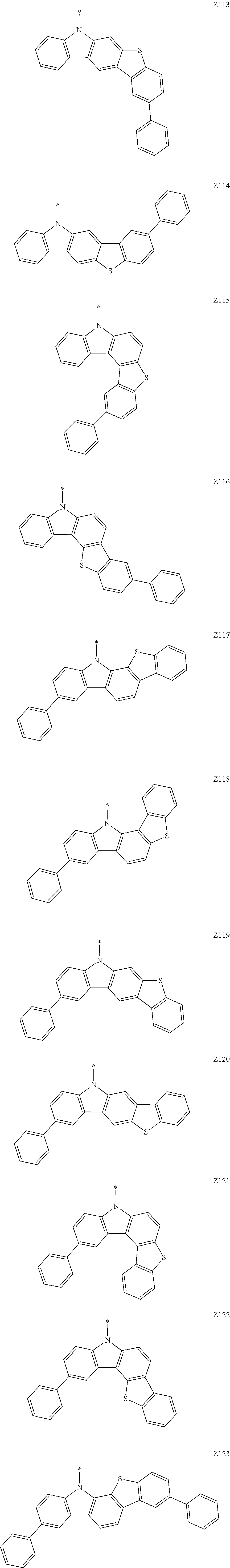

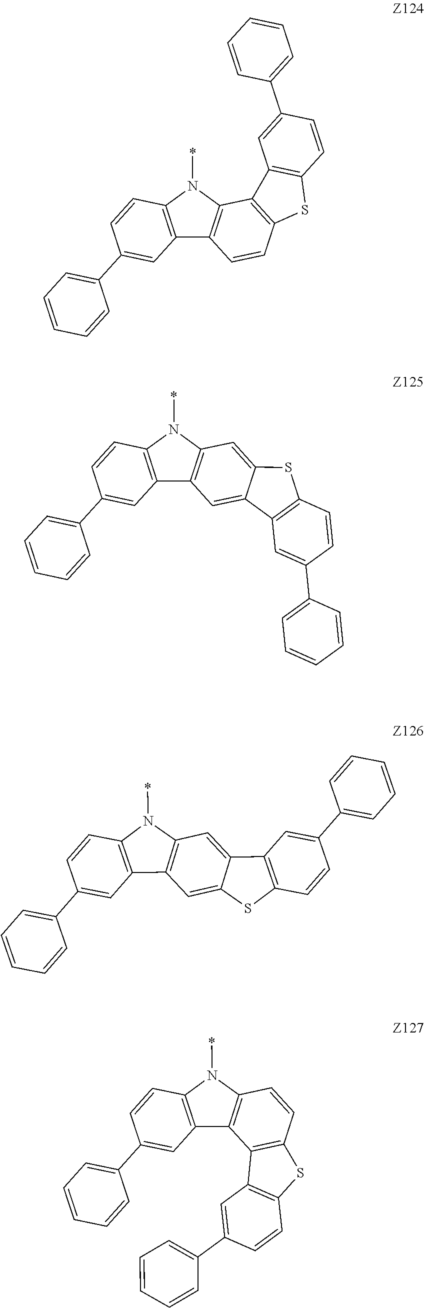 [SMILES: *n1c2ccc(-c3ccccc3)cc2c2ccc3sc4ccc(-c5ccccc5)cc4c3c21 ; *n1c2ccc(-c3ccccc3)cc2c2cc3c(cc21)sc1ccc(-c2ccccc2)cc13 ; *n1c2ccc(-c3ccccc3)cc2c2cc3sc4ccc(-c5ccccc5)cc4c3cc21 ; *n1c2ccc(-c3ccccc3)cc2c2c3c(ccc21)sc1ccc(-c2ccccc2)cc13]

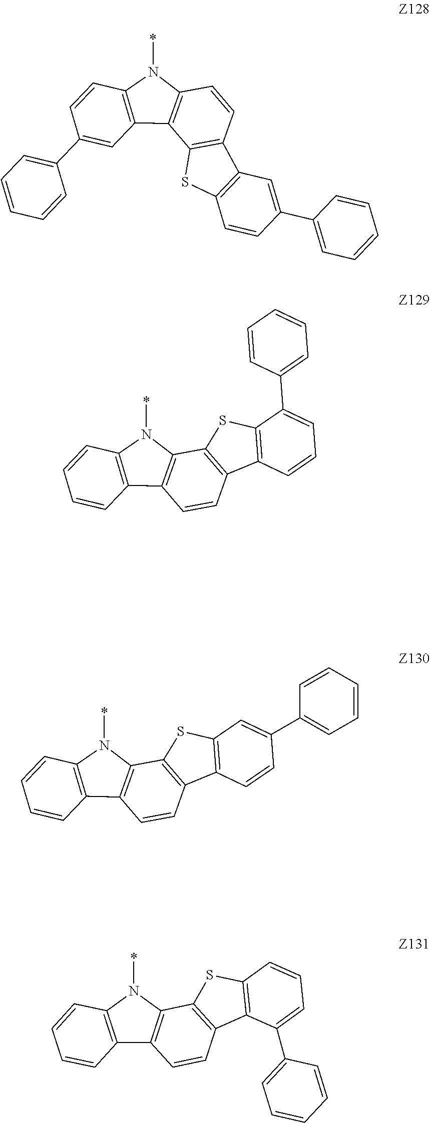 [SMILES: *n1c2ccc(-c3ccccc3)cc2c2c3sc4ccc(-c5ccccc5)cc4c3ccc21 ; *n1c2ccccc2c2ccc3c4cccc(-c5ccccc5)c4sc3c21 ; *n1c2ccccc2c2ccc3c4ccc(-c5ccccc5)cc4sc3c21 ; *n1c2ccccc2c2ccc3c(sc4cccc(-c5ccccc5)c43)c21]

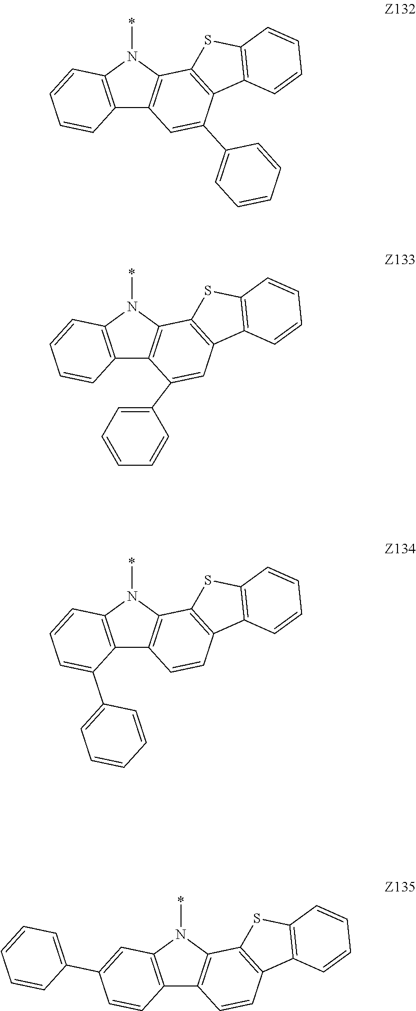 [SMILES: *n1c2ccccc2c2cc(-c3ccccc3)c3c4ccccc4sc3c21 ; *n1c2ccccc2c2c(-c3ccccc3)cc3c4ccccc4sc3c21 ; *n1c2cccc(-c3ccccc3)c2c2ccc3c4ccccc4sc3c21 ; *n1c2cc(-c3ccccc3)ccc2c2ccc3c4ccccc4sc3c21]

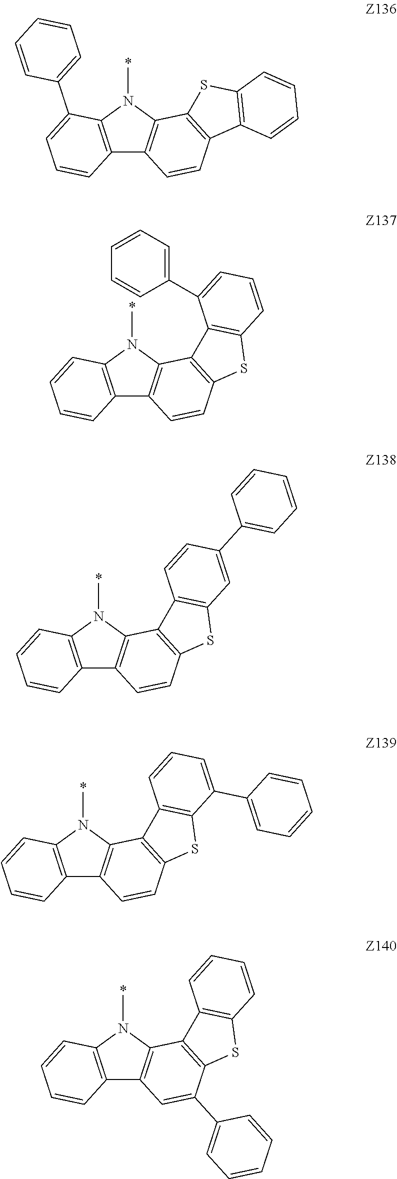 [SMILES: Cn1c2c(-c3ccccc3)cccc2c2ccc3c4ccccc4sc3c21 ; Cn1c2ccccc2c2ccc3sc4cccc(-c5ccccc5)c4c3c21 ; Cn1c2ccccc2c2ccc3sc4cc(-c5ccccc5)ccc4c3c21 ; Cn1c2ccccc2c2ccc3sc4c(-c5ccccc5)cccc4c3c21 ; Cn1c2ccccc2c2cc(-c3ccccc3)c3sc4ccccc4c3c21]

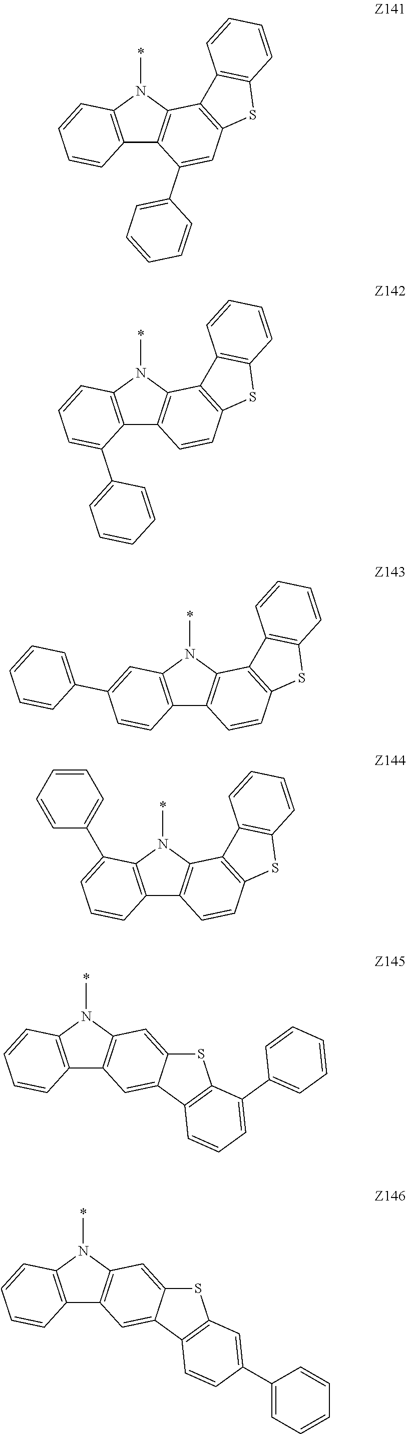 [SMILES: Cn1c2ccccc2c2c(-c3ccccc3)cc3sc4ccccc4c3c21 ; Cn1c2cccc(-c3ccccc3)c2c2ccc3sc4ccccc4c3c21 ; Cn1c2cc(-c3ccccc3)ccc2c2ccc3sc4ccccc4c3c21 ; Cn1c2c(-c3ccccc3)cccc2c2ccc3sc4ccccc4c3c21 ; Cn1c2ccccc2c2cc3c(cc21)sc1c(-c2ccccc2)cccc13 ; Cn1c2ccccc2c2cc3c(cc21)sc1cc(-c2ccccc2)ccc13]

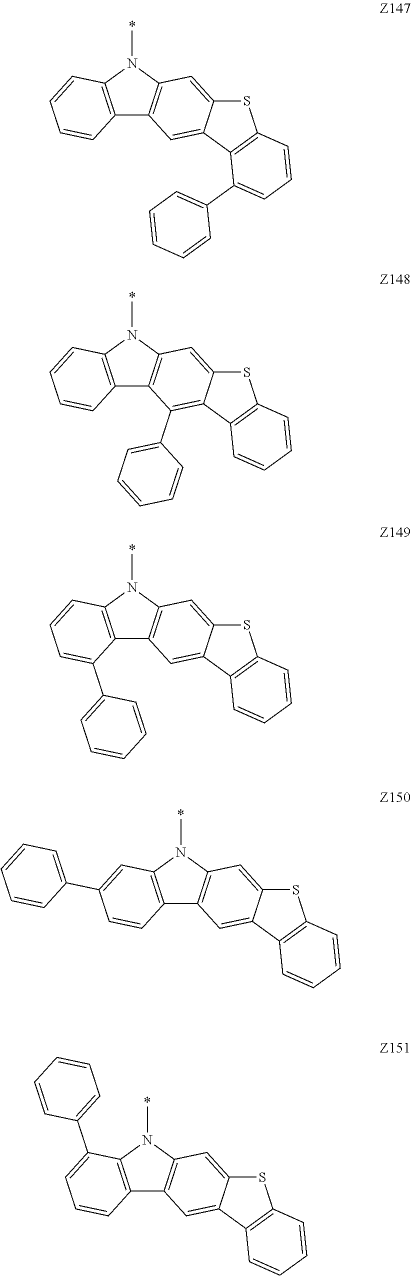 [SMILES: Cn1c2ccccc2c2cc3c(cc21)sc1cccc(-c2ccccc2)c13 ; Cn1c2ccccc2c2c(-c3ccccc3)c3c(cc21)sc1ccccc13 ; Cn1c2cc3sc4ccccc4c3cc2c2c(-c3ccccc3)cccc21 ; Cn1c2cc(-c3ccccc3)ccc2c2cc3c(cc21)sc1ccccc13 ; Cn1c2cc3sc4ccccc4c3cc2c2cccc(-c3ccccc3)c21]

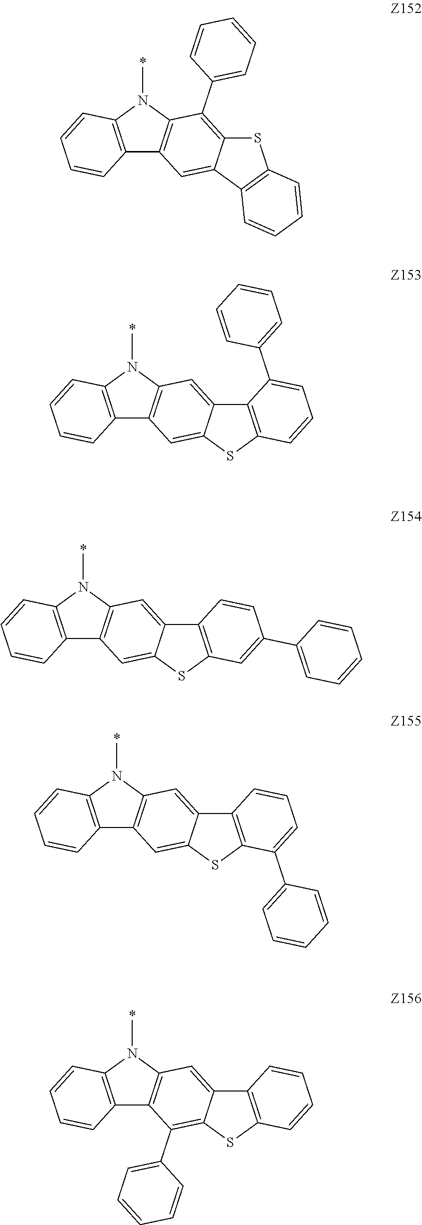 [SMILES: Cn1c2ccccc2c2cc3c(sc4ccccc43)c(-c3ccccc3)c21 ; Cn1c2ccccc2c2cc3sc4cccc(-c5ccccc5)c4c3cc21 ; Cn1c2ccccc2c2cc3sc4cc(-c5ccccc5)ccc4c3cc21 ; Cn1c2ccccc2c2cc3sc4c(-c5ccccc5)cccc4c3cc21 ; Cn1c2ccccc2c2c(-c3ccccc3)c3sc4ccccc4c3cc21]

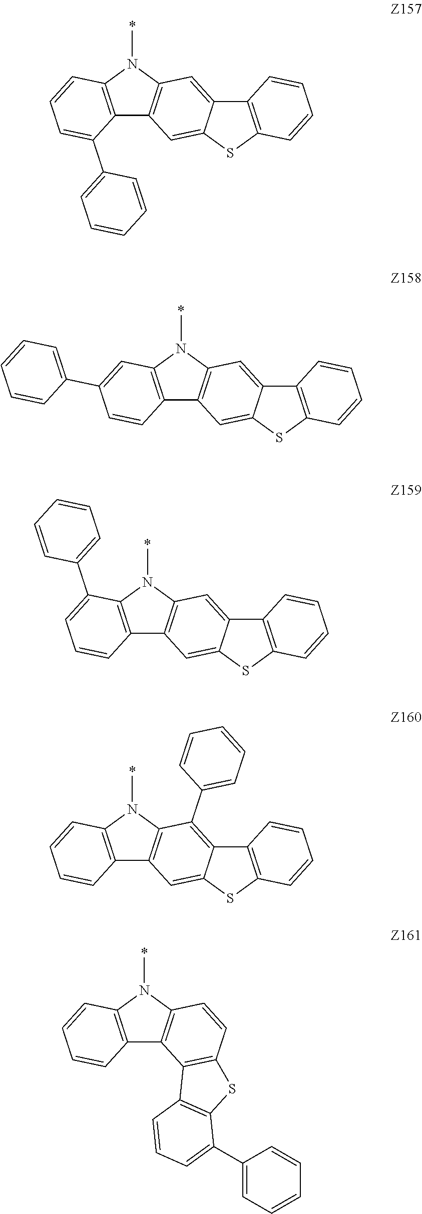 [SMILES: Cn1c2cc3c(cc2c2c(-c4ccccc4)cccc21)sc1ccccc13 ; Cn1c2cc(-c3ccccc3)ccc2c2cc3sc4ccccc4c3cc21 ; Cn1c2cc3c(cc2c2cccc(-c4ccccc4)c21)sc1ccccc13 ; Cn1c2ccccc2c2cc3sc4ccccc4c3c(-c3ccccc3)c21 ; Cn1c2ccccc2c2c3c(ccc21)sc1c(-c2ccccc2)cccc13]

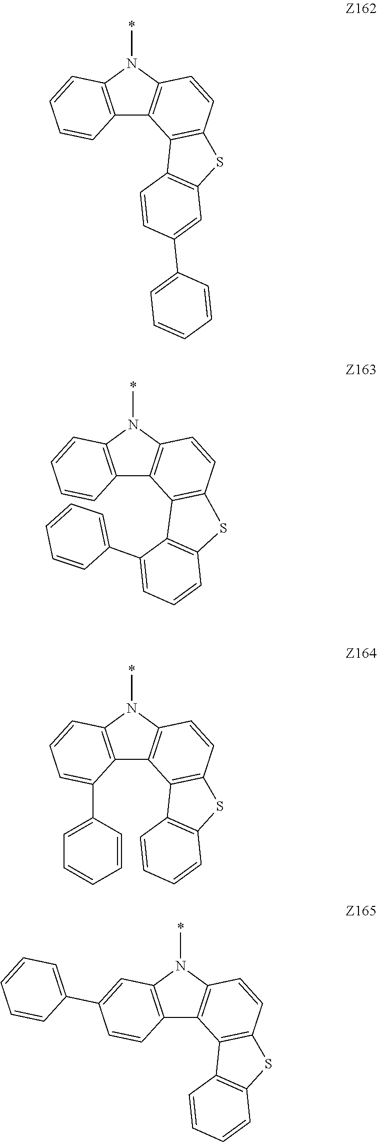 [SMILES: Cn1c2ccccc2c2c3c(ccc21)sc1cc(-c2ccccc2)ccc13 ; Cn1c2ccccc2c2c3c(ccc21)sc1cccc(-c2ccccc2)c13 ; Cn1c2cccc(-c3ccccc3)c2c2c3c(ccc21)sc1ccccc13 ; Cn1c2cc(-c3ccccc3)ccc2c2c3c(ccc21)sc1ccccc13]

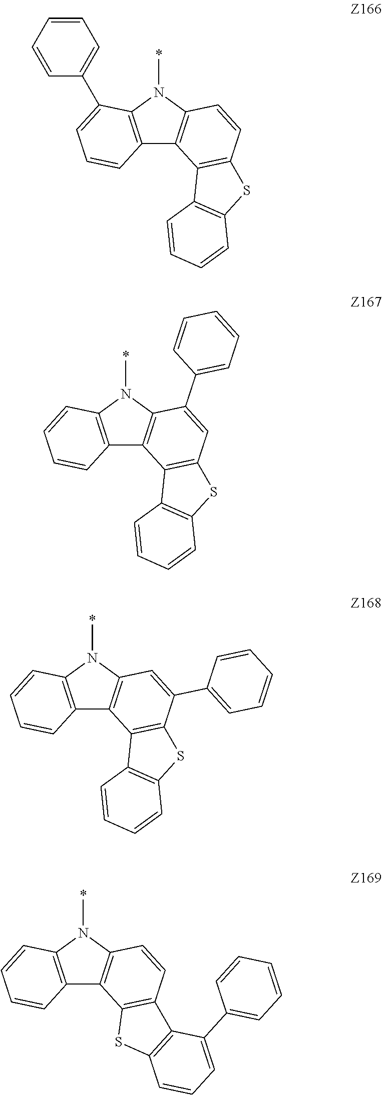

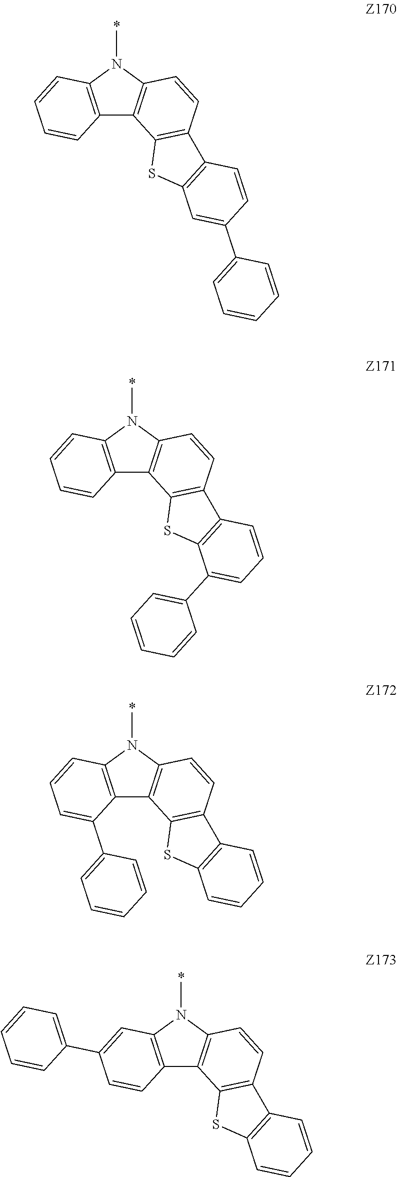 [SMILES: Cn1c2ccccc2c2c3sc4cc(-c5ccccc5)ccc4c3ccc21 ; Cn1c2ccccc2c2c3sc4c(-c5ccccc5)cccc4c3ccc21 ; Cn1c2cccc(-c3ccccc3)c2c2c3sc4ccccc4c3ccc21 ; Cn1c2cc(-c3ccccc3)ccc2c2c3sc4ccccc4c3ccc21]

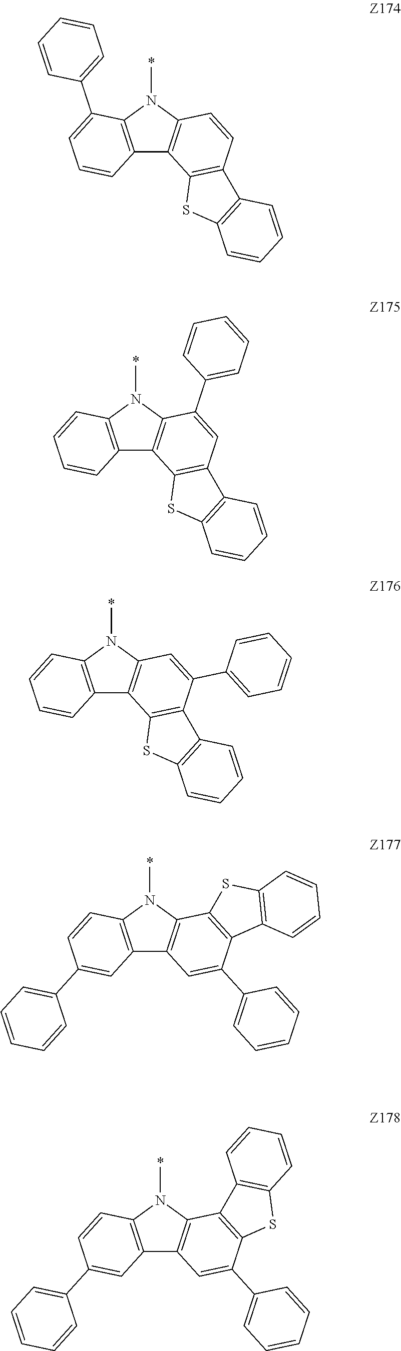 [SMILES: Cn1c2ccc3c4ccccc4sc3c2c2cccc(-c3ccccc3)c21 ; Cn1c2ccccc2c2c3sc4ccccc4c3cc(-c3ccccc3)c21 ; Cn1c2ccccc2c2c3sc4ccccc4c3c(-c3ccccc3)cc21 ; Cn1c2ccc(-c3ccccc3)cc2c2cc(-c3ccccc3)c3c4ccccc4sc3c21 ; Cn1c2ccc(-c3ccccc3)cc2c2cc(-c3ccccc3)c3sc4ccccc4c3c21]

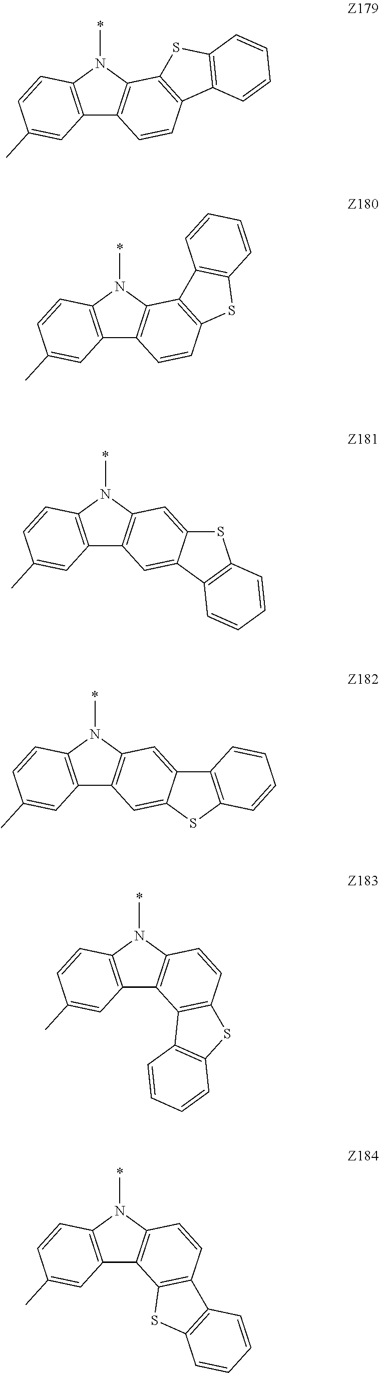 [SMILES: Cc1ccc2c(c1)c1ccc3c4ccccc4sc3c1n2C ; Cc1ccc2c(c1)c1ccc3sc4ccccc4c3c1n2C ; Cc1ccc2c(c1)c1cc3c(cc1n2C)sc1ccccc13 ; Cc1ccc2c(c1)c1cc3sc4ccccc4c3cc1n2C ; Cc1ccc2c(c1)c1c3c(ccc1n2C)sc1ccccc13 ; Cc1ccc2c(c1)c1c3sc4ccccc4c3ccc1n2C]

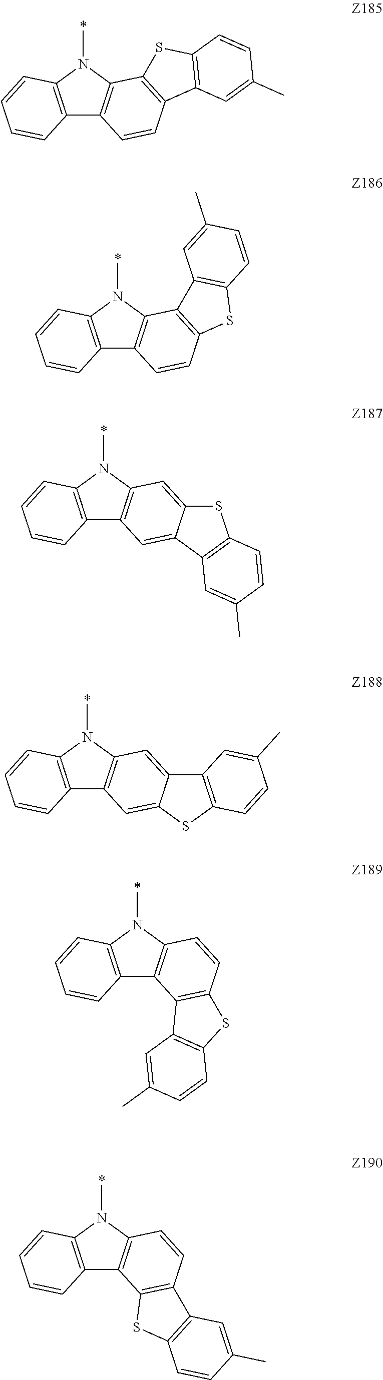 [SMILES: Cc1ccc2sc3c(ccc4c5ccccc5n(C)c43)c2c1 ; Cc1ccc2sc3ccc4c5ccccc5n(C)c4c3c2c1 ; Cc1ccc2sc3cc4c(cc3c2c1)c1ccccc1n4C ; Cc1ccc2sc3cc4c5ccccc5n(C)c4cc3c2c1 ; Cc1ccc2sc3ccc4c(c5ccccc5n4C)c3c2c1 ; Cc1ccc2sc3c(ccc4c3c3ccccc3n4C)c2c1]

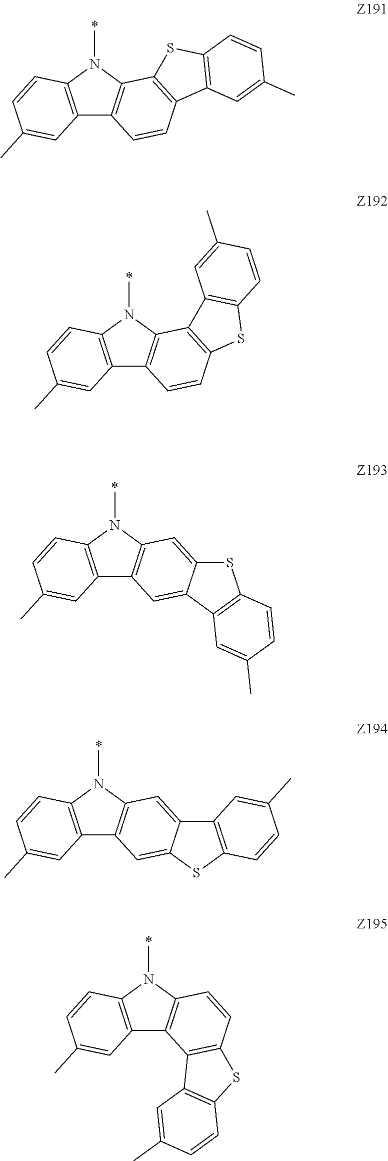

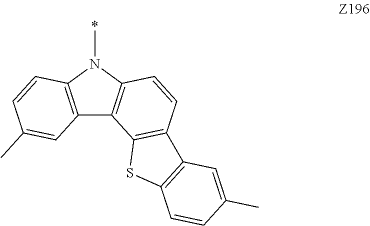

In addition to the above specific examples, groups in which all hydrogen atoms of alkyl groups of Z2, Z3, Z5, Z7 to Z12, Z87 to Z104, and Z179 to Z196 are substituted with deuterium atoms are exemplified herein as Z2(m), Z3(m), Z5(m), Z7(m) to Z12(m), Z87(m) to Z104(m), and Z179(m) to Z196(m), respectively. In addition, groups in which phenyl groups (C₆H₅) of Z4 to Z6, Z19 to Z86, and Z111 to Z178 are substituted with deuterated C₆D₅ are exemplified herein as Z4(p) to Z6(p), Z19(p) to Z86(p), and Z111(p) to Z178(p), respectively. Further, groups in which all hydrogen atoms of Z1 to Z196 are deuterated are exemplified herein as Z1(D) to Z196(D), respectively.

Specific examples of the group that can be adopted as the group bonded to L from the right side in the general formula (4) also include the following specific examples as well as Z2 to Z196 and deuterium atom substitutes thereof. Here, the groups that can be employed in the present invention shall not be construed as being limited by these specific examples. Also in the following specific examples, the expression of a methyl group is omitted. * indicates a bonding position to L.

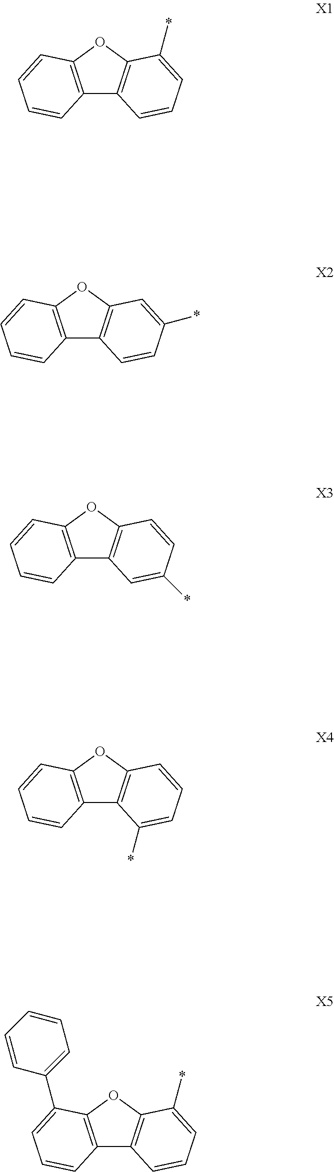 [SMILES: *c1cccc2c1oc1ccccc12 ; *c1ccc2c(c1)oc1ccccc12 ; *c1ccc2oc3ccccc3c2c1 ; *c1cccc2oc3ccccc3c12 ; *c1cccc2c1oc1c(-c3ccccc3)cccc12]

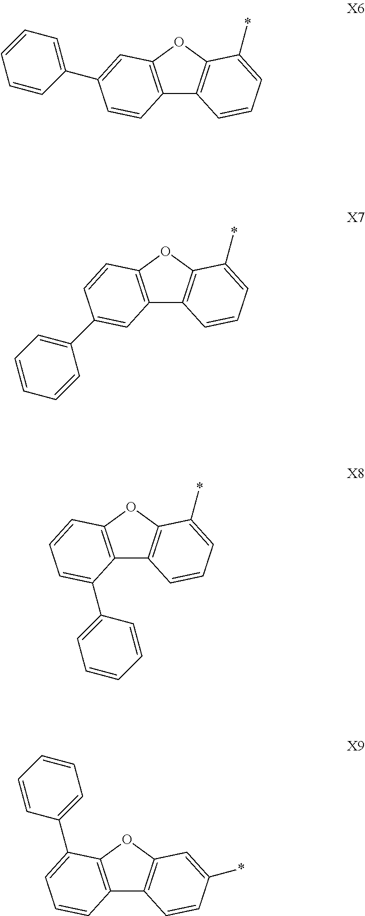 [SMILES: *c1cccc2c1oc1cc(-c3ccccc3)ccc12 ; *c1cccc2c1oc1ccc(-c3ccccc3)cc12 ; *c1cccc2c1oc1cccc(-c3ccccc3)c12 ; *c1ccc2c(c1)oc1c(-c3ccccc3)cccc12]

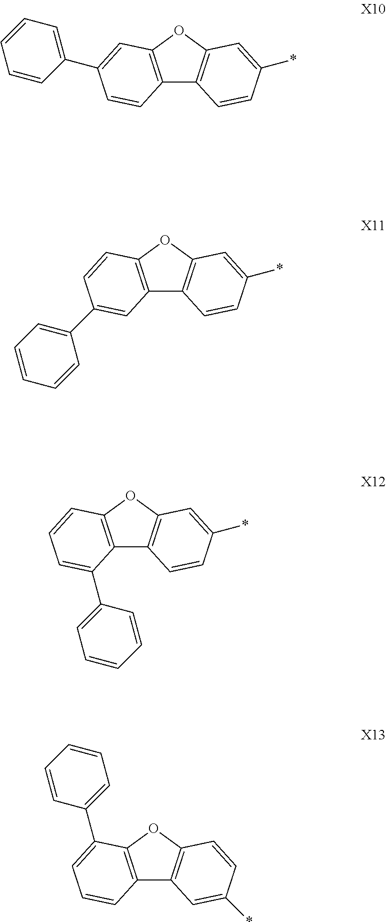 [SMILES: *c1ccc2c(c1)oc1cc(-c3ccccc3)ccc12 ; *c1ccc2c(c1)oc1ccc(-c3ccccc3)cc12 ; *c1ccc2c(c1)oc1cccc(-c3ccccc3)c12 ; *c1ccc2oc3c(-c4ccccc4)cccc3c2c1]

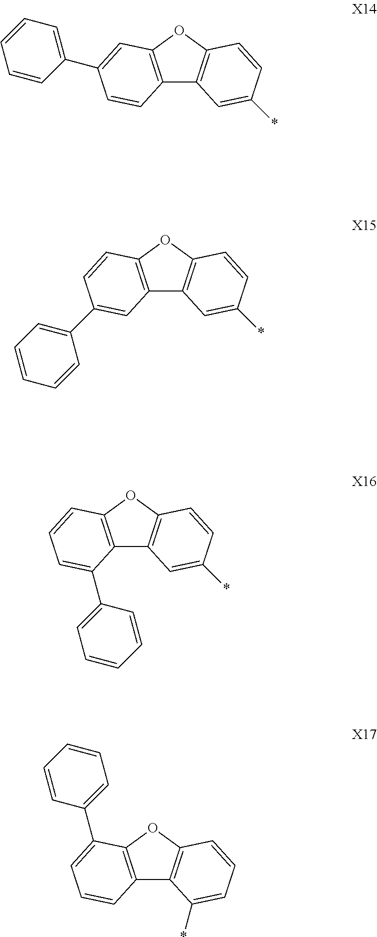 [SMILES: *c1ccc2oc3cc(-c4ccccc4)ccc3c2c1 ; *c1ccc2oc3ccc(-c4ccccc4)cc3c2c1 ; *c1ccc2oc3cccc(-c4ccccc4)c3c2c1 ; *c1cccc2oc3c(-c4ccccc4)cccc3c12]

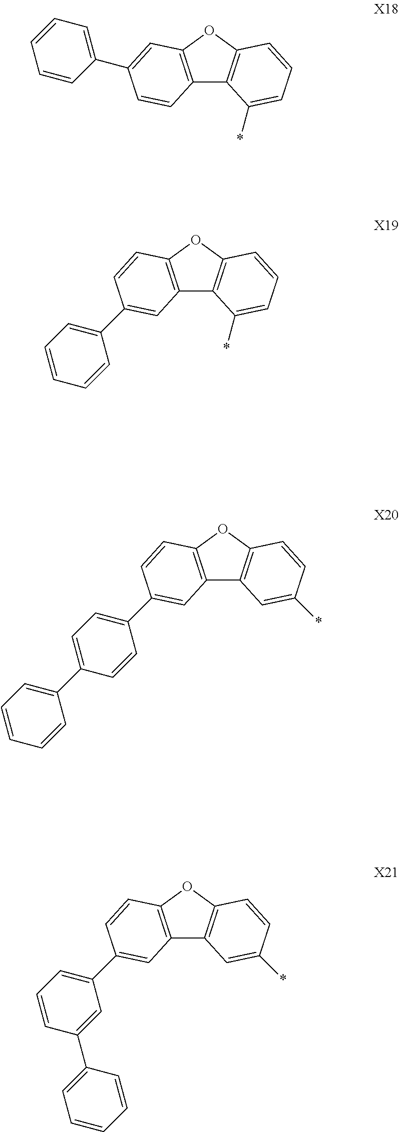 [SMILES: *c1cccc2oc3cc(-c4ccccc4)ccc3c12 ; *c1cccc2oc3ccc(-c4ccccc4)cc3c12 ; *c1ccc2oc3ccc(-c4ccc(-c5ccccc5)cc4)cc3c2c1 ; *c1ccc2oc3ccc(-c4cccc(-c5ccccc5)c4)cc3c2c1]

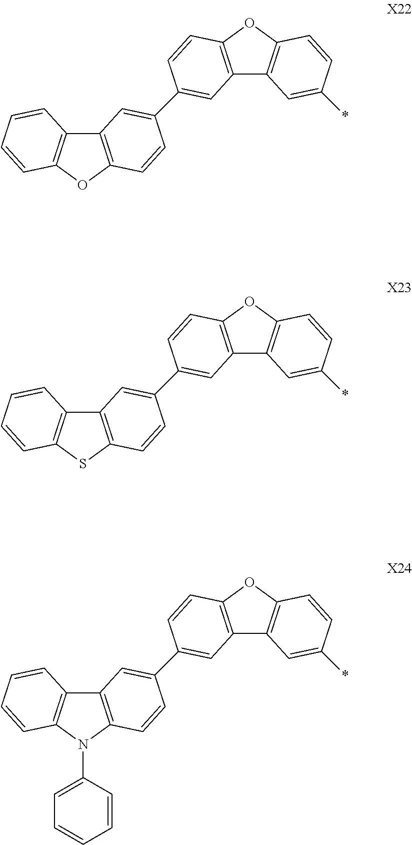 [SMILES: *c1ccc2oc3ccc(-c4ccc5oc6ccccc6c5c4)cc3c2c1 ; *c1ccc2oc3ccc(-c4ccc5sc6ccccc6c5c4)cc3c2c1 ; *c1ccc2oc3ccc(-c4ccc5c(c4)c4ccccc4n5-c4ccccc4)cc3c2c1]

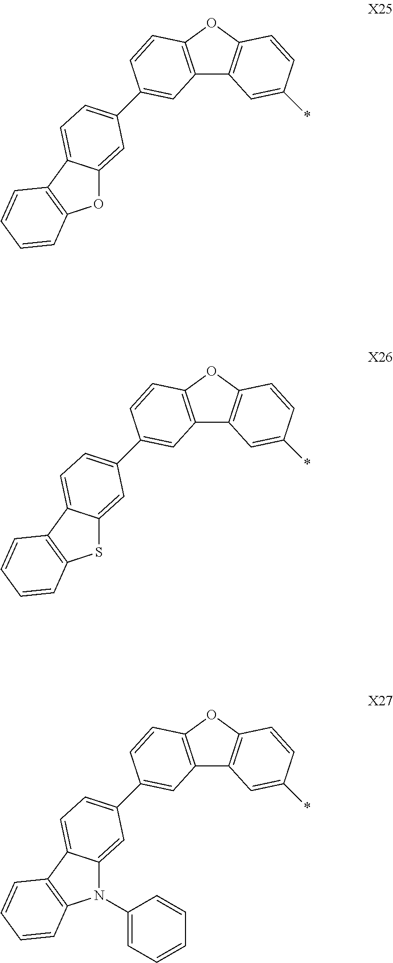 [SMILES: *c1ccc2oc3ccc(-c4ccc5c(c4)oc4ccccc45)cc3c2c1 ; *c1ccc2oc3ccc(-c4ccc5c(c4)sc4ccccc45)cc3c2c1 ; *c1ccc2oc3ccc(-c4ccc5c6ccccc6n(-c6ccccc6)c5c4)cc3c2c1]

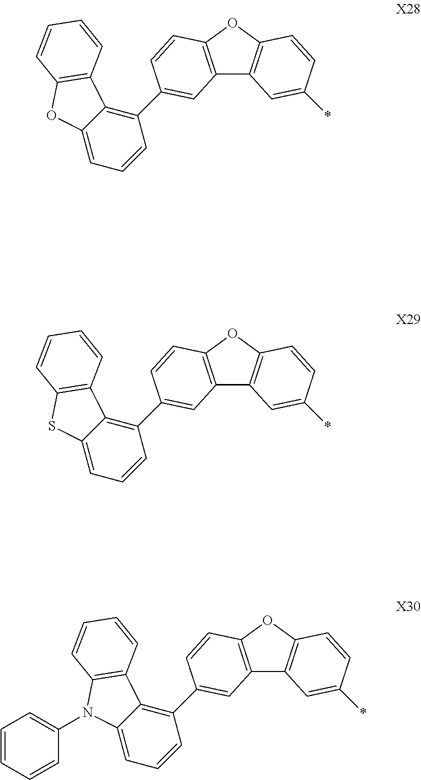 [SMILES: *c1ccc2oc3ccc(-c4cccc5oc6ccccc6c45)cc3c2c1 ; *c1ccc2oc3ccc(-c4cccc5sc6ccccc6c45)cc3c2c1 ; *c1ccc2oc3ccc(-c4cccc5c4c4ccccc4n5-c4ccccc4)cc3c2c1]

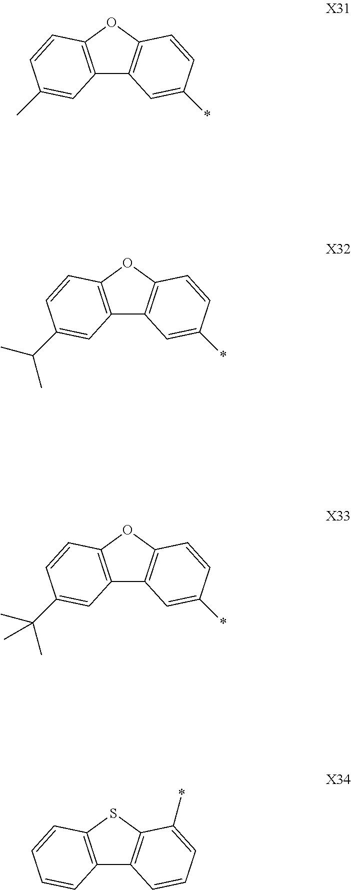 [SMILES: *c1ccc2oc3ccc(C)cc3c2c1 ; *c1ccc2oc3ccc(C(C)C)cc3c2c1 ; *c1ccc2oc3ccc(C(C)(C)C)cc3c2c1 ; *c1cccc2c1sc1ccccc12]

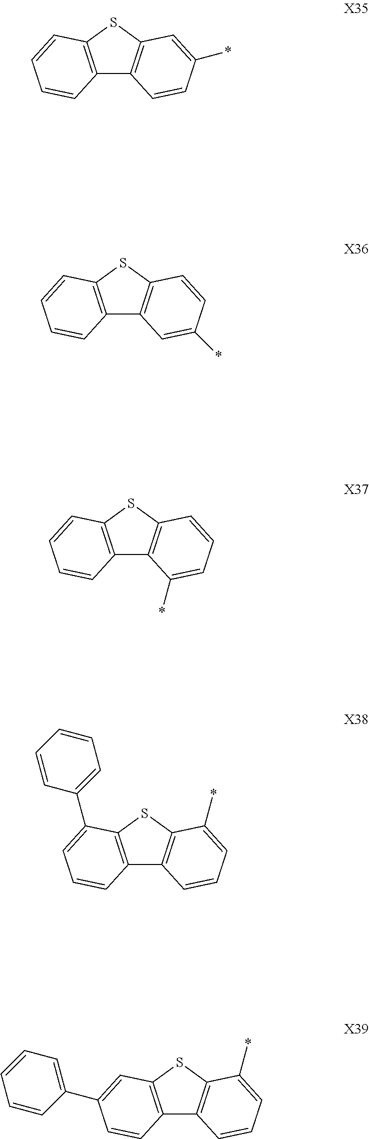 [SMILES: *c1ccc2c(c1)sc1ccccc12 ; *c1ccc2sc3ccccc3c2c1 ; *c1cccc2sc3ccccc3c12 ; *c1cccc2c1sc1c(-c3ccccc3)cccc12 ; *c1cccc2c1sc1cc(-c3ccccc3)ccc12]

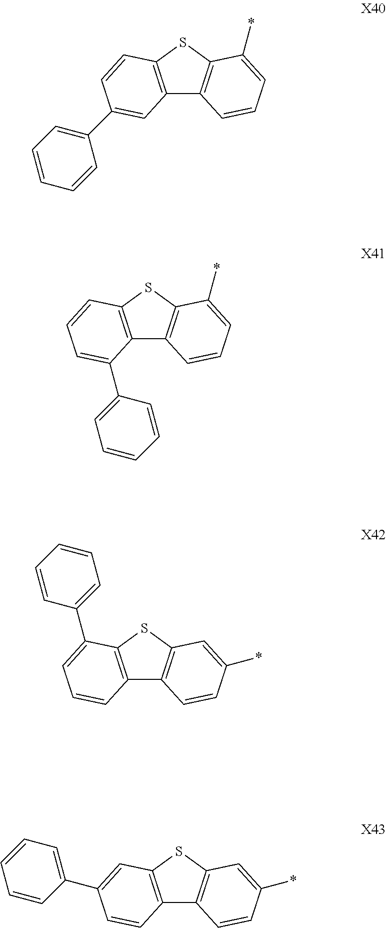 [SMILES: *c1cccc2c1sc1ccc(-c3ccccc3)cc12 ; *c1cccc2c1sc1cccc(-c3ccccc3)c12 ; *c1ccc2c(c1)sc1c(-c3ccccc3)cccc12 ; *c1ccc2c(c1)sc1cc(-c3ccccc3)ccc12]

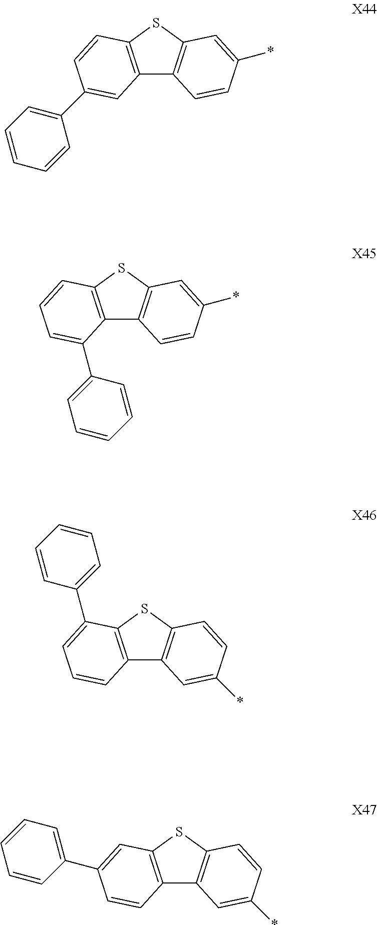 [SMILES: *c1ccc2c(c1)sc1ccc(-c3ccccc3)cc12 ; *c1ccc2c(c1)sc1cccc(-c3ccccc3)c12 ; *c1ccc2sc3c(-c4ccccc4)cccc3c2c1 ; *c1ccc2sc3cc(-c4ccccc4)ccc3c2c1]

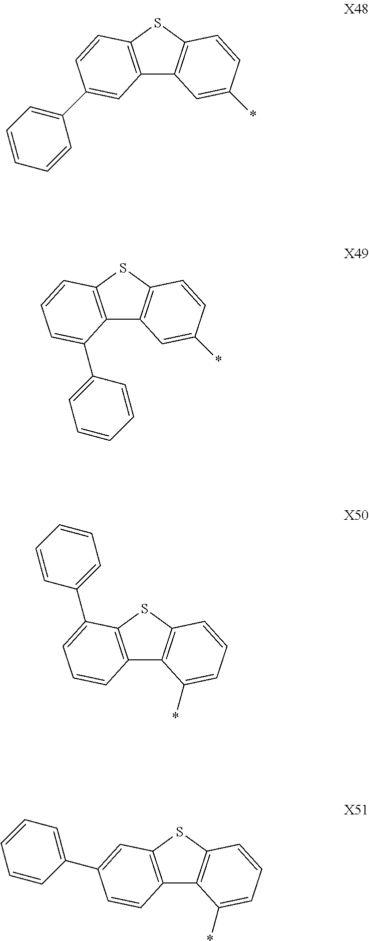 [SMILES: *c1ccc2sc3ccc(-c4ccccc4)cc3c2c1 ; *c1ccc2sc3cccc(-c4ccccc4)c3c2c1 ; *c1cccc2sc3c(-c4ccccc4)cccc3c12 ; *c1cccc2sc3cc(-c4ccccc4)ccc3c12]

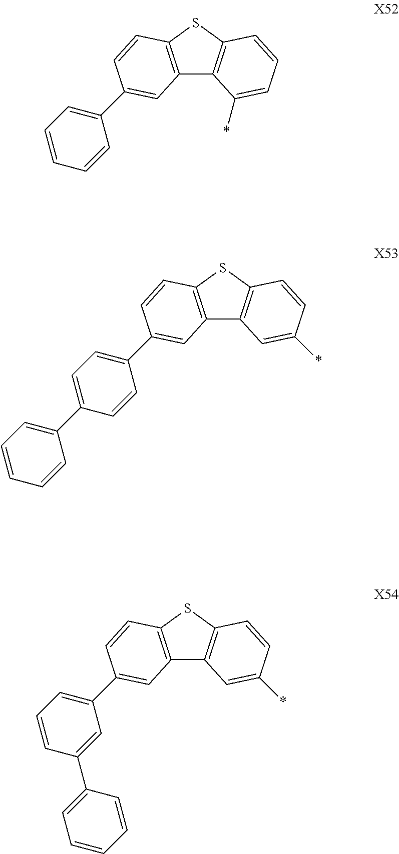 [SMILES: *c1cccc2sc3ccc(-c4ccccc4)cc3c12 ; *c1ccc2sc3ccc(-c4ccc(-c5ccccc5)cc4)cc3c2c1 ; *c1ccc2sc3ccc(-c4cccc(-c5ccccc5)c4)cc3c2c1]

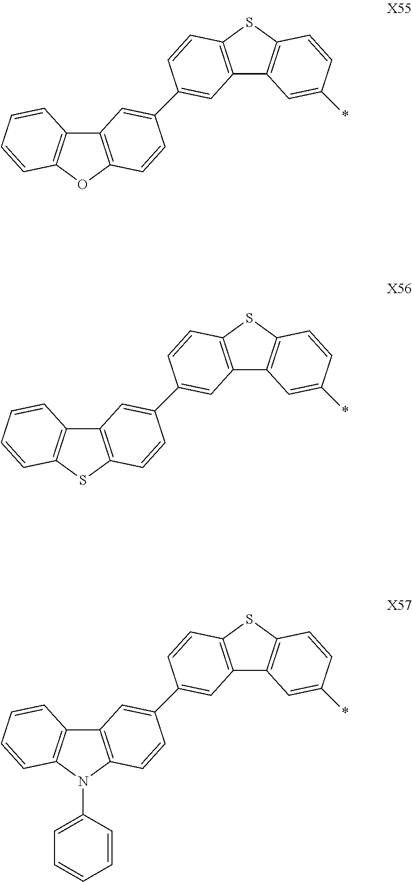 [SMILES: *c1ccc2sc3ccc(-c4ccc5oc6ccccc6c5c4)cc3c2c1 ; *c1ccc2sc3ccc(-c4ccc5sc6ccccc6c5c4)cc3c2c1 ; *c1ccc2sc3ccc(-c4ccc5c(c4)c4ccccc4n5-c4ccccc4)cc3c2c1]

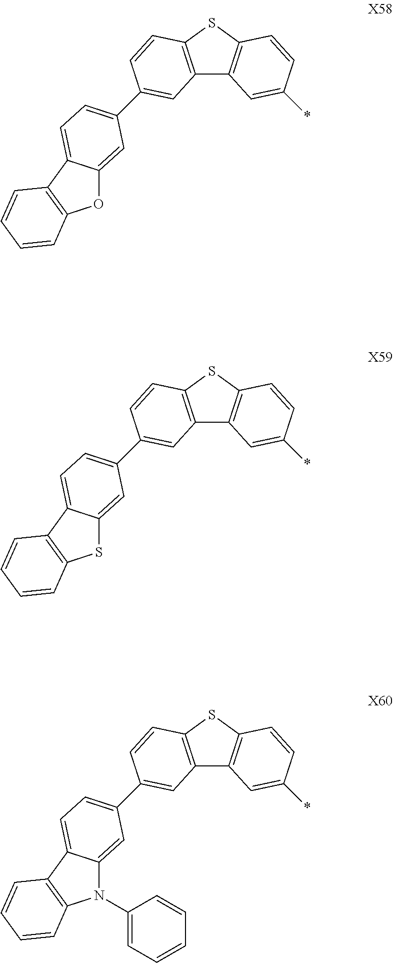 [SMILES: *c1ccc2sc3ccc(-c4ccc5c(c4)oc4ccccc45)cc3c2c1 ; *c1ccc2sc3ccc(-c4ccc5c(c4)sc4ccccc45)cc3c2c1 ; *c1ccc2sc3ccc(-c4ccc5c6ccccc6n(-c6ccccc6)c5c4)cc3c2c1]

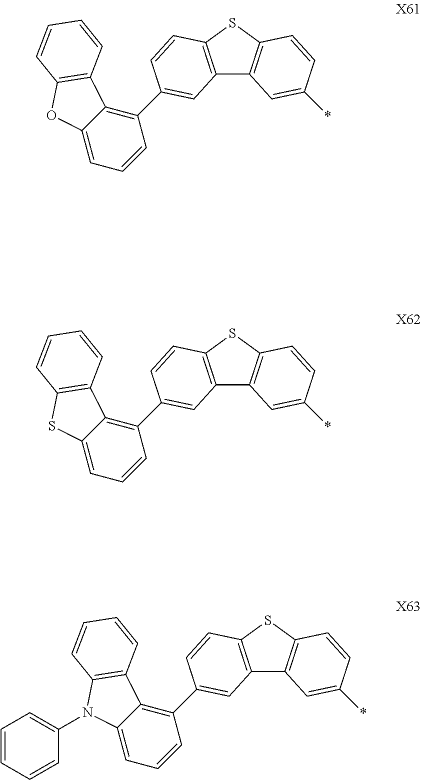 [SMILES: *c1ccc2sc3ccc(-c4cccc5oc6ccccc6c45)cc3c2c1 ; *c1ccc2sc3ccc(-c4cccc5sc6ccccc6c45)cc3c2c1 ; *c1ccc2sc3ccc(-c4cccc5c4c4ccccc4n5-c4ccccc4)cc3c2c1]

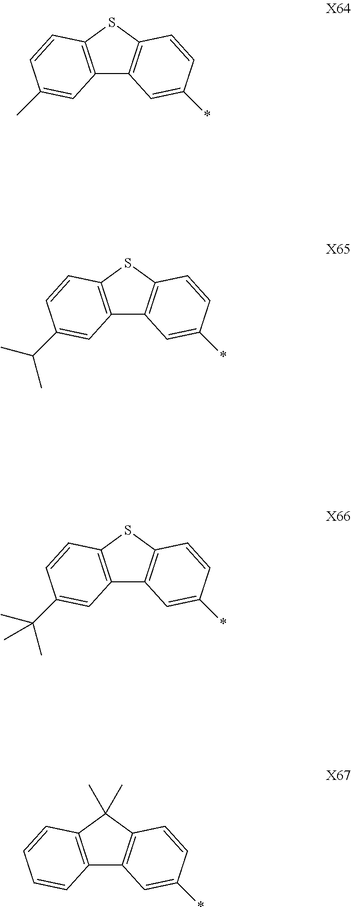 [SMILES: *c1ccc2sc3ccc(C)cc3c2c1 ; *c1ccc2sc3ccc(C(C)C)cc3c2c1 ; *c1ccc2sc3ccc(C(C)(C)C)cc3c2c1 ; *c1ccc2c(c1)-c1ccccc1C2(C)C]

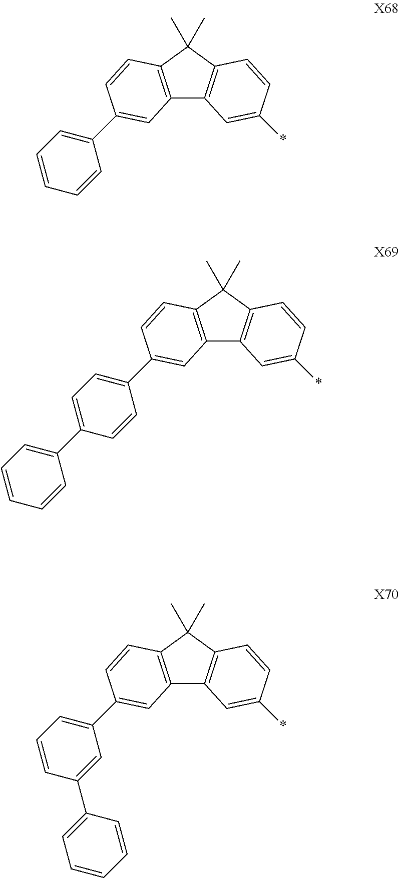 [SMILES: *c1ccc2c(c1)-c1cc(-c3ccccc3)ccc1C2(C)C ; *c1ccc2c(c1)-c1cc(-c3ccc(-c4ccccc4)cc3)ccc1C2(C)C ; *c1ccc2c(c1)-c1cc(-c3cccc(-c4ccccc4)c3)ccc1C2(C)C]

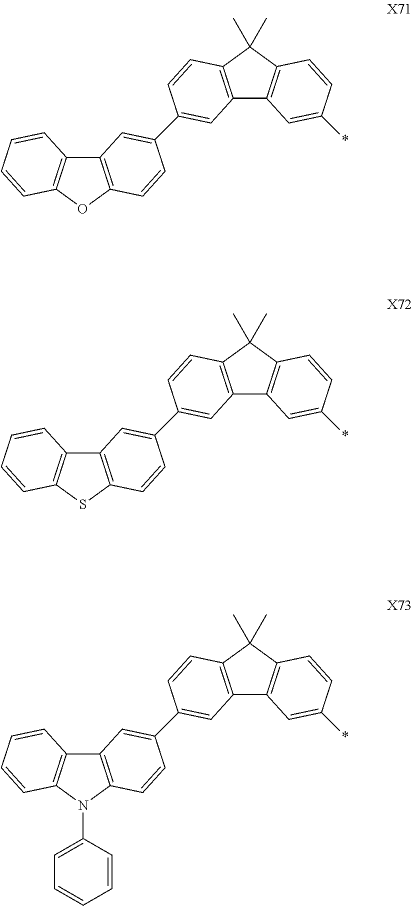 [SMILES: *c1ccc2c(c1)-c1cc(-c3ccc4oc5ccccc5c4c3)ccc1C2(C)C ; *c1ccc2c(c1)-c1cc(-c3ccc4sc5ccccc5c4c3)ccc1C2(C)C ; *c1ccc2c(c1)-c1cc(-c3ccc4c(c3)c3ccccc3n4-c3ccccc3)ccc1C2(C)C]

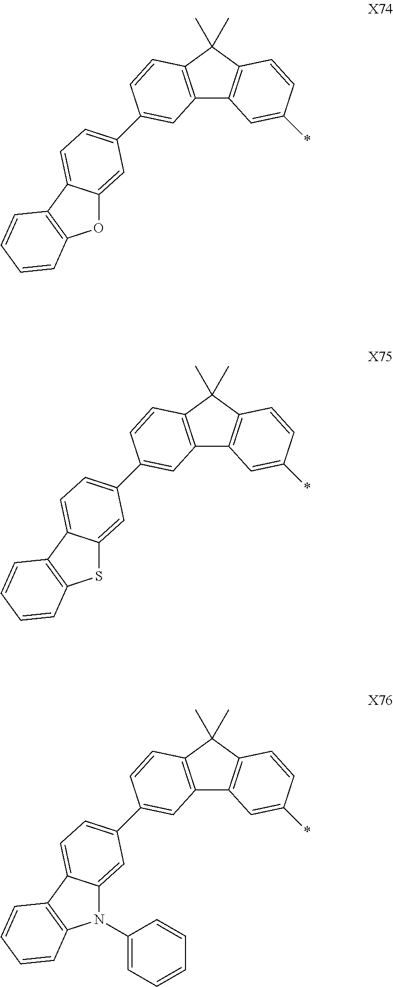 [SMILES: *c1ccc2c(c1)-c1cc(-c3ccc4c(c3)oc3ccccc34)ccc1C2(C)C ; *c1ccc2c(c1)-c1cc(-c3ccc4c(c3)sc3ccccc34)ccc1C2(C)C ; *c1ccc2c(c1)-c1cc(-c3ccc4c5ccccc5n(-c5ccccc5)c4c3)ccc1C2(C)C]

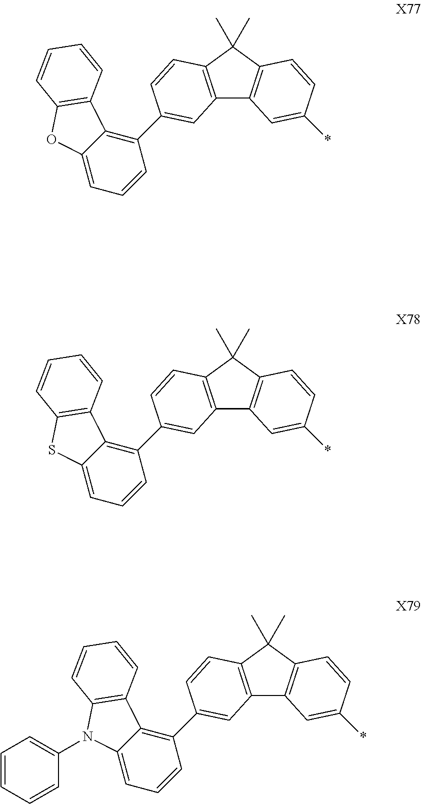 [SMILES: *c1ccc2c(c1)-c1cc(-c3cccc4oc5ccccc5c34)ccc1C2(C)C ; *c1ccc2c(c1)-c1cc(-c3cccc4sc5ccccc5c34)ccc1C2(C)C ; *c1ccc2c(c1)-c1cc(-c3cccc4c3c3ccccc3n4-c3ccccc3)ccc1C2(C)C]

Groups in which methyl groups (CH₃) of X31 to X33 and X64 to X79 are substituted with deuterated CD₃ are exemplified herein as X31(m) to X33(m), and X64(m) to X79(m), respectively, as well as the specific examples described above. Further, groups in which phenyl groups (C₆H₅) of X5 to X21, X38 to X54, and X68 to X70 are substituted with deuterated C₆D₅ are exemplified herein as X5(p) to X21(p), X38(p) to X54(p), and X68(p) to X70(p), respectively. Further, groups in which all hydrogen atoms of X1 to X79 are deuterated are exemplified herein as X1(D) to X79(D), respectively.

n in the general formula (4) represents an integer of 3 or 4. When X¹¹ is O, S, or C(R^{B})(R^{C}), L is bonded to a benzene ring to which (R¹¹⁵)n is bonded, and thus, n is 3. When X¹¹ is N(R^{A}), and L is bonded to a benzene ring to which (R¹¹⁵)n is bonded, n is 3, and further, when X¹¹ is N(R^{A}), and L is bonded to N represented by X¹¹, n is 4. n R¹¹⁵'s may be the same or different from each other.

L in the general formula (4) represents a single bond, a substituted or unsubstituted arylene group, a substituted or unsubstituted heteroarylene group, or a linking group in which two or more thereof are bonded. For an aryl structure of an arylene group and a heteroaryl structure of a heteroarylene group, the description on the “aryl group” and the “heteroaryl group” can be referred to. The arylene group and the heteroarylene group may be substituted, and in the case of being substituted, the groups are preferably substituted with one atom or group selected from the group consisting of a deuterium atom, an aryl group, a heteroaryl group, an alkyl group, and a cyano group, or a group formed by combining two or more thereof, and more preferably substituted with one atom or group selected from the group consisting of a deuterium atom, an aryl group, a heteroaryl group, and an alkyl group, or a group formed by combining two or more thereof. In the case where the groups are substituted, a methyl group, an ethyl group, an isopropyl group, a tert-butyl group, a phenyl group, or deuterated products thereof are preferable. In one aspect of the present invention, L is an unsubstituted arylene group.

Specific examples of L are shown below. Here, L that can be employed in the present invention shall not be construed as being limited by these specific examples. In the following specific examples, the expression of a methyl group is omitted. Therefore, for example, L3 to L5 are substituted with a methyl group. * indicates a bonding position. L1 is a single bond.

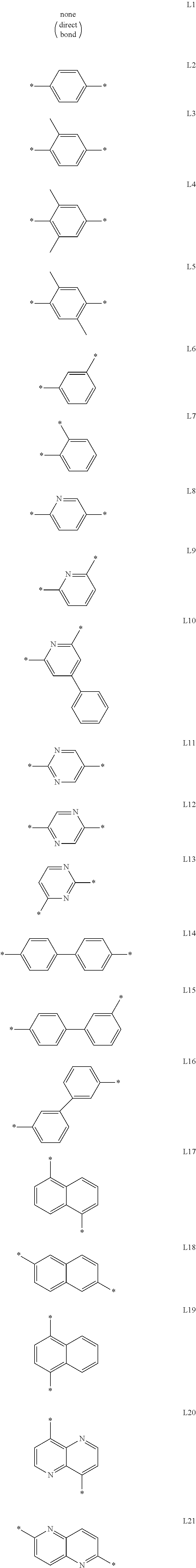

In one aspect of the present invention, in the general formula (4), the group bonded to L from the left side is selected from Z1 to Z196 and deuterated products thereof, and the group bonded to L from the right side is selected from X1 to X79 and deuterated products thereof (Aspect 1). In one aspect of the present invention, the group bonded to L from the left side is selected from Z1 to Z12 and deuterated products thereof, and the group bonded to L from the right side is selected from X1 to X79 and deuterated products thereof (Aspect 2). In one aspect of the present invention, the group bonded to L from the left side is selected from Z13 to Z196 and deuterated products thereof, and the group bonded to L from the right side is selected from X1 to X79 and deuterated products thereof (Aspect 3). In one aspect of the present invention, the group bonded to L from the left side is selected from Z1 to Z196 and deuterated products thereof, and the group bonded to L from the right side is selected from X1 to X66 and deuterated products thereof (Aspect 4). In one aspect of the present invention, the group bonded to L from the left side is selected from Z1 to Z196 and deuterated products thereof, and the group bonded to L from the right side is selected from X1 to X33 and deuterated products thereof (Aspect 5). In one aspect of the present invention, the group bonded to L from the left side is selected from Z1 to Z196 and deuterated products thereof, and the group bonded to L from the right side is selected from X1 to X21, X31 to X33, and deuterated products thereof (Aspect 6). In one aspect of the present invention, the group bonded to L from the left side is selected from Z1 to Z196 and deuterated products thereof, and the group bonded to L from the right side is selected from X22 to X30 and deuterated products thereof (Aspect 7).

In one aspect of the present invention, L in Aspect 1 is L1. In one aspect of the present invention, Lin Aspect 2 is L1. In one aspect of the present invention, Lin Aspect 3 is L1. In one aspect of the present invention, L in Aspect 4 is L1. In one aspect of the present invention, L in Aspect 5 is L1. In one aspect of the present invention, L in Aspect 6 is L1. In one aspect of the present invention, L in Aspect 7 is L1.

In one aspect of the present invention, L in Aspect 1 is L6. In one aspect of the present invention, Lin Aspect 2 is L6. In one aspect of the present invention, Lin Aspect 3 is L6. In one aspect of the present invention, L in Aspect 4 is L6. In one aspect of the present invention, L in Aspect 5 is L6. In one aspect of the present invention, L in Aspect 6 is L6. In one aspect of the present invention, L in Aspect 7 is L6.

In one aspect of the present invention, L in Aspect 1 is L14. In one aspect of the present invention, L in Aspect 2 is L14. In one aspect of the present invention, L in Aspect 3 is L14. In one aspect of the present invention, L in Aspect 4 is L14. In one aspect of the present invention, L in Aspect 5 is L14. In one aspect of the present invention, L in Aspect 6 is L14. In one aspect of the present invention, L in Aspect 7 is L14.

In one aspect of the present invention, L in Aspect 1 is L16. In one aspect of the present invention, L in Aspect 2 is L16. In one aspect of the present invention, L in Aspect 3 is L16. In one aspect of the present invention, L in Aspect 4 is L16. In one aspect of the present invention, L in Aspect 5 is L16. In one aspect of the present invention, L in Aspect 6 is L16. In one aspect of the present invention, L in Aspect 7 is L16.

Specific examples of the compound represented by the general formula (4) are shown below. Here, the compound represented by the general formula (4) that can be employed in the present invention shall not be construed as being limited by the following specific examples.

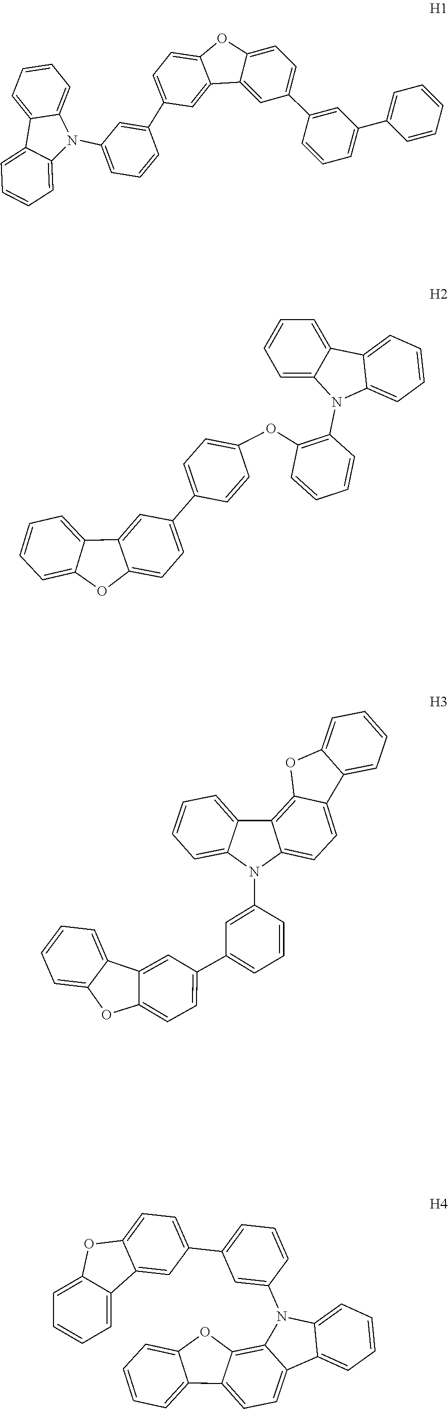

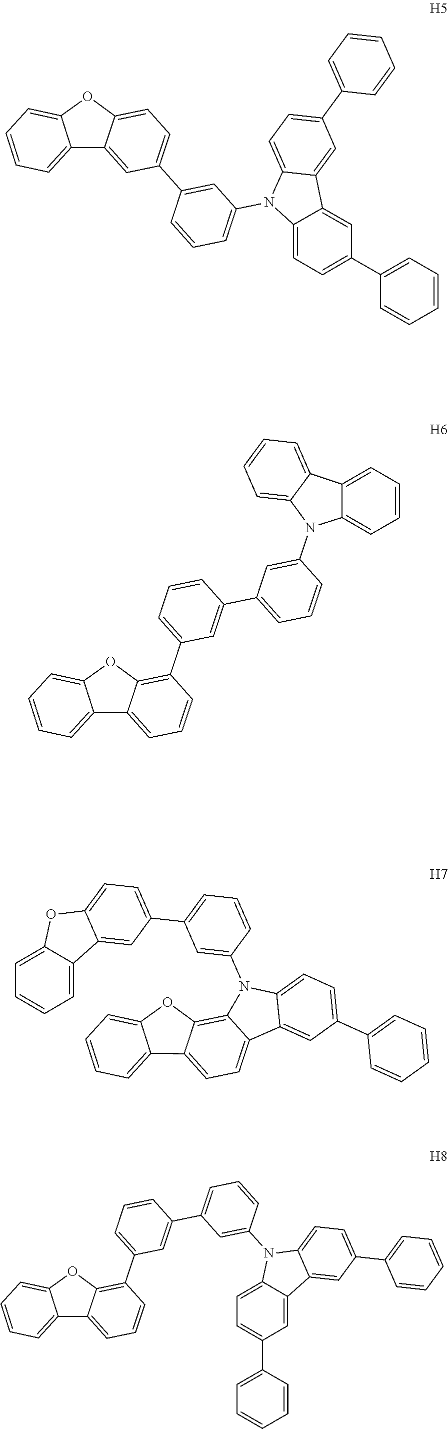

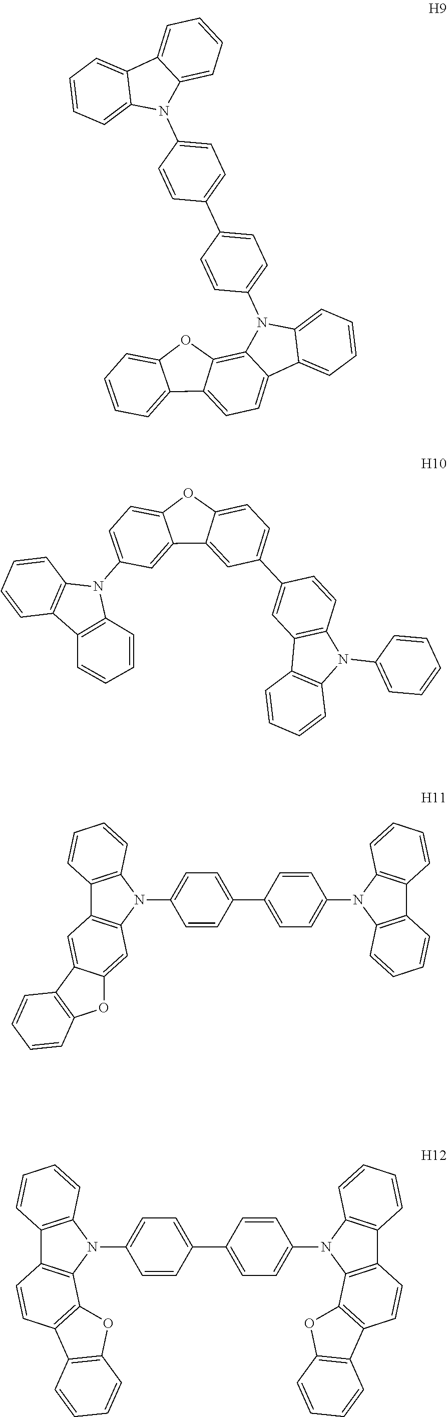

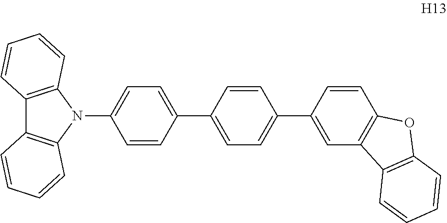

Compounds in which all hydrogen atoms present in substituted or unsubstituted carbazol-9-yl groups present in H1 to H13 are substituted with deuterium atoms are exemplified herein as H1(d) to H13(d), respectively, as well as the specific examples described above. Further, compounds in which all hydrogen atoms present in H1 to H13 are substituted with deuterium atoms are exemplified herein as H1(D) to H13(D), respectively.

The molecular weight of the compound represented by the general formula (4) is preferably 1500 or less, more preferably 1200 or less, further preferably 1000 or less, still further preferably 800 or less, and for example, may be 600 or less, for example, when there is an intention to form and use a film of an organic layer containing the compound represented by the general formula (4) through a vapor deposition method. The lower limit value of the molecular weight is the molecular weight of the smallest compound in the compound group represented by the general formula (4).

The compound represented by the general formula (4) with a smaller dipole moment is preferable because the orientation when a film is formed increases. The dipole moment is preferably smaller than 2.3, more preferably smaller than 2.0, further preferably smaller than 1.7, and still further preferably smaller than 1.4.

As the compound represented by the general formula (4), a compound composed of atoms selected from the group consisting of a carbon atom, a hydrogen atom, a deuterium atom, a nitrogen atom, an oxygen atom, and a sulfur atom can be selected. For example, as the compound represented by the general formula (4), a compound composed of atoms selected from the group consisting of a carbon atom, a hydrogen atom, a deuterium atom, a nitrogen atom, and an oxygen atom can be selected. For example, as the compound represented by the general formula (4), a compound composed of atoms selected from the group consisting of a carbon atom, a hydrogen atom, a deuterium atom, a nitrogen atom, and a sulfur atom can be selected. For example, as the compound represented by the general formula (4), a compound composed of atoms selected from the group consisting of a carbon atom, a hydrogen atom, a deuterium atom, and a nitrogen atom can be selected.

Hereinafter, the respective members and the respective layers other than the light-emitting layer of the organic electroluminescent device will be described.

### Substrate:

In some embodiments, the organic electroluminescent device of the present invention is supported by a substrate, and the substrate is not particularly limited, and may be any material commonly used in organic electroluminescent devices, for example, formed of glass, transparent plastic, quartz, and silicon.

### Anode:

In some embodiments, the anode of the organic electroluminescent device is made of a metal, an alloy, an electroconductive compound, or a combination thereof. In some embodiments, the metal, alloy, or electroconductive compound has a large work function (4 eV or more). In some embodiments, the metal is Au. In some embodiments, the conductive transparent material is selected from CuI, indium tin oxide (ITO), SnO₂, and ZnO. In some embodiments, an amorphous material capable of forming a transparent conductive film, such as IDIXO (In₂O₃—ZnO), is used. In some embodiments, the anode is a thin film. In some embodiments, the thin film is made by vapor deposition or sputtering. In some embodiments, the film is patterned by a photolithography method. In some embodiments, where the pattern may not require high accuracy (for example, approximately 100 μm or more), the pattern may be formed with a mask having a desired shape on vapor deposition or sputtering of the electrode material. In some embodiments, when a coating material, such as an organic electroconductive compound can be applied, a wet film forming method, such as a printing method and a coating method is used. In some embodiments, when the emitted light goes through the anode, the anode has a transmittance of more than 10%, and the anode has a sheet resistance of several hundred Ohm per unit area or less. In some embodiments, the thickness of the anode is 10 to 1,000 nm. In some embodiments, the thickness of the anode is 10 to 200 nm. In some embodiments, the thickness of the anode varies depending on the material to be used.

### Cathode:

In some embodiments, the cathode is made of an electrode material such as a metal having a small work function (4 eV or less) (referred to as an electron injection metal), an alloy, an electroconductive compound, or a combination thereof. In some embodiments, the electrode material is selected from sodium, a sodium-potassium alloy, magnesium, lithium, a magnesium-copper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth element. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per unit area or less. In some embodiments, the thickness of the cathode is 10 nm to 5 μm. In some embodiments, the thickness of the cathode is 50 to 200 nm. In some embodiments, for transmitting the emitted light, any one of the anode and the cathode of the organic electroluminescent device is transparent or translucent. In some embodiments, the transparent or translucent electroluminescent device enhances the light emission luminance.

In some embodiments, the cathode is formed with a conductive transparent material, as described for the anode, to form a transparent or translucent cathode. In some embodiments, a device includes an anode and a cathode, both being transparent or translucent.

### Injection Layer:

An injection layer is a layer between the electrode and the organic layer. In some embodiments, the injection layer decreases the drive voltage and enhances the light emission luminance. In some embodiments, the injection layer includes a hole injection layer and an electron injection layer. The injection layer can be positioned between the anode and the light-emitting layer or the hole transport layer, and between the cathode and the light-emitting layer or the electron transport layer. In some embodiments, an injection layer is present. In some embodiments, no injection layer is present.

Preferred compound examples for use as a hole injection material are shown below.

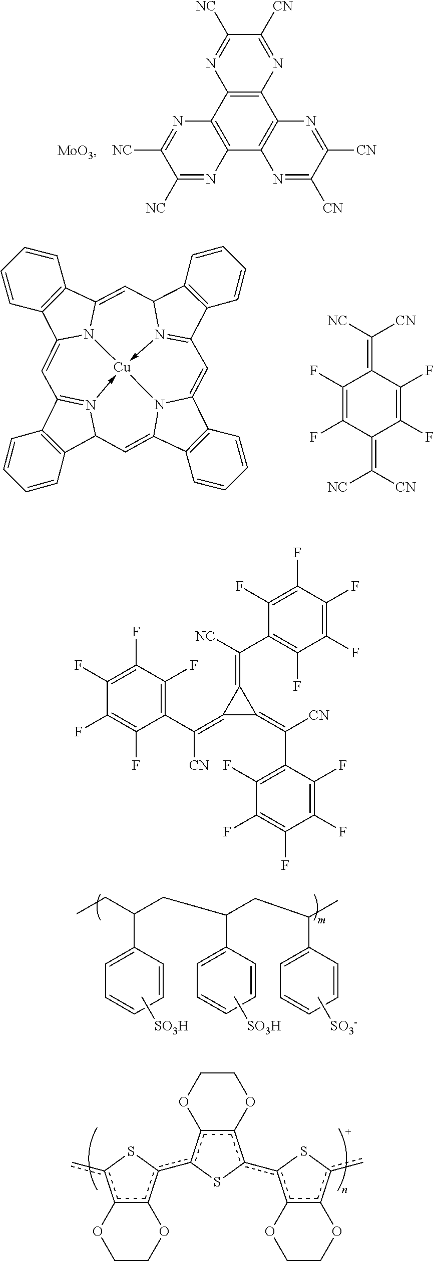

Next, preferred compound examples for use as an electron injection material are shown below.

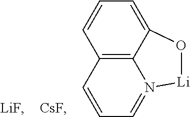

### Barrier Layer:

A barrier layer is a layer capable of inhibiting charges (electrons or holes) and/or excitons present in the light-emitting layer from being diffused outside the light-emitting layer. In some embodiments, the electron barrier layer is between the light-emitting layer and the hole transport layer, and inhibits electrons from passing through the light-emitting layer toward the hole transport layer. In some embodiments, the hole barrier layer is between the light-emitting layer and the electron transport layer, and inhibits holes from passing through the light-emitting layer toward the electron transport layer. In some embodiments, the barrier layer inhibits excitons from being diffused outside the light-emitting layer. In some embodiments, the electron barrier layer and the hole barrier layer are exciton barrier layers. The term “electron barrier layer” or “exciton barrier layer” used herein includes a layer that has both the function of an electron barrier layer and the function of an exciton barrier layer.

### Hole Barrier Layer:

A hole barrier layer acts as an electron transport layer. In some embodiments, the hole barrier layer inhibits holes from reaching the electron transport layer while transporting electrons. In some embodiments, the hole barrier layer enhances the recombination probability of electrons and holes in the light-emitting layer. The material for the hole barrier layer may be the same materials as the ones described for the electron transport layer.

Preferred compound examples for use for the hole barrier layer are shown below.

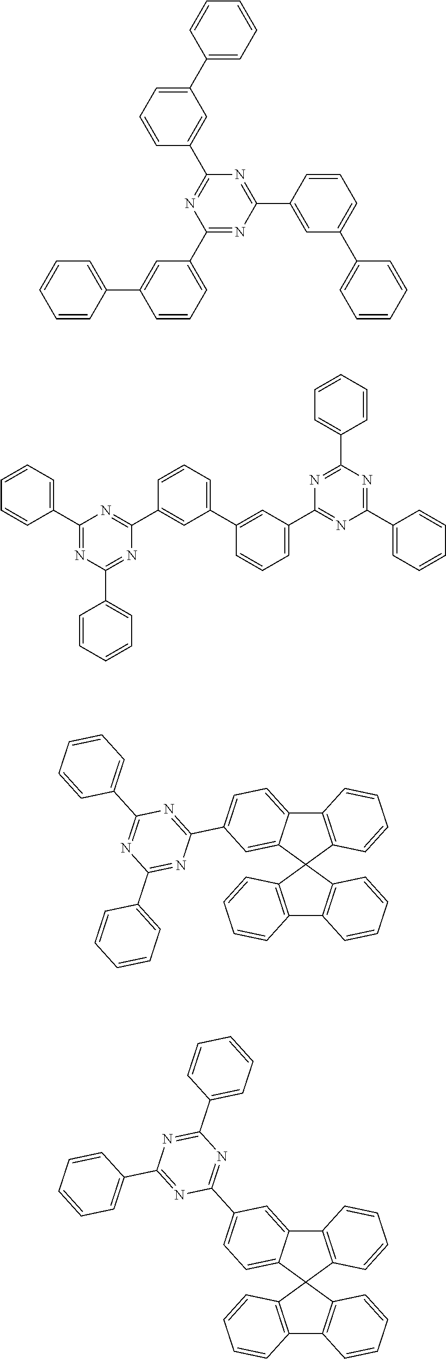 [SMILES: c1ccc(-c2cccc(-c3nc(-c4cccc(-c5ccccc5)c4)nc(-c4cccc(-c5ccccc5)c4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3ccc4c(c3)C3(c5ccccc5-c5ccccc53)c3ccccc3-4)n2)cc1 ; c1ccc(-c2nc(-c3ccccc3)nc(-c3ccc4c(c3)-c3ccccc3C43c4ccccc4-c4ccccc43)n2)cc1]

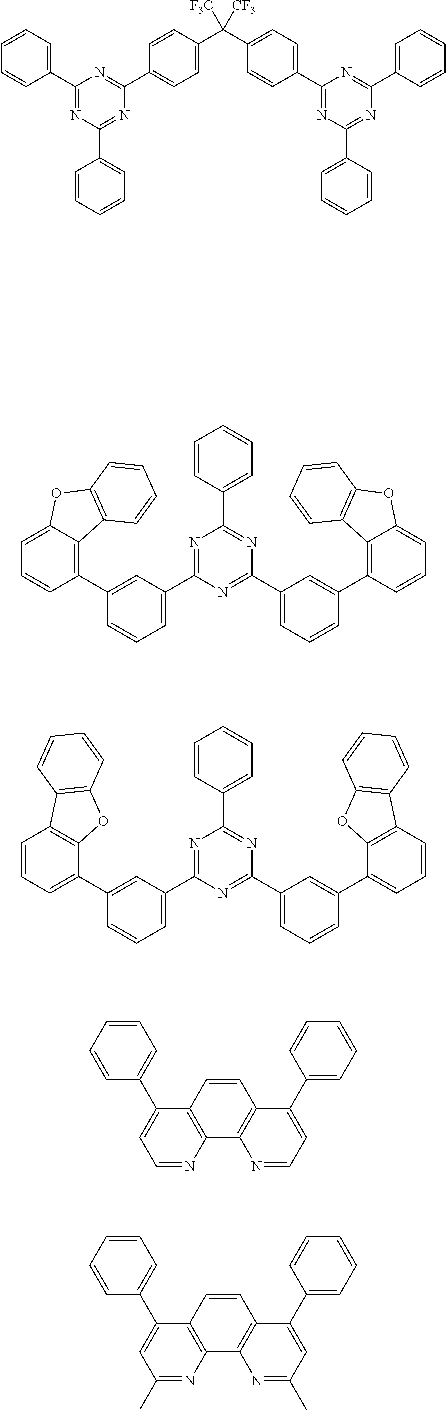 [SMILES: FC(F)(F)C(c1ccc(-c2nc(-c3ccccc3)nc(-c3ccccc3)n2)cc1)(c1ccc(-c2nc(-c3ccccc3)nc(-c3ccccc3)n2)cc1)C(F)(F)F ; c1ccc(-c2nc(-c3cccc(-c4cccc5oc6ccccc6c45)c3)nc(-c3cccc(-c4cccc5oc6ccccc6c45)c3)n2)cc1 ; c1ccc(-c2nc(-c3cccc(-c4cccc5c4oc4ccccc45)c3)nc(-c3cccc(-c4cccc5c4oc4ccccc45)c3)n2)cc1 ; c1ccc(-c2ccnc3c2ccc2c(-c4ccccc4)ccnc23)cc1 ; Cc1cc(-c2ccccc2)c2ccc3c(-c4ccccc4)cc(C)nc3c2n1]

### Electron Barrier Layer:

An electron barrier layer transports holes. In some embodiments, the electron barrier layer inhibits electrons from reaching the hole transport layer while transporting holes. In some embodiments, the electron barrier layer enhances the recombination probability of electrons and holes in the light-emitting layer. The materials for use for the electron barrier layer may be the same materials as those mentioned herein above for the hole transport layer.

Specific examples of preferred compounds for use as the electron barrier material are shown below.

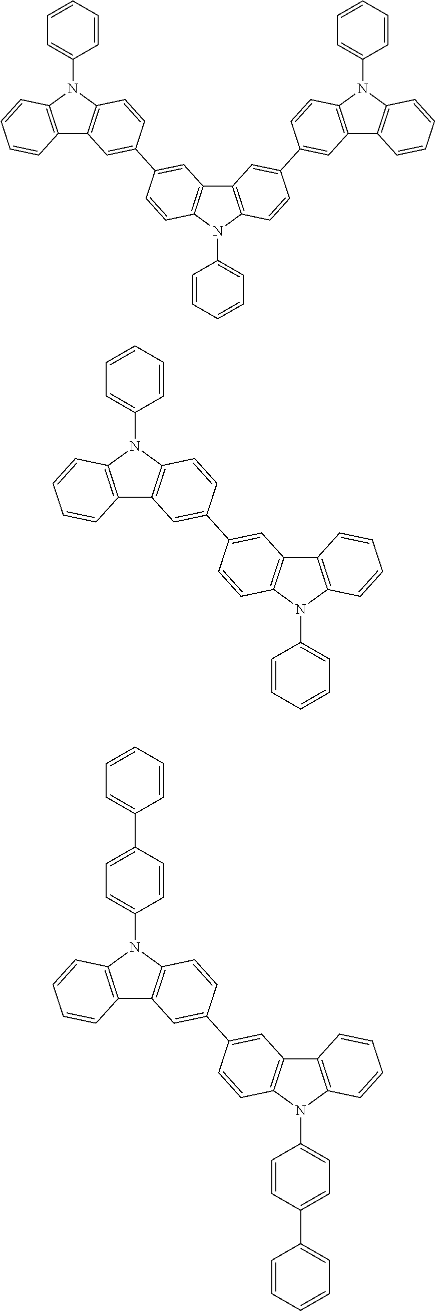 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

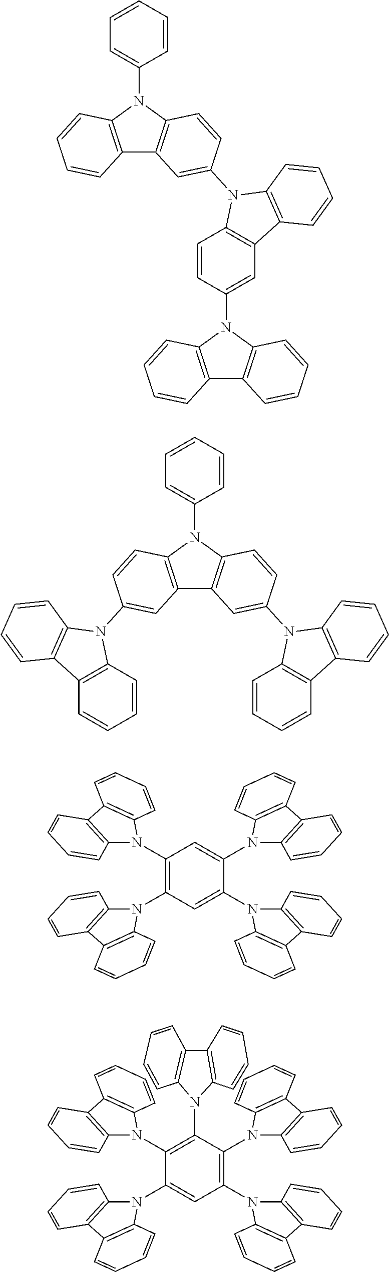

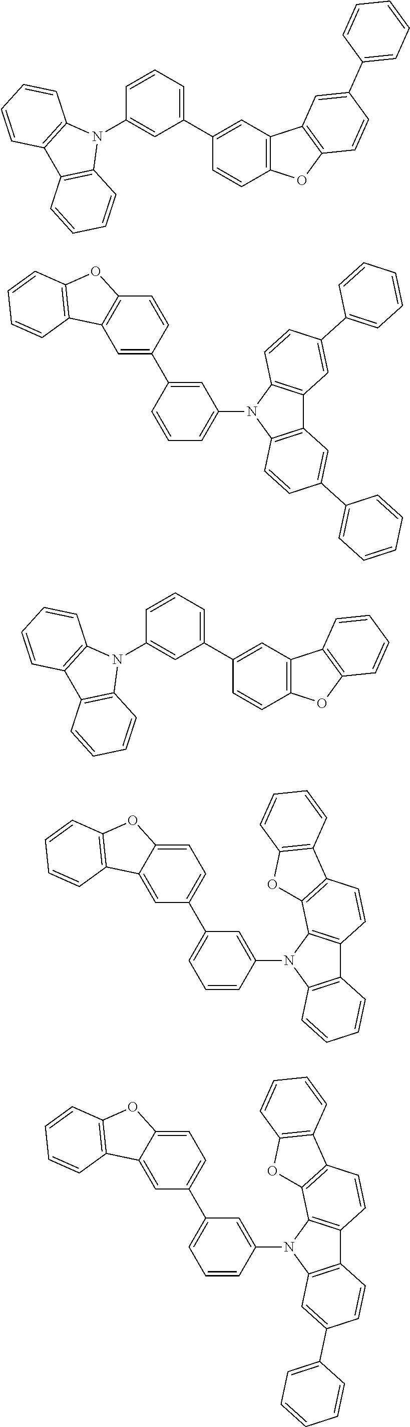

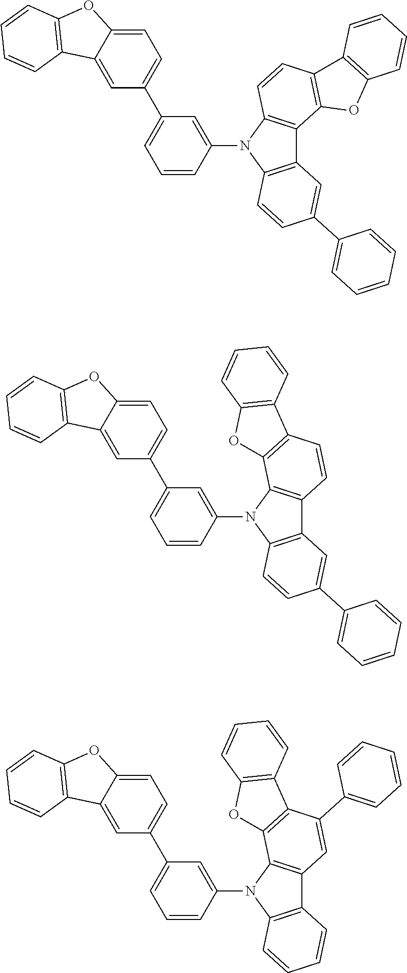 [SMILES: c1ccc(-c2ccc3c(c2)c2c4oc5ccccc5c4ccc2n3-c2cccc(-c3ccc4oc5ccccc5c4c3)c2)cc1 ; c1ccc(-c2ccc3c(c2)c2ccc4c5ccccc5oc4c2n3-c2cccc(-c3ccc4oc5ccccc5c4c3)c2)cc1 ; c1ccc(-c2cc3c4ccccc4n(-c4cccc(-c5ccc6oc7ccccc7c6c5)c4)c3c3oc4ccccc4c23)cc1]

### Exciton Barrier Layer:

An exciton barrier layer inhibits excitons generated through recombination of holes and electrons in the light-emitting layer from being diffused to the charge transport layer. In some embodiments, the exciton barrier layer enables effective confinement of excitons in the light-emitting layer. In some embodiments, the light emission efficiency of the device is enhanced. In some embodiments, the exciton barrier layer is adjacent to the light-emitting layer on any of the side of the anode and the side of the cathode, and on both the sides. In some embodiments, when the exciton barrier layer is present on the side of the anode, the layer may be present between the hole transport layer and the light-emitting layer and adjacent to the light-emitting layer. In some embodiments, when the exciton barrier layer is present on the side of the cathode, the layer may be present between the light-emitting layer and the cathode and adjacent to the light-emitting layer. In some embodiments, a hole injection layer, an electron barrier layer, or a similar layer is between the anode and the exciton barrier layer that is adjacent to the light-emitting layer on the side of the anode. In some embodiments, a hole injection layer, an electron barrier layer, a hole barrier layer, or a similar layer is between the cathode and the exciton barrier layer that is adjacent to the light-emitting layer on the side of the cathode. In some embodiments, the exciton barrier layer includes excited singlet energy and excited triplet energy, at least one of which is higher than the excited singlet energy and the excited triplet energy of the light-emitting material, respectively.

### Hole Transport Layer:

The hole transport layer contains a hole transport material. In some embodiments, the hole transport layer is a single layer. In some embodiments, the hole transport layer has a plurality of layers.

In some embodiments, the hole transport material has one of injection or transport property of holes and barrier property of electrons. In some embodiments, the hole transport material is an organic material. In some embodiments, the hole transport material is an inorganic material. Examples of known hole transport materials that can be used in the present invention include, but are not limited to, a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an allylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer (particularly, a thiophene oligomer), or a combination thereof. In some embodiments, the hole transport material is selected from a porphyrin compound, an aromatic tertiary amine compound, and a styryl amine compound. In some embodiments, the hole transport material is an aromatic tertiary amine compound. Specific examples of preferred compounds for use as the hole transport material are shown below.

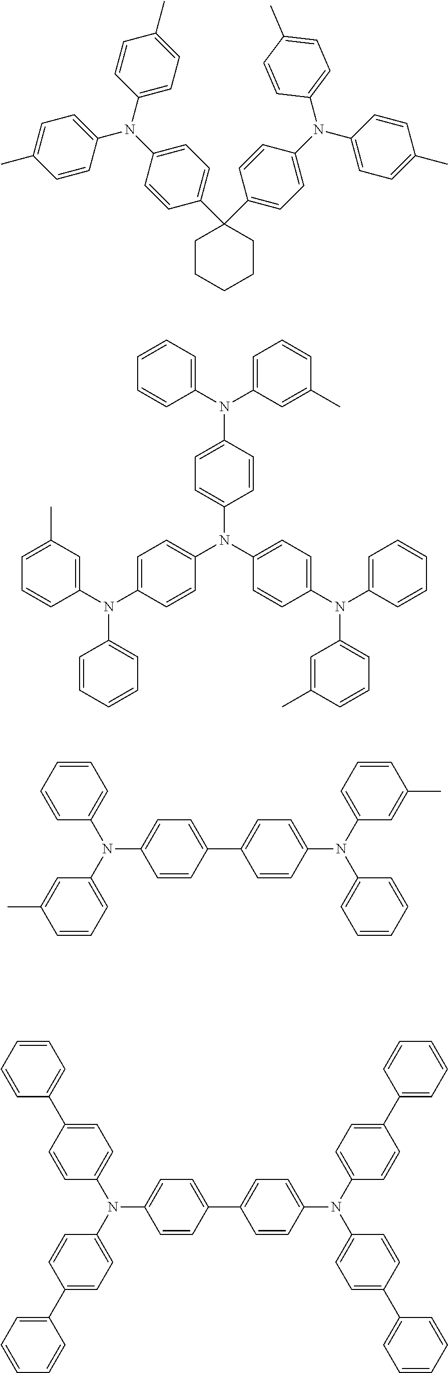

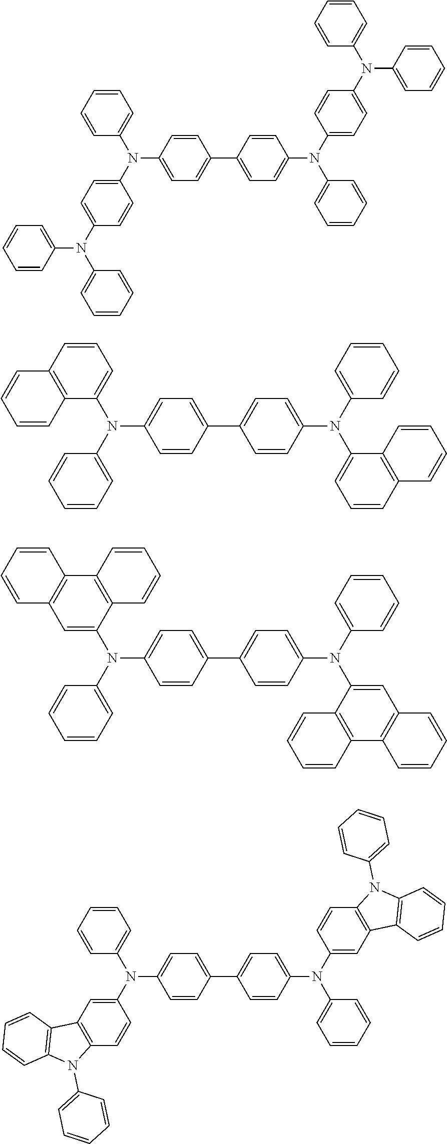

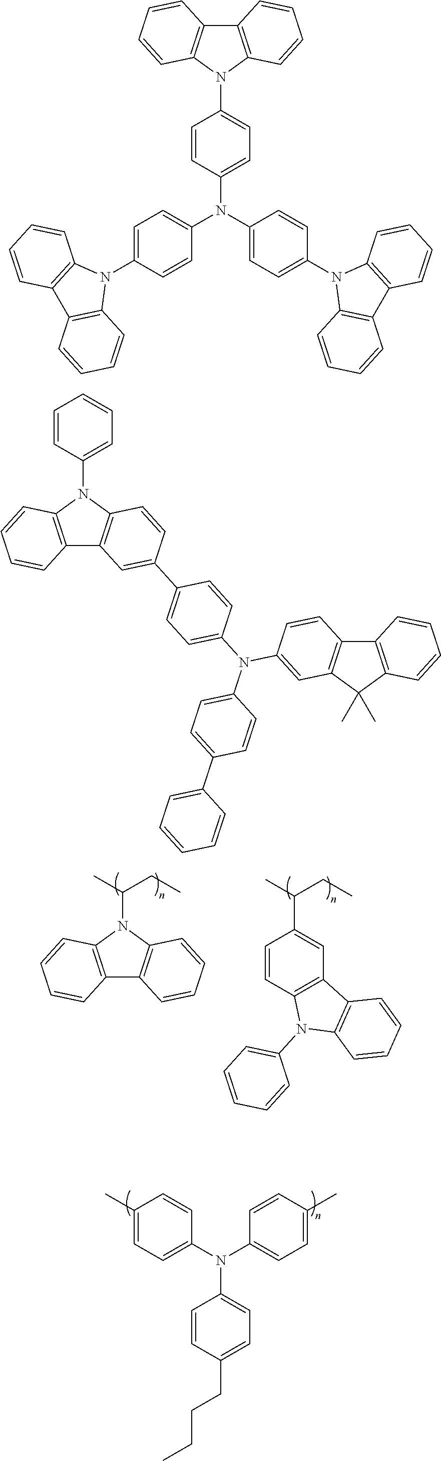

### Electron Transport Layer:

The electron transport layer contains an electron transport material. In some embodiments, the electron transport layer is a single layer. In some embodiments, the electron transport layer has a plurality of layers.

In some embodiments, the electron transport material needs only to have a function of transporting electrons, which are injected from the cathode, to the light-emitting layer. In some embodiments, the electron transport material also functions as a hole barrier material. Examples of the electron transport layer that can be used in the present invention include but are not limited to a nitro-substituted fluorene derivative, a diphenylquinone derivative, a thiopyran dioxide derivative, carbodiimide, a fluorenylidene methane derivative, anthraquinodimethane, an anthrone derivative, an oxadiazole derivative, an azole derivative, an azine derivative, or a combination thereof, or a polymer thereof. In some embodiments, the electron transport material is a thiadiazole derivative, or a quinoxaline derivative. In some embodiments, the electron transport material is a polymer material. Specific examples of preferred compounds for use as the electron transport material are shown below.

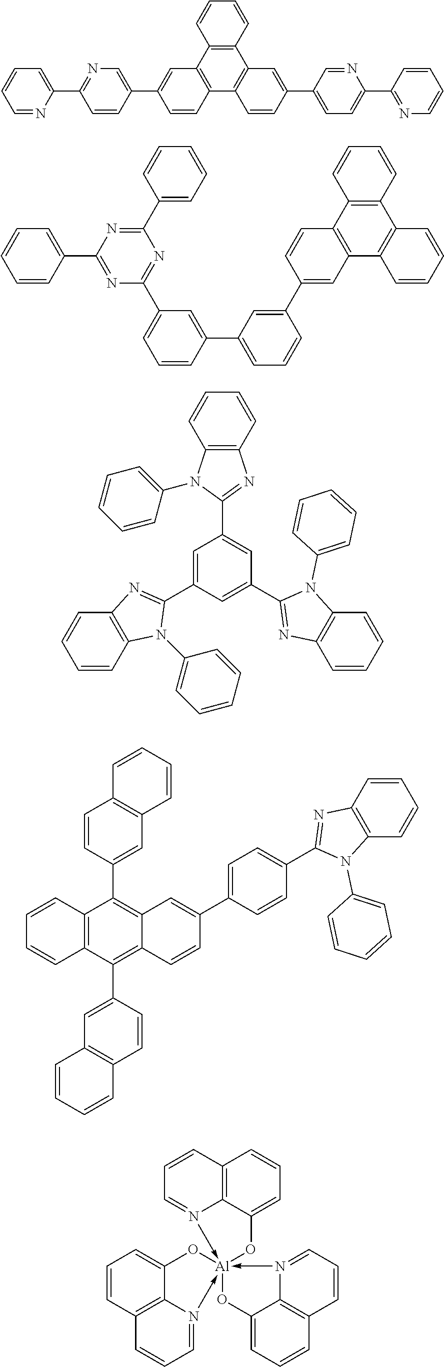

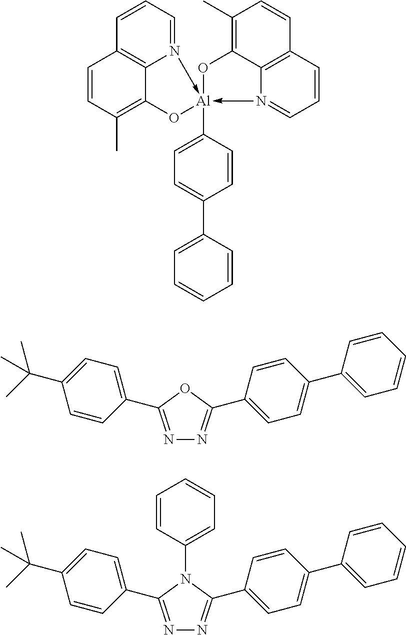

Preferred examples of compounds usable as materials that can be added to each organic layer are shown below. For example, the addition of a compound as a stabilizing material may be taken into consideration.

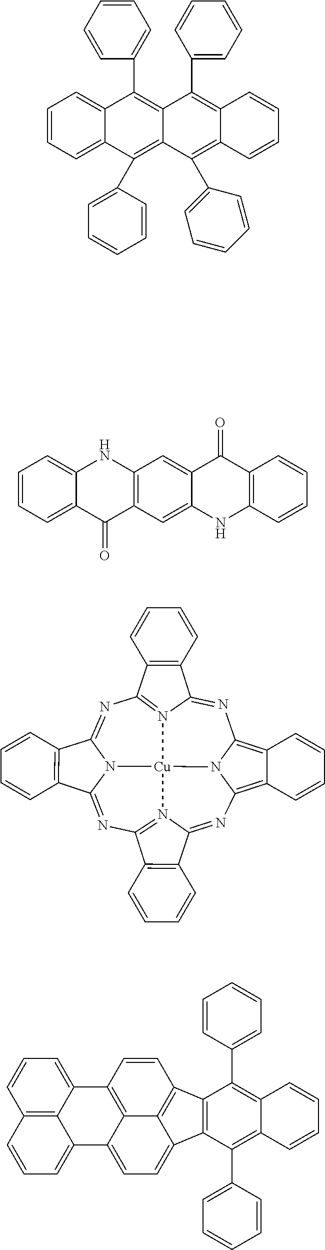

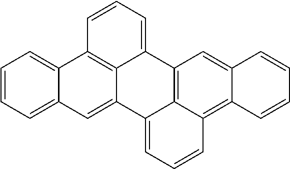 [SMILES: c1ccc2c(c1)cc1c3cccc4c5ccccc5cc(c5cccc2c51)c43]

Preferred materials that can be used in the organic electroluminescent device have been specifically exemplified. However, the materials that can be used in the present invention should not be construed as being limited to the following exemplary compounds. In addition, even the compounds exemplified as the material having a specific function can be diverted as a material having another function.

### Device:

In some embodiments, a light-emitting layer is incorporated into a device. For example, the device includes, but is not limited to an OLED bulb, an OLED lamp, a television screen, a computer monitor, a mobile phone, and a tablet.

In some embodiments, an electronic device includes an OLED having an anode, a cathode, and at least one organic layer including a light-emitting layer between the anode and the cathode.

In some embodiments, compositions described herein may be incorporated into various light-sensitive or light-activated devices, such as OLEDs or optoelectronic devices. In some embodiments, the composition may be useful in facilitating charge transfer or energy transfer within a device and/or as a hole transport material. Examples of the device include an organic light-emitting diode (OLED), an organic integrated circuit (OIC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC), or an organic laser diode (O-laser).

### Bulb or Lamp:

In some embodiments, an electronic device includes an OLED containing an anode, a cathode, and at least one organic layer containing a light-emitting layer between the anode and the cathode.

In some embodiments, a device includes OLEDs that differ in color. In some embodiments, a device includes an array containing a combination of OLEDs. In some embodiments, the combination of OLEDs is a combination of three colors (e.g., RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light including:
a circuit board having a first surface with a mounting surface and an opposing second surface, and defining at least one opening,
at least one OLED on the mounting surface, the at least one OLED configured to emit light, the at least one OLED including an anode, a cathode, and at least one organic layer including a light-emitting layer between the anode and the cathode,
a housing for the circuit board, and
at least one connector arranged at an end of the housing, the housing and the at least one connector defining a package adapted for installation in a light fixture.

In some embodiments, the OLED light has a plurality of OLEDs mounted on a circuit board such that light is emitted in a plurality of directions. In some embodiments, a portion of the light emitted in a first direction is polarized to emit in a second direction. In some embodiments, a reflector is used to polarize the light emitted in a first direction.

### Display or Screen:

In some embodiments, the light-emitting layer in the present invention can be used in a screen or a display. In some embodiments, the compounds according to the present invention are deposited onto a substrate using a process including, but not limited to, vacuum evaporation, deposition, vapor deposition, or chemical vapor deposition (CVD). In some embodiments, the substrate is a photoplate structure useful in a two-sided etching that provides a unique aspect ratio pixel. The screen (which may also be referred to as a mask) is used in a production process of an OLED display. The corresponding artwork pattern design facilitates a very steep and narrow tie-bar between the pixels in the vertical direction and a large, sweeping bevel opening in the horizontal direction. This allows the close patterning of pixels needed for high resolution displays while optimizing the chemical vapor deposition onto a TFT backplane.

The internal patterning of the pixel allows the construction of a three-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point, the entire pixel area is subjected to a similar etching rate but the depths are varying depending on the halftone pattern. Varying the size and spacing of the halftone pattern allows etching to be inhibited at different rates within the pixel allowing for a localized deeper etch needed to create steep vertical bevels.

A preferred material for the deposition mask is invar. Invar is a metal alloy that is cold rolled into long thin sheet in a steel mill. Invar cannot be electrodeposited onto a rotating mandrel as the nickel mask. A preferred and more cost feasible method for forming the open areas in the mask used for deposition is through a wet chemical etching.

In some embodiments, a screen or display pattern is a pixel matrix on a substrate. In some embodiments, a screen or display pattern is fabricated using lithography (e.g., photolithography and e-beam lithography). In some embodiments, a screen or display pattern is fabricated using a wet chemical etching. In further embodiments, a screen or display pattern is fabricated using plasma etching.

### Method for Producing Device:

An OLED display is generally produced by forming a large mother panel and then cutting the mother panel in units of cell panels. In general, each of the cell panels on the mother panel is formed by forming a thin film transistor (TFT) having an active layer and a source/drain electrode on a base substrate, applying a planarization film to the TFT, sequentially forming a pixel electrode, a light-emitting layer, a counter electrode, and an encapsulation layer in order, and is cut from the mother panel.

In another aspect of the present invention, provided herein is a method for producing an organic light-emitting diode (OLED) display, the method including:
a step of forming a barrier layer on a base substrate of a mother panel,
a step of forming a plurality of display units in units of cell panels on the barrier layer,
a step of forming an encapsulation layer on each of the display units of the cell panels, and
a step of applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light-emitting layer, a gate electrode, and a source/drain electrode. Each of the plurality of display units may include a thin film transistor (TFT) layer, a planarization film formed on the TFT layer, and a light-emitting unit formed on the planarization film, in which the organic film applied to the interface portion is formed of a same material as a material of the planarization film and is formed at a same time as the planarization film is formed. In some embodiments, a light-emitting unit is connected to the TFT layer with a passivation layer and a planarization film therebetween and an encapsulation layer that covers and protects the light-emitting unit. In some embodiments of the production method, the organic film is connected to neither the display units nor the encapsulation layer.

Each of the organic film and the planarization film can include any one of polyimide and acryl. In some embodiments, the barrier layer may be an inorganic film. In some embodiments, the base substrate may be formed of polyimide. The method may further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is produced. In some embodiments, the organic film may be formed on the edge portion of the barrier layer such that a portion of the organic film directly contacts the base substrate and a remaining portion of the organic film contacts the barrier layer while surrounding the edge portion of the barrier layer.

In some embodiments, the light-emitting layer includes a pixel electrode, a counter electrode, and an organic light-emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light-emitting layer emits light, thereby forming an image. Hereinafter, an image forming unit including the TFT layer and the light-emitting unit is referred to as a display unit.

In some embodiments, the encapsulation layer that covers the display unit and prevents penetration of external moisture may be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding an edge portion of the barrier layer.

In some embodiments, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In some embodiments, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the production method further includes a step of cutting along the interface portion, in which a groove is formed in the barrier layer, at least a portion of the organic film is formed in the groove, and the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is completely exposed without the organic film, an impact generated when each of the cell panels is cut along the groove at the interface portion is transferred to the barrier layer, thereby increasing the risk of cracks. However, in some embodiments, since the groove at the interface portion between the barrier layers is covered with the organic film and the organic film absorbs an impact that would otherwise be transferred to the barrier layer, each of the cell panels may be softly cut and cracks may be prevented from occurring in the barrier layer. In some embodiments, the organic film covering the groove at the interface portion and the planarization film are spaced apart from each other. For example, when the organic film and the planarization film are connected to each other as one layer, since external moisture may penetrate into the display unit through the planarization film and a portion where the organic film remains, the organic film and the planarization film are spaced apart from each other such that the organic film is spaced apart from the display unit.

In some embodiments, the display unit is formed by forming the light-emitting unit, and the encapsulation layer is disposed on the display unit to cover the display unit. As such, once the mother panel is completely produced, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks can be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### EXAMPLES

The features of the present invention will be described more specifically with reference to Synthesis Examples and Examples given below. The materials, processes, procedures and the like shown below can be appropriately modified unless they deviate from the substance of the present invention. Accordingly, the scope of the present invention is not construed as being limited to the specific examples shown below. Herein under the light emission characteristics were evaluated using a source meter (available from Keithley Instruments, Inc.: 2400 series), a semiconductor parameter analyzer (available from Agilent Technologies, Inc., E5273A), an optical power meter device (available from Newport Corporation, 1930C), an optical spectroscope (available from Ocean Optics, Inc., USB2000), a spectroradiometer (available from TOPCON CORPORATION, SR-3), and a streak camera (available from Hamamatsu Photonics K.K., Model C4334).

### (Synthesis Examples 1 to 3) Synthesis of Compounds P1, P2, and P3

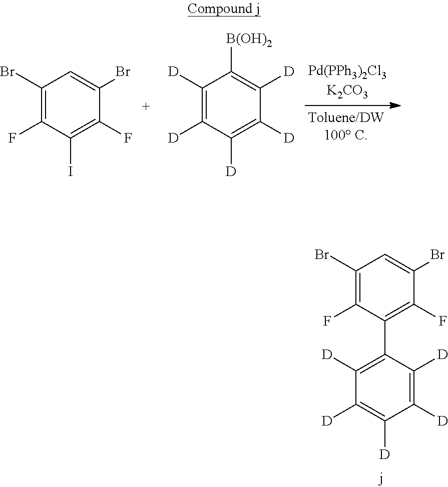

To a mixed solution of 1,5-dibromo-2,4-difluoro-3-iodobenzene (15.8 g, 39.9 mmol) in toluene (100 mL) and ion-exchanged water (30 mL), phenyl-d5-boronic acid (6.2 g, 48.8 mmol), bis(triphenylphosphine)palladium (II) dichloride (1.4 g, 2.0 mmol), and potassium carbonate (29.0 g, 210 mmol) were added, and the mixture was stirred at 100° C. for 23 hours under a nitrogen atmosphere. The reaction solution was cooled to room temperature, and saturated saline water was added thereto to separate the solution into an organic phase and an aqueous phase. After the aqueous phase was extracted with toluene, the combined organic phase was dried over anhydrous magnesium sulfate and filtered, and then the filtrate was concentrated. The crude product was purified by silica gel chromatography (hexane) to obtain Compound j as a white solid (9.0 g, 25.5 mmol, yield 64%).

¹H-NMR (400 MHz, CDCl₃): δ 7.77 (t, J=6.8 Hz, 1H).

ASAPP MS Spectral Analysis: C₁₂HD₅Br₂F₂: theoretical value 350.91, observed value 350.93 [M]

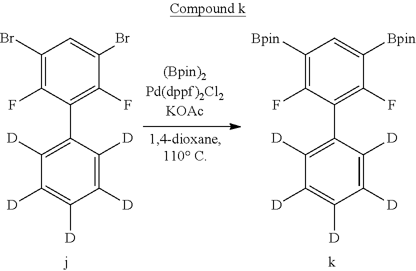

A reaction mixture of Compound j (2.1 g, 5.98 mmol), [1,1′-bis(diphenylphosphino)ferrocene]dichloropalladium (II) (0.22 g, 0.30 mmol), potassium acetate (4.1 g, 42.0 mmol), bis(pinacolato)diboron (7.1 g, 27.9 mmol), and 1,4-dioxane (60 mL) was stirred at 110° C. for 15 hours. The reaction mixture was cooled to room temperature, diluted with ethyl acetate, and filtered through silica. After the filtrate was concentrated, the obtained reaction mixture was dissolved in methylene chloride and subjected to silica filtration. The obtained solid was washed with hexane to obtain Compound k as a white solid (1.84 g, 4.11 mmol, yield 69%).

¹H-NMR (400 MHz, CDCl₃): δ 8.11 (t, J=7.2 Hz, 1H), 1.36 (s, 24H).

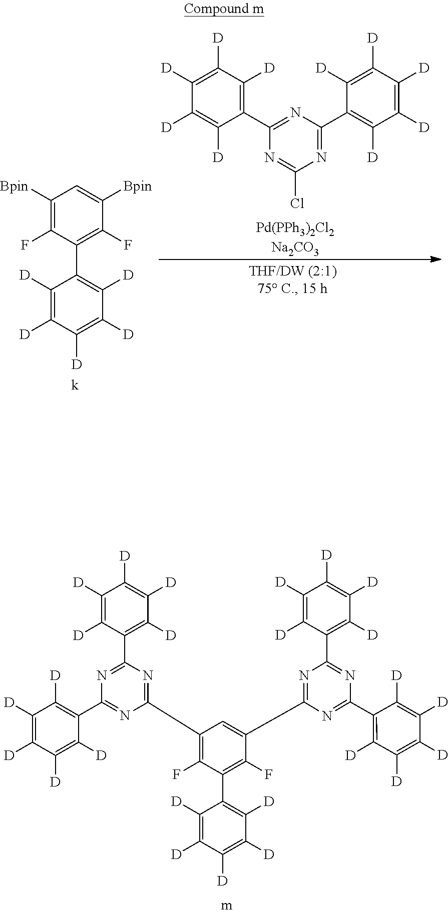

Compound k (8.00 g, 17.89 mmol) was dissolved in tetrahydrofuran (THF, 138 mL) and ion-exchanged water (46 mL), and 2-chloro-4,6-bis(phenyl-d5)-1,3,5-triazine (4.35 g, 35.78 mmol), bis(triphenylphosphine)palladium (II) dichloride (0.63 g, 0.89 mmol) and sodium carbonate (9.48 g, 89.46 mmol) were added thereto, and the mixture was stirred at 75° C. for 16 hours. The reaction vessel was cooled to room temperature, and the obtained gray solid was separated by filtration. The solid was washed with ion-exchanged water, methanol, and THF. The collected solid was stirred in hot toluene, and the solid was separated by filtration and washed with toluene to obtain Compound m as a black solid (4.1 g, 6.05 mmol, yield 34%).

ASAPP MS Spectral Analysis: C₄₂HD₂₅F₂N₆: theoretical value 677, observed value 678 [M+H⁺].

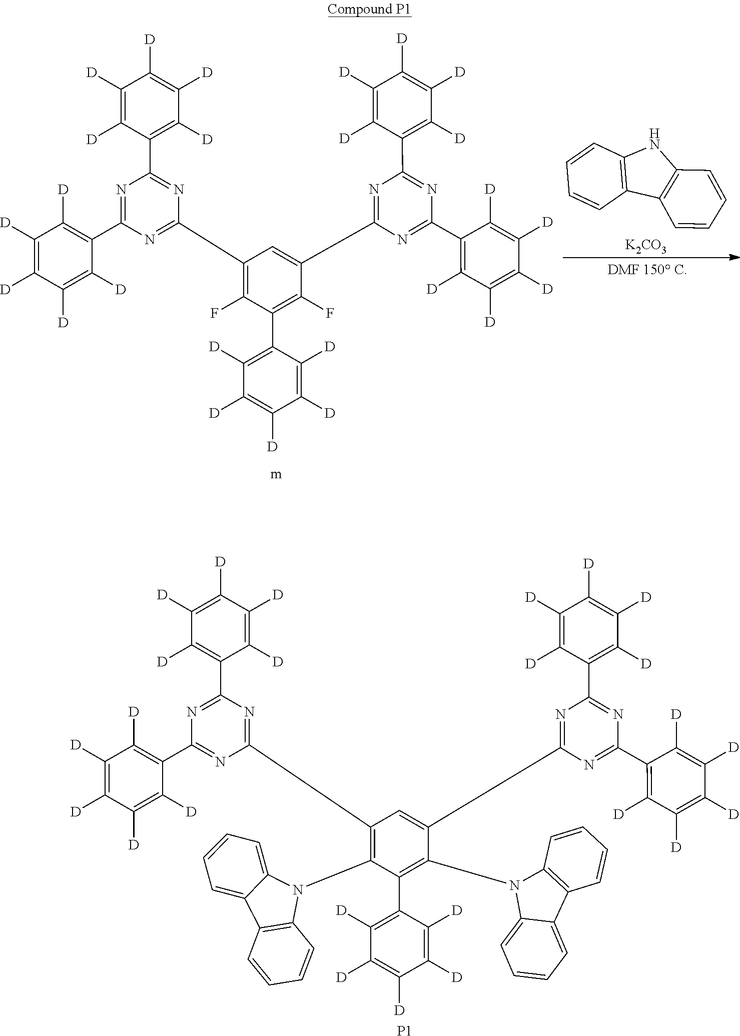

To a mixture of Compound m (1.30 g, 1.92 mmol) and N,N-dimethylformamide (DMF, 50 mL), 9H-carbazole (0.80 g, 4.79 mmol) and potassium carbonate (0.93 g, 6.71 mmol) were added, and the mixture was stirred at 150° C. for 3 hours. The reaction solution was cooled to room temperature, and the solid was filtered and washed with ethyl acetate. The obtained filtrate was concentrated, methanol was added thereto, and the resulting solid was separated by filtration and washed with methanol. The obtained crude product was purified by column chromatography (toluene/hexane=3:1), and the obtained solid was reprecipitated with ethyl acetate/hexane, thereby obtaining 1.60 g (1.65 mmol, yield 85.8%) of a pale green Compound P1.

ASAPP MS Spectral Analysis: C₆₆H₁₇D₂₅N₈: theoretical value 972, observed value 973 [M+H⁺].

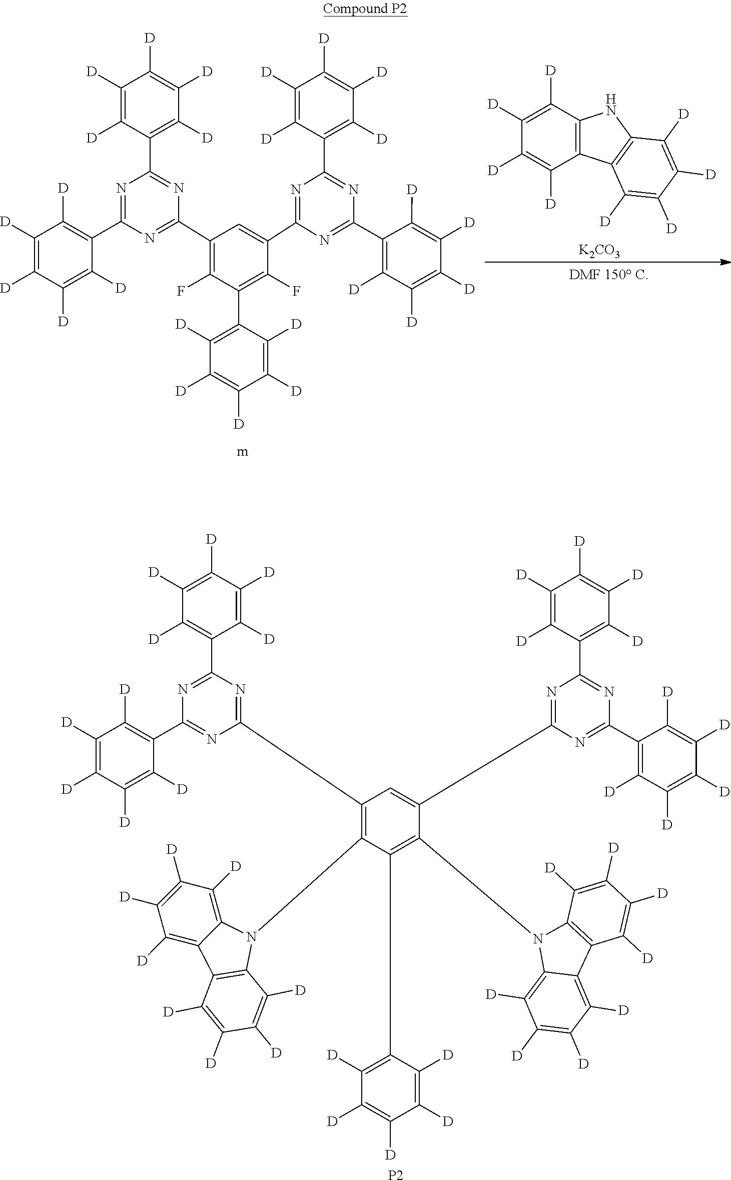

To a mixture of Compound m (1.30 g, 1.92 mmol) and DMF (50 mL), carbazole-1,2,3,4,5,6,7,8-d8 (0.84 g, 4.79 mmol) and potassium carbonate (0.93 g, 6.71 mmol) were added, and the mixture was stirred at 150° C. for 3 hours. The reaction solution was cooled to room temperature, and the solid was filtered and washed with ethyl acetate. The obtained filtrate was concentrated, methanol was added thereto, and the resulting solid was separated by filtration and washed with methanol. The obtained crude product was purified by column chromatography (toluene/hexane=3:1), and the obtained solid was reprecipitated with ethyl acetate/hexane, thereby obtaining 1.50 g (1.52 mmol, yield 79.1%) of a pale green Compound P2.

ASAP MS Spectral Analysis: C₆₆HD₄₁N₈: theoretical value 988, observed value 989 [M+H⁺].

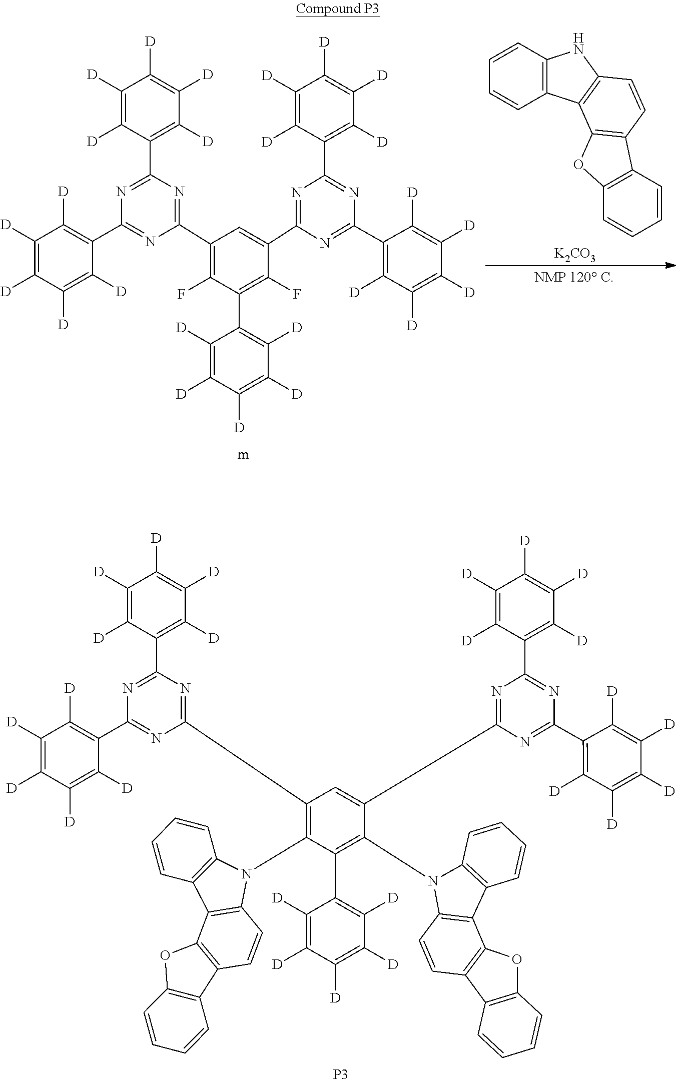

Under a nitrogen stream, potassium carbonate (1.07 g, 7.74 mmol) was added to a solution of Compound m (1.75 g, 2.58 mmol) and 5H-benzofuro[3,2-c]carbazole (1.60 g, 6.22 mmol) in N-methyl-2-pyrrolidone (NMP, 90 mL), and the mixture was stirred at 120° C. for 18 hours. The reaction solution was cooled to room temperature and quenched by the addition of water. The solution was filtered and the solid remaining in the funnel was washed with water. The solid was dissolved in dichloromethane and dried over magnesium sulfate. The solution was concentrated under reduced pressure in an evaporator, then, purified by column chromatography (toluene:hexane=1:1), washed with methanol, and recrystallized (toluene) to obtain a yellow Compound P3 (0.80 g, 0.69 mmol, yield 27%).

¹H-NMR (400 MHz, CDCl₃): δ 9.51 (s, 1H), 7.57 (d, J=7.2 Hz, 2H), 7.94-7.87 (m, 4H), 7.62 (d, J=7.2 Hz, 2H), 7.38-7.24 (m, 12H).

### (Synthesis Example 4) Synthesis of Compound P4

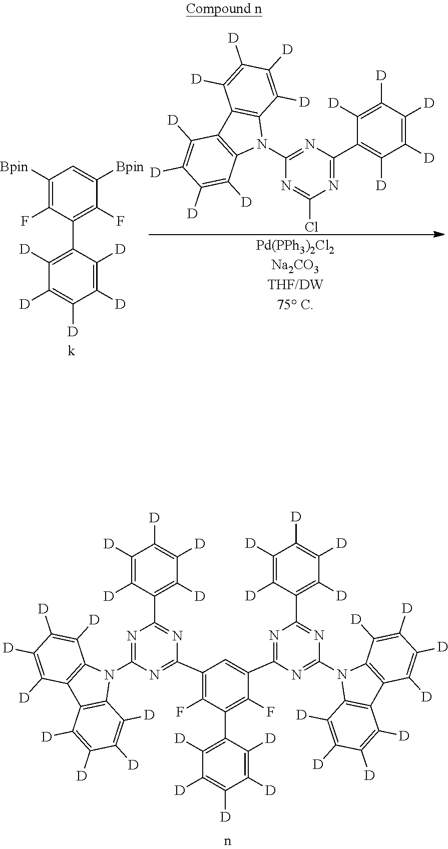

Compound k (1.81 g, 4.05 mmol) was dissolved in THF (30 mL) and ion-exchanged water (10 mL), and 9-(4-chloro-6-(phenyl-d5)-1,3,5-triazin-2-yl)-9H-carbazole-1,2,3,4,5,6,7,8-d8 (3.05 g, 8.25 mmol), bis(triphenylphosphine)palladium (II) dichloride (0.15 g, 0.21 mmol), and sodium carbonate (2.20 g, 20.8 mmol) were added thereto, and the mixture was stirred at 75° C. for 16 hours. The reaction vessel was cooled to room temperature, and the obtained gray solid was separated by filtration. The solid was washed with ion-exchanged water, methanol, and THF. The collected solid was stirred in hot toluene, and the solid was separated by filtration and washed with toluene to obtain Compound n as a black solid (3.30 g, 3.82 mmol, yield 94%).

ASAPP MS Spectral Analysis: C₅₄HD₃₁F₂N₈: theoretical value 861.47, observed value 862.72 [M+H⁺].

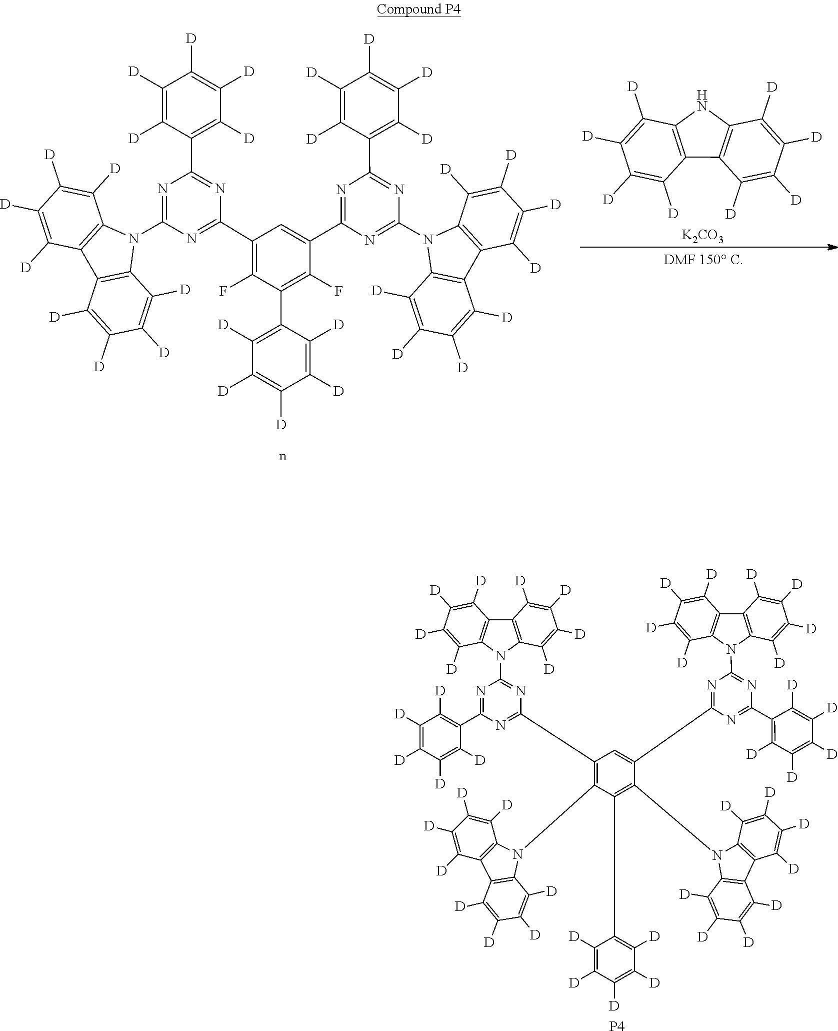

To a mixture of Compound n (2.85 g, 3.30 mmol) and DMF (65 mL), carbazole-1,2,3,4,5,6,7,8-d8 (1.28 g, 7.30 mmol) and potassium carbonate (1.37 g, 9.91 mmol) were added, and the mixture was stirred at 150° C. for 3 hours. The reaction solution was cooled to room temperature, and the solid was filtered and washed with ethyl acetate. The obtained filtrate was concentrated, methanol was added thereto, and the resulting solid was separated by filtration and washed with methanol. The obtained crude product was purified by column chromatography (toluene/hexane=3:1), and the obtained solid was reprecipitated with ethyl acetate/hexane, thereby obtaining 2.13 g (1.81 mmol, yield 55%) of a pale green Compound P4.

¹H-NMR (400 MHz, DMSO-d6): δ 9.20 (s, 1H).

ASAPP MS Spectral Analysis: C₇₈HD₄₇N₁₀: theoretical value 1171.70, observed value 1172.15 [M+H⁺].

### (Synthesis Example 5) Synthesis of Compound P5

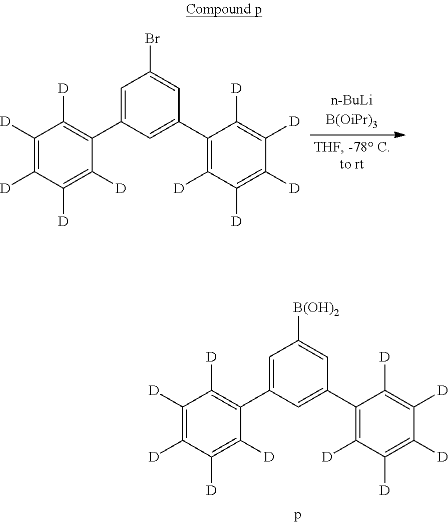 [SMILES: [2H]c1c([2H])c([2H])c(-c2cc(Br)cc(-c3c([2H])c([2H])c([2H])c([2H])c3[2H])c2)c([2H])c1[2H] ; [2H]c1c([2H])c([2H])c(-c2cc(B(O)O)cc(-c3c([2H])c([2H])c([2H])c([2H])c3[2H])c2)c([2H])c1[2H]]

A 2.3M solution of n-butyllithium in cyclohexane (21 mL, 48.3 mmol) was slowly added dropwise to a THF (75 mL) solution of 5′-bromo-1,1′:3′,1″-terphenyl-2,2″,3,3″,4,4″,5,5″,6,6″-d10 (12.0 g, 37.6 mmol) under a nitrogen atmosphere at −78° C. After stirring for 80 minutes, triisopropyl borate (12.8 mL, 56.4 mmol) was added, and the temperature of the reactor was raised to room temperature. After stirring for 2 hours, the mixture was cooled to 0° C., and a 4N hydrochloric acid aqueous solution (70 mL) was added thereto. After stirring for 30 minutes at room temperature, the organic and aqueous phases were separated. The aqueous phase was extracted with diethyl ether, and the combined organic phase was washed with ion-exchanged water and saturated saline water. The organic phase was concentrated, and the obtained white solid was washed with hexane to obtain a white Compound p (9.30 g, 32.7 mmol, yield 87%).

¹H-NMR (400 MHz, DMSO-d6): δ 8.26 (brs, 2H), 8.08 (d, J=1.6 Hz, 2H), 7.92-7.93 (m, 1H).

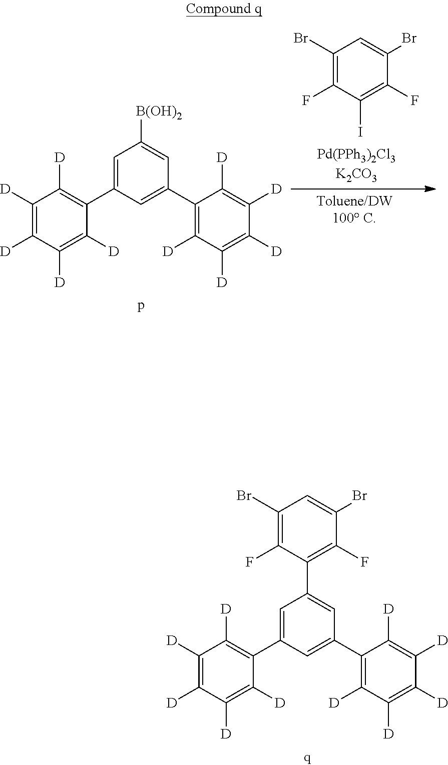 [SMILES: [2H]c1c([2H])c([2H])c(-c2cc(B(O)O)cc(-c3c([2H])c([2H])c([2H])c([2H])c3[2H])c2)c([2H])c1[2H] ; Fc1c(Br)cc(Br)c(F)c1I ; [2H]c1c([2H])c([2H])c(-c2cc(-c3c([2H])c([2H])c([2H])c([2H])c3[2H])cc(-c3c(F)c(Br)cc(Br)c3F)c2)c([2H])c1[2H]]

To a mixed solution of 1,5-dibromo-2,4-difluoro-3-iodobenzene (5.1 g, 13.0 mmol) in toluene (30 mL) and ion-exchanged water (10 mL), Compound p (4.0 g, 14.0 mmol), bis(triphenylphosphine)palladium (II) dichloride (0.46 g, 0.64 mmol), and potassium carbonate (3.60 g, 26.0 mmol) were added, and the mixture was stirred at 100° C. for 15 hours. The reaction solution was cooled to room temperature, and saturated saline water was added thereto to separate the solution into an organic phase and an aqueous phase. After the aqueous phase was extracted with ethyl acetate, the combined organic phase was dried over anhydrous magnesium sulfate and filtered, and then the filtrate was concentrated. The crude product was purified by silica gel chromatography (hexane/ethyl acetate=40:1) and (hexane/methylene chloride=10:1), and the obtained white solid was washed with hexane to obtain 4.84 g (9.48 mmol, yield 73%) of Compound q.

¹H-NMR (400 MHz, CDCl₃): δ 7.88 (t, J=2.0 Hz, 1H), 7.81 (t, J=6.8 Hz, 1H), 7.62 (q, J=2.0 Hz, 2H).

ASAPP MS Spectral Analysis: C₂₄H₄D₁₀Br₂F₂: theoretical value 508.00, observed value 509.03 [M+H⁺].

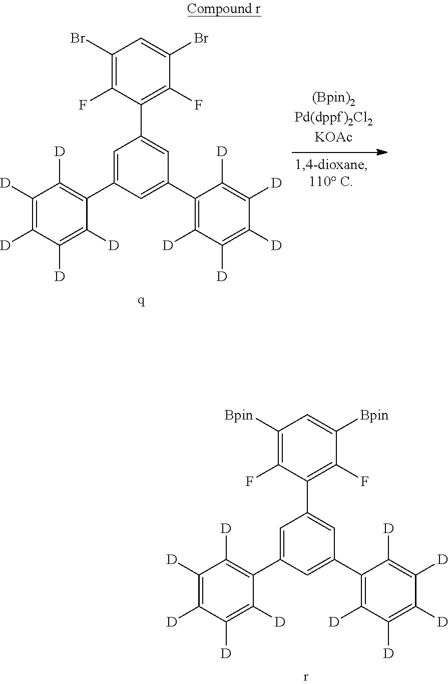

A reaction mixture of Compound q (4.8 g, 9.40 mmol), [1,1′-bis(diphenylphosphino)ferrocene]dichloropalladium (II) (0.36 g, 0.50 mmol), potassium acetate (6.5 g, 66.2 mmol), bis(pinacolato)diboron (12.1 g, 47.6 mmol), and 1,4-dioxane (100 mL) was stirred at 110° C. for 15 hours. The reaction mixture was cooled to room temperature, diluted with ethyl acetate, and filtered through silica. After the filtrate was concentrated, the obtained reaction mixture was dissolved in methylene chloride and subjected to silica filtration. The obtained solid was washed with hexane to obtain Compound r as a yellow solid (5.00 g, 8.27 mmol, yield 88%).

¹H-NMR (400 MHz, CDCl₃): δ 8.15 (t, J=6.8 Hz, 1H), 7.79-7.80 (m, 1H), 7.66 (brs, 2H), 1.26 (s, 24H).

ASAPP MS Spectral Analysis: C₃₆H₂₈D₁₀B₂F₂O₄: theoretical value 604.37, observed value 605.47 [M+H⁺].

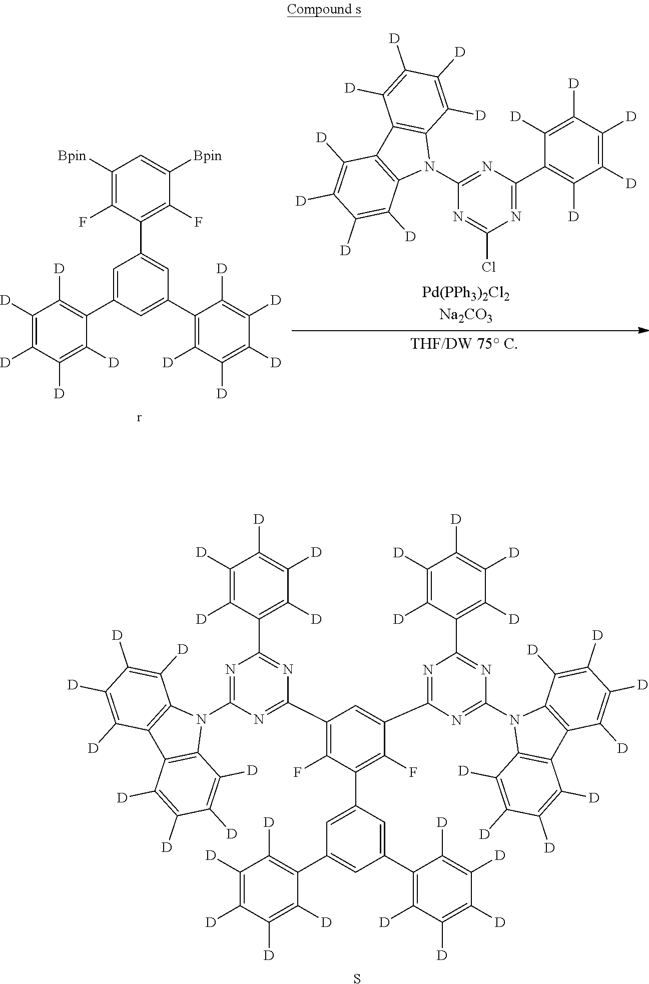

Compound r (4.98 g, 8.24 mmol) was dissolved in THF (60 mL) and ion-exchanged water (20 mL), and 9-(4-chloro-6-(phenyl-d5)-1,3,5-triazin-2-yl)-9H-carbazole-1,2,3,4,5,6,7,8-d8 (6.10 g, 16.5 mmol), bis(triphenylphosphine)palladium (II) dichloride (0.29 g, 0.41 mmol), and sodium carbonate (4.37 g, 41.2 mmol) were added thereto, and the mixture was stirred at 75° C. for 20 hours. The reaction vessel was cooled to room temperature, and the obtained gray solid was separated by filtration. The solid was washed with THF and ion-exchanged water. The collected solid was stirred in hot toluene, and the solid was separated by filtration, washed with toluene, and dried to obtain Compound s as a gray solid (7.98 g, 7.83 mmol, yield 95%).

ASAPP MS Spectral Analysis: C₆₆H₄D₃₆F₂N₈: theoretical value 1018.56, observed value 1019.83 [M+H⁺].

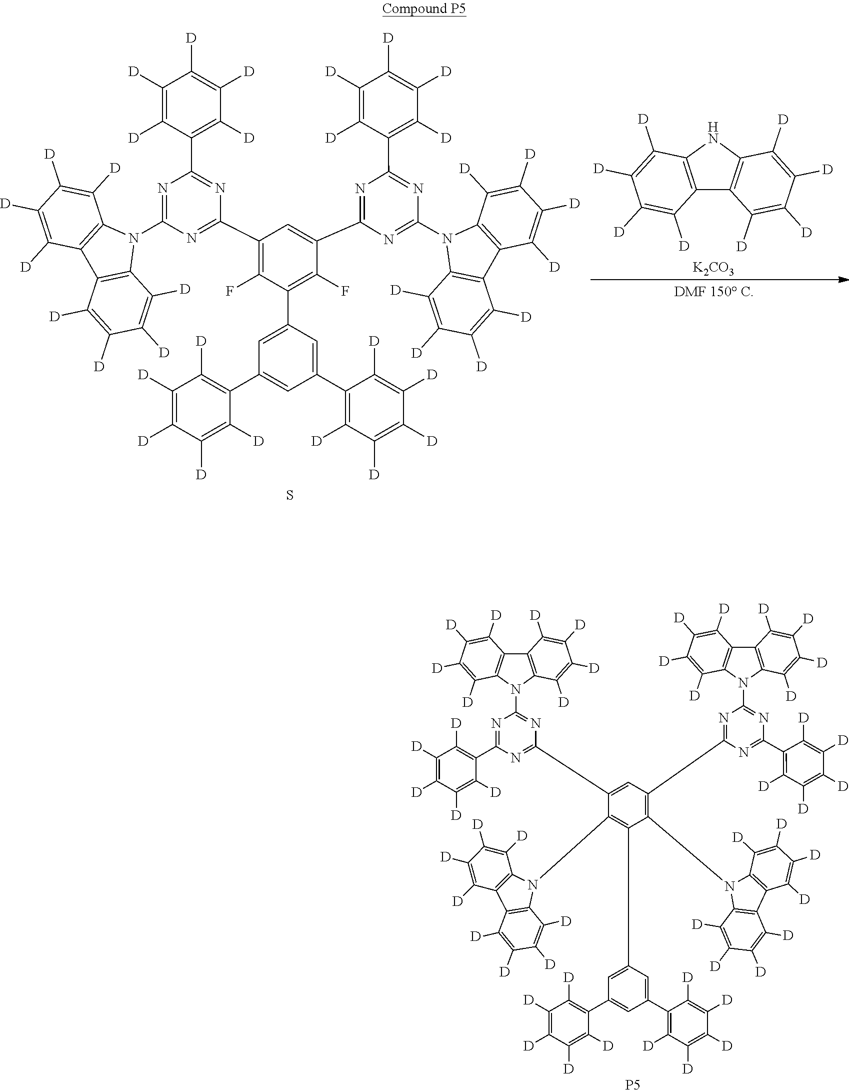

To a mixture of Compound s (3.06 g, 3.00 mmol) and DMF (60 mL), carbazole-1,2,3,4,5,6,7,8-d8 (1.16 g, 6.61 mmol) and potassium carbonate (1.24 g, 8.97 mmol) were added, and the mixture was stirred at 150° C. for 3 hours. The reaction solution was cooled to room temperature, and the solid was filtered and washed with ethyl acetate. The obtained filtrate was concentrated, methanol was added thereto, and the resulting solid was separated by filtration and washed with methanol. The obtained crude product was purified by column chromatography (toluene/hexane=1:1 to 2:1), and the obtained solid was reprecipitated with ethyl acetate/hexane, thereby obtaining 1.47 g (1.10 mmol, yield 37%) of a green Compound P5.

¹H-NMR (400 MHz, DMSO-d6): δ 9.40 (s, 1H), 7.14 (d, J=1.6 Hz, 2H), 7.07 (d, J=1.6 Hz, 1H).

ASAPP MS Spectral Analysis: C₉₀H₄D₅₂N₁₀: theoretical value 1328.80, observed value 1329.84 [M+H⁺].

### (Example 1) Preparation and Evaluation of Thin Film

Compound P1 was vapor-deposited on a quartz substrate by a vacuum deposition method under conditions of a vacuum degree of less than 1×10⁻³ Pa to form a neat thin film of Compound P1 having a thickness of 100 nm.

Separately, Compound P1 and PyD2Cz were vapor-deposited from different vapor deposition sources on a quartz substrate by a vacuum deposition method under conditions of a vacuum degree of less than 1×10⁻³ Pa to form a doped thin film having a content of Compound P1 of 20% by weight and a thickness of 100 nm.

A neat thin film and a doped thin film were formed in the same manner by using Compound P2, Compound P4, Comparative Compound A, Comparative Compound B, and Comparative Compound C instead of Compound P1, respectively.

Photoluminescence when each of the formed doped thin films was irradiated with excitation light of 302 nm was analyzed, and emission peak wavelengths (% max), photoluminescence quantum yields (PLQY), and lifetimes (τ2) of delayed fluorescent components were measured. In addition, HOMO energy and LUMO energy were also measured using each of the formed neat thin films. The results are shown in the following Table. When a neat thin film of Compound P5 was formed in the same manner and measured, the HOMO energy of Compound P5 was 6.13 eV and the LUMO energy thereof was 3.45 eV.

It was confirmed that the compound represented by the general formula (1) had a high light emission efficiency and a short delayed fluorescence lifetime, and exhibited preferable light emission characteristics. In addition, it was also confirmed that a compound in which at least one of Ar¹ to Ar⁴ is a heteroaryl group, such as Compound P4, exhibited particularly excellent light emission characteristics.

TABLE 5

| Compound used | λmax (nm) | PLQY (%) | τ2 (μ sec) | HOMO (eV) | LUMO (eV) |
|---|---|---|---|---|---|
| Compound P1 | 488 | 97 | 2.8 | 6.19 | 3.48 |
| Compound P2 | 489 | 96 | 3.45 | 6.19 | 3.49 |
| Compound P4 | 490 | 99 | 2.5 | 6.13 | 3.49 |
| Comparative | 490 | 89 | 18.6 | 6.17 | 3.49 |
| Compound A | | | | | |
| Comparative | 494 | 81 | 24.6 | 6.12 | 3.32 |
| Compound B | | | | | |
| Comparative | 474 | 84 | 9.0 | 6.06 | 3.29 |
| Compound C | | | | | |

### (Example 2) Preparation and Evaluation of Thin Film

Compound P1 and H1 were vapor-deposited from different vapor deposition sources on a quartz substrate by a vacuum deposition method under conditions of a vacuum degree of less than 1×10⁻³ Pa to form a doped thin film having a content of Compound P1 of 30% by weight and a thickness of 100 nm.

A doped thin film was formed in the same manner by using Compound P2, Compound P4, and Compound P5 instead of Compound P1, respectively.

The photoluminescence when the excitation light of 302 nm was irradiated on each of the formed doped thin films was analyzed to measure the lifetime (τ2) of the delayed fluorescent component and the orientation degree (S value). The orientation degree was determined by the method described in Scientific Reports 2017, 7, 8405. The results are shown in the following Table. It was confirmed that the compound represented by the general formula (1) had a short delayed fluorescence lifetime and exhibited good orientation properties. In addition, it was also confirmed that a compound in which at least one of Ar¹ to Ar⁴ is a heteroaryl group, such as Compound P4 and Compound P5, had a particularly short delayed fluorescence lifetime and exhibited excellent orientation properties.

TABLE 6

| Compound used | τ2 (μ sec) | S value |
|---|---|---|
| Compound P1 | 3.2 | −0.038 |
| Compound P2 | 3.2 | 0.087 |
| Compound P4 | 2.6 | −0.137 |
| Compound P5 | 2.5 | −0.249 |

### (Example 3) Production and Evaluation of Organic Electroluminescent Device

On a glass substrate on which an anode made of indium-tin oxide (ITO) having a film thickness of 50 nm was formed, each thin film was laminated by a vacuum deposition method at a vacuum degree of 5.0×10⁻⁵ Pa. First, HAT-CN was formed to a thickness of 10 nm on the ITO, NPD was formed to a thickness of 30 nm thereon, further Tris-PCz was formed to a thickness of 10 nm, and further H1 was formed to a thickness of 5 nm. Next, H1 and Compound P1 were co-deposited from different vapor deposition sources to form a layer with a thickness of 40 nm as a light-emitting layer. In the light-emitting layer, the content of H1 was 70% by mass and the content of Compound P1 was 30% by mass. Next, after SF3-TRZ was formed to a thickness of 10 nm, Liq and SF3-TRZ were co-deposited from different vapor deposition sources to form a layer with a thickness of 30 nm. The contents of Liq and SF3-TRZ in this layer were 30% by mass and 70% by mass, respectively. Furthermore, Liq was formed to a thickness of 2 nm, and aluminum (Al) was vapor-deposited to a thickness of 100 nm to form a cathode, thereby obtaining an organic electroluminescent device.

A doped thin film was formed in the same manner by using Compound P2, Compound P4, Compound P5, Comparative Compound B, and Comparative Compound C instead of Compound P1, respectively.

When each of the organic electroluminescent devices was driven, blue delayed fluorescence was observed. The external quantum efficiency (EQE) when driven at 2 mA/cm² and the time until the emission intensity became 95% of that at the start of driving (LT95) were measured. The results are shown in the following Table. LT95 is shown as a relative value in which the value of the organic electroluminescent device using Comparative Compound B is set to 1. It was confirmed that the organic light-emitting device using the compound represented by the general formula (1) had a high light emission efficiency and a long device life, which were excellent. It was also confirmed that the organic light-emitting device using the compound in which at least one of Ar¹ to Ar⁴ is a heteroaryl group as in Compound P4 or Compound P5 was particularly excellent in light emission efficiency and device life.

TABLE 7

| Compound used | EQE (%) | LT95 (relative value) |
|---|---|---|
| Compound P1 | 15.7 | 10.9 |
| Compound P2 | 16.2 | 13.6 |
| Compound P4 | 20.3 | 18.3 |
| Compound P5 | 23.7 | 22.0 |
| Comparative Compound B | 14.8 | 1 |
| Comparative Compound C | 10.5 | 1.1 |

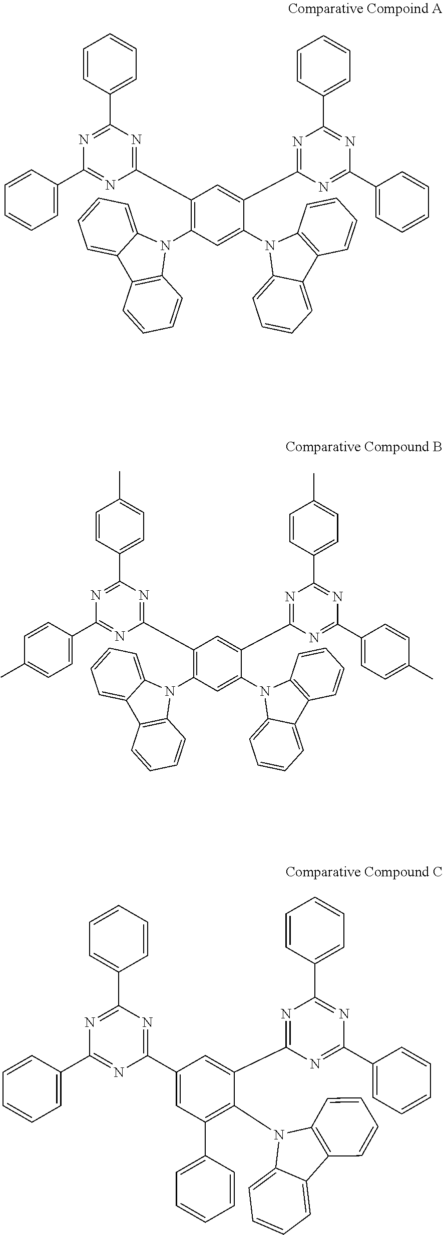

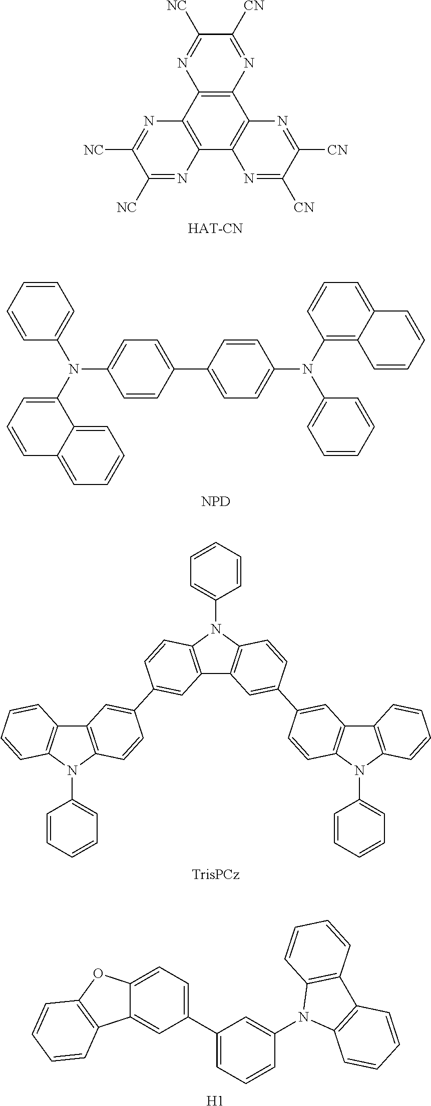

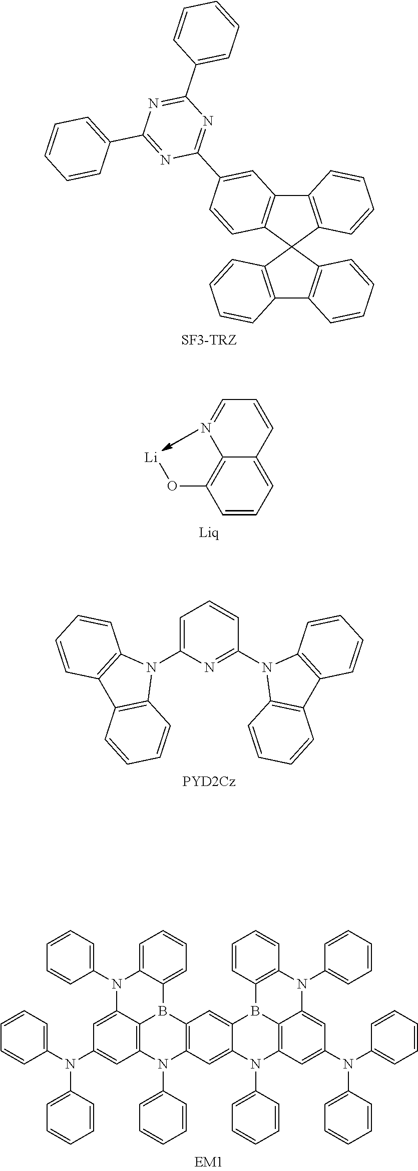

### INDUSTRIAL APPLICABILITY

The compound represented by the general formula (1) and the organic light-emitting device using the compound have good light emission characteristics. Therefore, the present invention has high industrial applicability.

1. A compound represented by the following general formula (1): 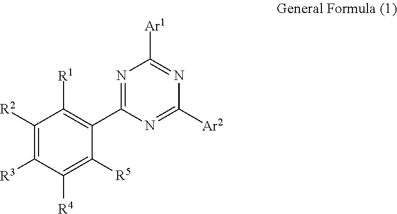
wherein R¹ represents a hydrogen atom or a deuterium atom; one of R² and R³ is a group represented by the following general formula (2), two selected from R⁴, R⁵ and the other of R² and R³ are donor groups, and the remaining one is a substituted or unsubstituted aryl group; Ar¹ and Ar² each independently represent a substituted or unsubstituted aryl group, or a substituted or unsubstituted heteroaryl group; 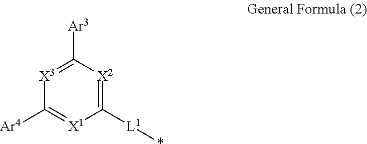
wherein X¹ to X³ each independently represent N or C(R), provided that at least one of X¹ to X³ is N; R represents a hydrogen atom, a deuterium atom, or a substituent; Ar³ and Ar⁴ each independently represent a substituted or unsubstituted aryl group, or a substituted or unsubstituted heteroaryl group; L¹ represents a single bond or a divalent linking group; and * represents a bonding position.

2. The compound according to claim 1, wherein R² is a group represented by the general formula (2).

3. The compound according to claim 1, wherein R³ is a group represented by the general formula (2).

4. The compound according to claim 1, wherein at least one of Ar¹ to Ar⁴ is a substituted or unsubstituted heteroaryl group excluding a nitrogen-containing 6-membered ring group.

5. The compound according to claim 4, wherein at least one of Ar¹ to Ar⁴ is a heteroaryl group having a 5-membered ring bonded via a nitrogen atom.

6. The compound according to claim 1, wherein the donor group is a substituted or unsubstituted carbazol-9-yl group.

7. The compound according to claim 1, wherein the donor group is a donor group having a fused ring structure of four or more rings.

8. The compound according to claim 1, wherein R⁵ is a donor group.

9. The compound according to claim 1, wherein R⁴ is a substituted or unsubstituted aryl group.

10. The compound according to claim 1, wherein the substituted or unsubstituted aryl group is an unsubstituted aryl group or an aryl group substituted with an aryl group.

11. The compound according to claim 1, wherein X¹ to X³ are N.

12. The compound according to claim 1, wherein L¹ is a single bond.

13. The compound according to claim 1, wherein R¹ is a hydrogen atom.

14. The compound according to claim 1, wherein the compound has at least one deuterium atom.

15. (canceled)

16. (canceled)

17. An organic light-emitting device comprising the compound according to claim 1 and a host material or a dopant material in the same layer.

18. The organic light-emitting device according to claim 17, wherein the layer comprises the dopant material.

19. The organic light-emitting device according to claim 17, wherein an amount of light emitted from the dopant material is larger than an amount of light emitted from the compound.

20. (canceled)

21. The organic light-emitting device according to claim 20, wherein the host material is a compound represented by the following general formula (4): 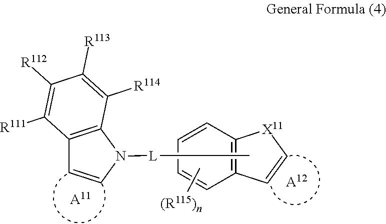
wherein X¹¹ represents O, S, N(R^{A}), or C(R^{B})(R^{C}); A¹¹ and A¹² each independently represent a benzene ring, a furan ring, a thiol ring, a pyrrole ring, or a cyclopentadiene ring, which may be further fused with another ring or substituted; R¹¹¹ to R¹¹⁴, R^{B}, and R^{C} each independently Represent a hydrogen atom, a deuterium atom, a substituted or unsubstituted aryl group, a substituted or unsubstituted heteroaryl group, a substituted or unsubstituted alkyl group, or a cyano group; each of R¹¹⁵'s independently represents a hydrogen atom, a deuterium atom, a substituted or unsubstituted aryl group, a substituted or unsubstituted heteroaryl group, a substituted or unsubstituted alkyl group, a cyano group, or a bond with L; R^{A} represents a substituted or unsubstituted aryl group, a substituted or unsubstituted heteroaryl group, a substituted or unsubstituted alkyl group, or a bond with L; R¹¹¹ and R¹¹², R¹¹² and R¹¹³, R¹¹³ and R¹¹⁴, two adjacent R¹¹⁵'s, and R^{B} and R^{C} may be bonded to each other to form a cyclic structure; n represents an integer of 3 or 4; and L represents a single bond, a substituted or unsubstituted arylene group, a substituted or unsubstituted heteroarylene group, or a linking group in which two or more of these groups are bonded.

22. The organic light-emitting device according to claim 20, wherein an amount of light emitted from the compound is largest among the materials contained in the layer.

23. The organic light-emitting device according to claim 17, which emits delayed fluorescence.
